(19) United States
(12) Patent Application Publication
(10) Pub. No.: US 2011/0190280 A1
(43) Pub. Date: Aug. 4, 2011

(54) Thiazole And Oxazole Kinase Inhibitors

(76) Inventors: George Adjabeng, Durham, NC (US); Neil Bifulco, Durham, NC (US); Ronda G. Davis-Ward, Durham, NC (US); Scott Howard Dickerson, Durham, NC (US); Kelly Horne Donaldson, Durham, NC (US); Philip Anthony Harris, Wayne, PA (US); Keith Hornberger, Durham, NC (US); Kimberly Petrov, Durham, NC (US); Tara Renae Rheault, Durham, NC (US); Gregory Schaaf, Durham, NC (US); John Stellwagen, Durham, NC (US); David Edward Uehling, Durham, NC (US); Alex Gregory Waterson, Durham, NC (US)

(21) Appl. No.: 12/674,016

(22) PCT Filed: Aug. 27, 2008
(86) PCT No.: PCT/US2008/074389

§ 371 Date: Feb. 18, 2010

Related U.S. Application Data
(60) Provisional application No. 60/968,687, filed on Aug. 29, 2007.

(51) Int. Cl. A61K 31/541; C07D 403/14; A61K 31/506; C07D 413/14; A61K 31/5377; C07D 417/14; C07D 498/04; A61K 31/5365; A61P 35/00

(52) U.S. Cl. 514/227.8; 544/295; 514/252.18; 544/331; 514/275; 544/122; 514/235.8; 544/58.2; 544/105; 514/230.5

(57) ABSTRACT

The present invention provides thiazole and oxazole compounds, compositions containing the same, as well as processes for the preparation and methods for their use as pharmaceutical agents.

### FIELD OF THE INVENTION

The present invention relates to thiazole and oxazole compounds, compositions containing the same, as well as processes for the preparation and methods of using such compounds and compositions.

### BACKGROUND OF THE INVENTION

Both receptor tyrosine kinases and serine/threonine kinases have been implicated in cellular signaling pathways that control cell function, division, growth, differentiation, and death (apoptosis) through reversible phosphorylation of the hydroxyl groups of tyrosine or serine and threonine residues, respectively, in proteins. In signal transduction, for example, extracellular signals are transduced via membrane receptor activation, with amplification and propagation using a complex choreography of cascades of protein phosphorylation, and protein dephosphorylation events to avoid uncontrolled signaling. These signaling pathways are highly regulated, often by complex and intermeshed kinase pathways where each kinase may itself be regulated by one or more other kinases and protein phosphatases. The biological importance of these finely tuned systems is such that a variety of cell proliferative disorders have been linked to defects in one or more of the various cell signaling pathways mediated by tyrosine or serine/threonine kinases.

Receptor tyrosine kinases (RTKs) catalyze phosphorylation of certain tyrosyl amino acid residues in various proteins, including themselves, which govern cell growth, proliferation and differentiation.

Downstream of the RTKs lie several signaling pathways, among them is the Ras-Raf-MEK-ERK kinase pathway. It is currently understood that activation of Ras GTPase proteins in response to growth factors, hormones, cytokines, etc. stimulates phosphorylation and activation of Raf kinases. These kinases then phosphorylate and activate the intracellular protein kinases MEK1 and MEK2, which in turn phosphorylate and activate other protein kinases, ERK1 and 2. This signaling pathway, also known as the mitogen-activated protein kinase (MAPK) pathway or cytoplasmic cascade, mediates cellular responses to growth signals. It's ultimate function is to link receptor activity at the cell membrane with modification of cytoplasmic or nuclear targets that govern cell proliferation, differentiation, and survival. Mutations in various Ras GTPases and the B-Raf kinase have been identified that can lead to sustained and constitutive activation of the MAPK pathway, ultimately resulting in increased cell division and survival. As a consequence, these mutations have been strongly linked with the establishment, development, and progression of a wide range of human cancers. The biological role of the Raf kinases, and specifically that of B-Raf, in signal transduction is described in Davies, H., et al., Nature (2002) 9:1-6; Garnett, M. J. & Marais, R., Cancer Cell (2004) 6:313-319; Zebisch, A. & Troppmair, J., Cell. Mol. Life. Sci. (2006) 63:1314-1330; Midgley, R. S. & Kerr, D. J., Crit. Rev. Onc/Hematol. (2002) 44:109-120; Smith, R. A., et al., Curr. Top. Med. Chem. (2006) 6:1071-1089; and Downward, J., Nat. Rev. Cancer (2003) 3:11-22.

Naturally occurring mutations of the B-Raf kinase that activate MAPK pathway signaling have been found in a large percentage of human melanomas (Davies (2002) supra) and thyroid cancers (Cohen et al J. Nat. Cancer Inst. (2003) 95(8) 625-627 and Kimura et al Cancer Res. (2003) 63(7) 1454-1457), as well as at lower, but still significant, frequencies in the following:
Barret's adenocarcinoma (Garnett et al., Cancer Cell (2004) 6 313-319 and Sommerer et al Oncogene (2004) 23(2) 554-558),
billiary tract carcinomas (Zebisch et al., Cell. Mol. Life. Sci. (2006) 63 1314-1330), breast cancer (Davies (2002) supra),
cervical cancer (Moreno-Bueno et al Clin. Cancer Res. (2006) 12(12) 3865-3866),
cholangiocarcinoma (Tannapfel et al Gut (2003) 52(5) 706-712),
central nervous system tumors including primary CNS tumors such as glioblastomas, astrocytomas and ependymomas (Knobbe et al Acta Neuropathol. (Berl.) (2004) 108(6) 467-470, Davies (2002) supra, and Garnett et al., Cancer Cell (2004) supra) and secondary CNS tumors (i.e., metastases to the central nervous system of tumors originating outside of the central nervous system),
colorectal cancer, including large intestinal colon carcinoma (Yuen et al Cancer Res. (2002) 62(22) 6451-6455, Davies (2002) supra and Zebisch et al., Cell. Mol. Life. Sci. (2006),
gastric cancer (Lee et al Oncogene (2003) 22(44) 6942-6945),
carcinoma of the head and neck including squamous cell carcinoma of the head and neck (Cohen et al J. Nat. Cancer Inst. (2003) 95(8) 625-627 and Weber et al Oncogene (2003) 22(30) 4757-4759),
hematologic cancers including leukemias (Garnett et al., Cancer Cell (2004) supra, particularly acute lymphoblastic leukemia (Garnett et al., Cancer Cell (2004) supra and Gustafsson et al Leukemia (2005) 19(2) 310-312), acute myelogenous leukemia (AML) (Lee et al Leukemia (2004) 18(1) 170-172, and Christiansen et al Leukemia (2005) 19(12) 2232-2240), myelodysplastic syndromes (Christiansen et al Leukemia (2005) supra) and chronic myelogenous leukemia (Mizuchi et al Biochem. Biophys. Res. Commun. (2005) 326(3) 645-651); Hodgkin's lymphoma (Figl et al Arch. Dermatol. (2007) 143(4) 495-499), non-Hodgkin's lymphoma (Lee et al Br. J. Cancer (2003) 89(10) 1958-1960), megakaryoblastic leukemia (Eychene et al Oncogene (1995) 10(6) 1159-1165) and multiple myeloma (Ng et al Br. J. Haematol. (2003) 123(4) 637-645),
hepatocellular carcinoma (Garnett et al., Cancer Cell (2004),
lung cancer (Brose et al Cancer Res. (2002) 62(23) 6997-7000, Cohen et al J. Nat. Cancer Inst. (2003) supra and Davies (2002) supra), including small cell lung cancer (Pardo et al EMBO J. (2006) 25(13) 3078-3088) and non-small cell lung cancer (Davies (2002) supra),
ovarian cancer (Russell & McCluggage J. Pathol. (2004) 203(2) 617-619 and Davies (2002) supr), endometrial cancer (Garnett et al., Cancer Cell (2004) supra, and Moreno-Bueno et al Clin. Cancer Res. (2006) supra),
pancreatic cancer (Ishimura et al Cancer Lett. (2003) 199(2) 169-173),
pituitary adenoma (De Martino et al J. Endocrinol. Invest. (2007) 30(1) RC1-3),
prostate cancer (Cho et al Int. J. Cancer (2006) 119(8) 1858-1862),
renal cancer (Nagy et al Int. J. Cancer (2003) 106(6) 980-981),
sarcoma (Davies (2002) supra), and
skin cancers (Rodriguez-Viciana et al Science (2006) 311(5765) 1287-1290 and Davies (2002) supra).
Overexpression of c-Raf has been linked to AML (Zebisch et al., Cancer Res. (2006) 66(7) 3401-3408, and Zebisch (Cell. Mol. Life. Sci. (2006)) and erythroleukemia (Zebisch et la., Cell. Mol. Life. Sci. (2006).

By virtue of the role played by the Raf family kinases in these cancers and exploratory studies with a range of preclinical and therapeutic agents, including one selectively targeted to inhibition of B-Raf kinase activity (King A. J., et al., (2006) Cancer Res. 66:11100-11105), it is generally accepted that inhibitors of one or more Raf family kinases will be useful for the treatment of such cancers.

Mutation of B-Raf has also been implicated in other conditions, including cardio-facio cutaneous syndrome (Rodriguez-Viciana et al Science (2006) 311(5765) 1287-1290) and polycystic kidney disease (Nagao et al Kidney Int. (2003) 63(2) 427-437).

PCT Publication No. WO2003/029249, published 10 Apr. 2003 to Syngenta, recites fungicidal compounds or a salt thereof having the formula:

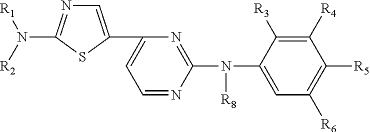

wherein the variables are defined therein.

PCT publication No. WO02/043467, published 27 May 2004 to Cyclacel Ltd., recites antiviral compounds and pharmaceutically acceptable salts having the formula

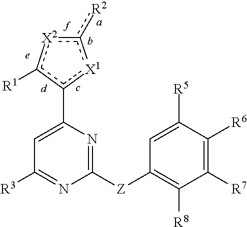

wherein the variables are as defined therein.

PCT Publication Nos. WO2004/043953, published 27 May 2004 and WO2005/116025, published 8 Dec. 2005, also to Cyclacel Lmtd., recite compounds of the same generic formula having differing variable definitions.

PCT Publication No. 2004/056368, published 8 Jul. 2004 to Cyclacel Lmtd., recites compounds for treating diabetes and CNS disorders, alopecia, CV disorders and stroke, having the formula:

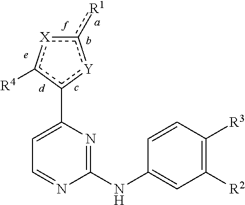

wherein the variables are defined therein.

### SUMMARY OF THE INVENTION

In a first aspect of the present invention, there is provided a compound of formula (I):

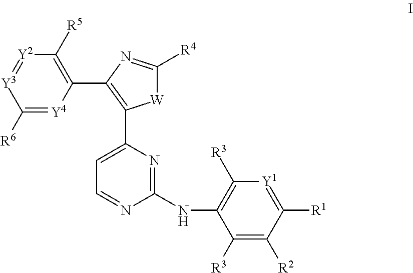

wherein:
R¹ is a moiety i, ii or iii:

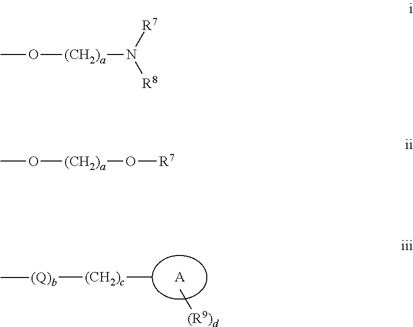

wherein:
a is 2, 3 or 4;
R⁷ and R⁸ are the same or different and are each independently selected from H, alkyl, haloalkyl, alkenyl, alkynyl, C₃₋₆cycloalkyl and C₃₋₆cycloalkenyl;
b is 0 or 1;
Q is selected from —O—, —N(H)— and —N(alkyl)-;
c is 0, 1, 2 or 3;
Ring A is a 4-10 membered N-heterocycle optionally having 1 or 2 additional heteroatoms selected from N, O and S, or 5-10 membered N-heteroaryl optionally having 1 or 2 additional heteroatoms selected from N, O and S;
d is 0, 1 or 2;
each R⁹ is the same or different and is independently selected from halo, alkyl, alkenyl, alkynyl, haloalkyl, oxo, OR¹⁰, R¹²—OR¹⁰, C(O)R¹⁰, CO₂R¹⁰,
C(O)₂-benzyl, CONR¹⁰R¹¹, COR¹²—NR¹⁰R¹¹, COR¹²—OR¹⁰, NR¹⁰R¹¹, R¹²—NR¹⁰R¹¹, N(R¹⁰)C(O)R¹¹, N(R¹⁰)S(O)₂R¹¹, N(R¹⁰)C(O)N(R¹¹), N(R¹⁰)C(S)N(R¹¹), S(O)₃H, R¹²—S(O)₃H, S(O)₂R¹⁰, R¹²—S(O)₂R¹⁰, S(O)₂NR¹⁰R¹¹, CN and R¹²—CN;
R² is selected from H, halo, alkyl, haloalkyl, OR¹⁰, CO₂R¹⁰, NR¹⁰R¹¹, S(O)₂R¹⁰, CN and 5-6 membered N-heterocycle optionally having 1 additional heteroatom selected from N, O and S and optionally substituted 1 or 2 times with alkyl or oxo; or
R¹ and R², together with the aromatic ring to which they are bound form a 9 or 10-membered fused, bicyclic heteroaryl having 1, 2 or 3 heteroatoms selected from N, O and S, wherein said fused bicyclic heteroaryl is optionally substituted 1 or 2 times with R⁹, and Y¹ is N or CH;
one R³ is H and the other R³ is H, halo, alkyl, OH or O-alkyl;
Y¹ is N or C—R^{b}, wherein R^{b} is selected from H, halo, alkyl, haloalkyl, OR¹⁰, CO₂R¹⁰, NR¹⁰R¹¹, S(O)₂R¹⁰ and CN;
W is O or S;
R⁴ is selected from H, alkyl, haloalkyl, alkylene-OH, R¹²—SO₂NR¹³R¹⁴, NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, N(R¹³)phenyl, and 5-6 membered N-linked heterocycle, wherein said N-linked heterocycle optionally includes 1 or 2 additional heteroatoms selected from N, O and S, and wherein said N-linked heterocycle is optionally substituted 1 or 2 times with a substituent selected from alkyl, oxo,
   O-alkyl, OH, R¹²—OH, NH₂, N(H)alkyl and N(alkyl)₂;
   e is 2, 3 or 4;
   each R¹³ is the same or different and is selected from H, alkyl and haloalkyl; and
   each R¹⁴ is the same or different and is selected from H, alkyl, haloalkyl, and C₃_{_{—}}₆cycloalkyl;
Y² is N or R⁶—C;
Y³ is N or R^{a}—C;
Y⁴ is N or R⁵—C;
wherein not more than one of Y², Y³ and Y⁴ is N;
each R⁵ is the same or different and is selected from H, halo and alkyl, wherein when Y⁴ is R⁵—C, at least one R⁵ is H;
R^{a} is selected from H, halo, alkyl, haloalkyl, R¹²—OH and OR¹⁰;
each R⁶ is the same or different and is independently selected from H, halo, alkyl, alkenyl, alkynyl, haloalkyl, R¹²—OH, OR¹⁰ and NR¹⁰R¹¹, wherein at least one R⁶ is not H;
or R⁶ and R^{a} together with the aromatic ring to which they are bonded form an indenyl, naphthyl or a 9 or 10-membered fused bicyclic heteroaryl having 1, 2 or 3 heteroatoms selected from N, O and S, wherein said indenyl, naphthyl or fused bicyclic heteroaryl is optionally substituted 1 or 2 times with an additional substituent selected from alkyl, oxo, O-alkyl, OH, R¹²—OH, NH₂, N(H)alkyl and N(alkyl)₂;
each R¹⁰ and each R¹¹ is the same or different and is independently selected from H, alkyl and haloalkyl; and
each R¹² is the same or different and is independently C₁₋₄alkylene;
   or a salt thereof, particularly a pharmaceutically acceptable salt thereof.

In a second aspect of the present invention, there is provided a pharmaceutical composition comprising a compound of formula (I) or a pharmaceutically acceptable salt thereof. In one embodiment, the pharmaceutical composition further comprises one or more pharmaceutically acceptable carriers, diluents or excipients.

In a third aspect of the present invention, there is provided a method of treating a susceptible neoplasm in a mammal in need thereof, comprising administering to said mammal a therapeutically effective amount of a compound of formula (I) or a pharmaceutically acceptable salt thereof. Susceptible neoplasms include e.g.,
Barret's adenocarcinoma;
billiary tract carcinomas;
breast cancer;
cervical cancer;
cholangiocarcinoma;
central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system);
colorectal cancer including large intestinal colon carcinoma;
endometrial cancer;
gastric cancer;
carcinoma of the head and neck including squamous cell carcinoma of the head and neck;
hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia;
hepatocellular carcinoma;
lung cancer including small cell lung cancer and non-small cell lung cancer;
ovarian cancer;
pancreatic cancer;
pituitary adenoma;
prostate cancer;
renal cancer;
sarcoma;
skin cancers including melanomas; and
thyroid cancers.

In a fourth aspect of the present invention, there is provided a process for preparing a compound of formula (I) or a salt thereof. The process comprises reacting a compound of formula (V):

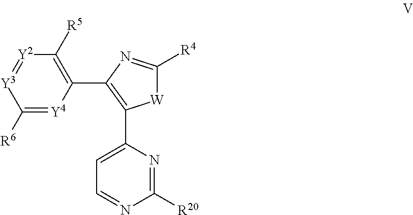

wherein R²⁰ is halo or thiomethyl;
   with an aniline of formula (VI):

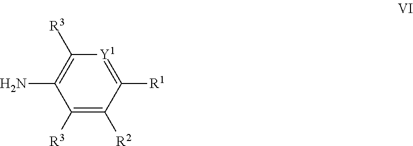

to prepare a compound of formula (I).

In a fifth aspect, the present invention provides a process for preparing a compound of formula (I) or a salt thereof. The process comprises reacting a compound of formula (VIII):

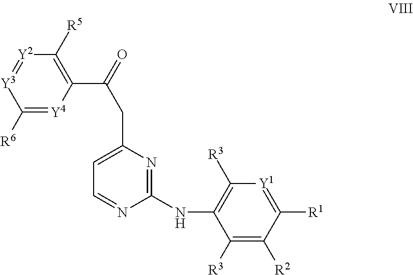

with a suitable brominating agent followed by reaction with one of:
i) a thiourea,
ii) a formamide,
iii) an amide,
iv) a thioamide, or
v) a urea;
   to prepare a compound of formula (I).

In a sixth aspect of the present invention, there is provided a compound of formula (I), or a pharmaceutically acceptable salt thereof for use in therapy.

In a seventh aspect of the present invention, there is provided the use of a compound of formula (I), or a pharmaceutically acceptable salt thereof in the preparation of a medicament for use in the treatment of a susceptible neoplasm (e.g.,
Barret's adenocarcinoma;
billiary tract carcinomas;
breast cancer;
cervical cancer;
cholangiocarcinoma;
central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system);
colorectal cancer including large intestinal colon carcinoma;
endometrial cancer;
gastric cancer;
carcinoma of the head and neck including squamous cell carcinoma of the head and neck;
hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia;
hepatocellular carcinoma;
lung cancer including small cell lung cancer and non-small cell lung cancer; ovarian cancer;
pancreatic cancer;
pituitary adenoma;
prostate cancer;
renal cancer;
sarcoma;
skin cancers including melanomas; and
thyroid cancers)
   in a mammal (e.g., human) in need thereof.

In another aspect of the present invention, there is provided the use of a compound of formula (I), or a pharmaceutically acceptable salt thereof in the preparation of a medicament for use in the treatment of a susceptible neoplasm (e.g., Barret's adenocarcinoma;
billiary tract carcinomas;
breast cancer;
cervical cancer;
cholangiocarcinoma;
central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system);
colorectal cancer including large intestinal colon carcinoma;
endometrial cancer;
gastric cancer;
carcinoma of the head and neck including squamous cell carcinoma of the head and neck;
hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia;
hepatocellular carcinoma;
lung cancer including small cell lung cancer and non-small cell lung cancer; ovarian cancer;
pancreatic cancer;
pituitary adenoma;
prostate cancer;
renal cancer;
sarcoma;
skin cancers including melanomas; and
thyroid cancers)
   in a mammal (e.g., human) in need thereof.

### DETAILED DESCRIPTION OF THE INVENTION

As used herein, the term “Raf family kinase” refers to Raf kinases including A-Raf, B-Raf and c-Raf (also known as Raf-1).

As used herein, “compound(s) of formula (I)” means any compound having the structural formula (I) as defined by the variable definitions provided, solvates, hydrates and amorphous and crystalline forms thereof, including one or more polymorphic forms and mixtures thereof. In the case of compounds of formula (I) which possess one or more chiral centers, the compounds may be in the form of a racemic mixture, or one or more isomerically enriched or pure stereoisomers, including enantiomers and diastereomers thereof. In such embodiments, “compound(s) of formula (I)” includes the racemic form as well as the enriched or pure enantiomers and diastereomers. Enantiomerically enriched or pure compounds will be designated using conventional nomenclature, including the designations +, −, R, S, d, I, D and L, according to the predominant isomer present. Where a compound of the invention contains an alkenyl or alkenylene group, cis (E) and trans (Z) isomerism may also occur. In such embodiments, “compound(s) of formula (I)” includes the individual stereoisomers of the compound, which will be indicated using conventional, cis/trans nomenclature. It should also be understood that compounds of formula (I) may exist in tautomeric forms other than that shown in the formula and alternative tautomeric forms are also included within “compound(s) of formula (I).”

As used herein, “compound(s) of the invention” means a compound of formula (I) (as defined above) in any version, i.e., as the free base or as a pharmaceutically acceptable salt thereof. The compound as any version may be in any form, including amorphous or crystalline forms, specific polymorphic forms, solvates, including hydrates (e.g., mono-, di-, and hemi-hydrates), and mixtures of various forms.

Intermediates may also be present as salts. In reference to intermediates, the phrase “compound(s) of formula (number)” means a compound having that structural formula or a salt, e.g., a pharmaceutically acceptable salt, thereof.

Throughout this application, where variables and/or terms are defined by a Markush group, e.g., methyl, ethyl, propyl, and isopropyl, a listing of members of the group is intended to describe each member of the Markush group individually. Hence, the foregoing list describes not only the group of four members, but also e.g., isopropyl specifically.

The term “alkyl” as used herein refers to linear or branched hydrocarbon chains having from 1 to 8 carbon atoms (i.e., C₁₋₈alkyl), unless a different number of atoms is specified. Examples of “alkyl” as used herein include, but are not limited to, methyl, ethyl, n-propyl, n-butyl, n-pentyl, isobutyl, isopropyl, and tert-butyl. Similarly, the term “alkylene” refers to linear or branched divalent hydrocarbon chains containing from 1 to 8 carbon atoms, unless a different number of atoms is specified. Examples of “alkylene” as used herein include, but are not limited to, methylene, ethylene, propylene, butylene, and isobutylene.

As used herein, the term “alkenyl” refers to linear or branched hydrocarbon chains having from 2 to 8 carbon atoms, unless a different number of atoms is specified, and at least one and up to three carbon-carbon double bonds. Examples of “alkenyl” as used herein include, but are not limited to ethenyl and propenyl. Similarly, the term “alkenylene” refers to linear or branched divalent hydrocarbon chains containing from 2 to 8 carbon atoms, unless a different number of atoms is specified, and at least one and up to three carbon-carbon double bonds. Examples of “alkenylene” as used herein include, but are not limited to, ethenylene, propenylene and butenylene.

As used herein, the term “alkynyl” refers to linear or branched hydrocarbon chains having from 2 to 8 carbon atoms, unless a different number of atoms is specified, and at least one and up to three carbon-carbon triple bonds. Examples of “alkynyl” as used herein include, but are not limited to ethynyl and propynyl.

As used herein, the term “cycloalkyl” refers to a saturated monocyclic carbocyclic ring having from 3 to 8 carbon atoms, unless a different number of atoms is specified. In one embodiment, “cycloalkyl” refers to a saturated monocyclic carbocyclic ring having from 3 to 6 carbon atoms, unless a different number is specified. “Cycloalkyl” includes by way of example cyclopropyl, cyclobutyl, cyclopentyl, cyclohexyl, cycloheptyl and cyclooctyl. Preferred cycloalkyl groups include substituted and unsubstituted C₃₋₆cycloalkyl.

As used herein, the term “cycloalkenyl” refers to a non-aromatic, monocyclic carbocyclic ring having from 3 to 8 carbon atoms, unless a different number of atoms is specified, and up to 3 carbon-carbon double bonds. In one embodiment, “cycloalkenyl” refers to a monocyclic carbocyclic ring having from 3 to 6 carbon atoms, unless a different number is specified, one or more carbon-carbon double bonds. “Cycloalkenyl” includes by way of example cyclopentenyl and cyclohexenyl. Preferred cycloalkenyl groups include substituted and unsubstituted C₅₋₆cycloalkenyl.

The terms “halo” or “halogen” are synonymous and refer to fluoro, chloro, bromo and iodo.

As used herein, “haloalkyl” refers to an alkyl, as defined above, substituted by one or more halogen atoms, fluoro, chloro, bromo or iodo. Where the haloalkyl group has fewer than 8 carbon atoms, the number of carbon atoms in the group is indicated as, for example, haloC₁₋₃alkyl, which indicates that the haloalkyl group has 1, 2 or 3 carbon atoms. Examples of haloalkyl as used herein include, but are not limited to fluoromethyl, difluoromethyl, trifluoromethyl, fluoroethyl, trifluoroethyl and the like. The term “oxo” as used herein refers to the group ═O attached directly to a carbon atom of a hydrocarbon ring (e.g., cycloalkyl or cycloalkenyl) or a C, N or S of a heterocyclic or heteroaryl ring to result in oxides, —N-oxides, sulfones and sulfoxides.

As used herein, the term “aryl” refers to aromatic monocyclic carbocyclic groups, aromatic fused bicyclic carbocyclic groups, and fused bicyclic carbocyclic groups which have both aromatic and non-aromatic rings, each having from 6 to 10 carbon atoms, unless a different number of atoms is specified. In all embodiments wherein the compound of formula (I) includes two or more aryl groups, the aryl groups may be the same or different and are independently selected. Examples of particular aryl groups include but are not limited to phenyl, indenyl and naphthyl. In one particular embodiment, “aryl” refers to phenyl.

As used herein, the terms “heterocycle” and “heterocyclic” are synonymous and refer to monocyclic saturated or unsaturated non-aromatic groups, fused bicyclic saturated or unsaturated non-aromatic groups, each having from 5 to 10 members (unless a different number of members is specified), and spiro systems having from 7 to 12 members (unless a different number of members is specified). The monocyclic, bicyclic and spiro systems, include 1, 2, 3 or 4 (particularly 1, 2 or 3) heteroatoms selected from N, O and S, unless a different number of heteroatoms is specified. In one embodiment, “heterocycle” and “heterocyclic” refer to monocyclic saturated or unsaturated non-aromatic groups and fused bicyclic saturated or unsaturated non-aromatic groups, each having from 5 to 10 members (unless a different number of members is specified) including 1, 2, 3 or 4 (particularly 1, 2 or 3) heteroatoms selected from N, O and S, unless a different number of heteroatoms is specified. In embodiments wherein the heterocycle has 6 or fewer members, it should be clear that such embodiments do not include 7-12 membered spiro systems. In all embodiments wherein the heterocycle includes 2 or more heteroatoms, the heteroatoms may be the same or different and are independently selected from N, O and S. In all embodiments wherein the compound of formula (I) includes two or more heterocyclic groups, the heterocyclic groups may be the same or different and are independently selected. Examples of particular heterocyclic groups include but are not limited to tetrahydrofuran, dihydropyran, tetrahydropyran, pyran, thietane, 1,4-dioxane, 1,3-dioxane, 1,3-dioxalane, piperidine, piperazine, pyrrolidine, morpholine, thiomorpholine, thiazolidine, oxazolidine, tetrahydrothiopyran, tetrahydrothiophene and the like.

As used herein, the term “N-heterocycle” refers to monocyclic saturated or unsaturated non-aromatic groups and fused bicyclic saturated or unsaturated non-aromatic groups, each having from 5 to 10 members (unless a different number of members is specified), and spiro systems having from 7 to 12 members. The monocyclic, bicyclic and spiro systems, include at least one N and optionally 1, 2 or 3 additional heteroatoms selected from N, O and S, unless a different number of additional heteroatoms is specified. In one embodiment, “N-heterocycle” refers to monocyclic saturated or unsaturated non-aromatic groups and fused bicyclic saturated or unsaturated non-aromatic groups, each having from 5 to 10 members (unless a different number of members is specified) including at least one N and optionally 1, 2 or 3 additional heteroatoms selected from N, O and S, unless a different number of additional heteroatoms is specified. In embodiments wherein the N-heterocycle has 6 or fewer members, it should be clear that such embodiments do not include 7-12 membered spiro systems. By “additional heteroatoms” is meant 1, 2 or 3 heteroatoms in addition to the N already specified in the N-heterocycle ring. In all embodiments wherein the heterocycle includes 1 or more additional heteroatoms, the heteroatoms may be the same or different and are independently selected from N, O and S. In all embodiments wherein the compound of formula (I) includes two or more N-heterocyclic groups, the N-heterocyclic groups may be the same or different and are independently selected. Examples of N-heterocycles include piperidine, piperazine, pyrrolidine, morpholine, thiomorpholine and the like.

As used herein, the term “N-linked heterocycle” refers to an N-heterocycle as defined above which is bound through the N of the N-heterocycle. As with N-heterocycles, the N-linked heterocycle may optionally include 1, 2 or 3 additional heteroatoms (typically, 1 or 2 additional heteroatoms) which are the same or different and are selected from N, O and S. Examples of N-linked heterocycles include, but are not limited to:

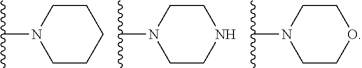

As used herein, the term “heteroaryl” refers to aromatic, monocyclic groups, aromatic fused bicyclic groups and fused bicyclic groups which have both aromatic and non-aromatic rings, each having from 5 to 10 members (unless a different number of members is specified) including 1, 2, 3, or 4 heteroatoms (particularly 1, 2 or 3 heteroatoms) selected from N, O and S, unless a different number of heteroatoms is specified. In all embodiments wherein the heteroaryl includes 2 or more heteroatoms, the heteroatoms may be the same or different and are independently selected from N, O and S. In all embodiments wherein the compound of formula (I) includes two or more heteroaryl groups, the heteroaryl groups may be the same or different and are independently selected. Examples of particular heteroaryl groups include but are not limited to furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, triazine, quinoline, tetrahydroquinoline, isoquinoline, tetrahydroisoquinoline, benzofuran, benzothiophene, indole, indoline, indazole, benzodioxane, benzodioxin, benzodithiane, benzoxazine, benzopiperidine and benzopiperazine.

As used herein, the term “N-heteroaryl” refers to aromatic, monocyclic groups, aromatic fused bicyclic groups and fused bicyclic groups which have both aromatic and non-aromatic rings, each having from 5 to 10 members (unless a different number of members is specified) including at least one N and optionally 1, 2 or 3 additional heteroatoms selected from N, O and S, unless a different number of heteroatoms is specified. By “additional heteroatoms” is meant 1, 2 or 3 heteroatoms in addition to the N already specified in the N-heteroaryl ring. In all embodiments wherein the heteroaryl includes 1 or more additional heteroatoms, the heteroatoms may be the same or different and are independently selected from N, O and S. In all embodiments wherein the compound of formula (I) includes two or more N-heteroaryl groups, the N-heteroaryl groups may be the same or different and are independently selected. Examples of N-heteroaryls include pyrrole, imidazole, pyrazole, thiazole, isoxazole, pyridine, pyridazine, pyrazine, pyrimidine, triazine, quinoline, isoquinoline, indole, indoline, benzopiperidine and benzopiperazine.

As used herein, the term “members” (and variants thereof e.g., “membered”) in the context of heterocyclic and heteroaryl groups refers to the total number of ring atoms, including carbon and heteroatoms N, O and/or S. Thus, an example of a 6-membered heterocyclic ring is piperidine and an example of a 6-membered heteroaryl ring is pyridine.

As used herein, the term “optionally” “means that the invention includes both embodiments wherein the described condition is and is not met. Thus, an N-heterocycle optionally having 1, 2 or 3 additional heteroatoms describes N-heterocycles including no additional heteroatoms as well as N-heterocycles including 1, 2 or 3 additional heteroatoms.

The present invention provides compounds of formula (I):

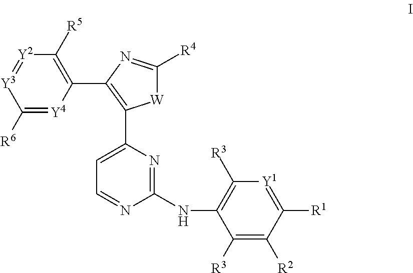

wherein:
R¹ is a moiety i, ii, or iii:

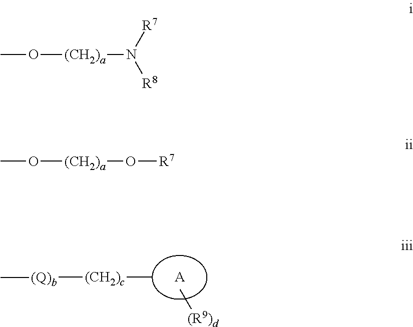

wherein:
a is 2, 3, or 4;
R⁷ and R⁸ are the same or different and are each independently selected from H, alkyl, haloalkyl, alkenyl, alkynyl, C₃₋₆cycloalkyl, and C₃₋₆cycloalkenyl;
b is 0 or 1;
Q is selected from —O—, —N(H)— and —N(alkyl)-;
c is 0, 1, 2 or 3;
Ring A is a 4-10 membered N-heterocycle optionally having 1 or 2 additional heteroatoms selected from N, O and S, or 5-10 membered N-heteroaryl optionally having 1 or 2 additional heteroatoms selected from N, O and S;
d is 0, 1 or 2;
each R⁹ is the same or different and is independently selected from halo, alkyl, alkenyl, alkynyl, haloalkyl, oxo, OR¹⁰, R¹²—OR¹⁰, C(O)R¹⁰, CO₂R¹⁰,
   C(O)₂-benzyl, CONR¹⁰R¹¹, COR¹²—NR¹⁰R¹¹, COR¹²—OR¹⁰, NR¹⁰R¹¹, R¹²—NR¹⁰R¹¹, N(R¹⁰)C(O)R¹¹, N(R¹⁰)S(O)₂R¹¹, N(R¹⁰)C(O)N(R¹¹), N(R¹⁰)C(S)N(R¹¹), S(O)₃H, R¹²—S(O)₃H, S(O)₂R¹⁰, R¹²—S(O)₂R¹⁰, S(O)₂NR¹⁰R¹¹, CN and R¹²—CN;
R² is selected from H, halo, alkyl, haloalkyl, OR¹⁰, CO₂R¹⁰, NR¹⁰R¹¹, S(O)₂R¹⁰, CN and
   5-6 membered N-heterocycle optionally having 1 additional heteroatom selected from N, O and S and optionally substituted 1 or 2 times with alkyl or oxo; or
R¹ and R², together with the aromatic ring to which they are bound form a 9 or 10-membered fused, bicyclic heteroaryl having 1, 2 or 3 heteroatoms selected from N, O and S, wherein said fused bicyclic heteroaryl is optionally substituted 1 or 2 times with R⁹, and Y¹ is N or CH;
one R³ is H and the other R³ is H, halo, alkyl, OH or O-alkyl;
Y¹ is N or C—R^{b}, wherein R^{b} is selected from H, halo, alkyl, haloalkyl, OR¹⁰, CO₂R¹⁰, NR¹⁰R¹¹, S(O)₂R¹⁰ and CN;
W is O or S;
R⁴ is selected from H, alkyl, haloalkyl, alkylene-OH, R¹²—SO₂NR¹³R¹⁴, NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, N(R¹³)phenyl, and 5-6 membered N-linked heterocycle, wherein said N-linked heterocycle optionally includes 1 or 2 additional heteroatoms selected from N, O and S, and wherein said N-linked heterocycle is optionally substituted 1 or 2 times with a substituent selected from alkyl, oxo,
   O-alkyl, OH, R¹²—OH, NH₂, N(H)alkyl and N(alkyl)₂;
   e is 2, 3 or 4;
   each R¹³ is the same or different and is selected from H, alkyl and haloalkyl; and
   each R¹⁴ is the same or different and is selected from H, alkyl, haloalkyl, and C₃_{_{—}}₆cycloalkyl;
Y² is N or R⁶—C;
Y³ is N or R^{a}—C;
Y⁴ is N or R⁵—C;
wherein not more than one of Y², Y³ and Y⁴ is N;
each R⁵ is the same or different and is selected from H, halo and alkyl, wherein when Y⁴ is R⁵—C, at least one R⁵ is H;
R^{a} is selected from H, halo, alkyl, haloalkyl, R¹²—OH and OR¹⁰;
each R⁶ is the same or different and is independently selected from H, halo, alkyl, alkenyl, alkynyl, haloalkyl, R¹²—OH, OR¹⁰ and NR¹⁰R¹¹, wherein at least one R⁶ is not H;
or R⁶ and R^{a} together with the aromatic ring to which they are bonded form an indenyl, naphthyl or a 9 or 10-membered fused bicyclic heteroaryl having 1, 2 or 3 heteroatoms selected from N, O and S, wherein said indenyl, naphthyl or fused bicyclic heteroaryl is optionally substituted 1 or 2 times with an additional substituent selected from alkyl, oxo, O-alkyl, OH, R¹²—OH, NH₂, N(H)alkyl and N(alkyl)₂;
each R¹⁰ and each R¹¹ is the same or different and is independently selected from H, alkyl and haloalkyl; and
each R¹² is the same or different and is independently C₁₋₄alkylene;
   and pharmaceutically acceptable salts thereof.

The compounds of the invention are described in the conventional manner employing variables to represent a number of possible substituents or groups. The broadest, particular and preferred definitions of variables described herein apply equally to compounds of formula (I) (including salts and pharmaceutically acceptable salts thereof) and compounds of the invention. For brevity, the following description will refer to “compounds of the invention” rather than to both, as compounds of the invention encompasses all compounds of formula (I). It should be understood that the definition of variables utilized to describe the compounds of the invention will be selected in light of the knowledge possessed by the ordinarily skilled organic chemist such that embodiments which such chemist would consider to be obviously inoperative or unstable are avoided. For example, the organic chemist of ordinary skill in the art would appreciate that moieties such as —N(H)CH₂F, —N(H)CH₂NH₂, —OCH₂NH₂, and the like, result in potentially unstable acetyls, aminals or iminium ions. As such, the present invention should be understood such that the variables are defined in a manner which avoids such embodiments.

Considering the definitions of R¹, the following formulas illustrate compounds within the scope of the invention.

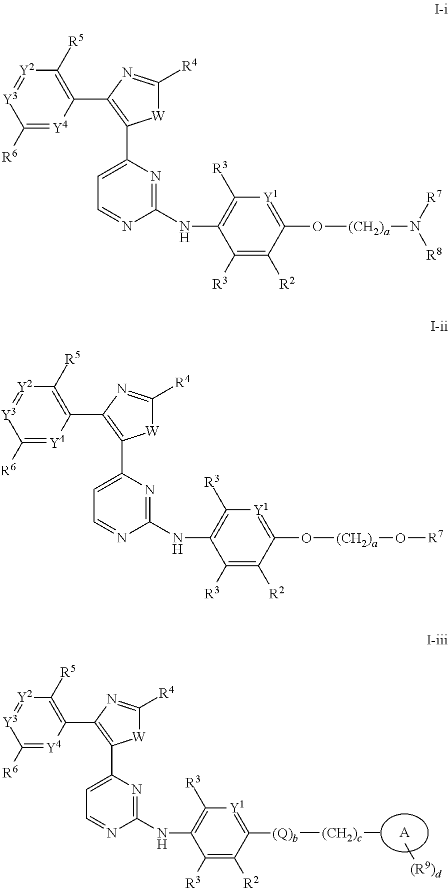

all variables defined as described herein.

In particular embodiments, the compounds of the invention are defined wherein R¹ is moiety i or iii, above (as illustrated in formulas I-i and I-iii).

In those embodiments wherein R¹ is moiety i, a is 2, 3 or 4, and particularly 2.

The moiety i is defined wherein R⁷ and R⁸ are the same or different and are each independently selected from H, alkyl, haloalkyl, alkenyl, alkynyl, C₃₋₆cycloalkyl and C₃₋₆cycloalkenyl, or any subset thereof. In particular embodiments, R⁷ and R⁸ are the same. In particular embodiments, the moiety i is defined wherein R⁷ and R⁸ are each the same or different and are independently selected from H, alkyl and haloalkyl, or any subset thereof; more particularly, H, C₁₋₃alkyl and haloC₁₋₃alkyl, or any subset thereof. In one particular embodiment, the moiety i is defined wherein R⁷ and R⁸ are the same and are selected from H, C₁₋₃alkyl and haloC₁₋₃alkyl, or any subset thereof. Specific examples of moiety i in the compounds of the present invention include but are not limited to: —O—(CH₂)₂—N(CH₃)₂, —O—(CH₂)₂—N(H)CH₃, —O—(CH₂)₂—N(CH₂CH₃)₂, —O—(CH₂)₂—N(H)(CH₂CH₃)₂, —O—(CH₂)₂—N(CH₃)—CH(CH₃)₂, —O—(CH₂)₂—N(CH₂CH₃)—(CH₂CH₃F), —O—(CH₂)₂—N(CH₃)—CH₂CF₃, and the like.

In those embodiments wherein R¹ is a moiety ii, a is 2, 3 or 4, preferably 2. In one embodiment, the moiety ii is defined wherein R⁷ is H, alkyl or haloalkyl, or any subset thereof, more particularly H, C₁₋₃alkyl or haloC₁₋₃alkyl, or any subset thereof.

In particular embodiments, R¹ is a moiety iii:

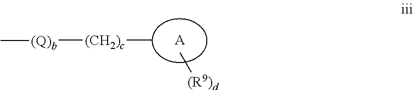

wherein:
b is 0 or 1;
Q is selected from —O—, —N(H)— and —N(alkyl)-;
c is 0, 1, 2 or 3;
Ring A is a 4-10 membered N-heterocycle optionally having 1 or 2 additional heteroatoms selected from N, O and S, or 5-10 membered N-heteroaryl optionally having 1 or 2 additional heteroatoms selected from N, O and S;
d is 0, 1 or 2;
each R⁹ is the same or different and is independently selected from halo, alkyl, alkenyl, alkynyl, haloalkyl, oxo, OR¹⁰, R¹²—OR¹⁰, C(O)R¹⁰, CO₂R¹⁰,
   C(O)₂-benzyl, CONR¹⁰R¹¹, COR¹²—NR¹⁰R¹¹, COR¹²—OR¹⁰, NR¹⁰R¹¹, R¹²—NR¹⁰R¹¹, N(R¹⁰)C(O)R¹¹, N(R¹⁰)S(O)₂R¹¹, N(R¹⁰)C(O)N(R¹¹), N(R¹⁰)C(S)N(R¹¹), S(O)₃H, R¹²—S(O)₃H, S(O)₂R¹⁰, R¹²—S(O)₂R¹⁰, S(O)₂NR¹⁰R¹¹, CN and R¹²—CN.

The variables defining moiety iii should be understood to be defined in view of each other so as to avoid embodiments which the organic chemist of ordinary skill would consider to be obviously unstable or inoperative. For example, in those embodiments wherein b is 1, Q is O, c is 0 and Ring A is an N-heterocycle bound to Q, then the N-heterocycle of Ring A should be bound to Q through a carbon suitable for binding to O (i.e., Q). Thus for example, when Q is O, the N-heterocycle of Ring A should not be bound to Q through a N of the ring.

In certain embodiments wherein R¹ is a moiety iii, b is 0. Other particular embodiments include those defined wherein, b is 1 and Q is selected from —O—, —N(H)— and —N(CH₃)—, or any subset thereof. These embodiments of moiety iii are illustrated as follows:

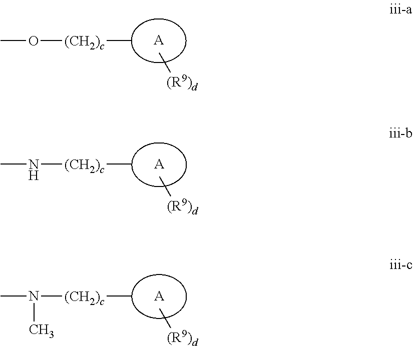

wherein all variables are as defined herein.

Compounds of the invention illustrating moiety iii in each of these embodiments include compounds of the following formulas:

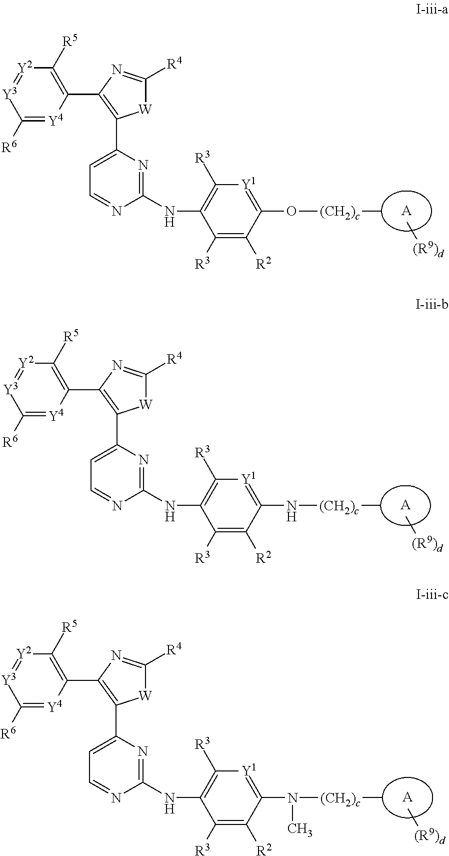

wherein all variables are as defined herein.

In one preferred embodiment, Q is —O— (illustrated in formula I-iii-a).

In certain embodiments wherein b is 0, c is defined as 0, 1 or 2; more particularly 0. Thus in one embodiment, the moiety iii is illustrated by moiety iii-d:

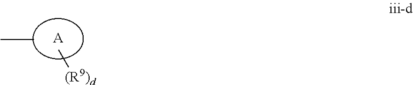

wherein Ring A, d and R⁹ are as defined herein.

This embodiment of compounds of formula (I) is illustrated in formula (I-iii-d):

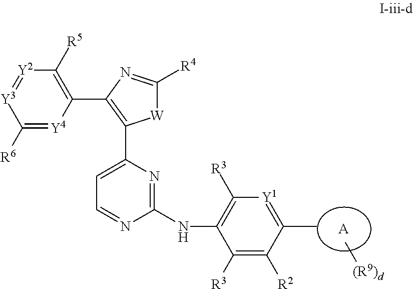

wherein all variables are as defined herein.

In one embodiment, moiety iii is defined wherein b is 1, Q is —O— and c is 0 or 2, thus, the moiety iii is a moiety iii-a1 or a moiety iii-a2:

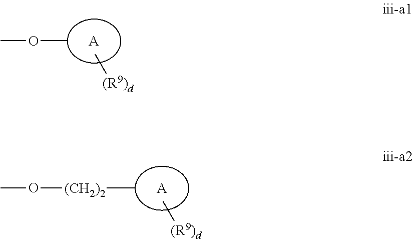

wherein Ring A, d and R⁹ are as defined herein.

Thus, a particular embodiment of the compounds of formula (I) is illustrated in formula (I-iii-a1):

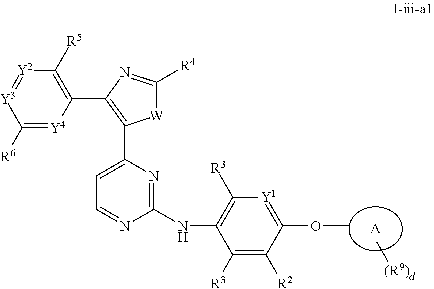

wherein all variables are as defined herein.

A further particular embodiment of the compounds of formula (I) is illustrated in formula (I-iii-a2):

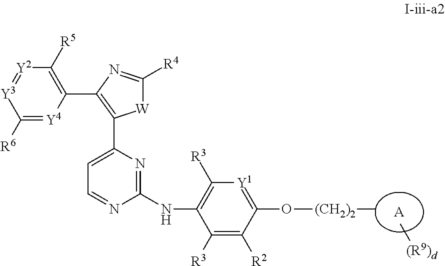

wherein all variables are as defined herein.

For the avoidance of doubt, the description referring to particular embodiments of variables defining moiety iii is intended to apply to the same variables illustrated in the subgeneric moeities iii-a, iii-a1, iii-a2, iii-b, iii-c and iii-d.

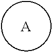

in moiety iii is referred to herein as “Ring A.” In certain embodiments, ring A is a monocyclic N-heterocycle or N-heteroaryl ring and in other embodiments Ring A is a bicyclic fused N-heterocycle or N-heteroaryl. In the embodiments wherein Ring A is the N-heterocycle, Ring A may be bound to the phenyl or pyridyl ring (when b and c are 0), Q (when b is 1 and c is 0) or the alkylene (when c is 1, 2 or 3) through a carbon or any suitable heteroatom of Ring A. Ring A may be bound through a nitrogen of the N-heterocycle.

In one embodiment, Ring A is a 4-10 membered N-heterocycle optionally having 1 or 2 additional heteroatoms selected from N, O and S. In one embodiment, Ring A is a 5-6 membered monocyclic N-heterocycle or N-heteroaryl, wherein each of said N-heterocycle and N-heteroaryl optionally has 1 additional heteroatom selected from N, O and S. In one particular embodiment, Ring A is 5-6 membered monocyclic N-heterocycle optionally having 1 additional heteroatom selected from N, O and S. In one embodiment Ring A is a 6-membered monocyclic N-heteroaryl optionally having 1 additional heteroatom selected from N, O and S.

Specific examples of N-heterocycles and N-heteroaryls within the definition of Ring A include but are not limited to:

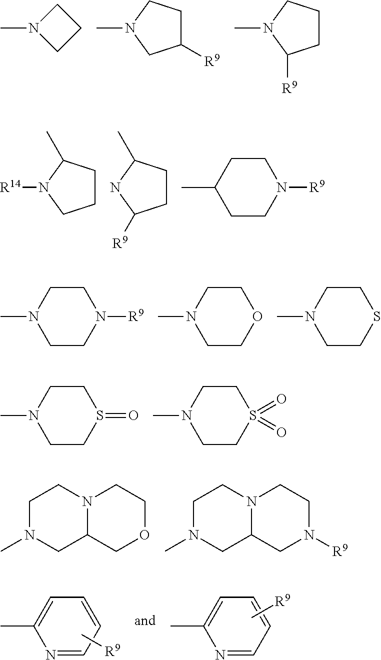

wherein the unfilled bond represents the point of attachment of Ring A.

The definition of the variable d, indicating the number of possible substituents R⁹ on Ring A should be understood to be consistent with and dependent upon the size of ring A. The substituents R⁹ may be bound to Ring A through any available carbon or heteroatom. In one embodiment, the moiety iii is defined wherein d is 0. In other particular embodiments, moiety iii is defined wherein d is 1 or 2, particularly 1.

In one embodiment, moiety iii is defined wherein R⁹ is selected from halo, alkyl, haloalkyl, oxo, OR¹⁰, R¹²—OR¹⁰, C(O)R¹⁰, CO₂R¹⁰, CONR¹⁰R¹¹, S(O)₂R¹⁰ and R¹²—S(O)₂R¹⁰, or any subset thereof. A particular embodiment of moiety iii is defined wherein R⁹ is selected from alkyl, R¹²—OR¹⁰, C(O)R¹⁰, CO₂R¹⁰ and R¹²—S(O)₂R¹⁰, or any subset thereof. Specific examples within the definition of R⁹ in moiety iii include but are not limited to methyl, ethyl, CH₂CH₂F, isopropyl, oxo, C(O)CH₃, CH₂CH₂—OCH₃, S(O)₂CH₃, and CH₂CH₂—S(O)₂CH₃, or any subset thereof.

The following description of particular and preferred embodiments defining variables of formula (I) are expressly intended to apply not only to compounds of formula (I), but also to each subgeneric formula described herein, individually (e.g., I-i, I-ii, I-iii, I-iii-a, I-iii-a1, I-iii-a2, I-iii-b, I-iii-c, I-iii-d, etc.).

In particular embodiments, the compounds of the invention (and each subgeneric formula described herein) are defined wherein R² is selected from H, halo, alkyl, haloalkyl, OR¹⁰, CO₂R¹⁰, NR¹⁰R¹¹, S(O)²R¹⁰ and CN, or any subset thereof. In other embodiments, R² is a 5-6 membered N-heterocycle optionally having 1 additional heteroatom selected from N, O and S. In particular embodiments, R² is selected from H, halo, alkyl, haloalkyl, OR¹⁰ and CO₂R¹⁰, or any subset thereof. In more particular embodiments, R² is selected from H, F, Cl, C₁₋₃alkyl, haloC₁₋₃alkyl, O—C₁₋₃alkyl, CO₂H and CO₂C₁₋₃alkyl, or any subset thereof. In specific examples, R² is selected from H, F, Cl, CH₃, CF₃, O—CH₃ and CO₂H, or any subset thereof. In a preferred embodiment, R² is F. In another preferred embodiment, R² is O—CH₃. In another preferred embodiment, R² is CF₃.

In other embodiments, the compounds of the invention are defined wherein R¹ and R², together with the aromatic ring to which they are bound form a 9 or 10-membered fused, bicyclic heteroaryl group having 1, 2 or 3 heteroatoms selected from N, O and S, which bicyclic heteroaryl group is optionally substituted 1 or 2 times with R⁹. Y¹ is defined as N or C—R^{b}. However, in these embodiments of the invention, Y¹ is preferably N or CH. These embodiments may be illustrated as compounds of formula (I-iv):

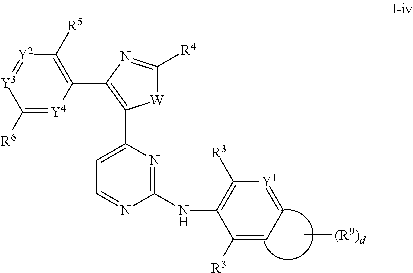

wherein optional additional heteroatoms in the fused ring are not depicted and all variables are as defined herein.

In examples of these embodiments, the compounds of the invention are defined wherein R¹ and R², together with the aromatic ring to which they are bound form a fused bicyclic heteroaryl group selected from:

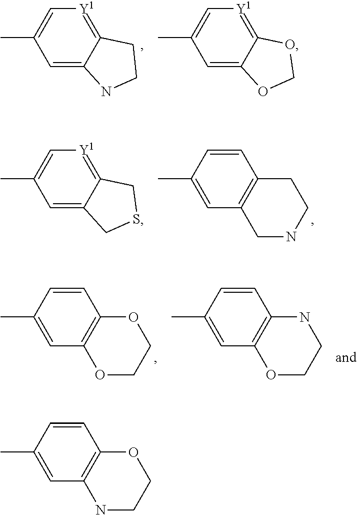

wherein Y¹ is N or CH, and each of the foregoing fused bicyclic heteroaryl groups is optionally substituted 1 or 2 times on any available C, N or S, with —R⁹.

In particular embodiments wherein R¹ and R² together with the aromatic ring to which they are bound form a fused bicyclic heteroaryl group, the fused bicyclic heteroaryl group is substituted once by R⁹. Specific examples of such embodiments include compounds wherein, R¹ and R² together with the aromatic ring to which they are bound form a fused bicyclic heteroaryl group substituted once by R⁹, wherein R⁹ is selected from CH₃, CH₂CH₃, CH(CH₃)₂, CF₃, oxo, OH, OCH₃, S(O)₃H, S(O)₂CH₃, C(O)CH₃, CH₂OH, CH₂OCH₃ and CH₂S(O)₂CH₃, or any subset thereof.

In particular embodiments wherein R¹ and R² together with the aromatic ring to which they are bound form a fused bicyclic heteroaryl group, Y¹ is CH.

Referring again to compounds of the invention as depicted by formula (I), and to each individual subgeneric formula of formula (I) illustrated herein, R³ is defined such that one R³ is H and the other R³ is H, halo, alkyl, OH or O-alkyl, or any subset thereof. In particular embodiments, one R³ is H and the other R³ is H, halo or C₁₋₃alkyl, or any subset thereof. In a preferred embodiment, both R³ are H.

In certain embodiments the compounds of the invention, are defined wherein Y¹ is N. In other embodiments, Y′ is C—R^{b}, wherein R^{b} is selected from H, halo, alkyl, haloalkyl, OR¹⁰, CO₂R¹⁰, NR¹⁰R¹¹, S(O)₂R¹⁰ and CN, or any subset thereof; more particularly, R^{b} is selected from H, halo, alkyl, haloalkyl, OR¹⁰ and CN, or any subset thereof. These embodiments of compounds of formula (I) may be illustrated as follows:

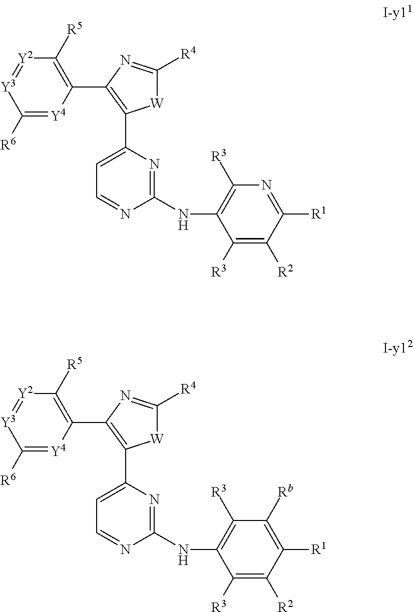

wherein all variables are as defined herein.

Additional, more specific subgeneric formulas, combining the specific definitions of Y¹ with the specific definitions of R¹, as illustrated in the subgeneric formulas above, and R² as described above will be apparent to those skilled in the art.

In certain embodiments, Y¹ is C—R^{b} and R^{b} is selected from H, F, Cl, C₁₋₃alkyl, haloC₁₋₃alkyl, OH and O—C₁₋₃alkyl, or any subset thereof; more particularly R^{b} is selected from H, F, Cl, CH₃, CF₃, and OCH₃, or any subset thereof. Specific examples of these embodiments include those wherein Y¹ is C—H, those wherein Y¹ is C—F or C—Cl, those wherein Y¹ is C—OCH₃, and those wherein Y¹ is C—CF₃. These embodiments of Y¹ may be combined with any of the embodiments of other variables of formula (I) including each of the individual subgeneric formulas illustrated herein.

In certain embodiments, the compounds of the invention are defined wherein W is O. In preferred embodiments, W is S. These embodiments of W may be combined with any of the embodiments of other variables of formula (I) including each of the individual subgeneric formulas illustrated herein. Examples of these embodiments in combination with particular embodiments of other variables described herein may be illustrated as follows. This list is illustrative of examples of certain combined elements defining embodiments of the compounds of the invention, but not exhaustive.

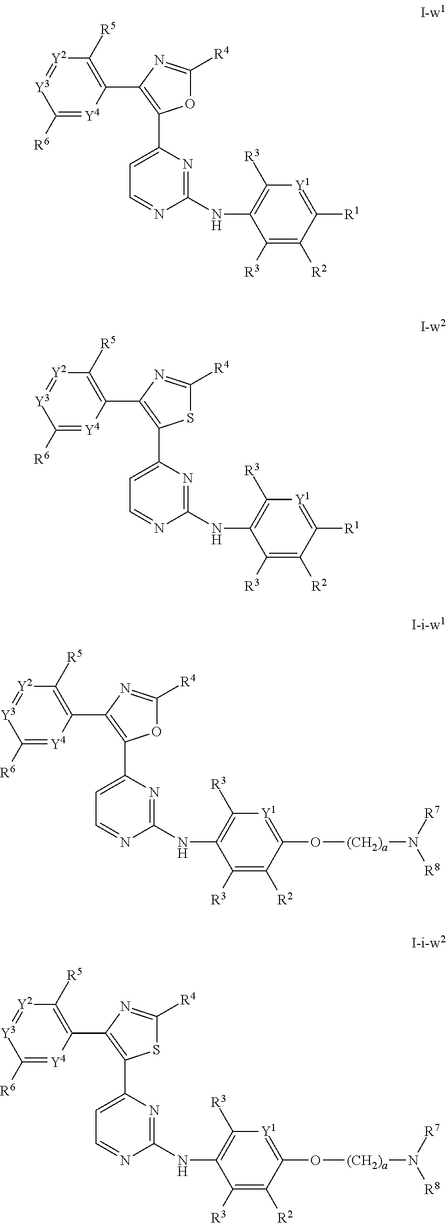

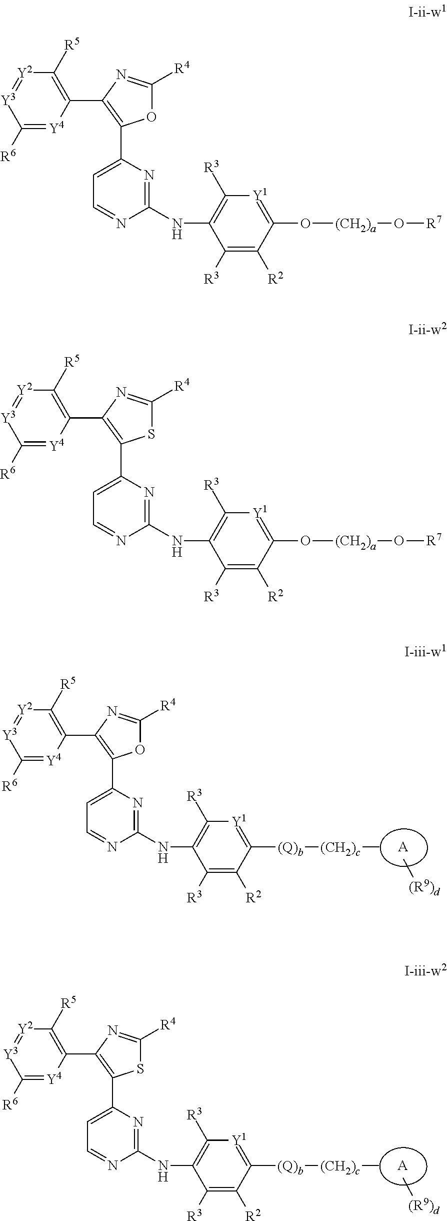

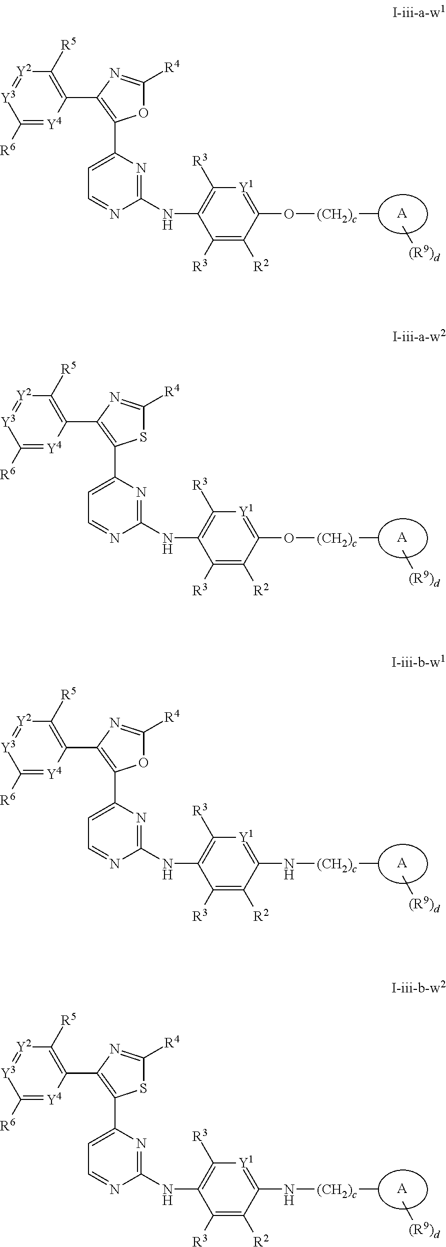

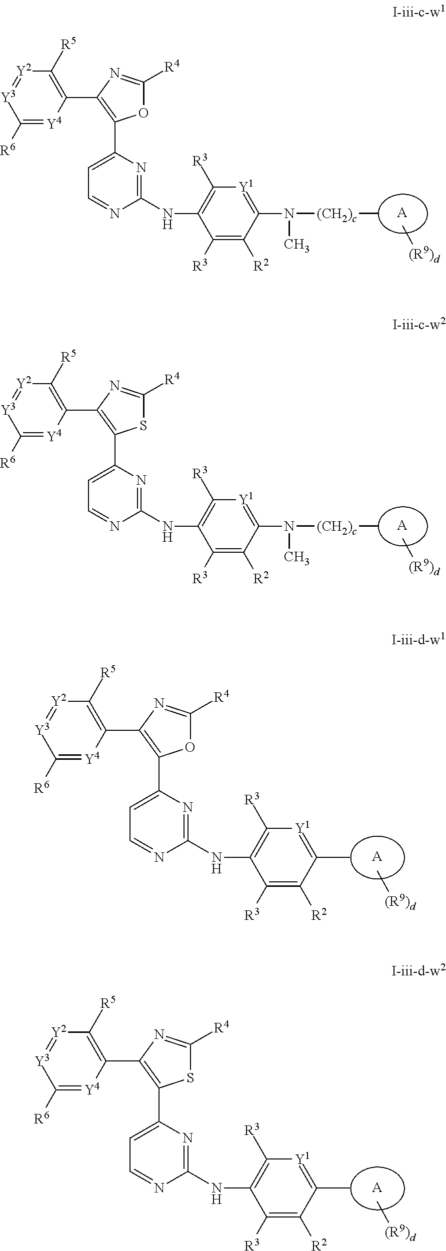

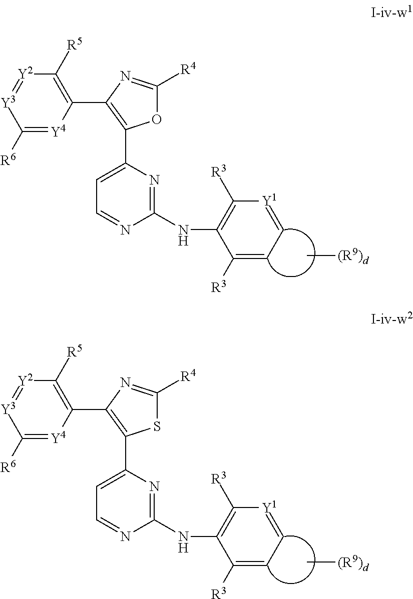

all variables being defined as described herein.

Referring again to compounds of the invention as depicted by formula (I), and to each individual subgeneric formula of formula (I) illustrated herein, in certain embodiments, the compounds are defined wherein R⁴ is selected from H, alkyl, haloalkyl, alkylene-OH, R¹²—SO₂NR¹³R¹⁴, NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, and N(R¹³)phenyl, or any subset thereof.

In other embodiments, R⁴ is a 5-6 membered N-linked heterocycle, optionally having 1 or 2 additional heteroatoms selected from N, O and S, wherein the N-linked heterocycle is optionally substituted 1 or 2 times with a substituent selected from alkyl, oxo, O-alkyl, OH, alkylene-OH, NH₂, N(H)alkyl and N(alkyl)₂, or any subset thereof. Particular embodiments include compounds wherein R⁴ is a 5-6 membered N-linked heterocycle having no additional heteroatoms, and optionally substituted 1 or 2 times with a substituent selected from alkyl, oxo, O-alkyl, OH, R¹²—OH, NH₂, N(H)alkyl and N(alkyl)₂, or any subset thereof.

In particular embodiments, R⁴ is selected from H, alkyl, R¹²—OH, R¹²—SO₂NR¹³R¹⁴, NR¹³R¹⁴, N(R¹³)(CH₂)ₑ—OR¹⁴, N(R¹³)(CH₂)ₑ—SO₂R¹⁴, R¹²—N(R¹³)SO₂R¹⁴, and 5-6 membered N-linked heterocycle, or any subset thereof, wherein said N-linked heterocycle is optionally substituted 1 or 2 times with a substituent selected from alkyl, oxo, O-alkyl, OH, alkylene-OH, NH₂, N(H)alkyl and N(alkyl)₂. More particular embodiments include compounds wherein R⁴ is selected from H, C₁₋₄alkyl, C₁₋₃alkylene-OH, C₁₋₂alkylene-SO₂NR¹³R¹⁴, NR¹³R¹⁴, N(H)(CH₂)ₑ—OR¹⁴, N(H)(CH₂)ₑ—SO₂R¹⁴ and C₁₋₂alkylene-N(R¹³)SO₂R¹⁴, and unsubstituted 5-6 membered N-linked heterocycle, or any subset thereof.

Within the definition of R⁴, particular embodiments of the invention are defined wherein e is 2.

Further within the definition of R⁴, particular embodiments of the invention are defined wherein R¹² is C₁₋₃alkylene.

In particular embodiments, R⁴ is defined wherein R¹³ and R¹⁴ are each the same or different and are independently H or alkyl.

Specific examples of embodiments of the invention include compounds wherein R⁴ is selected from H, C₁₋₄alkyl, R¹²—OH, C₁₋₂alkylene-SO₂N(H)R¹⁴, N(H)R¹⁴, N(C₁₋₄alkyl)R¹⁴, N(H)—C₂₋₃alkylene-OR¹⁴, N(H)—C₂₋₃alkylene-SO₂R¹⁴ and unsubstituted 5-6 membered N-linked heterocycle, or any subset thereof. In one preferred embodiment, R⁴ is selected from H, C₁₋₄alkyl, R¹²—OH, CH₂—SO₂NH(C₁₋₄alkyl), NH(C₁₋₄alkyl), NH(cyclopropyl), N(C₁₋₄alkyl)₂, N(H)—C₂₋₃alkylene-OH, NH—C₂₋₃alkylene-O(C₁₋₄alkyl), NH—C₂₋₃alkylene-SO₃H, NH—C₂₋₃alkylene-SO₂(C₁₋₄alkyl) and pyrrolidine, or any subset thereof. Examples of specific preferred embodiments include compounds wherein R⁴ is selected from H, CH₃, CH₂CH₃, CH(CH₃)₂, CH₂OH, CH₂—SO₂NH(CH₃), NH(CH₃), NH(CH₂CH₃), NH(CH(CH₃)₂), NH(cyclopropyl), N(CH₃)₂, N(H)—C₂₋₃alkylene-OH, NH—C₂₋₃alkylene-OCH₃, NH—C₂₋₃alkylene-SO₃H, and NH—C₂₋₃alkylene-SO₂(CH₃). Examples of more preferred embodiments include compounds wherein R⁴ is selected from CH₃, CH₂CH₃, CH(CH₃)₂, NH(CH₂CH₃), and NH(cyclopropyl). Any of the foregoing embodiments of R⁴ may be combined with any of the embodiments of other variables of formula (I) including each of the individual subgeneric formulas illustrated herein.

In the compounds of the invention, Y² is N or R⁶—C; Y³ is N or R^{a}—C, and Y⁴ is N or R⁵—C wherein not more than one of Y², Y³ and Y⁴ is N. These embodiments are illustrated within formula (I) by the following subgeneric formulas:

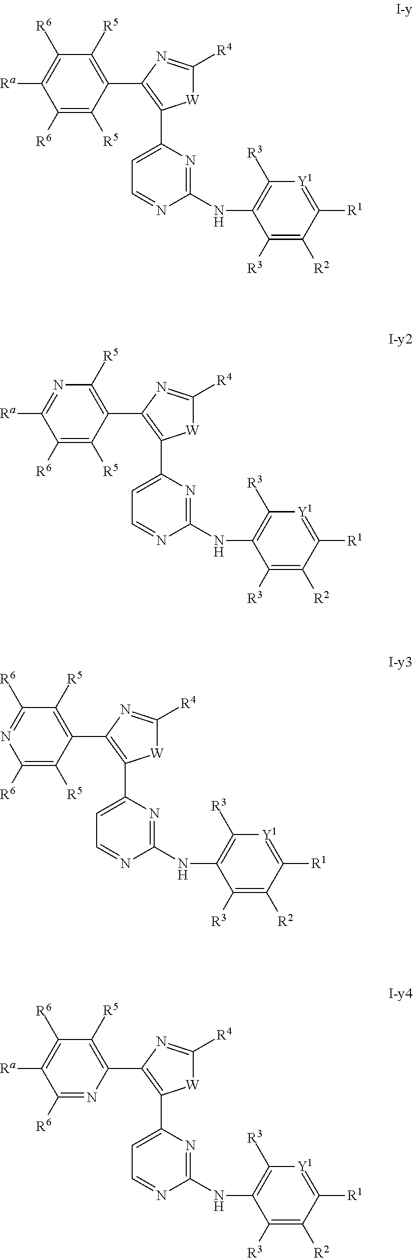

wherein all variables are as defined herein.

When Y⁴ is N, then Y² is R⁶—C and Y³ is R^{a}—C (illustrated as formula (I-y4). In particular embodiments, Y⁴ is R⁵—C (illustrated as formulas (I-y), (I-y2) and (I-y3)). When Y⁴ is R⁵—C, one R⁵ is H and the other R⁵ is H, halo or alkyl. Examples of particular embodiments include compounds wherein Y⁴ is R⁵—C, one R⁵ is H and the other R⁵ is H, F, Cl or methyl. In particular examples of these embodiments, the compounds of the invention are defined wherein Y⁴ is R⁵—C and both R⁵ are H. Any of the foregoing embodiments of Y⁴ may be combined with any of the embodiments of other variables of formula (I) including each of the individual subgeneric formulas illustrated herein.

In certain embodiments, the compounds of the invention are defined wherein Y² is N and thus Y³ is R^{a}—C and Y⁴ is R⁵—C (illustrated as formula (I-y2)). In particular embodiments, Y² is R⁶—C (illustrated as formulas (I-y), (I-y3) and (I-y4)). Examples of these embodiments include those wherein Y² is R⁶—C, and each R⁶ is the same or different and is independently selected from H, halo, alkyl, alkenyl, alkynyl, haloalkyl, R¹²—OH, OR¹⁰ and NR¹⁰R¹¹, or any subset thereof, wherein at least one R⁶ is not H. Particular embodiments include those wherein each R⁶ is the same or different and is independently selected from H, halo, alkyl, haloalkyl, R¹²—OH and OR¹⁰, or any subset thereof, wherein at least one R⁶ is not H. In particular embodiments wherein Y² is R⁶—C, each R⁶ is the same or different and is independently selected from H, halo, C₁₋₃alkyl, C₁₋₃alkylene-OH and OR¹⁰, or any subset thereof, wherein R¹⁰ is H or C₁₋₃alkyl and at least one R⁶ is not H. In specific examples of these embodiments, each R⁶ is the same or different and is independently selected from H, CH₃, CH₂OH, OH and O—CH₃ or any subset thereof, and at least one R⁶ is not H. Preferred embodiments wherein Y² is R⁶—C include those wherein each R⁶ is the same or different and is independently selected from H, CH₃, OH and OCH₃, wherein at least one R⁶ is not H. In particular embodiments wherein Y² is C—R⁶, both R⁶ are the same. In a preferred embodiment compounds of the invention are defined wherein Y² is C—R⁶, and both R⁶ are O—CH₃. In another preferred embodiment, Y² is C—R⁶, one R⁶ is O—CH₃ and other R⁶ is —CH₃. In another preferred embodiment, Y² is C—R⁶, one R⁶ is O—CH₃ and other R⁶ is H. In another preferred embodiment, Y² is C—R⁶, one R⁶ is OH and other R⁶ is H. Any of the foregoing embodiments of Y² may be combined with any of the embodiments of other variables of formula (I) including each of the individual subgeneric formulas illustrated herein.

In certain embodiments, the compounds of the invention are defined wherein Y³ is N. In embodiments wherein Y³ is N, then Y² is R⁶—C and Y⁴ is R⁵—C (illustrated in formula (I-y3). In particular embodiments, Y³ is R^{a}—C (illustrated in formulas (I-y), (I-y2) and (I-y4).

In the embodiments, wherein Y³ is R^{a}—C, R^{a} is selected from H, halo, alkyl, haloalkyl, R¹²—OH and —OR¹⁶, or any subset thereof. In particular embodiments, R^{a} is selected from H, halo, C₁₋₃alkyl, haloC₁₋₃alkyl, C₁₋₃alkylene-OH, OH and OC₁₋₃alkyl, or any subset thereof. In more particular embodiments, R^{a} is selected from H, halo, C₁₋₃alkyl, C₁₋₃alkylene-OH and OH, or any subset thereof. Examples of specific embodiments include those wherein, R^{a} is selected from H, F, C₁ and CH₂OH. In one preferred embodiment, Y³ is R^{a}—C and R^{a} is H.

Within the definitions of R⁶ and R^{a}, in certain embodiments each R¹⁰ and each R¹¹ are the same or different and are independently selected from H, C₁₋₃alkyl and haloC₁₋₃alkyl. In particular embodiment embodiments, each R¹⁰ and each R¹¹ in the definitions of R⁶ and R^{a}, are the same or different and are independently selected from H and C₁₋₃alkyl, more particularly H and methyl.

In certain embodiments, Y² is N or R⁶—C, Y³ is R^{a}—C, and one R⁶ and R^{a} together with the aromatic ring to which they are bonded form an indenyl, naphthyl or 9-10 membered fused bicyclic heteroaryl ring having 1, 2 or 3 heteroatoms selected from N, O and S. The indenyl, naphthyl or fused bicyclic heteroaryl may be substituted 1 or 2 times with a substituent selected from those described above. Specific examples of indenyl and fused bicyclic heteroaryl rings formed by R⁶ and R^{a} together with the aromatic ring to which they are bonded, include but are not limited to

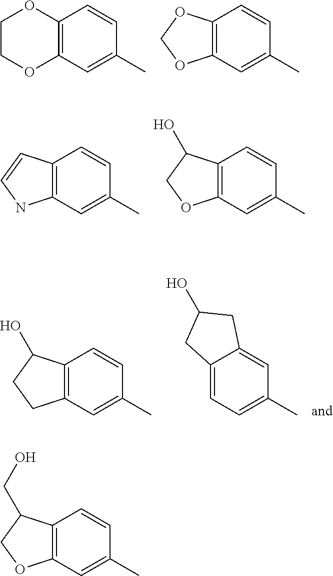

or any subset thereof. In one particular embodiment, one R⁶ and R^{a} together with the aromatic ring to which they are bonded form the group:

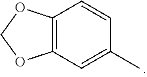

In one preferred embodiment of the invention, one R⁶ and R^{a} together with the aromatic ring to which they are bonded do not form an indenyl, naphthyl or 9-10 membered fused bicyclic heteroaryl ring.

Preferred embodiments of the invention include compounds wherein, Y² is R⁶—C, Y³ is R^{a}—C and Y⁴ is R⁵—C (illustrated as formula (I-y) above). More preferably, Y² is R⁶—C, Y³ is R^{a}—C and Y⁴ is R⁵—C, R^{a} is H, each R⁶ is the same and is selected from halo, alkyl, R¹²—OH and OR¹⁰ (particularly OH or O-alkyl, e.g., O-methyl), and both R⁵ are H. In another preferred embodiment, Y² is R⁶—C, Y³ is R^{a}—C, Y⁴ is R⁵—C, R^{a} is H, each R⁶ is the same and is OR¹⁰ (particularly OH or O-alkyl, e.g., O-methyl), and both R⁵ are H. In another preferred embodiment, Y² is R⁶—C, Y³ is R^{a}—C, Y⁴ is R⁵—C, R^{a} is H, one R⁶ is OR¹⁰ (particularly OH or O-alkyl, e.g., O-methyl), the other R⁶ is alkyl (e.g., methyl), and both R⁵ are H. In another preferred embodiment, Y² is R⁶—C, Y³ is R^{a}—C, Y⁴ is R⁵—C, R^{a} is H, one R⁶ is OR¹⁰ (particularly OH or O-alkyl, e.g., O-methyl), the other R⁶ is H, and both R⁵ are H.

In one embodiment, each R¹⁰ and each R¹¹ is the same or different and is independently selected from H, C₁₋₃alkyl and haloC₁₋₃alkyl, or any subset thereof.

In one embodiment, the compounds of the invention are defined wherein each R¹² is the same or different and is independently C₁₋₂alkylene.

One preferred set of compounds of the invention is defined by the formula (I-1):

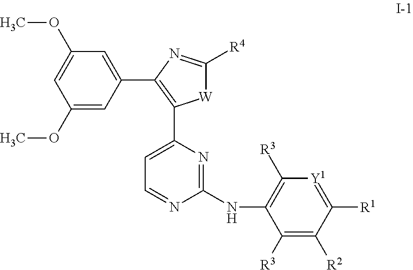

more particularly

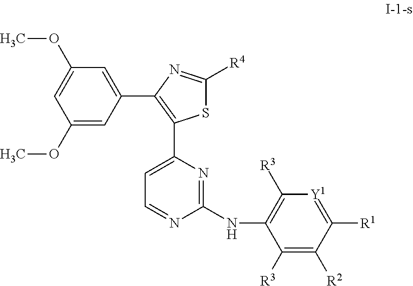

wherein all variables are as defined above.

More particularly, a preferred set of compounds of the invention is defined by formula (I-1a):

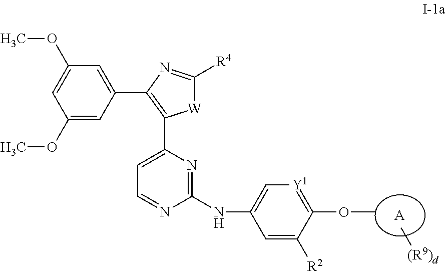

more particularly,

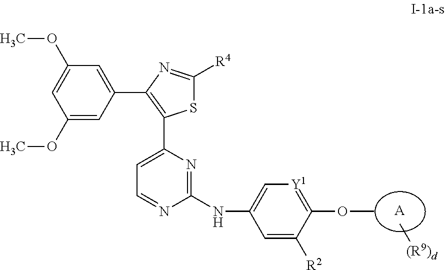

wherein all variables are as defined above.

Another preferred set of compounds of the invention is defined by the formula (I-1b):

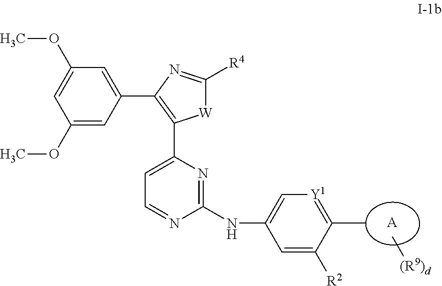

more particularly,

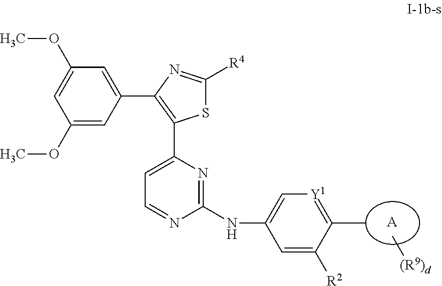

wherein all variables are as defined above.

Another preferred set of compounds of the invention is defined by the formula (I-1c):

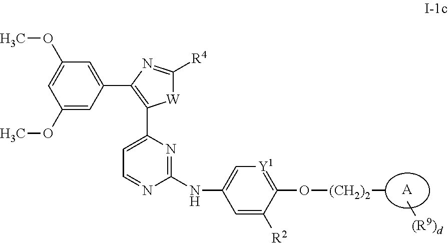

more particularly

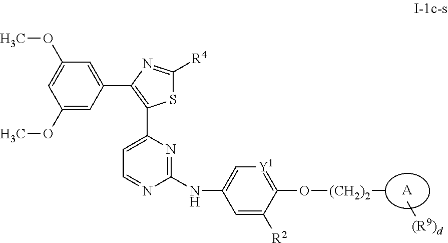

wherein all variables are as defined above.

Another preferred set of compounds of the invention is defined by the formula (I-2):

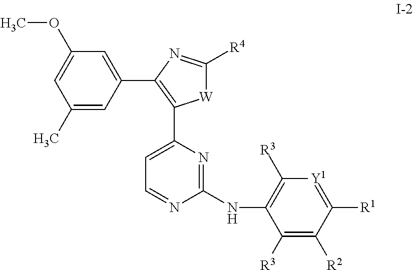

more particularly

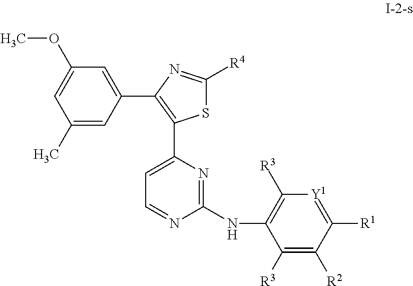

wherein all variables are as defined above.

More particularly, a preferred set of compounds of the invention is defined by formula (I-2a):

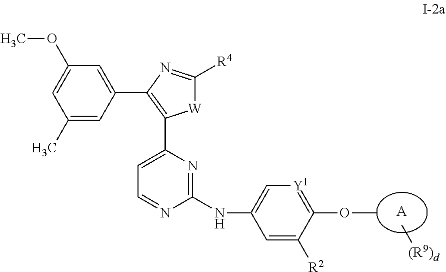

more particularly

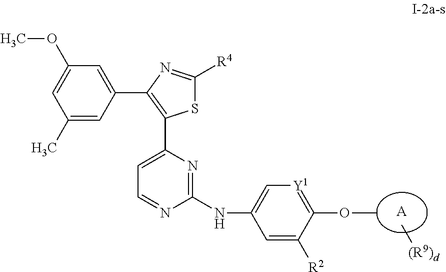

wherein all variables are as defined above.

Another preferred set of compounds of the invention is defined by formula (I-2b).

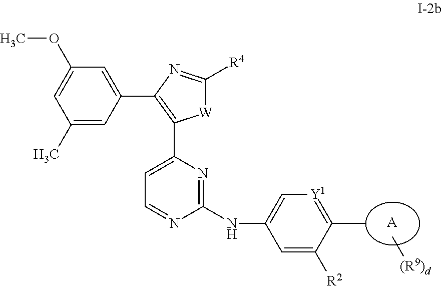

more particularly

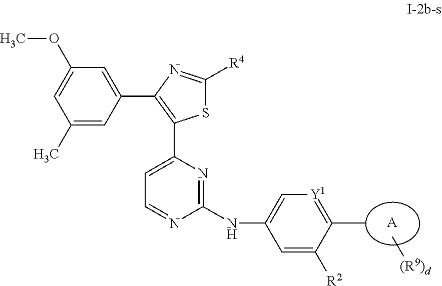

wherein all variables are as defined above.

Another preferred set of compounds of the invention is defined by formula (I-2c).

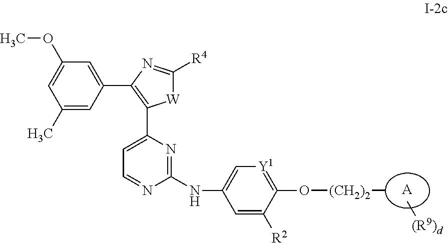

more particularly

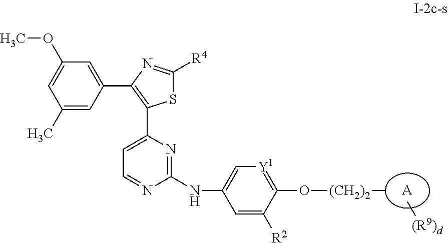

wherein all variables are as defined above.

Another preferred set of compounds of the invention is defined by formula (I-3):

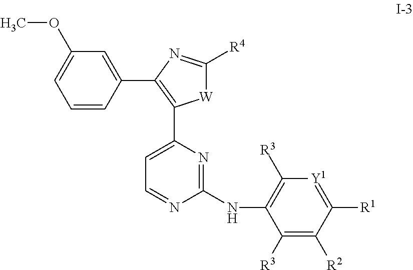

more particularly

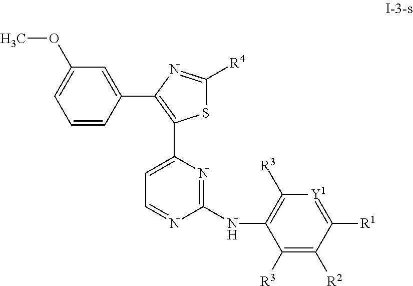

wherein all variables are as defined above.

More particularly, a preferred set of compounds of the invention is defined by formula (I-3a):

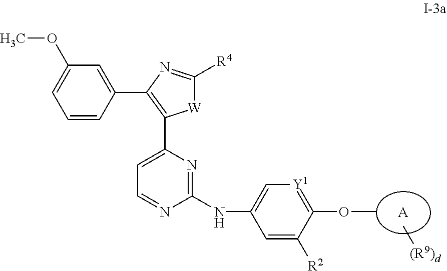

more particularly

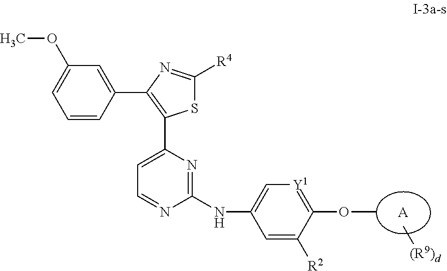

wherein all variables are as defined above.

Another preferred set of compounds of the invention is defined by formula (I-3b).

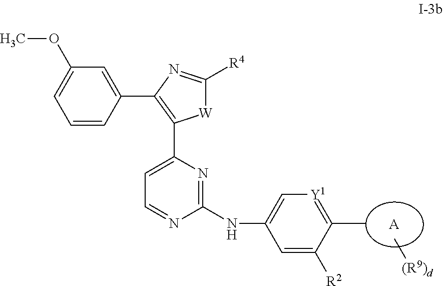

more particularly

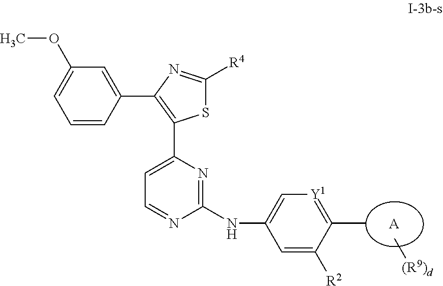

wherein all variables are as defined above.

Another preferred set of compounds of the invention is defined by formula (I-3c).

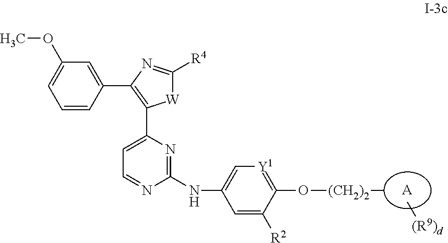

more particularly

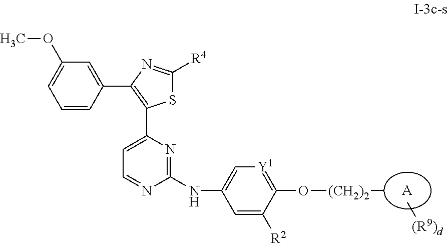

wherein all variables are as defined above.

Another preferred set of compounds of the invention is defined by formula (I-4):

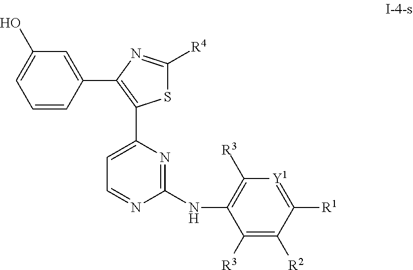

more particularly

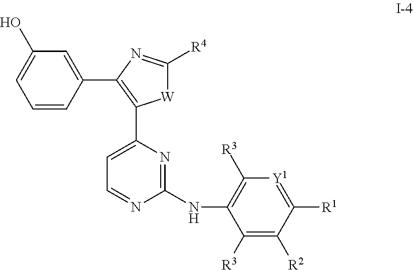

wherein all variables are as defined above.

More particularly, a preferred set of compounds of the invention is defined by formula (I-4a):

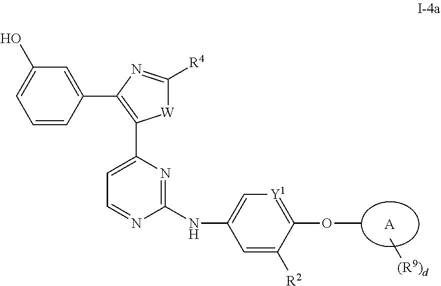

more particularly

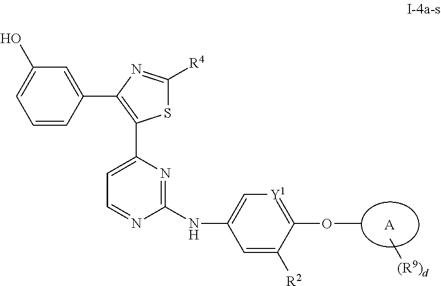

wherein all variables are as defined above.

Another preferred set of compounds of the invention is defined by formula (I-4-b):

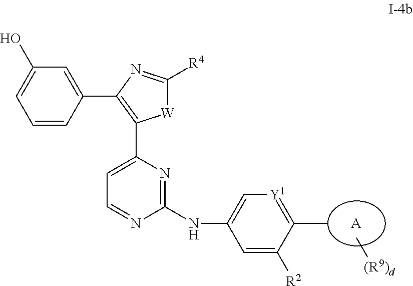

more particularly

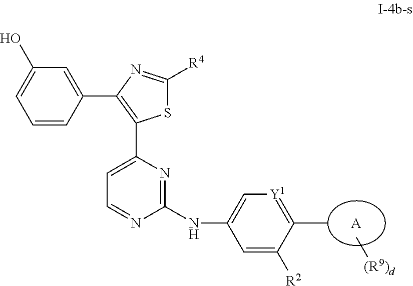

wherein all variables are as defined above.

Another preferred set of compounds of the invention is defined by formula (I-4c):

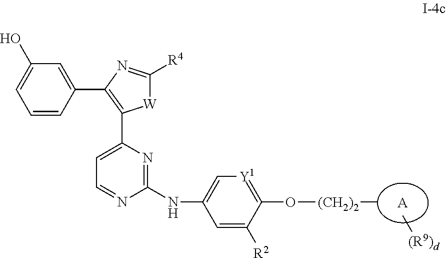

more particularly

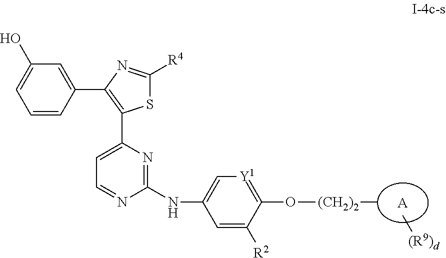

wherein all variables are as defined above.

Another preferred set of compounds of the invention is defined by the formula (I-5):

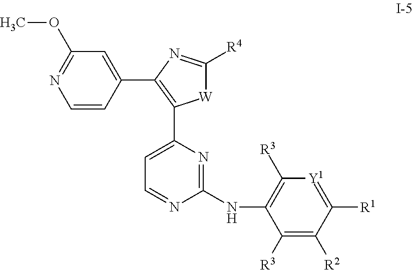

more particularly

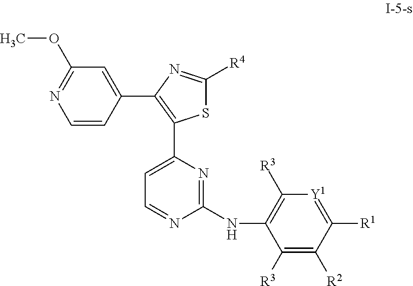

wherein all variables are as defined above.

In one embodiment, the present invention provides compounds of formula (I):

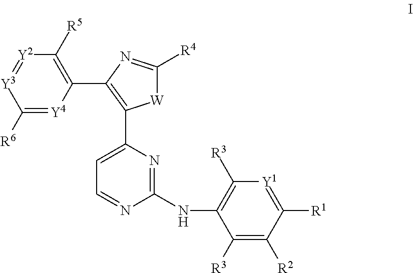

wherein:
R¹ is a moiety i, ii or iii:

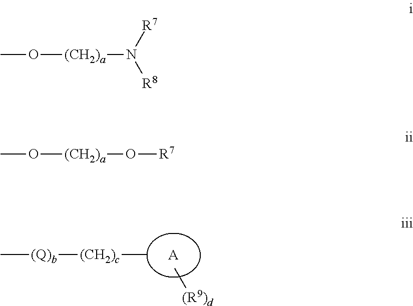

wherein:
a is 2, 3 or 4;
R⁷ and R⁸ are the same or different and are each independently selected from H, alkyl, haloalkyl, alkenyl, alkynyl, C₃₋₆cycloalkyl and C₃₋₆cycloalkenyl;
b is 0 or 1;
Q is selected from —O—, —N(H)— and —N(alkyl)-;
c is 0, 1, 2 or 3;
Ring A is a 4-10 membered N-heterocycle optionally having 1 or 2 additional heteroatoms selected from N, O and S, or 5-10 membered N-heteroaryl optionally having 1 or 2 additional heteroatoms selected from N, O and S;
d is 0, 1 or 2;
each R⁹ is the same or different and is independently selected from halo, alkyl, alkenyl, alkynyl, haloalkyl, oxo, OR¹⁰, R¹²—OR¹⁰, C(O)R¹⁰, CO₂R¹⁰,
   C(O)₂-benzyl, CONR¹⁰R₁₁, COR¹²—NR¹⁰R¹¹, COR¹²—OR¹⁰, NR¹⁰R¹¹, N(R¹⁰)C(S)N(R¹¹), N(R¹⁰)S(O)₂R¹¹, N(R¹⁰)C(O)N(R¹¹), N(R¹⁰)C(S)N(R¹¹), S(O)₃H, R¹²—S(O)₃H, S(O)₂R¹⁰, R¹²—S(O)₂R¹⁰, S(O)₂NR¹⁰R¹¹, CN and R¹²—CN;
R² is selected from H, halo, alkyl, haloalkyl, OR¹⁰, CO₂R¹⁰, NR¹⁰R¹¹, S(O)₂R¹⁰, CN and
   5-6 membered N-heterocycle optionally having 1 additional heteroatom selected from N, O and S and optionally substituted 1 or 2 times with alkyl or oxo; or
R¹ and R², together with the aromatic ring to which they are bound form a 9 or 10-membered fused, bicyclic heteroaryl group having 1, 2 or 3 heteroatoms selected from N, O and S, wherein said fused bicyclic heteroaryl group is optionally substituted 1 or 2 times with R⁹, and Y¹ is N or CH;
one R³ is H and the other R³ is H, halo, alkyl, OH or O-alkyl;
Y¹ is N or C—R^{b}, wherein R^{b} is selected from H, halo, alkyl, haloalkyl, OR¹⁰, CO₂R¹⁹, NR¹⁰R¹¹, S(O)₂R¹⁰ and CN;
W is O or S;
R⁴ is selected from H, alkyl, alkylene-OH, R¹²—SO₂NR¹³R¹⁴, NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴ wherein said N-linked heterocycle optionally includes 1 or 2 additional heteroatoms selected from N, O and S, and wherein said N-linked heterocycle is optionally substituted 1 or 2 times with a substituent selected from alkyl, oxo,
   O-alkyl, OH, alkylene-OH, NH₂, N(H)alkyl and N(alkyl)₂;
   e is 2, 3 or 4;
   each R¹³ and each R¹⁴ is the same or different and is each independently selected from H, alkyl, haloalkyl, and C₃₋₆cycloalkyl;
Y² is N or R⁶—C;
Y³ is N or R^{a}—C;
Y⁴ is N or R⁵—C;
wherein not more than one of Y², Y³ and Y⁴ is N;
each R⁵ is the same or different and is selected from H, halo and alkyl, wherein when Y⁴ is R⁵—C, at least one R⁵ is H;
R^{a} is selected from H, halo, alkyl, haloalkyl, alkylene-OH and —OR¹⁰;
each R⁶ is the same or different and is independently selected from H, halo, alkyl, alkenyl, alkynyl, haloalkyl, alkylene-OH, OR¹⁰ and NR¹⁰R¹¹, wherein at least one R⁶ is not H;
or R⁶ and R^{a} together with the aromatic ring to which they are bonded form naphthyl or a 9 or 10-membered fused bicyclic heteroaryl ring having 1, 2 or 3 heteroatoms selected from N, O and S, wherein said naphthyl or fused bicyclic heteroaryl ring is optionally substituted 1 or 2 times with an additional substituent selected from alkyl, oxo, O-alkyl, OH, alkylene-OH, NH₂, N(H)alkyl and N(alkyl)₂;
each R¹⁰ and each R¹¹ is the same or different and is independently selected from H, alkyl and haloalkyl; and
each R¹² is the same or different and is independently C₁₋₄alkylene;
   and pharmaceutically acceptable salts thereof.

It is to be understood that the present invention includes all combinations and subsets of the particular and preferred definitions of variables and subformulas described above.

Specific examples of compounds of the present invention include those recited in the Examples which follow as well as pharmaceutically acceptable salts of compounds exemplified as the free base and free base versions and other pharmaceutically acceptable salts of those compounds exemplified as salts.

Preferred compounds of formula (I) include but are not limited to:
N-[6-(4-Acetyl-1-piperazinyl)-3-pyridinyl]-4-[4-[3,5-bis(methyloxy)phenyl]-2-(ethylamino)-1,3-thiazol-5-yl]-2-pyrimidinamine;
4-[4-[3,5-Bis(methyloxy)phenyl]-2-(ethylamino)-1,3-thiazol-5-yl]-N-[3-fluoro-4-(4-morpholinyl)phenyl]-2-pyrimidinamine;
4-[4-[3,5-Bis(methyloxy)phenyl]-2-(ethylamino)-1,3-thiazol-5-yl]-N-[3-fluoro-4-({1-[2-(methylsulfonyl)ethyl]-4-piperidinyl}oxy)phenyl]-2-pyrimidinamine;
4-{2-(Ethylamino)-4-[3-methyl-5-(methyloxy)phenyl]-1,3-thiazol-5-yl}-N-(3-fluoro-4-{4-[2-(methylsulfonyl)ethyl]-1-piperazinyl}phenyl)-2-pyrimidinamine;
4-{2-(Ethylamino)-4-[3-methyl-5-(methyloxy)phenyl]-1,3-thiazol-5-yl}-N-[3-fluoro-4-(4-morpholinyl)phenyl]-2-pyrimidinamine;
4-{2-(Ethylamino)-4-[3-methyl-5-(methyloxy)phenyl]-1,3-thiazol-5-yl}-N-{3-fluoro-4-[4-(2-fluoroethyl)-1-piperazinyl]phenyl}-2-pyrimidinamine;
1-Acetyl-N-(4-{2-(ethylamino)-4-[3-methyl-5-(methyloxy)phenyl]-1,3-thiazol-5-yl}-2-pyrimidinyl)-2,3-dihydro-1H-indol-5-amine;
N-[6-(1,1-Dioxido-4-thiomorpholinyl)-3-pyridinyl]-4-{2-(ethylamino)-4-[3-methyl-5-(methyloxy)phenyl]-1,3-thiazol-5-yl}-2-pyrimidinamine;
4-{2-(Ethylamino)-4-[3-methyl-5-(methyloxy)phenyl]-1,3-thiazol-5-yl}-N-[6-(4-morpholinyl)-3-pyridinyl]-2-pyrimidinamine;
N-[6-(4-Acetyl-1-piperazinyl)-3-pyridinyl]-4-{2-(ethylamino)-4-[3-methyl-5-(methyloxy)phenyl]-1,3-thiazol-5-yl}-2-pyrimidinamine;
4-{2-(Ethylamino)-4-[3-methyl-5-(methyloxy)phenyl]-1,3-thiazol-5-yl}-N-(6-{4-[(methyloxy)acetyl]-1-piperazinyl}-3-pyridinyl)-2-pyrimidinamine;
4-[5-(2-{[6-(4-Acetyl-1-piperazinyl)-3-pyridinyl]amino}-4-pyrimidinyl)-2-ethyl-1,3-thiazol-4-yl]-2-(methyloxy)phenyl]methanol;
4-{2-(1-Methylethyl)-4-[3-methyl-5-(methyloxy)phenyl]-1,3-thiazol-5-yl}-N-[6-(4-morpholinyl)-3-pyridinyl]-2-pyrimidinamine;
N-[6-(4-Acetyl-1-piperazinyl)-3-pyridinyl]-4-{2-(1-methylethyl)-4-[3-methyl-5-(methyloxy)phenyl]-1,3-thiazol-5-yl}-2-pyrimidinamine;
N-({5-(2-{[6-(4-Acetyl-1-piperazinyl)-3-pyridinyl]amino}-4-pyrimidinyl)-4-[3-methyl-5-(methyloxy)phenyl]-1,3-thiazol-2-yl}methyl)methanesulfonamide; and
N-[6-(4-Acetyl-1-piperazinyl)-3-pyridinyl]-4-{2-(ethylamino)-4-[3-methyl-5-(methyloxy)phenyl]-1,3-oxazol-5-yl}-2-pyrimidinamine;
   and pharmaceutically acceptable salts thereof.

Particular preferred compounds of formula (I) include but are not limited to:
N-[6-(4-Acetyl-1-piperazinyl)-3-pyridinyl]-4-[4-[3,5-bis(methyloxy)phenyl]-2-(ethylamino)-1,3-thiazol-5-yl]-2-pyrimidinamine;
4-[4-[3,5-Bis(methyloxy)phenyl]-2-(ethylamino)-1,3-thiazol-5-yl]-N-[3-fluoro-4-({1-[2-(methylsulfonyl)ethyl]-4-piperidinyl}oxy)phenyl]-2-pyrimidinamine;
4-{2-(Ethylamino)-4-[3-methyl-5-(methyloxy)phenyl]-1,3-thiazol-5-yl}-N-(3-fluoro-4-{4-[2-(methylsulfonyl)ethyl]-1-piperazinyl}phenyl)-2-pyrimidinamine;
4-{2-(Ethylamino)-4-[3-methyl-5-(methyloxy)phenyl]-1,3-thiazol-5-yl}-N-[3-fluoro-4-(4-morpholinyl)phenyl]-2-pyrimidinamine;
4-{2-(Ethylamino)-4-[3-methyl-5-(methyloxy)phenyl]-1,3-thiazol-5-yl}-N-[6-(4-morpholinyl)-3-pyridinyl]-2-pyrimidinamine;
N-[6-(4-Acetyl-1-piperazinyl)-3-pyridinyl]-4-{2-(ethylamino)-4-[3-methyl-5-(methyloxy)phenyl]-1,3-thiazol-5-yl}-2-pyrimidinamine;
4-{2-(1-Methylethyl)-4-[3-methyl-5-(methyloxy)phenyl]-1,3-thiazol-5-yl}-N-[6-(4-morpholinyl)-3-pyridinyl]-2-pyrimidinamine;
N-[6-(4-Acetyl-1-piperazinyl)-3-pyridinyl]-4-{2-(1-methylethyl)-4-[3-methyl-5-(methyloxy)phenyl]-1,3-thiazol-5-yl}-2-pyrimidinamine; and
N-[6-(4-Acetyl-1-piperazinyl)-3-pyridinyl]-4-{2-(ethylamino)-4-[3-methyl-5-(methyloxy)phenyl]-1,3-oxazol-5-yl}-2-pyrimidinamine;
   and pharmaceutically acceptable salts thereof.

Particularly preferred compounds of formula (I) are selected from
N-[6-(4-Acetyl-1-piperazinyl)-3-pyridinyl]-4-[4-[3,5-bis(methyloxy)phenyl]-2-(ethylamino)-1,3-thiazol-5-yl]-2-pyrimidinamine;
4-[4-[3,5-Bis(methyloxy)phenyl]-2-(ethylamino)-1,3-thiazol-5-yl]-N-[3-fluoro-4-({1-[2-(methylsulfonyl)ethyl]-4-piperidinyl}oxy)phenyl]-2-pyrimidinamine;
4-{2-(Ethylamino)-4-[3-methyl-5-(methyloxy)phenyl]-1,3-thiazol-5-yl}-N-(3-fluoro-4-{4-[2-(methylsulfonyl)ethyl]-1-piperazinyl}phenyl)-2-pyrimidinamine; and
N-[6-(4-Acetyl-1-piperazinyl)-3-pyridinyl]-4-{2-(ethylamino)-4-[3-methyl-5-(methyloxy)phenyl]-1,3-oxazol-5-yl}-2-pyrimidinamine;
   and pharmaceutically acceptable salts thereof.

It will be appreciated by those skilled in the art that the compounds of formula (I) may be utilized as a pharmaceutically acceptable salt thereof. The pharmaceutically acceptable salts of the compounds of formula (I) include conventional salts formed from pharmaceutically acceptable (i.e., non-toxic) inorganic or organic acids or bases as well as quaternary ammonium salts. Representative salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, such as oxalic or trifluoroacetic acid salts, which are not themselves pharmaceutically acceptable, may be useful in the preparation of salts useful as intermediates in obtaining compounds of this invention and these form a further aspect of the invention. In one embodiment, the compound of formula (I) is in the form of the hydrochloride salt.

Processes for preparing pharmaceutically acceptable salts of compounds such as the compounds of formula (I) are conventional in the art. See, e.g., Burger's Medicinal Chemistry And Drug Discovery 5th Edition, Vol 1: Principles And Practice.

As will be apparent to those skilled in the art, in the processes described below for the preparation of compounds of formula (I), certain intermediates, may be in the form of pharmaceutically acceptable salts of the compound. Processes for preparing pharmaceutically acceptable salts of intermediates are known in the art and are analogous to the processes for preparing pharmaceutically acceptable salts of other compounds such as the compounds of formula (I).

Compounds of the invention are believed to inhibit one or more kinases and in particular one or more Raf family kinases (“Raf inhibitor”). Compounds of the invention may also inhibit one or more other kinases, and particularly tyrosine kinases. Certain compounds of the invention inhibit B-Raf (“B-Raf inhibitor”). It is well documented that Raf inhibitors, including B-Raf inhibitors, are believed to be useful as anticancer and antitumor agents. See, e.g., Davies (2002) supra, Garnett (2004) supra, and Zebisch (2006) supra. The anticancer and antitumor effects of these kinase inhibitors is currently believed to result from inhibition of one or more Raf family kinases, and the effect of such inhibition on cell lines whose growth and/or viability is dependent on the kinase activity of Raf family kinases. Compounds of the invention may be Raf inhibitors and also inhibit one or more ErbB family kinases (i.e., EGFR, ErbB2 and ErbB4). Certain compounds of the invention may inhibit B-Raf and also inhibit one or more ErbB family kinases (i.e., EGFR, ErbB2 and ErB4).

Some compounds of the invention may be selective inhibitors of Raf family kinases (“selective Raf inhibitor”), meaning that preferential inhibition of one or more Raf family kinases is significantly greater than that of any number of other kinases, for example by a factor of 5-fold or more.

However, the present invention is not limited to compounds which are selective inhibitors of one or more Raf family kinases rather, the present invention expressly contemplates that certain compounds of the invention may possess activity against multiple kinases, including kinases other than Raf family kinases. For example, particular compounds of the invention may possess activity against multiple other kinases, including but not limited to IGF-1R, IR, IRR, Src, VEGFR, PDGFR, Met, Lyn, Lck, Alk5, Aurora A and B, JNK, Syk, p38, BTK, FAK, Abl, CK1, cKit, Epherin receptors (for example EphB4), FGFR, Flt, Fyn, Hck, JAK, MLK, PKCμ, Ret, Yes, and BRK, as well. Particular compounds of the invention may be deemed to be unselective or non-selective, meaning that they are not considered by one skilled in the art to be selective for any particular kinase over others.

As used herein, a Raf inhibitor is a compound that inhibits one or more Raf family kinases and particularly a Raf inhibitor is a compound that exhibits a pIC₅₀ of greater than about 6 against at least one Raf family kinase in the Raf inhibition enzyme assay described below and/or an IC₅₀ of not greater than about 5 μM potency against at least one cell line that expresses mutated B-Raf kinase (e.g., A375P, Colo205, HT-29, SK-MEL-3, SK-MEL-28) in the cellular proliferation assay described below. In a particular embodiment, a Raf inhibitor refers to a compound of the invention that exhibits a pIC₅₀ of greater than about 6.5 against at least one Raf family kinase in the Raf inhibition enzyme assay described below and an IC₅₀ of not greater than about 500 nM potency against at least one cell line that expresses mutated B-Raf kinase in the cellular proliferation assay described below.

A “B-Raf inhibitor” refers to a compound that inhibits B-Raf and particularly a B-Raf inhibitor is a compound that exhibits a pIC₅₀ of greater than about 6.5 against B-Raf in the Raf inhibition enzyme assay described below and an IC₅₀ of not greater than about 500 nM potency against at least one cell line that expresses mutated B-Raf kinase in the cellular proliferation assay described below. A compound need to be selective for B-Raf to be considered a “B-Raf inhibitor.”

The present invention provides compounds for use in medical therapy in a mammal, e.g., a human, in need thereof. The present invention provides methods for the treatment of several conditions in a mammal in need thereof, all of which comprise the step of administering a therapeutically effective amount of a compound of the invention. All methods described herein are applicable to mammals, and particularly to humans. As used herein, the term “treatment” or “treating” in the context of therapeutic methods, refers to alleviating the specified condition, eliminating or reducing the symptoms of the condition, slowing or eliminating the progression, invasion, or metastatic spread of the condition and preventing or delaying the reoccurrence of the condition in a previously afflicted subject. The present invention further provides use of the compounds of the invention for the preparation of a medicament for the treatment of several conditions in a mammal (e.g., human) in need thereof.

More particularly, the present invention provides compounds for use in the treatment of a condition mediated by at least one Raf family kinase (e.g., B-Raf) in a mammal in need thereof. The present invention provides a method for treating a condition mediated by at least one Raf family kinase (e.g., B-Raf) in a mammal (e.g., a human) in need thereof, which method comprises administering to the mammal a therapeutically effective amount of the compound of the invention.

In another embodiment, the invention provides compounds for use in regulating, modulating, binding or inhibiting one or more Raf family kinases (e.g., B-Raf) in a mammal. The invention also provides methods of regulating, modulating, binding, or inhibiting at least one Raf family kinase (e.g., B-Raf) by administering a therapeutically effective amount of a compound of the invention. “Regulating, modulating, binding or inhibiting at least one Raf family kinase” refers to regulating, modulating, binding or inhibiting the activity of at least one Raf family kinase, as well as regulating, modulating, binding or inhibiting overexpression of an upstream regulator of at least one Raf family kinase in order to inhibit the cellular potency of its signaling ability.

In a particular embodiment, the invention provides compounds for use in the treatment of a condition mediated by inappropriate activity of one or more Raf family kinases (e.g., B-Raf), or an upstream activator of one or more Raf family kinases in a mammal. The invention further provides methods for the treatment of a condition mediated by inappropriate activity of one or more Raf family kinases (particularly B-Raf), in a mammal in need thereof, comprising administering to the mammal a therapeutically effective amount of a compound of the invention. In an additional aspect, the present invention provides the use of a compound of the invention for the preparation of a medicament for the treatment of a condition mediated by inappropriate activity of one or more Raf family kinases (particularly B-Raf), in a mammal. One example of a condition mediated by inappropriate activity of one or more Raf family kinases includes neoplasms.

By “inappropriate activity” is meant Raf family kinase activity that deviates from the expected activity for that kinase or for an upstream activator of that kinase in a particular mammal. The inappropriate activity of a Raf family kinase may arise from one or more of A-Raf, B-Raf or c-Raf or an upstream activator of a Raf family kinase. Inappropriate Raf family kinase activity may take the form of, for instance, an abnormal increase in activity, or an aberration in the timing and/or control of Raf family kinase activity. Such inappropriate activity may result, for example, from overexpression or mutation of the kinase, upstream activator, receptor or ligand leading to inappropriate or uncontrolled activation of the corresponding kinase or receptor. Furthermore, it is also contemplated that unwanted Raf family kinase activity may reside in an abnormal source, such as a neoplasm. Thus, the level of Raf family kinase activity does not need to be abnormal to be considered inappropriate in the case where the activity derives from an abnormal source including, but not limited to, upstream activators (e.g., activated mutant Ras GTPases) or neoplasm. In one example of inappropriate Raf family kinase activity not resulting from mutation or overexpression of a Raf family kinase, inappropriate activity of a Ras GTPase may result from mutation or overexpression of Ras GTPase, for example the G13D mutation in KRas2, and may lead to overactivation of the MAPK pathway mediated by Raf family kinase activity.

Thus, in one embodiment, the present invention provides compounds for use in the treatment of a condition which directly or indirectly results from a mutation of a Raf family kinase or overexpression of a Raf family kinase, or a mutation of an upstream activator of a Raf family kinase or overexpression of an upstream activator of a Raf family kinase in a mammal in need thereof. The present invention provides methods for the treatment of a condition which directly or indirectly results from mutation of a Raf family kinase or overexpression of a Raf family kinase, or a mutation of an upstream activator of a Raf family kinase or overexpression of an upstream activator of a Raf family kinase in a mammal in need thereof, comprising administering to the mammal a therapeutically effective amount of a compound of the invention. In an additional aspect, the present invention provides the use of a compound of the invention for the preparation of a medicament for the treatment of a condition which directly or indirectly results from mutation of a Raf family kinase or overexpression of a Raf family kinase, or a mutation of an upstream activator of a Raf family kinase or overexpression of an upstream activator of a Raf family kinase in a mammal. Conditions which are mediated by at least one Raf family kinase, and particularly conditions mediated by inappropriate activity of one or more Raf family kinases, including those which directly or indirectly result from mutation of a Raf family kinase, overexpression of a Raf family kinase, or mutation of an upstream activator of a Raf family kinase or overexpression of an upstream activator of a Raf family kinase are known in the art and include but are not limited to neoplasms.

Compounds of the invention may also be used in the treatment of conditions attenuated by inhibition of a Raf family kinase (particularly B-Raf). Further provided are methods for treating a condition attenuated by inhibition of a Raf family kinase (particularly B-Raf) in a mammal in need thereof, comprising administering to the mammal a therapeutically effective amount of a compound of the invention. Also provided is the use of a compound of the invention for the preparation of a medicament for the treatment of a condition attenuated by inhibition of a Raf family kinase (particularly B-Raf) in a mammal. Conditions attenuated by inhibition of a Raf family kinase (including B-Raf) include but are not limited to neoplasms.

Accordingly, compounds of the invention may be used in the treatment of a neoplasm, particularly a susceptible neoplasm (a cancer or tumor) in a mammal. The present invention also provides a method for treating a neoplasm, particularly a susceptible neoplasm in a mammal in need thereof, which method comprises administering to the mammal a therapeutically effective amount of the compound of the invention. The invention also provides the use of a compound of the invention for the preparation of a medicament for the treatment of neoplasm, particularly a susceptible neoplasm, in a mammal. “Susceptible neoplasm” as used herein refers to neoplasms which are susceptible to treatment by a kinase inhibitor and particularly neoplasms that are susceptible to treatment by a Raf inhibitor. Neoplasms which have been associated with inappropriate activity of one or more Raf family kinases and particularly neoplasms which exhibit mutation of a Raf family kinase, overexpression of a Raf family kinase, or mutation of an upstream activator of a Raf family kinase or overexpression of an upstream activator of a Raf family kinase, and are therefore susceptible to treatment with an Raf inhibitor are known in the art, and include both primary and metastatic tumors and cancers. See, Catalogue of Somatic Mutations in Cancer (COSMIC), the Wellcome Trust Sanger Institute, http://www.sanger.ac.ukigenetics/CGP/cosmic/ and those references cited in the background.

Specific examples of susceptible neoplasms within the scope of the invention include, but are not limited to:
Barret's adenocarcinoma;
billiary tract carcinomas;
breast cancer;
cervical cancer;
cholangiocarcinoma;
central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system),
colorectal cancer, including large intestinal colon carcinoma;
gastric cancer;
carcinoma of the head and neck including squamous cell carcinoma of the head and neck;
hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia;
hepatocellular carcinoma;
lung cancer including small cell lung cancer and non-small cell lung cancer;
ovarian cancer;
endometrial cancer;
pancreatic cancer;
pituitary adenoma;
prostate cancer;
renal cancer;
sarcoma;
skin cancers including melanomas; and
thyroid cancers.

Accordingly, in one embodiment, the present invention provides a method for the treatment of any one or more of the aforementioned neoplasms in a mammal in need thereof, the method comprising administering a therapeutically effective amount of a compound of the invention to the mammal.

The present invention also provides the a compound of formula (I) for use in the treatment of Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers, or any subset thereof, in a mammal in need thereof.

The present invention further provides the use of a compound of formula (I) for the preparation of a medicament for the treatment of Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers, or any subset thereof, in a mammal in need thereof.

As is well known in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As used herein, reference to uses or methods for treatment for “neoplasm,” “tumor” or “cancer” in a subject includes both use for and treatment of the primary neoplasm, tumor or cancer, and where appropriate, also the use for the treatment of metastases (i.e., metastatic tumor growth) as well.

In one particular embodiment, the invention provides a method for treating breast cancer in a mammal in need thereof, comprising administering to the mammal a therapeutically effective amount of a compound of the invention. In one particular embodiment, the invention provides a method for treating colorectal cancer in a mammal in need thereof, comprising administering to the mammal a therapeutically effective amount of a compound of the invention. In one particular embodiment, the invention provides a method for treating melanoma in a mammal in need thereof, comprising administering to the mammal a therapeutically effective amount of a compound of the invention. In one particular embodiment, the invention provides a method for treating non-small cell lung cancer in a mammal in need thereof, comprising administering to the mammal a therapeutically effective amount of a compound of the invention. In one particular embodiment, the invention provides a method for treating ovarian cancer in a mammal in need thereof, comprising administering to the mammal a therapeutically effective amount of a compound of the invention. In one particular embodiment, the invention provides a method for treating thyroid cancer in a mammal in need thereof, comprising administering to the mammal a therapeutically effective amount of a compound of the invention.

In one particular embodiment, the susceptible neoplasm is breast cancer and the invention provides compounds for use in the treatment of breast cancer in a mammal and the use of such compounds for the preparation of a medicament for the treatment of breast cancer in a mammal. In another embodiment, the susceptible neoplasm is colorectal cancer and the invention provides compounds for use in the treatment of colorectal cancer in a mammal and the use of such compounds for the preparation of a medicament for the treatment of colorectal cancer in a mammal. In another embodiment, the susceptible neoplasm is melanoma, and the invention provides compounds for use in the treatment of melanoma in a mammal and the use of such compounds for the preparation of a medicament for the treatment of melanoma in a mammal. In another embodiment, the susceptible neoplasm is non-small cell lung cancer, and the invention provides compounds for use in the treatment of non-small cell lung cancer in a mammal and the use of such compounds for the preparation of a medicament for the treatment of non-small cell lung cancer in a mammal. In another embodiment, the susceptible neoplasm is ovarian cancer and the invention provides compounds for use in the treatment of ovarian cancer in a mammal and the use of such compounds for the preparation of a medicament for the treatment of ovarian cancer in a mammal. In another embodiment, the susceptible neoplasm is thyroid cancer, and the invention provides compounds for use in the treatment of thyroid cancer in a mammal and the use of such compounds for the preparation of a medicament for the treatment of thyroid cancer in a mammal.

The compounds of the invention can be used alone in the treatment of each of the foregoing conditions or can be used to provide additive or potentially synergistic effects with certain existing chemotherapies, radiation, biological or immunotherapeutics (including monoclonal antibodies) and vaccines. The compounds of the invention may be useful for restoring effectiveness of certain existing chemotherapies and radiation and or increasing sensitivity to certain existing chemotherapies and/or radiation.

In addition to the treatment of susceptible neoplasms, the compounds of the invention may also be used in the treatment of other conditions attenuated by inhibition of a Raf family kinase, such as cardio-facio cutaneous syndrome and polycystic kidney disease.

As used herein, the term “therapeutically effective amount” means an amount of a compound of the invention which is sufficient, in the subject to which it is administered, to elicit the biological or medical response of a cell culture, tissue, system, mammal (including human) that is being sought, for instance, by a researcher or clinician. The term also includes within its scope amounts effective to enhance normal physiological function. For example, a therapeutically effective amount of a compound of the invention for the treatment of a condition mediated by at least one Raf family kinase is an amount sufficient to treat the condition in the particular subject. Similarly, a therapeutically effective amount of a compound of the invention for the treatment of a susceptible neoplasm is an amount sufficient to treat the particular susceptible neoplasm in the subject. In one embodiment of the present invention, a therapeutically effective amount of a compound of the invention is an amount sufficient to regulate, modulate, bind or inhibit at least one Raf family kinase. More particularly, in such embodiment, the therapeutically effective amount of a compound of the invention is an amount sufficient to regulate, modulate, bind or inhibit B-Raf.

The precise therapeutically effective amount of the compounds of the invention will depend on a number of factors. There are variables inherent to the compounds including, but not limited to, the following: molecular weight, inhibitory activity at the target kinase, absorption, bioavailability, distribution in the body, tissue penetration, half-life, metabolism, protein binding, and excretion. These variables determine what dose of compound needs to be administered in order to inhibit the target kinase by a sufficient percentage and for a sufficient amount of time to have the desired effect on the tumor. In general, the goal will be to inhibit the target kinase by 50% or more for as long as possible. The duration of drug exposure will be limited only by the compound half-life, and side effects from treatment requiring cessation of dosing. The amount of compound administered will also depend on factors related to patients and disease including, but not limited to, the following: the age, weight, concomitant medications, and medical condition of the subject being treated, the precise condition requiring treatment and its severity, the nature of the formulation, and the route of administration. Ultimately the dose will be at the discretion of the attendant physician or veterinarian. Typically, the compound of the invention will be given for treatment in the range of 0.01 to 30 mg/kg body weight of recipient (mammal) per day and more usually in the range of 0.1 to 10 mg/kg body weight per day. Thus, for a 70 kg adult human being treated for a condition mediated by at least one Raf family kinase, the actual amount per day would usually be from 1 to 2000 mg and this amount may be given in a single or multiple doses per day. Dosing regimens may vary significantly and will be determined and altered based on clinical experience with the compound. The full spectrum of dosing regimens may be employed ranging from continuous dosing (with daily doses) to intermittent dosing. A therapeutically effective amount of a pharmaceutically acceptable salt of a compound of formula (I) may be determined as a proportion of the therapeutically effective amount of the compound of formula (I) per se (e.g., as a free base or acid). It is envisaged that similar dosages would be appropriate for treatment of the susceptible neoplasms described above.

While it is possible that, for use in therapy, a therapeutically effective amount of a compound of the invention may be administered as the raw chemical, it is typically presented as the active ingredient of a pharmaceutical composition or formulation. Accordingly, the invention further provides a pharmaceutical composition comprising a compound of the invention. The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the invention with one or more pharmaceutically acceptable carriers, diluents and/or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the invention (as a free-base, solvate (including hydrate) or salt, in any form), depending on the condition being treated, the route of administration, the bioavailability of the specific compound, the species being treated, and the age, weight and condition of the patient. Preferred unit dosage formulations are those containing a daily dose, weekly dose, monthly dose, a sub-dose, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including capsules, tablets, liquid-filled capsules, disintegrating tablets, immediate, delayed and controlled release tablets, oral strips, solutions, syrups, buccal and sublingual), rectal, nasal, inhalation, topical (including transdermal), vaginal or parenteral (including subcutaneous, intramuscular, intravenous or intradermal) route. Such formulations may be prepared by any method known in the art of pharmacy, for example by bringing into association the active ingredient with the carrier(s), excipient(s) or diluent. Generally, the carrier, excipient or diluent employed in the pharmaceutical formulation is “non-toxic,” meaning that it/they is/are deemed safe for consumption in the amount delivered in the pharmaceutical composition, and “inert” meaning that it/they does/do not appreciably react with or result in an undesired effect on the therapeutic activity of the active ingredient.

Pharmaceutical formulations adapted for oral administration may be presented as discrete units such as liquid-filled or solid capsules; immediate, delayed or controlled release tablets; powders or granules; solutions or suspensions in aqueous or non-aqueous liquids; edible foams or whips; oil-in-water liquid emulsions, water-in-oil liquid emulsions or oral strips, such as impregnated gel strips.

For instance, for oral administration in the form of a tablet or capsule, the active drug component can be combined with an oral, pharmaceutically acceptable carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent may also be present.

Solid capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Solutions and syrups can be prepared by dissolving the compound in a suitably flavored aqueous solution, while elixirs are prepared through the use of a pharmaceutically acceptable alcoholic vehicle. Suspensions can be formulated by dispersing the compound in a pharmaceutically acceptable vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, unit dosage formulations for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropyl-methacrylamidephenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polylacetic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research (1986) 3(6):318.

Pharmaceutical formulations adapted for topical administration may be formulated as ointments, creams, suspensions, lotions, powders, solutions, pastes, gels, sprays, aerosols or oils. For treatments of external tissues, such as skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical formulations adapted for topical administrations to the eye include eye drops wherein the active ingredient is dissolved or suspended in a suitable carrier, especially an aqueous solvent. Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, metered dose inhalers, dry powder inhalers, nebulizers or insufflators.

Pharmaceutical formulations adapted for vaginal administration may be presented as pessaries, tampons, creams, gels, pastes, foams or spray formulations. Pharmaceutical formulations adapted for parenteral administration include aqueous and non-aqueous sterile injection solutions which may contain anti-oxidants, buffers, bacteriostats and solutes which render the formulation of pharmaceutically acceptable tonicity with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

In the above-described methods of treatment and uses, a compound of the invention may be employed alone, in combination with one or more other compounds of the invention or in combination with other therapeutic methods or agents. In particular, in methods of treating a condition improved by inhibition of at least one Raf family kinase and in methods of treating susceptible neoplasms, combination with other chemotherapeutic, biologic, hormonal, antibody and supportive care agents is envisaged as well as combination with surgical therapy and radiotherapy. Supportive care agents include analgesics, anti-emetics, and agents used to treat heamatologic side effects such as neutropenia. Analgesics are well known in the art. Anti-emetics include but are not limited to 5HT₃ antagonists such as ondansetron, granisetron, dolasetron, palonosetron and the like; prochlorperazine, metaclopromide, diphenhydramine, promethazine; dexamethasone, lorazepam; haloperidol, dronabinol, olanzapine; and neurokinin-1 antagonists such as aprepitant, fosaprepitant and casopitant administered alone or in various combinations.

The term “chemotherapeutic” as used herein refers to any chemical agent having a therapeutic effect on the subject to which it is administered. “Chemotherapeutic” agents include but are not limited to anti-neoplastic agents. As used herein, “anti-neoplastic agents” include both cytotoxic and cytostatic agents including biological, immunological and vaccine therapies. Combination therapies according to the invention thus comprise the administration of at least one compound of the invention and the use of at least one other treatment method. In one embodiment, combination therapies according to the invention comprise the administration of at least one compound of the invention and surgical therapy. In one embodiment, combination therapies according to the invention comprise the administration of at least one compound of the invention and radiotherapy. In one embodiment, combination therapies according to the invention comprise the administration of at least one compound of the invention and at least one supportive care agent (e.g., at least one anti-emetic agent). In one embodiment, combination therapies according to the present invention comprise the administration of at least one compound of the invention and at least one other chemotherapeutic agent. In one particular embodiment, the invention comprises the administration of at least one compound of the invention and at least one anti-neoplastic agent.

As an additional aspect, the present invention provides the methods of treatment and uses as described above, which comprise administering a compound of the invention together with at least one chemotherapeutic agent. In one particular embodiment, the chemotherapeutic agent is an anti-neoplastic agent. In another embodiment, the invention provides a pharmaceutical composition as described above further comprising at least one other chemotherapeutic agent, more particularly, the chemotherapeutic agent is an anti-neoplastic agent. The invention also provides methods of treatment and uses as described above, which comprise administering a compound of the invention together with at least one supportive care agent (e.g., anti-emetic agent).

The compounds of the invention and at least one additional anti-neoplastic or supportive care therapy may be employed in combination concomitantly or sequentially in any therapeutically appropriate combination. The administration of a compound of the invention with one or more other anti-neoplastic agents may be in combination in accordance with the invention by administration concomitantly in (1) a unitary pharmaceutical composition including both or all compounds or (2) separate pharmaceutical compositions each including one or more of the compounds. The components of the combination may be administered separately in a sequential manner wherein one active ingredient is administered first and the other(s) second or vice versa. Such sequential administration may be close in time or remote in time.

When a compound of the invention is used in combination with an anti-neoplastic and/or supportive care agent, the dose of each compound may differ from that when the compound is used alone. Appropriate doses will be readily appreciated by those skilled in the art. The appropriate dose of the compound(s) of the invention and the other therapeutically active agent(s) and the relative timings of administration will be selected in order to achieve the desired combined therapeutic effect, and are within the expertise and discretion of the attendant clinician.

Typically, any chemotherapeutic agent that has activity against a susceptible neoplasm being treated may be utilized in combination with the compounds the invention, provided that the particular agent is clinically compatible with therapy employing a compound of the invention. Typical anti-neoplastic agents useful in the present invention include, but are not limited to: alkylating agents, anti-metabolites, antitumor antibiotics, antimitotic agents, topoisomerase I and II inhibitors, hormones and hormonal analogues; signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Alkylating agents may be employed in combination with the compounds of the invention in the compositions and methods described above. Examples of alkylating agents include but are not limited to: nitrogen mustards such as cyclophosphamides, temozolamide, melphalan, and chlorambucil; oxazaphosphorines; alkyl sulfonates such as busulfan; nitrosoureas such as carmustine; triazenes such as dacarbazine; and platinum coordination complexes such as cisplatin, oxaliplatin and carboplatin.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. The end result of discontinuing S phase is cell death. Antimetabolite neoplastic agents may be employed in combination with the compounds of the invention in the compositions and methods described above. Examples of antimetabolite anti-neoplastic agents include but are not limited to purine and pyrimidine analogues and anti-folate compounds, and more specifically, hydroxyurea, cytosine, arabinoside, ralitrexed, tegafur, fluorouracil (e.g., 5FU), methotrexate, cytarabine, mercaptopurine and thioguanine.

Antitumor antibiotic agents are non-phase specific agents, which bind to or intercalate with DNA. Typically, such action disrupts ordinary function of the nucleic acids, leading to cell death. Antitumor antibiotics may be employed in combination with the compounds of the invention in the compositions and methods described above. Examples of antitumor antibiotic agents include, but are not limited to, actinomycins such as dactinomycin; anthracyclines such as daunorubicin, doxorubicin, idarubicin, epirubicin and mitoxantrone; mitomycin C and bleomycins.

Antimicrotubule or antimitotic agents are phase specific agents active against the microtubules of tumor cells during M or the mitosis phase of the cell cycle. Antimitotic agents may be employed in combination with the compounds of the invention in the compositions and methods described above. Examples of antimitotic agents include, but are not limited to, diterpenoids, vinca alkaloids, polo-like kinase (PLK) inhibitors and CenpE inhibitors. Examples of diterpenoids include, but are not limited to, paclitaxel and its analog docetaxel. Examples of vinca alkaloids include, but are not limited to, vinblastine, vincristine, vindesine and vinorelbine. PLK inhibitors are discussed further below.

Topoisomerase inhibitors include inhibitors of Topoisomerase II and inhibitors of Topoisomerase I. Topoisomerase II inhibitors, such as epipodophyllotoxins are anti-neoplastic agents derived from the mandrake plant, that typically affect cells in the S and G₂ phases of the cell cycle by forming a ternary complex with topoisomerase II and DNA, causing DNA strand breaks. The strand breaks accumulate and cell death follows. Examples of epipodophyllotoxins include, but are not limited to, etoposide and teniposide. Camptothecins, including camptothecin and camptothecin derivatives are available or under development as Topoisomerase I inhibitors. Examples of camptothecins include, but are not limited to amsacrine, irinotecan, topotecan, and the various optical forms of 7-(4-methylpiperazino-methylene)-10,11-ethylenedioxy-20-camptothecin. Topoisomerase inhibitors may be employed in combination with the compounds of the invention in the compositions and methods described above.

Hormones and hormonal analogues are useful compounds for treating cancers in which there is a relationship between the hormone(s) and growth and/or lack of growth of the cancer. Antitumor hormones and hormonal analogues may be employed in combination with the compounds of the invention in the compositions and methods described above. Examples of hormones and hormonal analogues believed to be useful in the treatment of neoplasms include, but are not limited to antiestrogens, such as tamoxifen, toremifene, raloxifene, fulvestrant, iodoxyfene and droloxifene; anti-androgens; such as flutamide, nilutamide, bicalutamide and cyproterone acetate; adrenocorticosteroids such as prednisone and prednisolone; aminoglutethimide and other aromatase inhibitors such as anastrozole, letrazole, vorazole, and exemestane; progestrins such as megestrol acetate; 5α-reductase inhibitors such as finasteride and dutasteride; and gonadotropin-releasing hormones (GnRH) and analogues thereof, such as Leutinizing Hormone-releasing Hormone (LHRH) agonists and antagonists such as goserelin luprolide, leuprorelin and buserelin.

Signal transduction pathway inhibitors are those inhibitors which block or inhibit a chemical process which evokes an intracellular change. As used herein this change is cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3-OH kinases, myoinositol signaling, and Ras oncogenes. Signal transduction pathway inhibitors may be employed in combination with the compounds of the invention in the compositions and methods described above.

Several protein tyrosine kinases catalyze the phosphorylation of specific tyrosine residues in various proteins involved in the regulation of cell growth. Such protein tyrosine kinases can be broadly classified as receptor or non-receptor kinases.

Receptor tyrosine kinase inhibitors which may be combined with the compounds of the invention include those involved in the regulation of cell growth, which receptor tyrosine kinases are sometimes referred to as “growth factor receptors.” Examples of growth factor receptor inhibitors, include but are not limited to inhibitors of: insulin growth factor receptors (IGF-1R, IR and IRR); epidermal growth factor family receptors (EGFR, ErbB2, and ErbB4); platelet derived growth factor receptors (PDGFRs), vascular endothelial growth factor receptors (VEGFRs), tyrosine kinase with immunoglobulin-like and epidermal growth factor homology domains (TIE-2), macrophage colony stimulating factor (c-fms), c-kit, c-met, fibroblast growth factor receptors (FGFRs), hepatocyte growth factor receptors (HGFRs), Trk receptors (TrkA, TrkB, and TrkC), ephrin (Eph) receptors and the RET protooncogene.

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-erbB2 antibody inhibitor of growth factor function. One example of an anti-erbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb™) and erlotinib (TARCEVA®). Imatinib (GLEEVEC®) is one example of a PDGFR inhibitor. Examples of VEGFR inhibitors include pazopanib, ZD6474, AZD2171, PTK787, sunitinib and sorafenib.

In one embodiment, the invention provides methods of treatment of any of the various conditions enumerated above comprising administering a compound of the invention in combination with an EGFR or erbB inhibitor. In one particular embodiment, the methods of the present invention comprise administering a compound of the invention in combination with lapatinib. In one particular embodiment, the methods of the present invention comprise administering a compound of the invention in combination with trastuzumab. In one particular embodiment, the methods of the present invention comprise administering a compound of the invention in combination with erlotinib. In one particular embodiment, the methods of the present invention comprise administering a compound of the invention in combination with gefitinib.

In another embodiment, the present invention provides methods of treatment of any of the various conditions enumerated above comprising administering a compound of the invention in combination with a VEGFR inhibitor. In one particular embodiment, the methods of the present invention comprise administering a compound of the invention in combination with pazopanib.

Tyrosine kinases that are not transmembrane growth factor receptor kinases are termed non-receptor, or intracellular tyrosine kinases. Inhibitors of non-receptor tyrosine kinases are sometimes referred to as “anti-metastatic agents” and are useful in the present invention. Targets or potential targets of anti-metastatic agents, include, but are not limited to, c-Src, Lck, Fyn, Yes, Jak, Abl kinase (c-Abl and Bcr-Abl), FAK (focal adhesion kinase) and Bruton's tyrosine kinase (BTK). Non-receptor kinases and agents, which inhibit non-receptor tyrosine kinase function, are described in Sinha, S, and Corey, S. J., (1999) J. Hematother. Stem Cell Res. 8:465-80; and Bolen, J. B. and Brugge, J. S., (1997) Annu. Rev. of Immunol. 15:371-404.

SH2/SH3 domain blockers are agents that disrupt SH2 or SH3 domain binding in a variety of enzymes or adaptor proteins including, but not limited to, PI3-K p85 subunit, Src family kinases, adaptor molecules (Shc, Crk, Nck, Grb2) and Ras-GAP. Examples of Src inhibitors include but are not limited to dasatinib and BMS-354825 (J. Med. Chem. (2004) 47:6658-6661).

Inhibitors of serine/threonine kinases may also be used in combination with the compounds of the invention in any of the compositions and methods described above. Examples of serine/threonine kinase inhibitors that may also be used in combination with a compound of the present invention include, but are not limited to polo-like kinase inhibitors (Plk family e.g., Plk1, Plk2, and Plk3), which play critical roles in regulating processes in the cell cycle including the entry into and the exit from mitosis; MAP kinase cascade blockers, which include other Ras/Raf kinase inhibitors, mitogen or extracellular regulated kinases (MEKs), and extracellular regulated kinases (ERKs); Aurora kinase inhibitors (including inhibitors of Aurora A and Aurora B); protein kinase C (PKC) family member blockers, including inhibitors of PKC subtypes (alpha, beta, gamma, epsilon, mu, lambda, iota, zeta); inhibitors of kappa-B (IkB) kinase family (IKK-alpha, IKK-beta); PKB/Akt kinase family inhibitors; and inhibitors of TGF-beta receptor kinases. Examples of Plk inhibitors are described in PCT Publication No. WO04/014899 and WO07/03036 both to GlaxoSmithKline. Other examples of serine/threonine kinase inhibitors are known in the art. In another embodiment, the present invention provides methods of treatment of any of the various conditions enumerated above comprising administering a compound of the invention in combination with a Plk inhibitor. In one particular embodiment, the methods of the present invention comprise administering a compound of the invention in combination with 5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide.

Urokinase, also referred to as urokinase-type Plasminogen Activator (uPA), is a serine protease. Activation of the serine protease plasmin triggers a proteolysis cascade which is involved in thrombolysis or extracellular matrix degradation. Elevated expression of urokinase and several other components of the plasminogen activation system have been correlated with tumor malignancy including several aspects of cancer biology such as cell adhesion, migration and cellular mitotic pathways as well. Inhibitors of urokinase expression may be used in combination with the compounds of the invention in the compositions and methods described above.

Inhibitors of Ras oncogene may also be useful in combination with the compounds of the present invention. Such inhibitors include but are not limited to, inhibitors of farnesyltransferase, geranyl-geranyl transferase, and CAAX proteases as well as anti-sense oligonucleotides, ribozymes and immunotherapy. Such inhibitors have been shown to block Ras activation in cells containing mutant Ras, thereby acting as antiproliferative agents.

Inhibitors of kinases involved in the IGF-1R signaling axis may also be useful in combination with the compounds of the present invention. Such inhibitors include but are not limited to inhibitors of JNK1/2/3, PI3K, AKT and MEK, and 14.3.3 signaling inhibitors. Examples of AKT inhibitors are described in PCT Publication No. WO 2007/058850, published 24 May 2007 which corresponds to PCT Application No. PCT/US2006/043513, filed 9 Nov. 2006, to GlaxoSmithKline. One particular AKT inhibitor disclosed therein is 4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol.

Cell cycle signaling inhibitors, including inhibitors of cyclin dependent kinases (CDKs) are also useful in combination with the compounds of the invention in the compositions and methods described above. Examples of cyclin dependent kinases, including CDK2, CDK4, and CDK6 and inhibitors for the same are described in, for instance, Rosania G. R., et al., Exp. Opin. Ther. Patents (2000) 10:215-230.

Receptor kinase angiogenesis inhibitors may also find use in the present invention. Inhibitors of angiogenesis related to VEGFR and TIE-2 are discussed above in regard to signal transduction inhibitors (both are receptor tyrosine kinases). Other inhibitors may be used in combination with the compounds of the invention. For example, anti-VEGF antibodies, which do not recognize VEGFR (the receptor tyrosine kinase), but bind to the ligand; small molecule inhibitors of integrin (alpha, beta₃) that inhibit angiogenesis; endostatin and angiostatin (non-RTK) may also prove useful in combination with the compounds of the invention. One example of a VEGFR antibody is bevacizumab (AVASTIN®).

Inhibitors of phosphatidyl inositol-3-OH kinase family members including blockers of PI3-kinase, ATM, DNA-PK, and Ku may also be useful in combination with the present invention.

Also of potential use in combination with the compounds of the invention are myoinositol signaling inhibitors such as phospholipase C blockers and myoinositol analogues.

Antisense therapies may also be used in combination with the compounds of the invention. Examples of such antisense therapies include those directed towards the targets described above such as ISIS 2503 and gene therapy approaches such as those using thymidine kinase or cytosine deaminase.

Agents used in immunotherapeutic regimens may also be useful in combination with the compounds of the invention. Immunotherapeutic regimens include ex-vivo and in-vivo approaches to increasing immunogenicity of patient tumor cells such as transfection with cytokines (IL-2, IL-4, GMCFS and MCFS), approaches to increase T-cell activity, approaches with transfected immune cells and approaches with anti-idiotypic antibodies. Another potentially useful immunotherapeutic regimen is monoclonal antibodies with wild-type Fc receptors that may illicit an immune response in the host (e.g., IGF-1R monoclonal antibodies).

Agents used in proapoptotic regimens (e.g., Bcl-2 antisense oligonucleotides) may also be used in combination with the compounds of the invention. Members of the Bcl-2 family of proteins block apoptosis. Upregulation of Bcl-2 has therefore been linked to chemoresistance. Studies have shown that the epidermal growth factor (EGF) stimulates anti-apoptotic members of the Bcl-2 family (i.e., mcl-1). Therefore, strategies designed to downregulate the expression of Bcl-2 in tumors have demonstrated clinical benefit and are now in Phase II/III trials, namely Genta's G3139 bcl-2 antisense oligonucleotide. Such proapoptotic strategies using the antisense oligonucleotide strategy for Bcl-2 are discussed in Water, J. S., et al., J. Clin. Oncol. (2000) 18:1812-1823; and Kitada, S., et al., Antisense Res. Dev. (1994) 4:71-79.

Compounds of formula (I) may be prepared using the processes described below. In all of the schemes described below, it is understood that protecting groups may be employed where necessary in accordance with general principles known to those of skill in the art, for example, see Green, T. W. and Wuts, P. G. M. (1991) Protecting Groups in Organic Synthesis, John Wiley & Sons. The selection of a particular protecting group and processes for installation and removal of protecting groups is within the skill of those in the art. The selection of processes for installation and removal of protecting groups as well as the reaction conditions and order of their execution shall be consistent with the preparation of compounds of formula (I).

Compounds of formula (I) may be conveniently prepared by the methods outlined in Scheme 1 below.

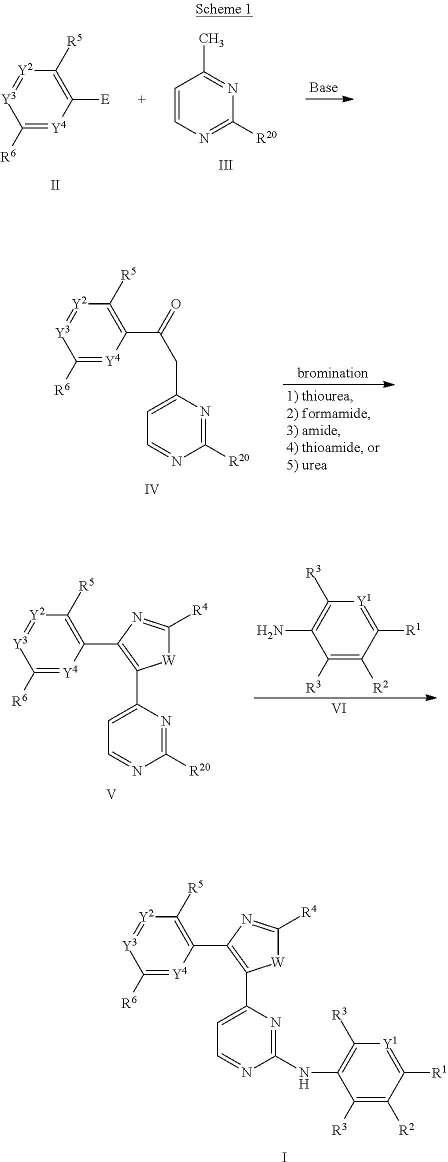

wherein:
R²⁰ is halo (preferably chloro) or thiomethyl;
E is a suitable carboxylic ester or ester equivalent, particularly a methyl ester, ethyl ester, or Weinreb's amide; and
all other variables are as defined above.

Generally, the process for preparing the compounds of formula (I) (all formulas and all variables having been defined above) comprises the step of: reacting a compound of formula (V) with an aniline of formula (VI) to prepare a compound of formula (I).

More specifically, the process for preparing compounds of formula (I) comprises the steps of:
a) condensing the compound of formula (II) with a substituted pyrimidine compound of formula (III) to prepare a compound of formula (IV);
b) reacting the compound of formula (IV) with a suitable brominating agent followed by one of:
   i) a thiourea,
   ii) a formamide,
   iii) an amide,
   iv) a thioamide, or
   v) a urea;
   to prepare a compound of formula (V);
c) reacting the compound of formula (V) with an aniline of formula (VI) to prepare a compound of formula (I);
d) optionally converting the compound of formula (I) to a pharmaceutically acceptable salt thereof; and
e) optionally converting the compound of formula (I) or a pharmaceutically acceptable salt thereof to a different compound of formula (I) or a pharmaceutically acceptable salt thereof.

As will be apparent to those skilled in the art, the order of the foregoing steps is not critical to the process of the present invention, and the process may be carried out using any suitable order of steps.

Compounds of formula (I) are prepared by reacting a compound of formula (V) with an aniline of formula (VI). wherein all variables are as defined above.

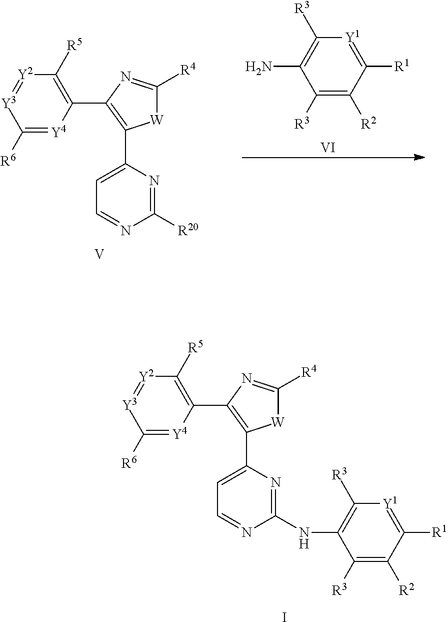

wherein all variables are as defined above.

Those skilled in the art will recognize that the conditions required for the above reaction will differ depending upon the definition of R²⁰. When R²⁰ is halo (preferably chloro), the reaction is generally performed in a solvent. Suitable solvents include but are not limited to isopropanol, 1,4-dioxane, ethanol, dimethylacetamide, trifluoroethanol, and N,N-dimethylformamide. The reaction is typically carried out under reflux conditions or in a microwave apparatus at a temperature of from about 90° C. to about 220° C., preferably from about 160° C. to about 190° C. As will be apparent to those skilled in the art of organic chemistry, it may be desirable to catalyze this reaction for the preparation of certain compounds of formula (I). For example, it may be desirable to carry out the reaction in the presence of a catalytic amount of an acid such as hydrochloric acid, hydrobromic acid, or paratoluenesulfonic acid. As will further be apparent to those skilled in the art, it may also be desirable to install appropriate protecting groups prior to reacting the compound of formula (V) with the compound of formula (VI). For example, in the embodiment wherein R⁶ is an OH, the addition is preferably carried out when the phenol is protected as, for example, its corresponding paramethoxybenzyl ether. Also, in the embodiment wherein R¹ or R² of a compound of formula (VI) contains a primary or secondary amine, the addition is preferably carried out when the amine is protected as, for example, its corresponding trifluoroacetamide or benzylcarbamate. The choice, installation and removal of appropriate protecting groups for reactions such as this is conventional in the art. Compounds of formula (VI) are commercially available or may be synthesized using techniques conventional in the art.

When R²⁰ is thiomethyl, the thiomethyl may first be converted to a more suitable leaving group, for example sulfoxide, sulfone, or chloride. The thiomethyl can be converted into a sulfoxide or sulfone by oxidation with an appropriate oxidizing agent, for example oxone, sodium periodate, or meta-chloroperbenzoic acid, in an appropriate solvent, for example dichloromethane, methanol, or water. Those skilled in the art will recognize that this will produce an analogue of the compound of formula (V) in which R²⁰ is a sulfoxide or sulfone. The oxidized product can then be reacted with an aniline of formula (VI) to prepare a compound of formula (I).

These reactions are generally performed in a suitable solvent, for example 2-propanol, dimethylacetamide, or dioxane, optionally with the addition of acid, for example hydrochloric acid, and at a temperature of 25-110° C., preferably 70-90° C., or in a microwave reactor at a temperature of 90-220° C., preferably 160-190° C.

Alternately, the pyrimidinyl sulfoxide or sulfone can be converted to the corresponding hydroxyl pyrimidine by reaction with an appropriate aqueous acid, for example hydrochloric acid or acetic acid, at a temperature of 25-110° C., preferably 70-90° C. The hydroxyl pyrimidine can then be converted to a chloride using an appropriate chlorinating reagent, for example phosphorous oxychloride or thionyl chloride, optionally in a solvent, for example dichloromethane, at a temperature of 25-120° C., preferably 60-80° C. Those skilled in the art will recognize that this process will produce a compound of formula (V) wherein R²⁰ is chloro, which can be reacted with an aniline of formula (VI) as described above.

Compounds of formula (V) may be prepared by reacting a compound of formula (IV) with a suitable brominating reagent, particularly bromine or NBS, followed by reacting with one of: 1) a thiourea, 2) a formamide 3) an amide 4) a thioamide or 5) a urea depending upon whether the thiazole or oxazole, and which particular substituent R⁴, is desired.

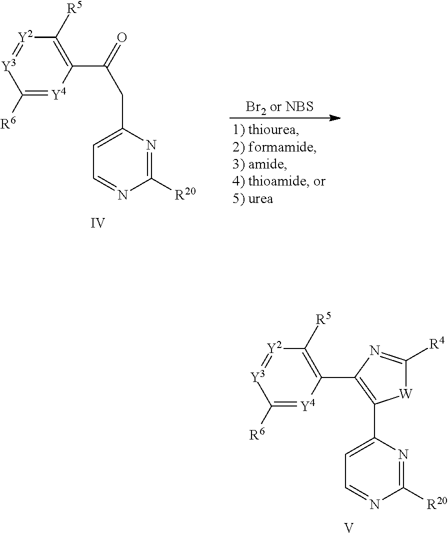

wherein all variables are as defined above.

The thiourea, formamide, amide, thioamide or urea may be substituted with the desired group R⁴. In this and subsequent Schemes, reference to thiourea, formamide, amide, thioamide or urea in connection with this type of reaction refers to unsubstituted thiourea, formamide, amide, thioamide or urea and substituted analogs thereof. In particular, the thiourea, formamide, amide, thioamide or urea may be substituted with the desired group R⁴. Suitably substituted analogs of thiourea, formamide, amide, thioamide or urea are commercially available or may be prepared using conventional techniques.

When an aminothiazole (i.e., the compound of formula (V) wherein W is S and R⁴ is selected from NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, N(R¹³)phenyl, and 5-6 membered N-linked heterocycle) is desired, the reaction can be accomplished by the initial bromination of a compound of formula (IV) using an appropriate brominating reagent, for example bromine or N-bromosuccinimide.

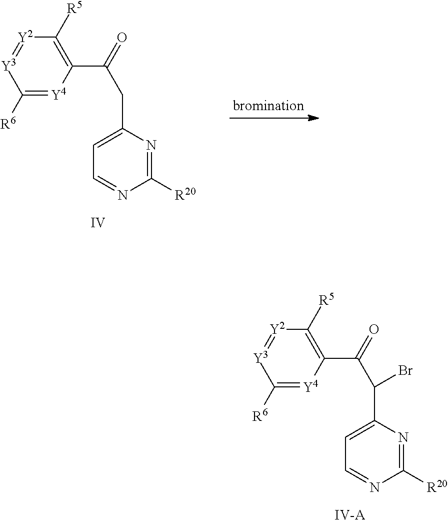

wherein all variables are as defined above.

The reaction is typically carried out in an appropriate solvent, for example dichloromethane or acetic acid, and at a temperature of 25-50° C., particularly 25° C. The brominated analog (i.e., the compound of formula (IV-A) is then reacted with an appropriately substituted thiourea.

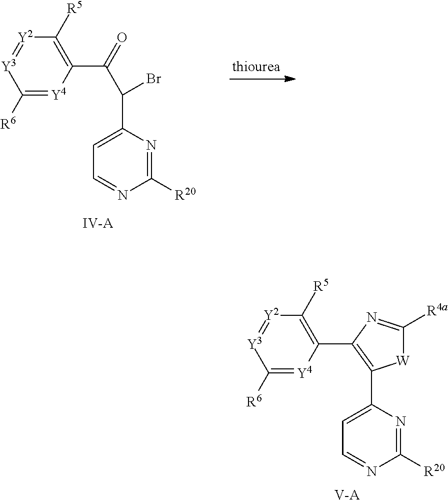

wherein W is S and R^{4a} is selected from NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, N(R¹³)phenyl, and 5-6 membered N-linked heterocycle;
and all other variables are as defined above.

The reaction is typically carried out in an appropriate solvent, for example, dichloromethane, THF, dioxane, or acetonitrile, optionally in the presence of a suitable base, for example magnesium carbonate or sodium bicarbonate, and at a temperature of 25-90° C., particularly 25-50° C. Those skilled in the art will recognize that the thiourea can be unsubstituted, thus resulting in a compound of formula (V-A) wherein R⁴ is NH₂; or the thiourea may bear one or more additional substituents on one of the nitrogen atoms, for example as in N-[2-(4-morpholinyl)ethyl]thiourea.

In this and subsequent reactions, a compound, such as a compound of formula (V), wherein R⁴ is an amino group (or substituted amino), may be further converted to a corresponding compound wherein R⁴ is other than amino (or substituted amino) using the techniques described herein and those conventional in the art. For example, the aminothiazole compound of formula (V-A) prepared according to the preceding description may be converted to an unsubstituted thiazole (i.e., a compound of formula (V) wherein R⁴ is H) using methods familiar to those of skill in the art. For example, the thiazole may be prepared by reacting the aminothiazole with an appropriate reagent, for example t-butyl nitrite, in an appropriate solvent, for example THF, and at a temperature of 35-75° C., particularly 40-60° C.

When a substituted thiazole is desired, an aminothiazole of formula (V-A) may be modified according to methods that will be familiar to those skilled in the art. For example, the aminothiazole compound of formula (V-A) may be converted to a compound of formula (V-B) by reaction with reagents capable of replacing the amino group with a halide, preferably a bromide.

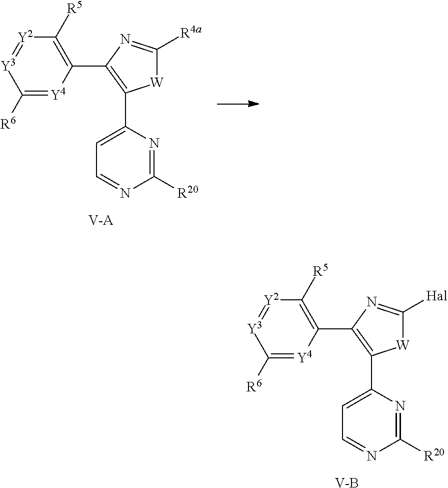

wherein Hal is halo, preferably Br; and all other variables are as defined above.

The conversion to a halo-thiazole of formula (V-B) may be carried out by reaction with for example, t-butyl nitrite and copper (II) bromide in a suitable solvent, such as tetrahydrofuran or acetonitrile, and at a temperature from −10° C. to 50° C., preferably 0° C. to 25° C. The halo-thiazole of formula (V-B), may then be reacted under a variety of conditions known to those in the art to produce different thiazole compounds of formula (V) wherein R⁴ can be a variety of substituents consistent with the definition of R⁴ in reference to compounds of Formula (I).

One example of such a reaction is similar to the method of J. Tsuji “Palladium Reagents and Catalysts: Innovations in Organic Synthesis”, Wiley, Chichester, UK, 1995, involving reaction of the halo-thiazole of formula (V-B) with a reagent capable of undergoing palladium-based coupling to prepare compounds of formula (V-C) wherein R^{4c} is alkyl or hydroxyalkyl.

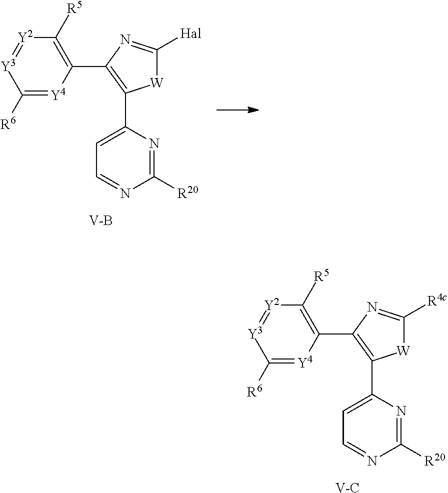

wherein Hal is halogen;
R^{4c} is alkyl, or hydroxyl alkyl; and
all other variables are as defined above.

For example the halo-thiazole of formula (V-B) may be reacted with a boronic acid, boronate ester, alkyl tin, alkyl zinc or Grignard reagent, in an appropriate solvent, for example tetrahydrofuran, dioxane, or dimethylformamide, in the presence of a catalyst capable of inducing such a transformation, particularly a palladium catalyst, for example palladiumdicholorobistriphenylphosphine, and at a temperature of 25-150° C., preferably 25-60° C. Those skilled in the art will recognize that these coupling reactions will often require the addition of a suitable base, such as aqueous sodium carbonate, cesium carbonate, or triethylamine and/or the addition of a suitable ligand for the palladium species, for example a trialkylphosphine or a triarylphosphine, for example triphenylphosphine. Those of skill in the art will also recognize that when the compound of formula (V-C) is desired wherein R^{4c} is an hydroxyalkyl, the alcohol may be protected, for example as the benzyl ether or pivolate ester. The choice, installation and removal of appropriate protecting groups for reactions such as this is conventional in the art.

Another example of such a reaction involves the reaction of the halo-thiazole of formula (V-B) with a reagent capable of displacing the bromide, for example an amine, such as piperidine, methylamine, methyl piperazine and anilines.

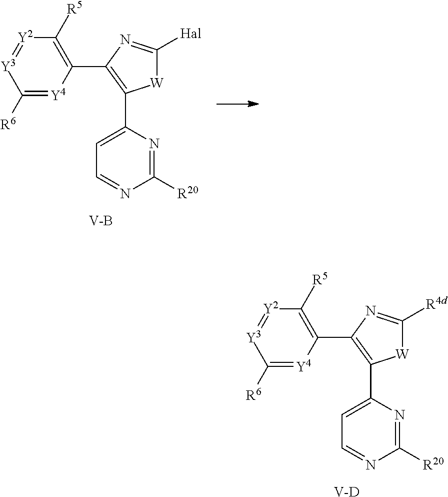

wherein Hal is halogen;
R^{4d} is selected from NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, N(R¹³)phenyl, and 5-6 membered N-linked heterocycle; and
all other variables are as defined above.

In the case of reacting a halo-thiazole of formula (V-B) with an amine, substituted amine (e.g., dimethylamine) or N-containing heterocycle (e.g., morpholine or N-methyl piperidine, the reaction is generally performed by reacting the compound of formula (V-B) with the amine, substituted amine or N-containing heterocycle, optionally in a suitable solvent, such as 2-propanol, dioxane, or dimethylformamide, at a temperature of 25° C. to 150° C., preferably 50-90° C., optionally in the presence of a suitable acid, for example hydrochloric acid.

According to another process of producing a substituted thiazole of formula (V), a compound of formula (IV-A) is reacted with a thioamide, for example thioacetamide, to prepare a compound of formula (V-E) wherein R^{4e} is selected from alkyl or alkylene-OH.

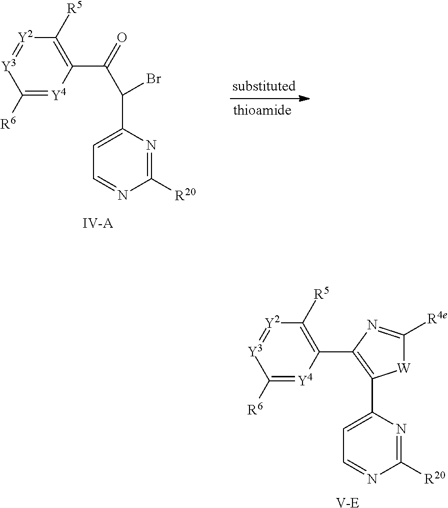

wherein all variables are as defined above.

Alkyl and hydroxyalkyl substituted thioamides for use in this process are commercially available or may be prepared using conventional techniques. Typically, the reaction is carried out in an appropriate solvent, for example, dichloromethane, tetrahydrofuran, dimethylformamide, or acetonitrile, particularly dimethylformamide, optionally in the presence of a suitable base, for example magnesium carbonate or sodium bicarbonate, and at a temperature of 35-100° C., preferably 50-70° C. Those of skill in the art will recognize that when the compound of formula (V-E) is desired wherein R^{4e} is alkylene-OH, the alcohol may be protected, for example as the benzyl ether or pivolate ester. The choice, installation and removal of appropriate protecting groups for reactions such as this is conventional in the art.

In the embodiment wherein an oxazole of formula (V) is desired wherein R⁴ is H, the reaction can be accomplished by reacting the compound of formula (IV-A) with formamide in the presence of an acid, such as sulfuric acid, and at a temperature of 60-150° C., preferably 100-130° C.

A substituted oxazole of formula (V-F) may be prepared from the compound of formula (IV-A).

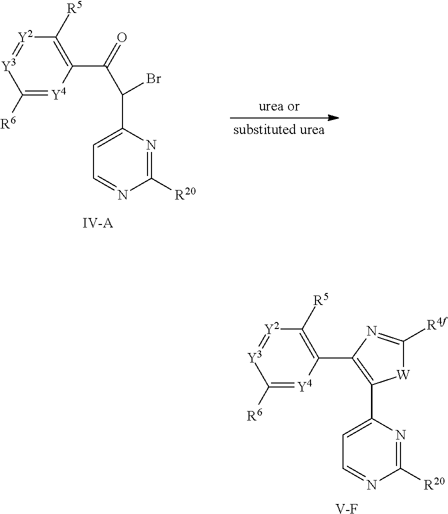

wherein R^{4f} is selected from NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, N(R¹³)phenyl, and 5-6 membered N-linked heterocycle; and
all other variables are as defined above.

The reaction may be carried out by reacting the compound of formula (IV-A) with a urea or substituted urea in an appropriate solvent, for example, dichloromethane, tetrahydrofuran, dioxane, or acetonitrile, optionally in the presence of a suitable base, for example magnesium carbonate or sodium bicarbonate, and at a temperature of 25-170° C., particularly 60-150° C. or in a microwave reactor at a temperature of 100-190° C., particularly 120-160° C. Those skilled in the art will envision substituted ureas that may be employed in the foregoing method to prepare compounds of formula (V-F) wherein R³ is as defined above. One example of a substituted urea for use in this method is N-[2-(4-morpholinyl)ethyl]urea. Suitable substituted ureas are commercially available or can be made using techniques known to those skilled in the art.

A substituted oxazole of formula (V-G), may also be prepared from a compound of formula (IV-A).

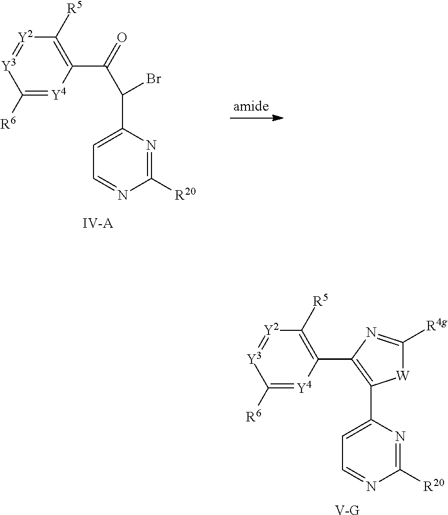

wherein R^{4g} is alkyl and all other variables are as defined above.

Typically, the reaction may be carried out by reacting the compound of formula (IV-A) with an amide (i.e., a compound of formula R^{4g}—C(O)NH₂), for example acetamide, in an appropriate solvent, for example, dichloromethane, tetrahydrofuran, dimethylformamide, or acetonitrile, particularly dimethylformamide, optionally in the presence of a suitable base, for example magnesium carbonate or sodium bicarbonate, and at a temperature of 35-170° C., preferably 60-150° C. or in a microwave reactor at a temperature of 100-190° C., particularly 130-170° C. Suitable amides for use in this reaction will be apparent to those skilled in the art and are commercially available or may be prepared using conventional techniques.

As will be appreciated by those skilled in the art a bromo-substituted oxazole of formula (V-H),

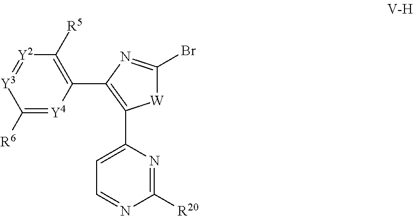

wherein all other variables are as defined above;
   may also be prepared by conversion of an oxazole of formula (V-F) (wherein R⁴ is an amine) to the bromo analog using techniques known to those of skill in the art, including those described above.

Those of skill in the art will recognize that some of the reactions described above may be incompatible with compounds of formula (V) in which R²⁰ is chloride. In such embodiments, the foregoing reactions may be performed using compounds of formula (V) wherein R²⁰ is thiomethyl, and subsequently converting the thiomethyl to a more suitable leaving group, such as a sulfoxide, sulfone or chloride using techniques conventional in the art, including those described above.

Compounds of formula (IV) may be prepared by reacting a compound of formula (II) with a substituted pyrimidine of formula (III).

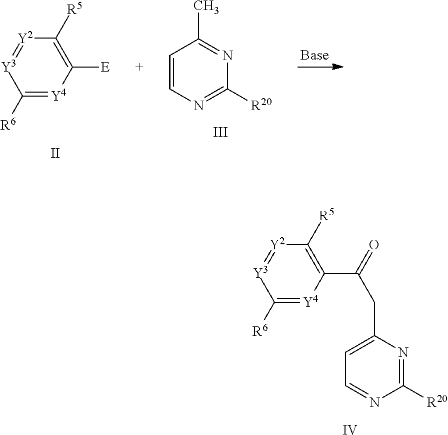

wherein all variables are as defined above.

These reactions are generally performed by reacting a compound of formula (II) and a compound of formula (III) in the presence of a suitable base capable of deprotonating a compound of formula (III), for example lithium hexamethyldisilazide (LHMDS), sodium hexamethyldisilazide (NaHMDS), or lithium diisopropylamide (LDA), particularly LHMDS, in an appropriate solvent, such as THF, and at a temperature of from about −78° C. to about 25° C., particularly about 0 to about 25° C. Those of skill in the art will recognize that when the compound of formula (IV) is desired wherein R⁶ is a hydroxy, the phenol may be protected, for example as the benzyl ether or paramethoxy benzyl ether. The choice, installation and removal of appropriate protecting groups for reactions such as this is conventional in the art.

The compound of formula (II) may be commercially available or may be prepared by methods known to those of skill. In some cases, a desired compound of formula (II) may be prepared by conversion from a different compound of formula (II) using conventional organic synthesis methods. The preparation of the compound of formula (II) may require modification of a commercially available or chemically accessible carboxylic acid of formula (VII).

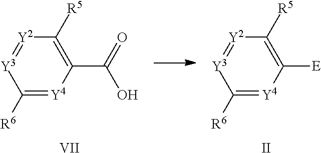

wherein all variables are as defined above.

Methods for preparing a compound of formula (II) from the carboxylic acid of formula (VII) are well known in the art. For example, when a compound of formula (II) wherein E is an ester, particularly a methyl or ethyl ester, is desired, a carboxylic acid of formula (VII) may be reacted with an appropriate alcohol, particularly methanol or ethanol in the presence of a suitable acid, for example hydrochloric acid or para toluenesulfonic acid. The reaction may be optionally performed in a suitable solvent, such as dichloromethane or tetrahydrofuran, and at a temperature of ambient temperature up to reflux.

A compound of formula (II) wherein E is a methyl ester may also be prepared by reacting a carboxylic acid of formula (VII) with an appropriate alkylating agent, particularly trimethyl silyl diazomethane or methyl iodide. The reaction is typically performed in a solvent, such as ether, tetrahydrofuran, or methanol at a temperature of 0° C. to reflux, optionally in the presence of a suitable base, for example potassium carbonate.

A compound of formula (II) wherein E is a Weinreb amide may be prepared by reacting a carboxylic acid of formula (VII) with N,O-dimethylhydroxyamine using conditions well known to those of skill in the art.

Carboxylic acids of formula (VII) are commercially available or may be prepared using methods known to those of skill. In some cases, a desired compound of formula (VII) may be prepared by conversion from a different compound of formula (VII) using conventional organic synthesis methods.

As noted above, the order of the foregoing steps is not critical to the practice of the present invention. For example, compounds of formula (I) may also be prepared according to Scheme 2.

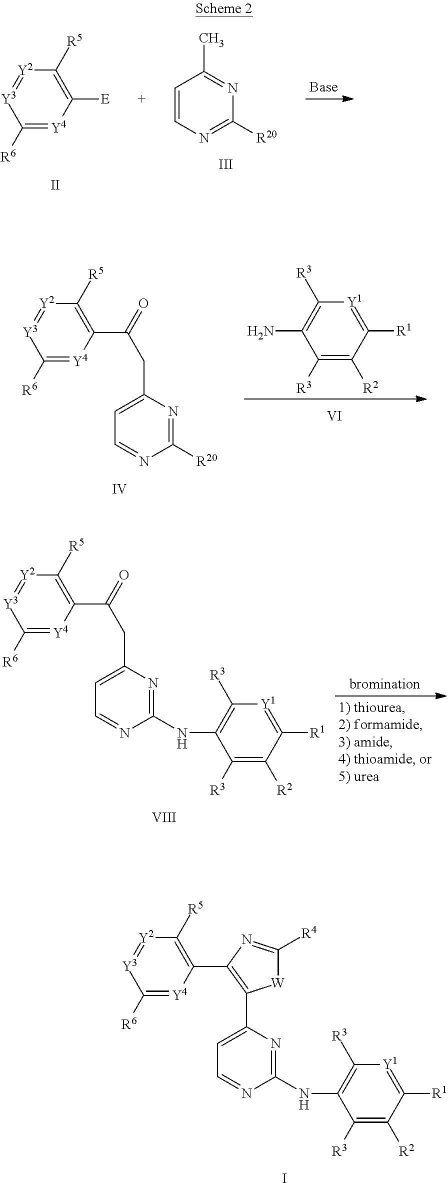

wherein:
R²⁰ is halo (preferably chloro) or thiomethyl; and
all other variables are as defined above.

In general, the process for preparing compounds of formula (I) according to Scheme 2 comprises the steps of:
a) reacting the compound of formula (VIII) with a suitable brominating agent followed by one of:
   a) a thiourea,
   b) a formamide,
   c) an amide,
   d) a thioamide, or
   e) a urea
      to prepare a compound of formula (I).

More specifically, a compound of formula (I) may be prepared by a process comprising the steps of:
a) condensing the compound of formula (II) with a substituted pyrimidine compound of formula (III) to prepare a compound of formula (IV);
b) reacting the compound of formula (V with an aniline of formula (VI) to prepare a compound of formula (VIII);
c) reacting the compound of formula (VIII) with a suitable brominating agent followed by one of:
   1) a thiourea,
   2) a formamide,
   3) an amide,
   4) a thioamide, or
   5) a urea;
   to prepare a compound of formula (I);
d) optionally converting the compound of formula (I) to a pharmaceutically acceptable salt thereof; and
e) optionally converting the compound of formula (I) or a pharmaceutically acceptable salt thereof to a different compound of formula (I) or a pharmaceutically acceptable salt thereof.

Compounds of formula (I) may be prepared by reacting a compound of formula (VIII) with a suitable brominating reagent, particularly bromine or NBS, followed by reacting with one of: 1) a thiourea, 2) a formamide 3) an amide 4) a thioamide or 5) a urea depending upon whether the thiazole or oxazole and which particular substituents R⁴, are desired. This reaction may be carried out in a manner similar to that described in Scheme 1 for the preparation of a compound of formula (V) using a compound of formula (IV).

Compounds of formula (VIII) may be prepared by reacting a compound of formula (IV) with an aniline of formula (VI). This reaction may be carried out in a manner similar to that described in Scheme 1 for the reaction of a compound of formula (V) with an aniline of formula (VI). Those of skill in the art will recognize that an additional equivalent of an aniline of formula (VI) may be required for this transformation and that this may produce an imine or enamine. This imine or enamine may be hydrolyzed to produce a compound of formula (VIII) using acidic conditions well known to those of skill.

As a further example, compounds of formula (I) may also be prepared according to Scheme 3.

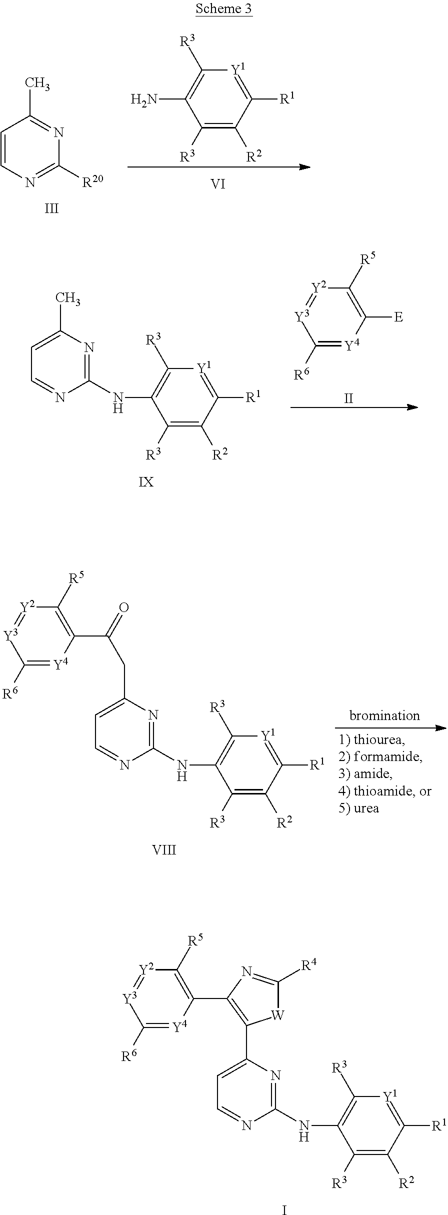

wherein:
R²⁰ is halo (preferably chloro) or thiomethyl; and
all other variables are as defined above.

In general, the process for preparing compounds of formula (I) according to Scheme 3 comprises the steps of:
a) reacting the compound of formula (III) with an aniline of formula (VI) to prepare a compound of formula (IX);
b) condensing the compound of formula (IX) with a compound of formula (II) to prepare a compound of formula (VIII)
c) reacting the compound of formula (VIII) with a suitable brominating agent followed by one of:
   1) a thiourea,
   2) a formamide,
   3) an amide,
   4) a thioamide, or
   5) a urea;
   to prepare a compound of formula (I);
d) optionally converting the compound of formula (I) to a pharmaceutically acceptable salt thereof; and
e) optionally converting the compound of formula (I) or a pharmaceutically acceptable salt thereof to a different compound of formula (I) or a pharmaceutically acceptable salt thereof.

The reaction of a compound of formula (VIII) with a brominating reagent followed by one of: 1) a thiourea, 2) a formamide 3) an amide 4) a thioamide or 5) a urea to prepare a compound of formula (I) is described above in Scheme 2.

According to this process, compounds of formula (VIII) may be prepared by condensing a compound of formula (II) with a compound of formula (IX) in the presence of a suitable base. This reaction may be carried out in a manner similar to that described above for the preparation of a compound of formula (IV) by the condensation of a substituted pyrimidine of formula (III) with a compound of formula (II).

Compounds of formula (IX) may be prepared by reacting a substituted pyrimidine of formula (III) with an aniline of formula (VI). The reaction may be carried out in a manner similar to the reaction of a compound of formula (V) with the aniline of formula (VI) as described above in Scheme 1.

It will be appreciated by those skilled in the art that the optimal choice of the reaction sequence employed to prepare a particular compound of formula (I) may depend upon the specific compound of formula (I) that is desired as well as the preference and availability of starting materials.

As will be apparent to those skilled in the art, a compound of formula (I) may be converted to another compound of formula (I) using techniques well known in the art. For example, compounds of formula (I) may be modified using conventional techniques to modify or diversify the groups defined by the variable R⁴ and there by provide different compounds of formula (I). Specifically, a compound of formula (I-11) (wherein R⁴ is —NH₂) may be converted to a compound of formula (I-12) by reductive amination of the amine with acetone and sodium cyanoborohydride.

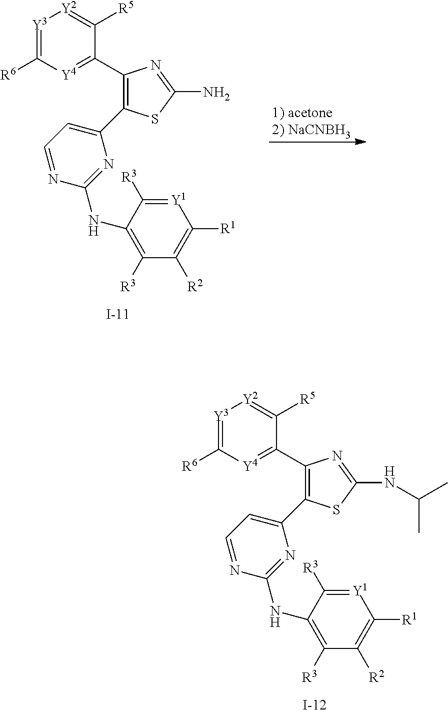

A compound of formula (I-11) may also be converted to a compound of formula (I-13) by reacting with mesyl chloride.

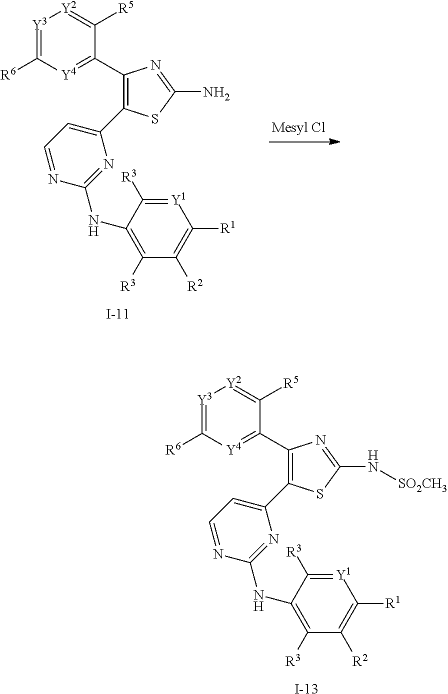

A specific example of this transformation is described below at Example 64

A compound of formula (I-14) may be converted to a compound of formula (I-15) by reacting with pyrrolidine.

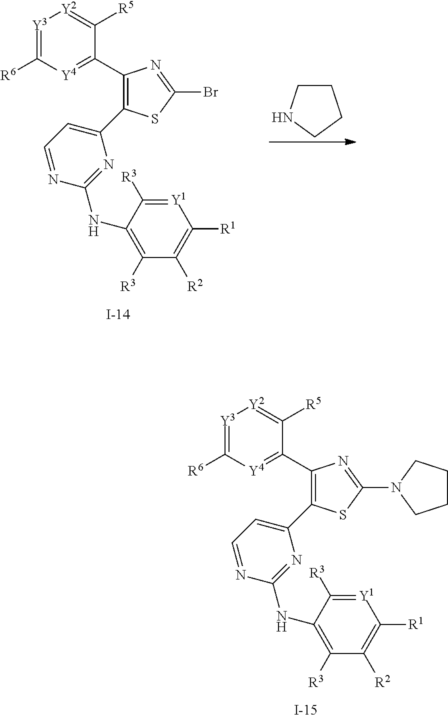

An ester compound of formula (I-16) may be converted to an alcohol compound of formula (I-17) by reacting with methanol and a suitable base such as sodium methoxide.

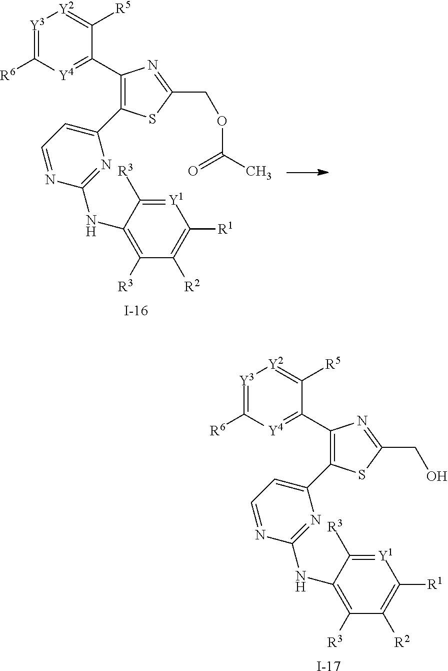

A specific example of this transformation is described below at Example 60.

Compounds of formula (I) may also be diversified in the position defined by R² using conventional techniques to convert a compound of formula (I) to a different compound of formula (I). For example, a compound of formula (I-18) may be converted to a compound of formula (I-19) by reacting with morpholine.

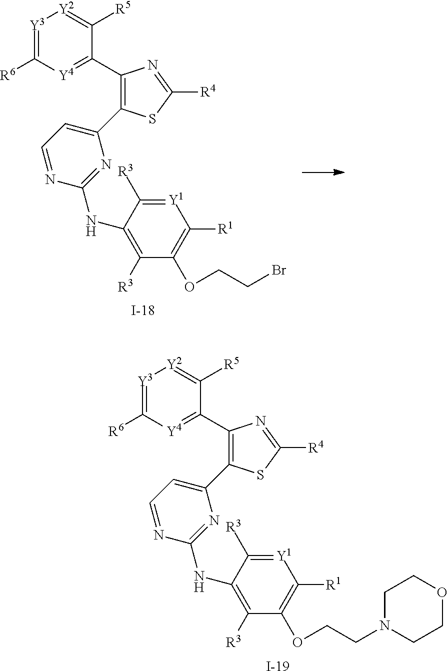

A specific example of this transformation is described below at Example 13.

A compound of formula (I-20) may be converted to a compound of formula (I-21) by reacting with acetic anhydride.

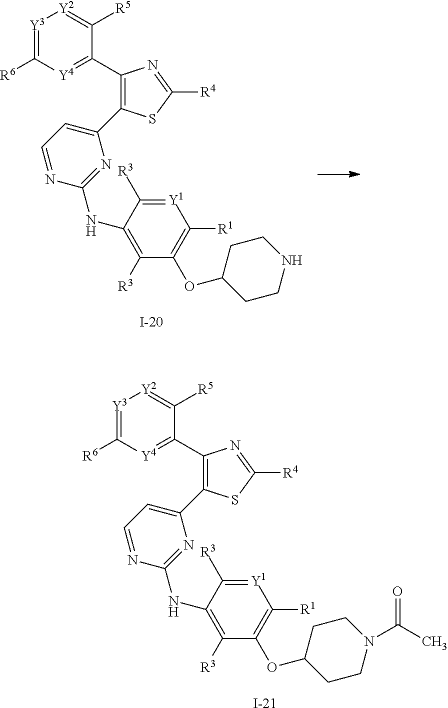

A specific example of this transformation is described below at Example 12. Compounds of formula (I) may also be diversified in the position defined by R⁶ using conventional techniques to convert a compound of formula (I) to a different compound of formula (I). For example, a compound of formula (I-22) may be converted to a compound of formula (I-23) using conventional dealkylation techniques such as reaction with boron tribromide.

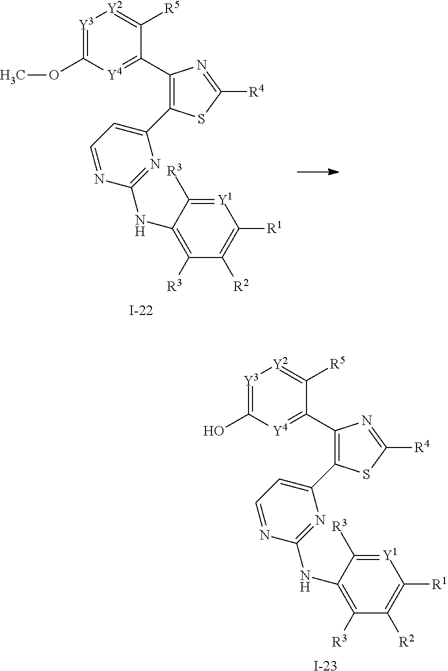

As another example, a compound of formula (I-24) may be converted to a compound of formula (I-23) using conventional techniques, including reduction with conventional hydrogenation techniques, oxidation with a suitable reagent such as DDQ, or reaction with an acid such as HCl.

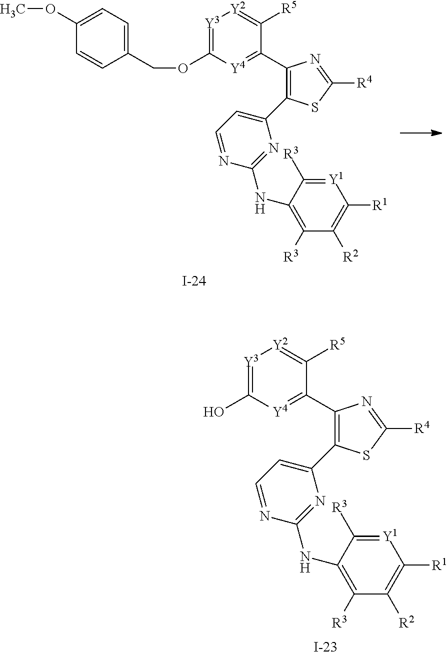

Based upon this disclosure and the examples contained herein one skilled in the art can readily convert a compound of the invention into a different compound of the invention.

The present invention also provides radiolabeled compounds of formula (I) and biotinylated compounds of formula (I) and solid-support-bound versions thereof, i.e. a compound of formula (I) having a radiolabel or biotin bound thereto. Radiolabeled compounds of formula (I) and biotinylated compounds of formula (I) can be prepared using conventional techniques. For example, radiolabeled compounds of formula (I) can be prepared by reacting the compound of formula (I) with tritium gas in the presence of an appropriate catalyst to produce radiolabeled compounds of formula (I). In one embodiment, the compounds of formula (I) are tritiated.

The radiolabeled compounds of formula (I) and biotinylated compounds of formula (I) are useful in assays for the identification of compounds which inhibit at least one Raf family kinase for the identification of compounds for the treatment of a condition capable of being treated with a Raf inhibitor, e.g., for the treatment of neoplasms susceptible to treatment with a Raf inhibitor. The present invention also provides an assay method for identifying such compounds, which method comprises the step of specifically binding a radiolabeled compound of the invention or a biotinylated compound of the invention to the target protein or cellular homogenate. More specifically, suitable assay methods will include competition binding assays. The radiolabeled compounds of formula (I) and biotinylated compounds of formula (I) and solid-support-bound versions thereof, can also be employed in assays according to the methods conventional in the art.

As another aspect, the present invention further provides compounds of formula (I) wherein one or more hydrogen atoms is replaced by a deuterium (²H) atom. In one embodiment, all hydrogen atoms of a compound of formula (I) are replaced with deuterium atoms. Deuterated compounds are known to be useful in chemistry and biochemistry as non-radioactive isotopic tracers to facilitate the study of chemical reactions and metabolic pathways. See, D. Kushner, et al., Pharmacological uses and Perspectives of Heavy Water and Deuterated Compounds, Canadian J. Physiology and Pharmacology (1999) 77(2):79-88. Deuterated compounds of formula (I), including salts thereof, may be prepared using the methods described herein for preparing compounds of formula (I) and conventional techniques for preparing deuterated molecules.

The following examples are intended for illustration only and are not intended to limit the scope of the invention in any way. The invention is defined by the claims which follow.

### EXAMPLES

As used herein, the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
atm (atmosphere);
g (grams);
mg (milligrams);
h (hour(s));
min (minutes);
Hz (Hertz);
MHz (megahertz);
i.v. (intravenous);
L (liters);
mL (milliliters);
μL (microliters);
M (molar);
mM (millimolar);
mol (moles);
mmol (millimoles);
mp (melting point);
psi (pounds per square inch);
rt (room temperature);
TLC (thin layer chromatography);
Tᵣ (retention time);
RP (reverse phase;
H₂ (hydrogen);
N₂ (nitrogen)
Ac (acetyl);
Ac₂O (acetic anhydride);
ATP (adenosine triphosphate);
BOC (tert-butyloxycarbonyl);
BOP (bis(2-oxo-3-oxazolidinyl)phosphinic chloride);
BSA (bovine serum albumin)
CBZ (benzyloxycarbonyl);
CDI (1,1-carbonyldiimidazole);
CHCl₃ (chloroform);
mCPBA (meta-chloroperbenzoic acid);
DCC (dicyclohexylcarbodiimide);
DCE (dichloroethane);
DCM (CH₂Cl₂; dichloromethane);
DMA (dimethyl acetyl);
DMAP (4-dimethylaminopyridine);
DME (1,2-dimethoxyethane);
DMEM (Dulbecco's modified Eagle medium);
DMF (N,N-dimethylformamide);
DMPU (N,N′-dimethylpropyleneurea);
DMSO (dimethylsulfoxide);
DPPA (diphenylphosphoryl azide);
EDC (ethylcarbodiimide hydrochloride);
EDTA (ethylenediaminetetraacetic acid);
EtOH (ethanol);
EtOAc (ethyl acetate);
FMOC (9-fluorenylmethoxycarbonyl);
HBTU (O-Benzotriazole-1-yl-N,N,N′,N′-tetramethyluronium hexafluorophosphate);
HCl (hydrochloric acid)
HEPES (4-(2-hydroxyethyl)-1-piperazine ethane sulfonic acid);
HOAc (acetic acid);
HOBT (1-hydroxybenzotriazole);
HOSu (N-hydroxysuccinimide);
fHNO₃ (fumed nitric acid);
HPLC (high pressure liquid chromatography);
HRP (horseradish peroxidase);
IBCF (isobutyl choroformate);
i-PrOH (isopropanol);
K₂CO₃ (potassium carbonate);
KOH (potassium hydroxide);
LAH (lithium aluminum hydride)
LHMDS (lithium hexamethyldisilazide);
LiOH.H₂O (lithium hydroxide monohydrate);
Me (methyl; —CH₃)
MeOH (methanol);
MgCO₃ (magnesium carbonate);
MgSO₄ (magnesium sulfate);
NaCNBH₃ (Sodium cyanoborohydride);
Na₂CO₃ (sodium carbonate);
NaHCO₃ (sodium bicarbonate);
NaH (sodium hydride)
Na₂SO₄ (sodium sulfate);
NBS is N-bromosuccinamide;
NH₄OH (ammonium hydroxide);
Pd₂ dba₃ (Tris(dibenzylidineacetone)-dipalladium (0));
TBAF (tetra-N-butylammonium fluoride);
TBS (t-butyldimethylsilyl);
TEA (triethylamine);
TFA (trifluoroacetic acid);
TFAA (trifluoroacetic anhydride);
THF (tetrahydrofuran);
TIPS (triisopropylsilyl);
TMS (trimethylsilyl);
TMSE (2-(trimethylsilyl)ethyl); and
TMSCl (Chlorotrimethylsilane).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at rt unless otherwise noted.

¹H NMR spectra were recorded on a Varian VXR-300, a Varian Unity-300, a Varian Unity-400 instrument, a General Electric QE-300, a Bruker 300, or a Bruker 400. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, a SCIEX-APIiii, a Finnegan MSQ, Waters SQD, Waters ZQ, or a Finnegan LCQ spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60E-254), visualized with UV light, 5% ethanolic phosphomolybdic acid or p-anisaldehyde solution or mass spectrometry (electrospray or AP). Flash column chromatography was performed on silica gel (230-400 mesh, Merck) or using automated silica gel chromatography (Isco, Inc. Sq 16× or 100 sg Combiflash).

### Example 1

### 4-[4-[3,5-Bis(methyloxy)phenyl]-2-(ethylamino)-1,3-thiazol-5-yl]-N-(6-{4-[2-(methylsulfonyl)ethyl]-1-piperazinyl}-3-pyridinyl)-2-pyrimidinamine

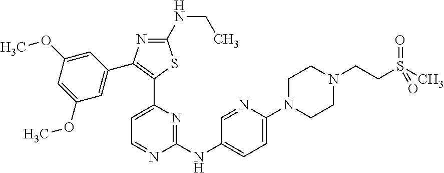

### Step A: tert-Butyl 4-(5-nitro-2-pyridinyl)-1-piperazinecarboxylate

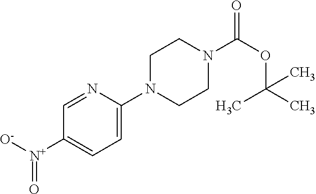

To a solution containing 5.8 g (31.5 mmol) of tert-butyl 1-piperazinecarboxylate and 20 mL of THF at 0° C. was added 1.5 g (37 mmol) of a 60% dispersion of NaH in mineral oil. The reaction mixture was allowed to stir for 20 min and 5.0 g (31.5 mmol) of 2-chloro-5-nitropyridine was added. The reaction mixture was heated at 50° C. overnight, quenched by the addition of water, and extracted with DCM. The combined organic layers were dried over MgSO₄ and the solvent was removed under reduced pressure. The residue was subjected to silica gel chromatography to give 4.89 g (50%) of tert-butyl 4-(5-nitro-2-pyridinyl)-1-piperazinecarboxylate as a yellow solid: ¹H NMR (400 MHz, DMSO-d₆) δ 8.25 (dd, J=9.5 and 2.9 Hz, 1H), 6.93 (d, J=9.5 Hz, 1H), 3.76-3.78 (m, 4H), 3.41-3.48 (m, 4H), and 1.42 (s, 9H).

### Step B: 1-(5-Nitro-2-pyridinyl)piperazine bis trifluoroacetate

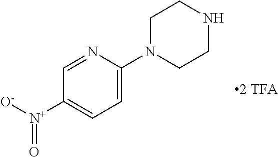

To a solution containing 4.8 g (15.6 mmol) of tert-butyl 4-(5-nitro-2-pyridinyl)-1-piperazinecarboxylate and 50 mL of DCM was added 5 mL of TFA. The reaction mixture was allowed to stir for 3 days and the solvents were removed under reduced pressure to give 6.21 g (91%) of 1-(5-nitro-2-pyridinyl)piperazine bis trifluoroacetate as a yellow solid: ¹H NMR (400 MHz, DMSO-d₆) δ 9.00 (d, J=2.8 Hz), 8.85 (brs, 3H), 8.33 (dd, J=9.5 and 2.8 Hz, 1H), 7.04 (d, J=9.5 Hz, 1H), 3.94-3.99 (m, 4H), and 3.23 (brs, 4H).

### Step C: 1-[2-(Methylsulfonyl)ethyl]-4-(5-nitro-2-pyridinyl)piperazine

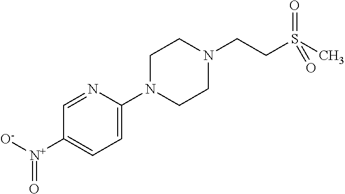

To a solution containing 1.5 g (3.5 mmol) of 1-(5-nitro-2-pyridinyl)piperazine bis trifluoroacetate and 25 mL of THF was added 0.34 mL (3.85 mmol) of methyl vinyl sulfone, followed by 1.6 mL (11.6 mmol) of TEA. The reaction mixture was heated at 60° C. overnight and partitioned between DCM and water. The combined organic layers were dried over MgSO₄ and the solvent was removed under reduced pressure. The residue was subjected to silica gel chromatography to give 0.83 g (75%) of 1-[2-(methylsulfonyl)ethyl]-4-(5-nitro-2-pyridinyl)piperazine as a yellow solid: ¹H NMR (400 MHz, DMSO-d₆) δ 8.22 (dd, J=9.7 and 2.9 Hz, 1H), 6.97 (d, J=9.7 Hz, 1H), 3.73-3.79 (m, 4H), 3.34 (t, J=6.8 Hz, 2H), 3.05 (s, 3H), 2.76 (t, J=6.8 Hz, 2H), and 2.52-2.55 (m, 4H). MS (ESI): 314.14 (M+H⁺).

### Step D: 6-{4-[2-(Methylsulfonyl)ethyl]-1-piperazinyl}-3-pyridinamine

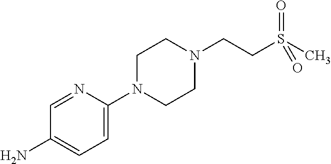

A mixture containing 0.83 g (2.6 mmol) of 1-[2-(methylsulfonyl)ethyl]-4-(5-nitro-2-pyridinyl)piperazine, 85 mg of 5% Pt on carbon, and 20 mL of EtOH was subjected to a 50 psi atmosphere of H₂ overnight. The reaction mixture was filtered over a pad of Celite to give 6-{4-[2-(methylsulfonyl)ethyl]-1-piperazinyl}-3-pyridinamine as a white solid: ¹H NMR (400 MHz, DMSO-d₆) δ 7.59 (d, J=2.9 Hz, 1H), 6.90 (dd, J=8.8 and 2.9 Hz, 1H), 6.63 (d, J=9.0 Hz, 1H), 4.56 (br s, 2H), 3.30-3.32 (m, 4H), 3.18-3.25 (m, 4H), 3.03 (s, 3H), 2.73 (t, J=6.9 Hz, 2H), and 2.52-2.54 (m, 2H).

### Step E: 1-[3,5-Bis(methyloxy)phenyl]-2-(2-chloro-4-pyrimidinyl)ethanone

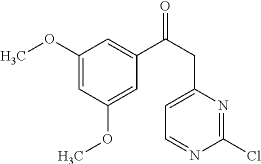

To a solution containing 37 g (188 mmol) of methyl 3,5-bis(methyloxy)benzoate and 300 mL of THF at 0° C. was added 395 mL (395 mmol) of a 1.0 M solution of LHMDS in THF. To this mixture, a solution containing 29 g (226 mmol) of 2-chloro-4-methylpyrimidine and 100 mL was added dropwise over about 30 min. The reaction mixture was allowed to stir for an additional 30 min and quenched by the addition of 100 mL of MeOH. The solvents were removed under reduced pressure and the residue was partitioned between EtOAc and water. The combined organic layers were dried over MgSO₄ and filtered, washing the filter cake with copious amounts of EtOAc and DCM to dissolve precipitated product. The solvents were removed under reduced pressure and the resulting orange solid was triturated from EtOAc to give 46.5 g (85%) of 1-[3,5-bis(methyloxy)phenyl]-2-(2-chloro-4-pyrimidinyl)ethanone as a light tan solid, which exists as a mixture of ketone and enol tautomers: MS (ESI): 293.29 (M+H⁺).

### Step F: 4-[3,5-Bis(methyloxy)phenyl]-5-(2-chloro-4-pyrimidinyl)-N-ethyl-1,3-thiazol-2-amine

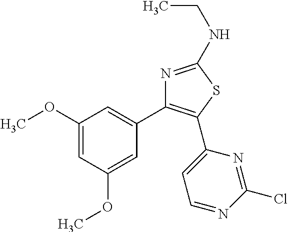

To a mixture containing 15.0 g (51.2 mmol) of 1-[3,5-bis(methyloxy)phenyl]-2-(2-chloro-4-pyrimidinyl)ethanone and 100 mL of DCM was added 9.1 g (51.2 mmol) of NBS. The reaction mixture was allowed to stir for 15 min until a clear solution was obtained. The solvents were removed under reduced pressure and the residue was taken up in 100 mL of dioxane. To this solution was added 6.4 g (61.4 mmol) of N-ethylthiourea and 15.0 g of MgCO₃. The reaction mixture was heated at 50° C. overnight and partitioned between DCM and a 10% aqueous solution of HCl. The aqueous layer was further extracted and the combined organic layers were dried over MgSO₄. The solvents were removed under reduced pressure and the residue was triturated twice from EtOAc to give 13.3 g (69%) of 4-[3,5-bis(methyloxy)phenyl]-5-(2-chloro-4-pyrimidinyl)-N-ethyl-1,3-thiazol-2-amine as a yellow solid: ¹H NMR (400 MHz, DMSO-d₆) δ 8.56 (s, 1H), 8.30 (d, J=5.7 Hz, 1H), 6.83 (d, J=5.7 Hz, 1H), 6.62-6.64 (m, 2H), 6.60-6.62 (m, 1H), 3.74 (s, 6H), 3.27-3.33 (m, 2H), and 1.19 (t, J=7.2 Hz, 3H). MS (ESI): 376.07 (M+H⁺).

### Step G: 4-[4-[3,5-Bis(methyloxy)phenyl]-2-(ethylamino)-1,3-thiazol-5-yl]-N-(6-{4-[2-(methylsulfonyl)ethyl]-1-piperazinyl}-3-pyridinyl)-2-pyrimidinamine

### General Procedure for the Aniline Displacement

To a solution containing 1-4 mL of i-PrOH or trifluoroethanol, optionally with 1,4-dioxane or DMA added as a co-solvent to improve solubility, and 1 equiv of the pyrimidyl chloride is added 1-1.5 equiv of the desired aniline and 1 drop of conc HCl, 4-5 drops of a 4 M solution of HCl in dioxane, or 0.1-2.1 eq of para toluene sulfonic acid. The reaction mixture is heated at 70-90° C. for 12-72 h, or heated in a microwave reactor at 150-180° C. for 10-120 min, then allowed to cool to rt. The displacement product is purified by neutralization by the addition of an aqueous solution of NaOH or NaHCO₃, or by the addition of 1-5 equiv of TEA and extracted into an organic solvent such as EtOAc or DCM. The residue from this extraction, or directly from evaporation of solvents from the reaction mixture, is then subjected to silica gel chromatography and/or HPLC purification. In certain instances, precipitation from an organic solvent, or treatment of a solution of the compound with MP-isocyante is utilized to remove excess aniline or other lingering impurities.

Specific procedure for 4-[4-[3,5-bis(methyloxy)phenyl]-2-(ethylamino)-1,3-thiazol-5-yl]-N-(6-{4-[2-(methylsulfonyl)ethyl]-1-piperazinyl}-3-pyridinyl)-2-pyrimidinamine To a solution containing 0.1 g (0.27 mmol) of 4-[3,5-bis(methyloxy)phenyl]-5-(2-chloro-4-pyrimidinyl)-N-ethyl-1,3-thiazol-2-amine, 0.09 g (0.33 mmol) of 6-{4-[2-(methylsulfonyl)ethyl]-1-piperazinyl}-3-pyridinamine, and 2 mL of iPrOH was added 0.1 mL of a 4.0 M solution of HCl in dioxane. The reaction mixture was heated at 90° C. overnight in a sealed tube and the solvent was removed under reduced pressure. The residue was subjected to silica gel chromatography and further triturated from MeOH to give 87 mg (51%) of 4-[4-[3,5-bis(methyloxy)phenyl]-2-(ethylamino)-1,3-thiazol-5-yl]-N-(6-{4-[2-(methylsulfonyl)ethyl]-1-piperazinyl}-3-pyridinyl)-2-pyrimidinamine as a yellow solid: ¹H NMR (400 MHz, DMSO-d₆) δ 9.23 (s, 1H), 8.47 (d, J=2.4 Hz, 1H), 8.22 (t, J=5.4 Hz, 1H), 8.06 (d, J=5.3 Hz, 1H), 7.86 (dd, J=9.2 and 2.8 Hz, 1H), 6.81 (d, J=9.2 Hz, 1H), 6.61 (d, J=2.2 Hz, 2H), 6.57 (t, J=2.3 Hz, 1H), 6.28 (d, J=5.3 Hz, 1H), 3.74 (s, 6H), 3.38-3.42 (m, 4H), 3.32-3.36 (m, 2H), 3.29 (dd, J=7.2 and 5.4 Hz, 2H), 3.05 (s, 3H), 2.75 (t, J=6.9 Hz, 2H), 2.52-2.56 (m, 4H), and 1.19 (t, J=7.2 Hz, 3H). HRMS Calcd for C₂₉H₃₇N₈O₄S₂ (M+H⁺): 625.2379. Found: 625.2352.

### Example 2

### 4-[4-[3,5-bis(methyloxy)phenyl]-2-(ethylamino)-1,3-thiazol-5-yl]-N-{6-[4-(methylsulfonyl)-1-piperazinyl]-3-pyridinyl}-2-pyrimidinamine

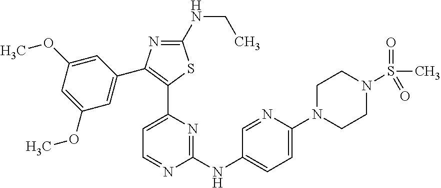

### Step A: 1-(Methylsulfonyl)-4-(5-nitro-2-pyridinyl)piperazine

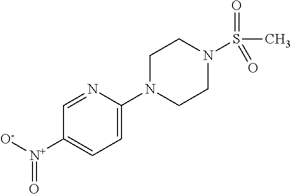

To a solution containing 1.5 g (3.5 mmol) of 1-(5-nitro-2-pyridinyl)piperazine bis trifluoroacetate, prepared by a procedure analogous to Example 1, Step B, and 25 mL of THF at 0° C. was added 0.3 mL (3.85 mmol) of methanesulfonyl chloride, followed by 1.6 mL (11.6 mmol) of TEA. The reaction was allowed to stir at rt overnight, quenched by the addition of water, and extracted with DCM. The organic layers were combined and the solvent was removed under reduced pressure. The resulting solid was triturated to give 0.6 g (60%) of 1-(methylsulfonyl)-4-(5-nitro-2-pyridinyl)piperazine as a yellow solid: ¹H NMR (400 MHz, DMSO-d₆) δ 8.99 (d, J=2.9 Hz, 1H), 8.28 (dd, J=9.5 and, 2.9 Hz, 1H), 7.02 (d, J=9.5 Hz, 1H), 3.86-3.93 (m, 4H), 3.20-3.25 (m, 4H), and 2.91 (s, 3H). MS (ESI): 287.26 (M+H⁺).

### Step B: 6-[4-(Methylsulfonyl)-1-piperazinyl]-3-pyridinamine

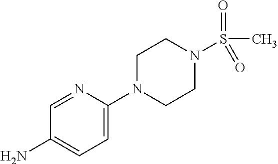

A mixture containing 0.6 g (2.09 mmol) of 1-(methylsulfonyl)-4-(5-nitro-2-pyridinyl)piperazine, 60 mg of 5% Pt on carbon, and 20 mL of EtOH was subjected to a 50 psi H₂ atmosphere overnight. The mixture was filtered through a pad of Celite and the solvent was removed under reduced pressure to give 6-[4-(methylsulfonyl)-1-piperazinyl]-3-pyridinamine as a white solid: ¹H NMR (400 MHz, DMSO-d₆) δ 7.61 (s, 1H), 6.93 (d, J=8.1 Hz, 1H), 6.68 (d, J=12.3 Hz, 1H), 4.63 (brs, 2H), 3.18 (brs, 4 H), 2.98 (brs, 1H), 2.89 (brs, 3H), 2.83 (brs, 1H), and 2.74 (brs, 1H).

### Step C: 4-[4-[3,5-Bis(methyloxy)phenyl]-2-(ethylamino)-1,3-thiazol-5-yl]-N-{6-[4-(methylsulfonyl)-1-piperazinyl]-3-pyridinyl}-2-pyrimidinamine

To a solution containing 0.1 g (0.29 mmol) of 4-[3,5-bis(methyloxy)phenyl]-5-(2-chloro-4-pyrimidinyl)-N-ethyl-1,3-thiazol-2-amine, prepared by a procedure analogous to Example 1, Step F, 0.09 g (0.35 mmol) of 6-[4-(methylsulfonyl)-1-piperazinyl]-3-pyridinamine, and 2 mL of iPrOH was added 0.1 mL of a 4.0 M solution of HCl in dioxane. The reaction mixture was heated at 90° C. in a sealed tube for 16 h and the solvent was removed under reduced pressure. The residue was subjected to silica gel chromatography and further purified by trituration to give 37 mg (22%) of 4-[4-[3,5-bis(methyloxy)phenyl]-2-(ethylamino)-1,3-thiazol-5-yl]-N-{6-[4-(methylsulfonyl)-1-piperazinyl]-3-pyridinyl}-2-pyrimidinamine as a yellow-orange solid: ¹H NMR (400 MHz, DMSO-d₆) δ 9.28 (s, 1H), 8.50 (d, J=2.2 Hz, 1H), 8.24 (t, J=5.3 Hz, 1H), 8.06 (d, J=5.3 Hz, 1H), 7.91 (dd, J=9.0, 3.0 Hz, 1H), 6.87 (d, J=9.2 Hz, 1H), 6.62 (d, J=2.2 Hz, 2H), 6.58 (t, J=2.3 Hz, 1H), 6.29 (d, J=5.5 Hz, 1H), 3.74 (s, 6H), 3.50-3.56 (m, 4H), 3.26-3.32 (m, 2H), 3.19-3.23 (m, 4H), 2.91 (s, 3H), and 1.19 (t, J=7.2 Hz, 3H). HRMS calcd for C₂₇H₃₃N₈O₄S₂ (M+H⁺): 597.2066. Found: 597.2074.

### Example 3

### 4-[4-[3,5-Bis(methyloxy)phenyl]-2-(ethylamino)-1,3-thiazol-5-yl]-N-[6-({(3S)-1-[2-(methylsulfonyl)ethyl]-3-pyrrolidinyl}oxy)-3-pyridinyl]-2-pyrimidinamine

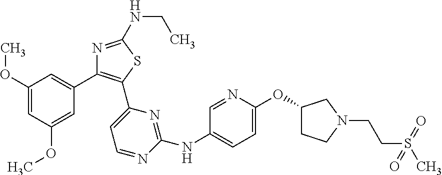

### Step A: 1,1-Dimethylethyl (3S)-3-[(5-nitro-2-pyridinyl)oxy]-1-pyrrolidinecarboxylate

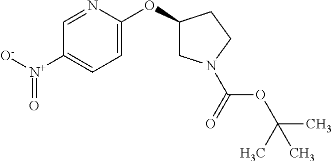

To a solution containing 3.5 g (18.9 mmol) of 1,1-dimethylethyl (35)-3-hydroxy-1-pyrrolidinecarboxylate and 20 mL of THF was slowly added 0.9 g (23 mmol) of a 60% dispersion of NaH in mineral oil. The reaction mixture was allowed to stir at rt for 10 min, then 3.0 g of 2-chloro-5-nitropyridine was added. The reaction mixture was heated at 50° C. overnight, quenched by the addition of water, and extracted with DCM. The combined organic layers were dried over MgSO₄ and the solvents were removed under reduced pressure. The residue was subjected to silica gel chromatography to give 3.3 g (57%) of 1-dimethylethyl (3S)-3-[(5-nitro-2-pyridinyl)oxy]-1-pyrrolidinecarboxylate as a brown oil: ¹H NMR (400 MHz, DMSO-d₆) δ 9.10 (d, J=2.8 Hz, 1H), 8.48 (dd, J=9.1 and 2.8 Hz, 1H), 7.05 (d, J=9.2 Hz, 1H), 5.61 (brs, 1H), 3.63 (td, J=13.2 and 4.3 Hz, 1H), 3.39-3.49 (m, 2H), 3.33-3.36 (m, 1H), 2.15-2.27 (m, 1H), 2.05-2.15 (m, 1H), and 1.39 (s, 9H).

### Step B: 5-Nitro-2-[(3S)-3-pyrrolidinyloxy]pyridine bis(trifluoroacetate)

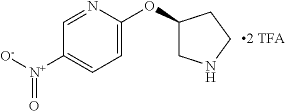

To a solution containing 3.3 g (4.2 mmol) of 1-dimethylethyl (3S)-3-[(5-nitro-2-pyridinyl)oxy]-1-pyrrolidinecarboxylate and 15 mL of DCM was added 2.0 mL of TFA. The reaction mixture was allowed to stir at rt overnight and the solvents were removed under reduced pressure to give 5-nitro-2-[(3S)-3-pyrrolidinyloxy]pyridine bis(trifluoroacetate) as a sticky brown semi-solid: ¹H NMR (400 MHz, DMSO-d₆) δ 9.16 (brs, 1H), 9.11 (d, J=2.4 Hz, 1H), 9.04 (brs, 1H), 8.53 (dd, J=9.2 and 2.9 Hz, 1H), 7.04 (d, J=9.2 Hz, 1H), 5.69-5.73 (m, 1H), 3.55 (td, J=12.5 and 6.8 Hz, 1H), 3.40-3.48 (m, 1H), 3.29-3.39 (m, 2H), 2.27-2.37 (m, 1H), and 2.15-2.24 (m, 1H).

### Step C: 2-({(3S)-1-[2-(Methylsulfonyl)ethyl]-3-pyrrolidinyl}oxy)-5-nitropyridine

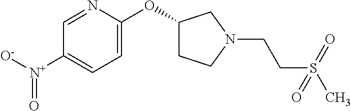

To a solution containing 1.5 g (3.5 mmol) of 5-nitro-2-[(3S)-3-pyrrolidinyloxy]pyridine bis(trifluoroacetate) and 25 mL of THF was added 0.34 mL (3.9 mmol) of methyl vinyl sulfone, followed by 1.6 mL (11.6 mmol) of TEA. The reaction mixture was heated at 60° C. overnight and portioned between water and EtOAc. The aqueous layer was further extracted with EtOAc and the combined organic layers were dried over MgSO₄. The solvents were removed under reduced pressure and the residue was subjected to silica gel chromatography to give 0.62 g (56%) of 2-({(3S)-1-[2-(methylsulfonyl)ethyl]-3-pyrrolidinyl}oxy)-5-nitropyridine as a white solid: ¹H NMR (400 MHz, DMSO-d₆) δ 9.04 (d, J=2.9 Hz, 1H), 8.47 (dd, J=9.2 and 2.9 Hz, 1H), 7.00 (d, J=9.2 Hz, 1H), 5.44-5.54 (m, 1H), 3.22-3.31 (m, 2H), 3.02 (s, 3H), 2.78-2.89 (m, 5H), 2.38-2.47 (m, 1H), 2.28-2.36 (m, 1H), and 1.80-1.90 (m, 1H). MS (ESI): 316.09 (M+H⁺).

### Step D: 6-({(3S)-1-[2-(Methylsulfonyl)ethyl]-3-pyrrolidinyl}oxy)-3-pyridinamine

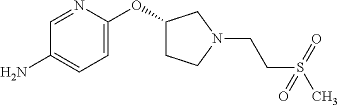

A mixture containing 0.62 g (2.0 mmol) of 2-({(3S)-1-[2-(methylsulfonyl)ethyl])-3-pyrrolidinyl}oxy)-5-nitropyridine, 60 mg of 5% Pt on carbon, and 20 mL of EtOH was subjected to a 50 psi H₂ atmosphere overnight. The reaction mixture was filtered through a pad of Celite and the solvents were removed under reduced pressure to give 6-({(3S)-1-[2-(methylsulfonyl)ethyl]-3-pyrrolidinyl}oxy)-3-pyridinamine as a sticky brown solid, which was stored as its corresponding HCl salt. Data is for the freebase: ¹H NMR (400 MHz, DMSO-d₆) δ 7.47 (dd, J=2.9 and 0.55 Hz, 1H), 6.98 (dd, J=8.6 and 2.9 Hz, 1H), 6.49 (dd, J=8.6 and 0.6 Hz, 1H), 5.15-5.20 (m, J=7.7, 6.0, 3.1, and 3.1 Hz, 1H), 4.72 (brs, 2H), 3.27 (td, J=6.9 and 2.8 Hz, 2H), 3.01 (s, 3H), 2.77-2.83 (m, 3H), 2.73 (td, J=8.2 and 6.0 Hz, 1H), 2.63 (dd, J=10.4 and 2.9 Hz, 1H), 2.38-2.46 (m, 1H), 2.13-2.23 (m, J=13.5, 7.7, 7.7, and 5.9 Hz, 1H), and 1.68-1.77 (m, 1H). and. MS (ESI): 286.41 (M+H⁺).

### Step E: 4-[4-[3,5-Bis(methyloxy)phenyl]-2-(ethylamino)-1,3-thiazol-5-yl]-N-[6-({(3S)-1-[2-(methylsulfonyl)ethyl]-3-pyrrolidinyl}oxy)-3-pyridinyl]-2-pyrimidinamine

To a suspension containing 0.1 g (0.29 mmol) of 4-[3,5-bis(methyloxy)phenyl]-5-(2-chloro-4-pyrimidinyl)-N-ethyl-1,3-thiazol-2-amine, prepared by a procedure analogous to Example 1, Step F, 0.11 g (0.35 mmol) of 6-({(3S)-1-[2-(methylsulfonyl)ethyl]-3-pyrrolidinyl}oxy)-3-pyridinamine and 2 mL of iPrOH was added 0.1 mL of a 4.0 M solution of HCl in dioxane. The reaction mixture was heated at 90° C. in a sealed tube for 12 h and the solvent was removed under reduced pressure. The residue was subjected to silica gel chromatography and further purified by HPLC to give 45 mg (25%) of 4-[4-[3,5-bis(methyloxy)phenyl]-2-(ethylamino)-1,3-thiazol-5-yl]-N-[6-({(3S)-1-[2-(methylsulfonyl)ethyl]-3-pyrrolidinyl}oxy)-3-pyridinyl]-2-pyrimidinamine as a yellow-orange solid: ¹H NMR (400 MHz, DMSO-d₆) δ 9.39 (s, 1H), 8.46 (d, J=2.9 Hz, 1H), 8.24 (t, J=5.4 Hz, 1H), 8.09 (d, J=5.5 Hz, 1H), 7.98 (dd, J=9.0 and 2.8 Hz, 1H), 6.72 (d, J=9.0 Hz, 1H), 6.62 (d, J=2.4 Hz, 2H), 6.58 (t, J=2.3 Hz, 1H), 6.32 (d, J=5.5 Hz, 1H), 5.28-5.34 (m, 1H), 3.74 (s, 6H), 3.26-3.31 (m, 4H), 3.03 (s, 3H), 2.77-2.87 (m, 4H), 2.66-2.74 (m, 1H), 2.42-2.47 (m, 1H), 2.21-2.31 (m, 1H), 1.75-1.84 (m, 1H), and 1.19 (t, J=7.2 Hz, 3H). HRMS calcd for C₂₉H₃₆N₇O₅S₂ (M+H⁺): 626.2219. Found: 626.2225.

### Example 4

### N-[6-(4-Acetyl-1-piperazinyl)-3-pyridinyl]-4-[4-[3,5-bis(methyloxy)phenyl]-2-(ethylamino)-1,3-thiazol-5-yl]-2-pyrimidinamine

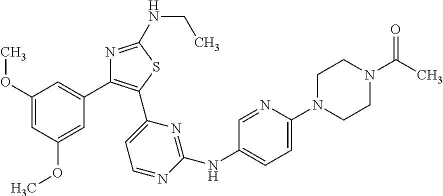

### Step A: 1-Acetyl-4-(5-nitro-2-pyridinyl)piperazine

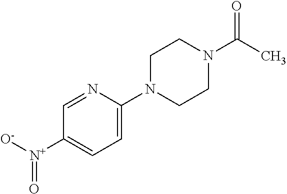

To a solution containing 1.5 g (3.5 mmol) of 1-(5-nitro-2-pyridinyl)piperazine bis trifluoroacetate, prepared by a procedure analogous to Example 1, Step B, and 20 mL of THF was added 0.37 mL (3.85 mmol) of Ac₂O, followed by 1.6 mL (11.6 mmol) of TEA. The reaction mixture was heated at 50° C. overnight and the solvent was removed under reduced pressure. The residue was subjected to silica gel chromatography to give 0.9 g (100%) of 1-acetyl-4-(5-nitro-2-pyridinyl)piperazine as a yellow solid: ¹H NMR (400 MHz, DMSO-d₆) δ 8.99 (d, J=2.8 Hz), 8.25 (dd, J=9.6 and 2.8 Hz, 1H), 6.95 (d, J=9.5 Hz, 1H), 3.79-3.86 (m, 2H), 3.73-3.79 (m, 2H), 3.57 (dt, J=6.8 and 3.5 Hz, 4H), 2.05 (s, 3H).

### Step B: 6-(4-Acetyl-1-piperazinyl)-3-pyridinamine

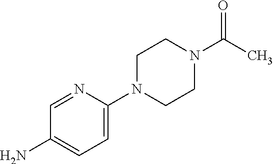

A mixture containing 0.9 g (3.5 mmol) of 1-acetyl-4-(5-nitro-2-pyridinyl)piperazine, 0.09 g of 5% Pt on carbon, and 20 mL of EtOH was subjected to a 60 psi H₂ atmosphere for 6 h. The reaction mixture was filtered through a pad of Celite, eluting with EtOH and EtOAc and the solvent was removed under reduced pressure to give 0.77 g (100%) of 6-(4-acetyl-1-piperazinyl)-3-pyridinamine as a purple solid: ¹H NMR (400 MHz, DMSO-d₆) δ 7.60 (s, 1H), 6.89 (d, J=9.0 Hz, 1H), 6.66 (d, J=9.0 Hz, 1H), 4.60 (brs, 2H), 3.51 (brs, 4H), 3.24 (brs, 2H), 3.17 (brs, 2H), 2.03 (brs, 3H).

### Step C: N-[6-(4-Acetyl-1-piperazinyl)-3-pyridinyl]-4-[4-[3,5-bis(methyloxy)phenyl]-2-(ethylamino)-1,3-thiazol-5-yl]-2-pyrimidinamine

To a suspension containing 0.1 g (0.29 mmol) of 4-[3,5-bis(methyloxy)phenyl]-5-(2-chloro-4-pyrimidinyl)-N-ethyl-1,3-thiazol-2-amine, prepared by a procedure analogous to Example 1, Step F, 0.08 g (0.35 mmol) of 6-(4-acetyl-1-piperazinyl)-3-pyridinamine and 2 mL of iPrOH was added 0.1 mL of a 4.0 M solution of HCl in dioxane. The reaction mixture was heated at 90° C. in a sealed tube for 12 h and the solvent was removed under reduced pressure. The residue was subjected to silica gel chromatography and further purified by HPLC to give 74 mg (46%) of N-[6-(4-acetyl-1-piperazinyl)-3-pyridinyl]-4-[4-[3,5-bis(methyloxy)phenyl]-2-(ethylamino)-1,3-thiazol-5-yl]-2-pyrimidinamine as a yellow solid: ¹H NMR (400 MHz, DMSO-d₆) δ ppm 9.26 (s, 1H), 8.49 (d, J=2.8 Hz, 1H), 8.23 (t, J=5.4 Hz, 1H), 8.06 (d, J=5.5 Hz, 1H), 7.89 (dd, J=9.2 and 2.8 Hz, 1H), 6.83 (d, J=9.2 Hz, 1H), 6.62 (d, J=2.2 Hz, 2H), 6.57 (t, J=2.3 Hz, 1H), 6.28 (d, J=5.3 Hz, 1H), 3.74 (s, 6H), 3.53-3.57 (m, 4H), 3.43-3.47 (m, 2H), 3.36-3.40 (m, 2H), 3.28 (td, J=7.2 and 5.6 Hz, 2H), 2.05 (s, 3H), 1.19 (t, J=7.2 Hz, 3H). HRMS Calcd for C₂₈H₃₃N₈O₃S: 561.2396 (M+H⁺). Found: 561.2399.

### Example 5

### 4-[4-[3,5-Bis(methyloxy)phenyl]-2-(ethylamino)-1,3-thiazol-5-yl]-N-[4-(4-ethyl-1-piperazinyl)phenyl]-2-pyrimidinamine trifluoroacetate

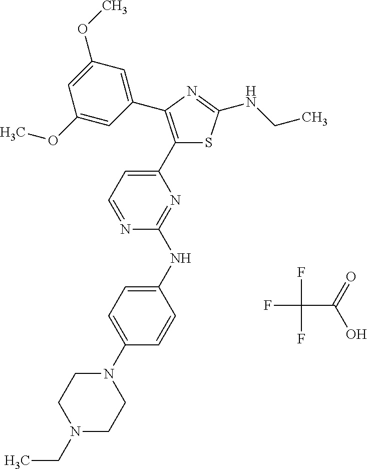

The title compound of Example 5 was synthesized using standard microwave displacement conditions analogous to Example 1, Step G, in trifluoroethanol using 4-[3,5-bis(methyloxy)phenyl]-5-(2-chloro-4-pyrimidinyl)-N-ethyl-1,3-thiazol-2-amine (0.085 g, 0.23 mmol), prepared in a procedure analogous to Example 1, Step F, and [4-(4-ethyl-1-piperazinyl)phenyl]amine (0.051 g, 0.25 mmol). Solvent was removed and the residue was taken up in DMSO/MeOH (2:1) and purified via HPLC to give 0.089 g, 71% yield, of desired product as a yellow solid. ¹H NMR (400 MHz, DMSO-d₆) δ ppm 9.34 (s, 2H), 8.26 (s, 1H), 8.26 (s, 1H), 8.06 (d, J=5.5 Hz, 1H), 7.61 (d, J=9.2 Hz, 2H), 6.94 (d, J=9.2 Hz, 2H), 6.5-6.6 (m, 3H), 6.28 (d, J=5.5 Hz, 1H), 3.73 (s, 7H), 3.57 (dd, J=12.28, 1.28 Hz, 2H), 3.24-3.32 (m, 2H), 3.20 (dd, J=7.33, 5.13 Hz, 2H), 3.12 (dd, J=10.6, 2.20 Hz, 1H), 2.83-2.94 (m, 2H), 1.24 (t, J=7.3 Hz, 3H), 1.18 (t, J=7.2 Hz, 3H). ES-LCMS m/z 546 (M+H).

### Example 6

### 4-[4-[3,5-Bis(methyloxy)phenyl]-2-(ethylamino)-1,3-thiazol-5-yl]-N-(3-fluoro-4-{4-[2-(methylsulfonyl)ethyl]-1-piperazinyl}phenyl)-2-pyrimidinamine trifluoroacetate

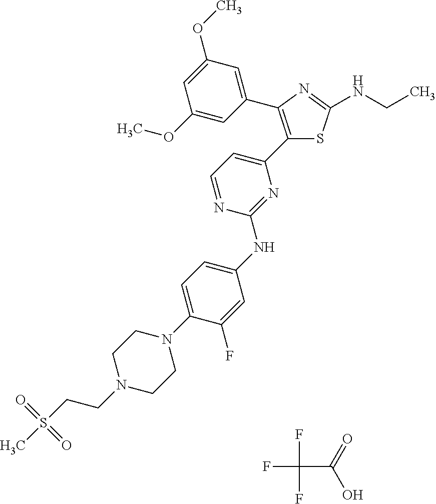

### Step A: 1-(2-Fluoro-4-nitrophenyl)-4-[2-(methylsulfonyl)ethyl]piperazine

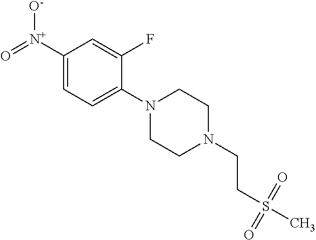

To a solution of 1-(2-fluoro-4-nitrophenyl)piperazine (0.500 g, 2.22 mmol) in iPrOH (25 mL), methyl vinyl sulfone (0.354 g, 3.33 mmol) was added. The reaction was heated to reflux for 18 h, cooled to rt, and loaded directly onto silica. Purification via flash chromatography (EtOAc/EtOAc:MeOH:NH₄OH (80:19:1) 0-100% gradient over 15 min) gave 0.500 g of a yellow solid. ¹H NMR (400 MHz, DMSO-d₆) δ ppm 7.93-8.00 (m, 2H), 7.13 (td, J=9.06, 0.73 Hz, 1H), 3.22-3.31 (m, 6H), 3.00 (s, 3H), 2.73 (t, J=6.59 Hz, 2H), 2.56 (br. s., 4H).

### Step B: (3-Fluoro-4-{4-[2-(methylsulfonyl)ethyl]-1-piperazinyl}phenyl)amine

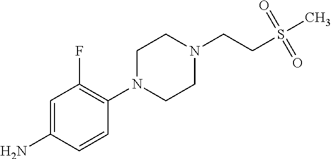

1-(2-Fluoro-4-nitrophenyl)-4-[2-(methylsulfonyl)ethyl]piperazine (0.500 g, 1.51 mmol) was taken up in EtOH (15 mL) and 10% Palladium/Carbon (0.050 g) was added. The mixture was stirred under H₂, 60 psi, for 3 h, filtered through a celite plug, and the solvent was removed to give 0.450 g, 98% yield, of a yellow solid. ¹H NMR (400 MHz, DMSO-d₆) δ ppm 6.67-6.74 (m, 1H), 6.21-6.31 (m, 2H), 4.92 (s, 2H), 3.22-3.28 (m, 3H), 2.97-3.01 (m, 3H), 2.77 (br s, 4H), 2.66-2.72 (m, 2H), and 2.50 (br s, 3H).

### Step C: 4-[4-[3,5-Bis(methyloxy)phenyl]-2-(ethylamino)-1,3-thiazol-5-yl]-N-(3-fluoro-4-{4-[2-(methylsulfonyl)ethyl]-1-piperazinyl}phenyl)-2-pyrimidinamine trifluoroacetate

The title compound of Example 6 was synthesized using standard microwave displacement conditions analogous to Example 1, Step G in trifluoroethanol using 4-[3,5-bis(methyloxy)phenyl]-5-(2-chloro-4-pyrimidinyl)-N-ethyl-1,3-thiazol-2-amine (0.085 g, 0.23 mmol), prepared in a procedure analogous to Example 1, Step F, and (3-fluoro-4-{4-[2-(methylsulfonyl)ethyl]-1-piperazinyl}phenyl)amine (0.075 g, 0.25 mmol). The solvent was removed and the residue was taken up in DMSO/MeOH (2:1) and purified via HPLC. Desired fractions were combined and the solvent was removed to give 0.053 g, 37% yield, of title compound of Example 6 as a solid. ¹H NMR (400 MHz, DMSO-d₆) δ ppm 8.30 (t, J=4.8 Hz, 1H), 8.10 (d, J=5.5 Hz, 1H), 7.80 (dd, J=15.6, 2.4 Hz, 1H), 7.38 (dd, J=8.8, 1.8 Hz, 1H), 6.97-7.07 (m, 1H), 6.53-6.61 (m, 3H), 6.32 (d, J=5.5 Hz, 1H), 3.72 (s, 6H), 3.67 (s, 3H), 3.54 (s, 2 H), 3.27 (d, J=12.8 Hz, 4H), 3.11 (s, 3H), 3.07 (dd, J=7.2, 5.0 Hz, 1H), 2.51 (s, 3 H), and 1.13-1.20 (m, 4H). ES-LCMS m/z 642 (M+H).

### Example 7

### 4-[4-[3,5-Bis(methyloxy)phenyl]-2-(ethylamino)-1,3-thiazol-5-yl]-N-[3-fluoro-4-(4-morpholinyl)phenyl]-2-pyrimidinamine

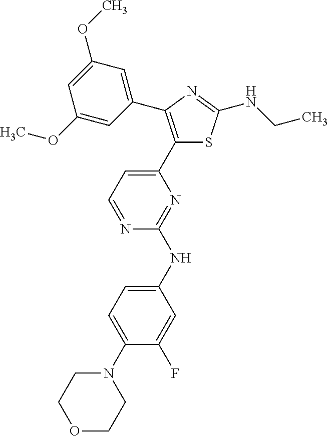

### Step A: 4-(2-Fluoro-4-nitrophenyl)morpholine

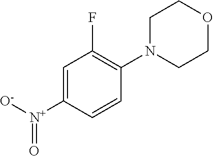

To a solution of 1,2-difluoro-4-nitrobenzene (5.500 g, 34.57 mmol) in acetonitrile (10 mL), morpholine (3.94 g, 38.03 mmol) was added, followed by N-ethyl-N-(1-methylethyl)-2-propanamine (6.70 g, 51.86 mmol). The reaction was heated in the microwave to 140° C. for 15 min. The solvent was removed in vacuo, and the residue was dissolved in EtOAc and washed with NaHCO₃ and brine. The organic layers were dried over MgSO₄ and the solvent was removed to give 7.70 g, 98% yield, of the title compound of Step A. ES-LCMS m/z 227 (M+H).

### Step B: [3-Fluoro-4-(4-morpholinyl)phenyl]amine

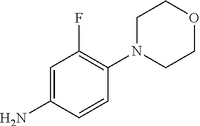

4-(2-Fluoro-4-nitrophenyl)morpholine (7.70 g, 33.62 mmol) was taken up in EtOH (100 mL) and 10% Palladium/Carbon (0.500 g) was added. The mixture was stirred under H₂, 60 psi, for 3 h. The reaction mixture was filtered through a celite plug and the solvent was removed to give 6.40 g, 95% yield, of a yellow solid. ¹H NMR (400 MHz, DMSO-d₆) δ ppm 6.71-6.80 (m, 1H), 6.27-6.35 (m, 2H), 4.97 (s, 2H), 3.65-3.68 (m, 4H), and 2.77-2.80 (m, 4H).

### Step C: 4-[4-[3,5-Bis(methyloxy)phenyl]-2-(ethylamino)-1,3-thiazol-5-yl]-N-[3-fluoro-4-(4-morpholinyl)phenyl]-2-pyrimidinamine

The title compound of Example 7 was synthesized using standard microwave displacement conditions analogous to Example 1, Step G, in trifluoroethanol using 4-[3,5-bis(methyloxy)phenyl]-5-(2-chloro-4-pyrimidinyl)-N-ethyl-1,3-thiazol-2-amine (0.085 g, 0.23 mmol), prepared by a procedure analogous to Example 1, Step F, and [3-fluoro-4-(4-morpholinyl)phenyl]amine (0.049 g, 0.25 mmol). The solvent was removed and the residue was taken up in DMSO/MeOH (2:1) and purified via HPLC. Desired fractions were combined, diluted with EtOAc, and washed twice with NaHCO₃. The organic layer was dried over MgSO₄ and the solvent was removed to give 0.047 g, 39% yield, of desired product as a solid. ¹H NMR (400 MHz, DMSO-d₆) δ ppm 9.50 (s, 1H), 8.23-8.28 (m, 1H), 8.10 (d, J=5.5 Hz, 1H), 7.75 (dd, J=16.0, 2.4 Hz, 1H), 7.34-7.41 (m, 1H), 6.90-6.98 (m, 1H), 6.61 (d, J=2.20 Hz, 2 H), 6.56 (t, J=2.20 Hz, 1H), 6.32 (d, J=5.50 Hz, 1H), 3.70-3.75 (m, 10H), 3.24-3.29 (m, 2H), 2.89-2.95 (m, 4H), 1.15-1.21 (m, 3H). ES-LCMS m/z 537 (M+H).

### Example 8

### 4-[4-[3,5-Bis(methyloxy)phenyl]-2-(ethylamino)-1,3-thiazol-5-yl]-N-{3-fluoro-4-[4-(2-fluoroethyl)-1-piperazinyl]phenyl}-2-pyrimidinamine

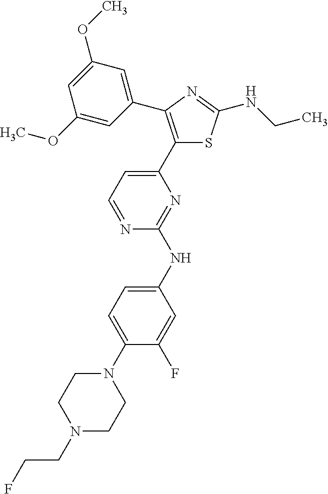

### Step A: 1-(2-Fluoroethyl)-4-(2-fluoro-4-nitrophenyl)piperazine

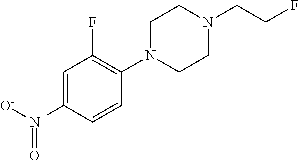

To a solution of 1-(2-fluoro-4-nitrophenyl)piperazine (0.500 g, 2.22 mmol) in DMF (25 mL), 1-bromo-2-fluoroethane (0.423 g, 3.33 mmol) and Na₂CO₃ (0.706 g, 6.66 mmol) were added and heated to 70° C. for 18 h. The reaction was cooled to rt, diluted with EtOAc, and washed twice with NaHCO₃ and brine. The organic layer was dried over MgSO₄, loaded onto silica, and purified via flash chromatography using EtOAc/Hex gradient 0-100%. The desired fractions were combined and the solvent was removed to give 0.510 g, 85% yield, of the title compound of Step A. ¹H NMR (400 MHz, DMSO-d₆) δ ppm 8.00 (s, 2H), 7.16 (s, 1H), 4.62 (s, 1H), 4.48 (s, 1H), 3.28 (s, 4 H), 2.70 (s, 1H), 2.61 (s, 5H).

### Step B: {3-Fluoro-4-[4-(2-fluoroethyl)-1-piperazinyl]phenyl}amine

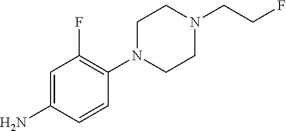

1-(2-Fluoroethyl)-4-(2-fluoro-4-nitrophenyl)piperazine was taken up in MeOH (25 mL) and 10% Palladium/Carbon (0.050 g) was added. The mixture was stirred under H₂, 60 psi, for 3 h and filtered through a celite plug. The solvent was removed to give 0.474 g, 91% yield, of a solid. ES-LCMS m/z 242 (M+H).

### Step C: 4-[4-[3,5-Bis(methyloxy)phenyl]-2-(ethylamino)-1,3-thiazol-5-yl]-N-{3-fluoro-4-[4-(2-fluoroethyl)-1-piperazinyl]phenyl}-2-pyrimidinamine

The title compound of Example 8 was synthesized using standard microwave displacement conditions analogous to Example 1, Step G in trifluoroethanol using 4-[3,5-bis(methyloxy)phenyl]-5-(2-chloro-4-pyrimidinyl)-N-ethyl-1,3-thiazol-2-amine (0.085 g, 0.23 mmol), prepared by a procedure analogous to Example 1, Step F, and {3-fluoro-4-[4-(2-fluoroethyl)-1-piperazinyl]phenyl}amine (0.060 g, 0.25 mmol). The solvent was removed and the residue was taken up in DMSO/MeOH (2:1) and purified via HPLC. Desired fractions were combined, diluted with EtOAc, and washed twice with NaHCO₃. The organic layer was dried over MgSO₄ and the solvent was removed to give 0.064 g, 52% yield, of desired product as a solid. ¹H NMR (400 MHz, DMSO-d₆) δ ppm 9.48 (s, 1H), 8.25 (s, 1H), 8.08 (s, 1H), 7.72 (d, J=15.6 Hz, 1H), 7.34 (d, J=8.2 Hz, 1H), 6.92 (t, J=8.3 Hz, 1H), 6.53-6.62 (m, 3 H), 6.30 (s, 1H), 4.60 (s, 1H), 4.48 (s, 1H), 3.72 (s, 6H), 3.26 (s, 2H), 2.94 (s, 4H), 2.68 (br. s., 1H), 2.58 (br. s., 5H), and 1.17 (s, 3H). ES-LCMS m/z 582 (M+H).

### Example 9

### 4-[4-[3,5-Bis(methyloxy)phenyl]-2-(ethylamino)-1,3-thiazol-5-yl]-N-[4-(1,1-dioxido-4-thiomorpholinyl)-3-fluorophenyl]-2-pyrimidinamine

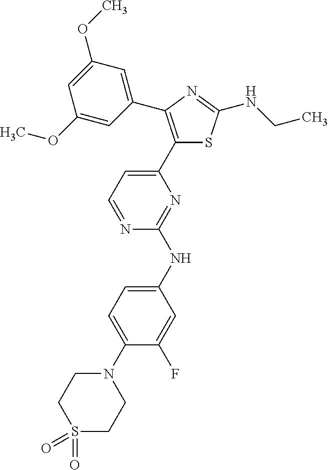

### Step A: 4-(2-Fluoro-4-nitrophenyl)thiomorpholine

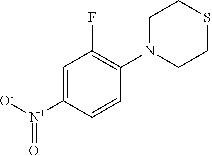

To a solution of 1,2-difluoro-4-nitrobenzene (5.00 g, 31.43 mmol) in acetonitrile (10 mL), thiomorpholine (3.57 g, 34.57 mmol) was added followed by N-ethyl-N-(1-methylethyl)-2-propanamine (6.09 g, 47.14 mmol). The reaction was heated in the microwave to 140° C. for 15 min. The solvent was removed in vacuo, and the residue was dissolved in EtOAc and washed with NaHCO₃ and brine. The organic layer was dried over MgSO₄ and the solvent was removed to give 7.56 g, 99% yield, of the title compound of Step A. ES-LCMS m/z 243 (M+H).

### Step B: 4-(2-Fluoro-4-nitrophenyl)thiomorpholine 1,1-dioxide

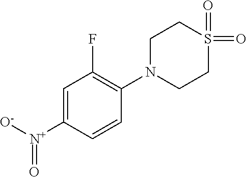

4-(2-Fluoro-4-nitrophenyl)thiomorpholine (7.56 g, 31.24 mmol) was taken up in 100 mL MeOH and 100 mL of water. Oxone (48.05 g, 78.10 mmol) was added and the mixture was stirred at rt for 18 h. The mixture was concentrated in vacuo then diluted with EtOAc and washed with NaHCO₃ and brine. The organic layer was dried over MgSO₄ and the solvent was removed to give 3.89 g, 45% yield, of the title compound of Step B. ES-LCMS m/z 275 (M+H).

### Step C: [4-(1,1-Dioxido-4-thiomorpholinyl)-3-fluorophenyl]amine

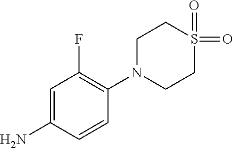

4-(2-Fluoro-4-nitrophenyl)thiomorpholine 1,1-dioxide was taken up in EtOH (100 mL) and 10% Palladium/Carbon (0.500 g) was added. The mixture was stirred under H₂, 60 psi, for 3 h, filtered through celite plug, and the solvent was removed to give 6.40 g, 95% yield, of a yellow solid. ¹H NMR (400 MHz, DMSO-d₆) δ ppm 6.86 (dd, J=9.9, 8.4 Hz, 1H), 6.26-6.35 (m, 2H), 5.11 (s, 2H), 3.23-3.29 (m, 4H), 3.15-3.21 (m, 4H).

### Step D: 4-[4-[3,5-Dis(methyloxy)phenyl]-2-(ethylamino)-1,3-thiazol-5-yl]-N-[4-(1,1-dioxido-4-thiomorpholinyl)-3-fluorophenyl]-2-pyrimidinamine

The title compound of Example 9 was synthesized using standard microwave displacement conditions analogous to Example 1, Step G in trifluoroethanol using 4-[3,5-bis(methyloxy)phenyl]-5-(2-chloro-4-pyrimidinyl)-N-ethyl-1,3-thiazol-2-amine (0.085 g, 0.23 mmol), prepared by a procedure analogous to Example 1, Step F, and [4-(1,1-dioxido-4-thiomorpholinyl)-3-fluorophenyl]amine (0.061 g, 0.25 mmol). The solvent was removed and the residue was taken up in DMSO/MeOH (2:1) and purified via HPLC. Desired fractions were combined, diluted with EtOAc, and washed twice with NaHCO₃. The organic layer was dried over MgSO₄ and the solvent was removed to give 0.062 g, 46% yield, of desired product of Example 9 as a solid. ¹H NMR (400 MHz, DMSO-d₆) δ ppm 9.57 (s, 1H), 8.28 (s, 1H), 8.11 (d, J=5.1 Hz, 1H), 7.81 (d, J=15.4 Hz, 1H), 7.36 (d, J=7.7 Hz, 1H), 7.08 (t, J=9.2 Hz, 1H), 6.54-6.65 (m, 2H), 6.33 (d, J=5.5 Hz, 1H), 3.73 (s, 6H), 3.41 (br. s., 5H), 3.24 (br. s., 6H), 1.15-1.26 (m, 3H). ES-LCMS m/z 585 (M+H).

### Example 10

### 4-[4-[3,5-Bis(methyloxy)phenyl]-2-(ethylamino)-1,3-thiazol-5-yl]-N-[3-fluoro-4-({1-[2-(methylsulfonyl)ethyl]-4-piperidinyl}oxy)phenyl]-2-pyrimidinamine

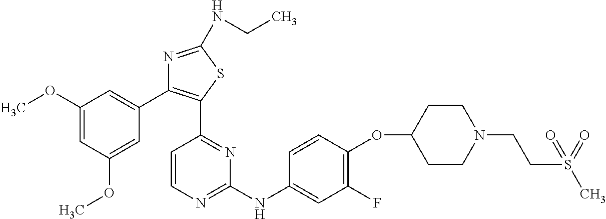

### Step A: Phenylmethyl 4-[(2-fluoro-4-nitrophenyl)oxy]-1-piperidinecarboxylate

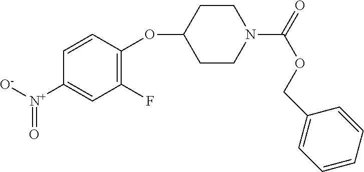

To a chilled (0° C.) stirring solution of 2-fluoro-4-nitrophenol (15 g, mmol), 1-benzyl-4-hydroxy-1-piperidinecarboxylate and PS-triphenylphosphine in DCM was added di-tert-butylazodicarboxylate. The reaction was equilibrated to rt and stirred for 19 h. The reaction was concentrated directly onto silica gel and purified in batches to yield a crude material. Each crude batch was dissolved in ether and washed with 2.0 N NaOH solution (2×50 mL). All batches were combined to afford the title compound of Step A as an opaque syrup (30 g; 84%). ¹H NMR (400 MHz, CDCl₃) δ 7.93-8.13 (m, 2H), 7.30-7.49 (m, 3H), 7.05 (t, J=8.3 Hz, 1H), 6.36 (s, 2H), 5.15 (s, 2H), 4.60-4.77 (m, 1H), 3.66-3.84 (m, 2H), 3.41-3.60 (m, 2H), 1.98 (s, 2H), 1.88 (s, 2H).

### Step B: 4-[(2-Fluoro-4-nitrophenyl)oxy]piperidine hydrobromide

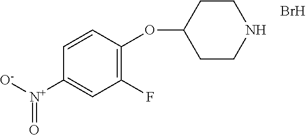

Hydrobromic acid (30 weight % in acetic acid, 26.9 g, 99.73 mmol) was added to phenylmethyl 4-[(2-fluoro-4-nitrophenyl)oxy]-1-piperidinecarboxylate (30 g, 80.13 mmol) and the mixture was stirred at rt for 0.5 h. Ether (400 mL) was slowly added and a white solid was collected by filtration. The solid was washed with ether (100 mL) and hexanes (150 mL) and air-dried to afford the title compound of Step B (12.95 g, 50%): ¹H NMR (400 MHz, DMSO-d₆) δ 8.19 (dd, J=11.2, 2.7 Hz, 1H), 8.06-8.14 (m, 1H), 7.47-7.58 (m, 1H), 4.87-4.98 (m, 1H), 3.42 (s, 1H), 3.18-3.30 (m, J=3.7 Hz, 2H), 3.02-3.18 (m, 2H), 2.07-2.21 (m, 2H), 1.77-1.96 (m, 2 H).

### Step C: 4-[(2-Fluoro-4-nitrophenyl)oxy]-1-[2-(methylsulfonyl)ethyl]piperidine

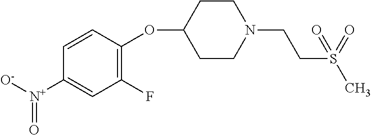

To a mixture of 4-[(2-fluoro-4-nitrophenyl)oxy]piperidine hydrobromide (1.25 g, 3.89 mmol) and Na₂CO₃ (1 g, 9.43 mmol) stirring in THF (15 mL) was added methyl vinyl sulfone (0.9 g, 8.48 mmol). The resulting reaction was heated to 65° C. for 2-3 h. The reaction was poured into EtOAc (100 mL) and washed with water (20 mL) and brine (20 mL), filtered through Whatman 1 PS paper, and concentrated in vacuo to the title compound of Step C (1.42 g; 100%) contaminated with a small amount of excess methyl vinyl sulfone. The reported data represents the major component of the reaction. ¹H NMR (400 MHz, DMSO-d₆) δ 8.16 (dd, J=11.2, 2.7 Hz, 1H), 8.05-8.11 (m, 1H), 7.49 (t, 1H), 4.65-4.75 (m, 1H), 3.26-3.31 (m, 2H), 3.00-3.05 (m, J=1.5 Hz, 3H), 2.67-2.78 (m, J=6.8, 6.8 Hz, 4H), 2.25-2.41 (m, 2H), 1.91-2.05 (m, J=3.3 Hz, 2H), 1.60-1.76 (m, 2H).

### Step D: [3-Fluoro-4-({1-[2-(methylsulfonyl)ethyl]-4-piperidinyl}oxy)phenyl]amine

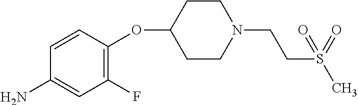

To a stirring mixture of 4-[(2-fluoro-4-nitrophenyl)oxy]-1-[2-(methylsulfonyl)ethyl]piperidine (1.35 g 3.90 mmol) in MeOH (75 mL) was added nickel (II) chloride hexahydrate (0.5 g; 2.1 mmol). The reaction mixture was chilled to 0° C. and sodium borohydride (0.3 g; 7.93 mmol) was added, causing a vigorous foaming action. The resulting reaction mixture turned black. The reaction was stirred for 0.5 h and more sodium borohydride (0.3 g; 7.93 mmol) was added. The reaction was stirred for 0.25 h and then quenched with a 2.0 N NaOH solution (75 mL). The reaction was partitioned between EtOAc and water/3:1. The mixture was filtered, the phases were separated, and the organic phase was washed with brine. The resulting organic phase was filtered through Whatman 1 PS paper and concentrated in vacuo to the title compound of Step D as an amber oil that was slightly contaminated (1.28 g, 100%). The data represents the title compound of Step D as the major product of the reaction. ¹H NMR (400 MHz, DMSO-d₆) δ 6.71-6.92 (m, 1 H), 6.36 (dd, J=13.6, 2.6 Hz, 1H), 6.27 (dd, J=8.1, 2.2 Hz, 1H), 4.99 (s, 2H), 3.87-4.01 (m, 1H), 3.26 (t, J=6.6 Hz, 2H), 3.02 (s, 3H), 2.63-2.76 (m, 4H), 2.13-2.27 (m, 2H), 1.77-1.89 (m, 2H), 1.49-1.64 (m, 2H).

### Step E: 4-[4-[3,5-Bis(methyloxy)phenyl]-2-(ethylamino)-1,3-thiazol-5-yl]-N-[3-fluoro-4-({1-[2-(methylsulfonyl)ethyl]-4-piperidinyl}oxy)phenyl]-2-pyrimidinamine

The general procedure (iPrOH (4 mL), 180° C.; 0.25 h; microwave, with 5 drops of conc. HCl), analogous to Example 1, Step G, was used for the reaction of 4-[3,5-bis(methyloxy)phenyl]-5-(2-chloro-4-pyrimidinyl)-N-ethyl-1,3-thiazol-2-amine (0.1 g, 0.27 mmol), prepared by a procedure analogous to Example 1, Step F, and [3-fluoro-4-({1-[2-(methylsulfonyl)ethyl]-4-piperidinyl}oxy)phenyl]amine (0.1 g, 1.19 mmol) to give a crude product. Purification by reverse phase HPLC gave the salt of the desired title compound of Example 10. This crude product was dissolved in DCM, washed with 50% saturated sodium bicarbonate solution, filtered through Whatman 1 PS paper and concentrated in vacuo to a mustard yellow solid (0.063 g, 36%). ¹H NMR (400 MHz, DMSO-d₆) δ 9.53 (s, 1H), 8.28 (t, J=5.3 Hz, 1H), 8.12 (d, J=5.5

Hz, 1H), 7.84 (d, J=14.5 Hz, 1H), 7.34 (d, J=9.0 Hz, 1H), 7.10 (t, J=9.3 Hz, 1H), 6.62 (d, J=2.2 Hz, 2H), 6.58 (t, J=2.3 Hz, 1H), 6.34 (d, J=5.3 Hz, 1H), 4.12-4.35 (m, 1H), 3.74 (s, 6H), 3.24-3.31 (m, 4H), 3.03 (s, 3H), 2.68-2.78 (m, J=6.8, 6.8 Hz, 4H), 2.26 (t, J=9.1 Hz, 2H), 1.85-1.95 (m, 2H), 1.57-1.70 (m, 2H), 1.20 (t, J=7.1 Hz, 3H). MS (ESI): 657.27 [M+H].

### Example 11

### 4-[4-[3,5-Bis(methyloxy)phenyl]-2-(ethylamino)-1,3-thiazol-5-yl]-N-[3-chloro-4-(4-methyl-1-piperazinyl)phenyl]-2-pyrimidinamine

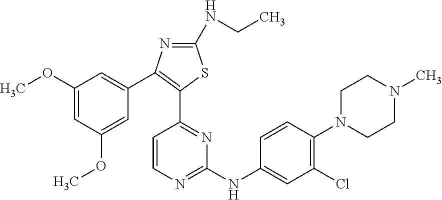

The general procedure (iPrOH (4 mL), 180° C.; 0.25 h; microwave, with 5 drops of conc. HCl), analogous to Example 1, Step G, was used for the reaction of 4-[3,5-bis(methyloxy)phenyl]-5-(2-chloro-4-pyrimidinyl)-N-ethyl-1,3-thiazol-2-amine (0.08 g, 0.21 mmol), prepared by a procedure analogous to Example 1, Step F, and [3-chloro-4-(4-methyl-1-piperazinyl)phenyl]amine (0.055 g, 0.24 mmol) to give a crude product. Purification by reverse phase HPLC gave the salt of the desired title compound of Example 11. This crude product was dissolved in DCM, washed with 50% saturated NaHCO₃ solution, filtered through Whatman 1 PS paper and concentrated in vacuo to a yellow solid (0.062 g, 52%). ¹H NMR (400 MHz, DMSO-d₆) δ 9.53 (s, 1H), 8.29 (t, J=5.3 Hz, 1H), 8.12 (d, J=5.5 Hz, 1H), 8.01-8.07 (m, 1H), 7.47-7.59 (m, 1H), 7.08 (d, J=9.0 Hz, 1H), 6.63 (dd, J=2.4 Hz, 2H), 6.58 (t, J=2.3 Hz, 1H), 6.34 (d, J=5.5 Hz, 1H), 3.74 (s, 6H), 3.24-3.30 (m, 2H), 2.85-2.97 (m, 4H), 2.42-2.50 (m, 4H), 2.23 (s, 3H), 1.20 (t, J=7.2 Hz, 3H). MS (ESI): 564.37 [M−H].

### Example 12

### N-{4-[(1-Acetyl-4-piperidinyl)oxy]-3-fluorophenyl}-4-[4-[3,5-bis(methyloxy)phenyl]-2-(ethylamino)-1,3-thiazol-5-yl]-2-pyrimidinamine

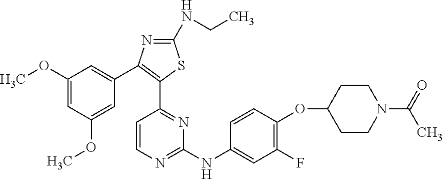

### Step A: 1,1-Dimethylethyl 4-[(2-fluoro-4-nitrophenyl)oxy]-1-piperidinecarboxylate

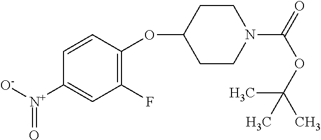

To a cooled (0° C.) solution of 1,1-dimethylethyl 4-hydroxy-1-piperidinecarboxylate (10.06 g, 49.98 mmol) in THF (125 mL) was added dropwise potassium tert-butoxide (1.0 M solution in THF, 54 mL, 54 mmol). The reaction was stirred for 30 min at 0° C. and then 1,2-difluoro-4-nitrobenzene (5.37 mL, 48.5 mmol) was added and the reaction was warmed to rt. After stirring overnight, more potassium tert-butoxide (1.0 M solution in THF, 2.5 mL, 2.5 mmol) was added. The reaction was poured into water (1000 mL) and extracted with DCM (3×400 mL). The combined organic fractions were washed with water (1×1000 mL), dried over Na₂SO₄, filtered, and concentrated. The residual orange oil was triturated with EtOAc:hexanes (1:10, 20 mL) and precipitation occurred. The resultant solid was collected by filtration, washed with hexanes (2×30 mL), and dried under vacuum to afford 9.76 g (59%) of the title compound of Step A. ¹H NMR (400 MHz, CDCl₃): δ 8.03 (m, 2H), 7.05 (t, 1H, J=8.3 Hz), 4.66 (m, 1H), 3.71 (m, 2H), 3.41 (m, 2H), 1.97 (m, 2H), 1.84 (m, 2H), 1.47 (s, 9H); MS (ESI): 363.34 [M+Na]⁺.

### Step B: 4-[(2-Fluoro-4-nitrophenyl)oxy]piperidine trifluoroacetate

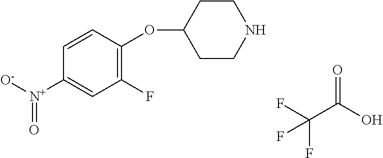

To a solution of 1,1-dimethylethyl 4-[(2-fluoro-4-nitrophenyl)oxy]-1-piperidinecarboxylate (10.55 g, 31 mmol—prepared from multiple batches) in DCM (50 mL) was added TFA (5 mL; 67.31 mmol) at rt and the reaction was stirred for 1 h. The resulting reaction was concentrated in vacuo to give the crude syrup. The crude syrup was triturated with ether (150 mL) and a precipitation occurred. The solid was collected by filtration to give 7.0 g (64%) of the title compound of Step B. ¹H NMR (400 MHz, DMSO-d₆) δ 8.20 (dd, J=11.0, 2.7 Hz, 1H), 8.08-8.14 (m, 1H), 7.52 (t, J=8.9 Hz, 1H), 4.87-4.97 (m, 1H), 3.43 (s, 1H), 3.19-3.32 (m, 2H), 3.11 (s, 2 H), 2.07-2.22 (m, 2H), 1.78-1.94 (m, 2H); MS (ESI): 241 [M+H].

### Step C: [3-Fluoro-4-(4-piperidinyloxy)phenyl]amine

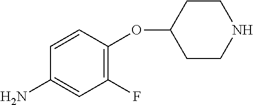

To a stirring mixture of 4-[(2-fluoro-4-nitrophenyl)oxy]piperidine trifluoroacetate (1.02 g; 2.88 mmol) in MeOH (25 mL) was added nickel (II) chloride hexahydrate (0.45 g; 0.66 mmol). The reaction mixture was chilled to 0° C. and sodium borohydride (0.25 g; 6.61 mmol) was added cautiously, causing a vigorous foaming action and the resulting reaction mixture turned black. The reaction was stirred for 0.5 h before more sodium borohydride (0.25 g; 6.61 mmol) was added. The reaction was stirred for 0.25 h and then concentrated in vacuo to a black crude residue. The residue was triturated with EtOAc (50 mL) and quenched with 2.0 N NaOH solution. The phases were separated and the aqueous phase was extracted again with EtOAc. The combined organic phase was washed with brine, filtered through Whatman 1 PS paper and concentrated in vacuo to give the title compound of Step C (0.45 g; 74%). ¹H NMR (400 MHz, DMSO-d₆) δ 6.72-6.89 (m, J=9.5 Hz, 1H), 6.36 (dd, J=13.6, 2.9 Hz, 1H), 6.23-6.29 (m, 1H), 4.97 (s, 2H), 3.88-3.99 (m, 1H), 2.81-2.98 (m, 2H), 2.39-2.48 (m, 2H), 1.99 (br. s., 1H), 1.74-1.86 (m, 2H), 1.32-1.45 (m, 2H).

### Step D: 4-[4-[3,5-Bis(methyloxy)phenyl]-2-(ethylamino)-1,3-thiazol-5-yl]-N-[3-fluoro-4-(4-piperidinyloxy)phenyl]-2-pyrimidinamine

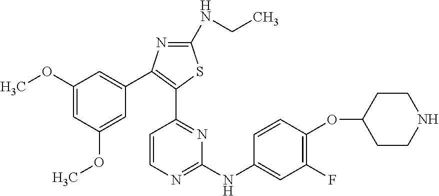

The general procedure (iPrOH (4 mL), 180° C.; 0.25 h; microwave, with 5 drops of conc. HCl) analogous to Example 1, Step G was used for the reaction of 4-[3,5-bis(methyloxy)phenyl]-5-(2-chloro-4-pyrimidinyl)-N-ethyl-1,3-thiazol-2-amine (0.325 g, 0.86 mmol), prepared by a procedure analogous to Example 1, Step F, and [3-fluoro-4-(4-piperidinyloxy)phenyl]amine (0.25 g, 1.19 mmol) to give a crude product. Purification by chromatography (0-100%) DCM:MeOH:NH₄OH/84:15:1 to EtOAc gave the desired title compound of Step D as a yellow solid (0.247 g, 52%). ¹H NMR (400 MHz, DMSO-d₆) δ 9.52 (s, 1H), 8.28 (t, J=5.3 Hz, 1H), 8.12 (d, J=5.5 Hz, 1H), 7.84 (dd, J=14.3, 2.6 Hz, 1H), 7.28-7.40 (m, 1H), 7.01-7.16 (m, 1H), 6.63 (d, J=2.6 Hz, 2H), 6.59 (t, J=2.4 Hz, 1H), 6.35 (d, J=5.5 Hz, 1H), 4.20-4.30 (m, 1 H), 3.75 (s, 6H), 3.26-3.31 (m, 2H), 2.94-3.02 (m, 2H), 2.53-2.62 (m, 2H), 1.85-1.95 (m, 2H), 1.43-1.55 (m, 2H), 1.21 (t, J=7.1 Hz, 3H). MS (ESI): 551 [M+H], 549 [M−H].

### Step E: N-{4-[(1-Acetyl-4-piperidinyl)oxy]-3-fluorophenyl}-4-[4-[3,5-bis(methyloxy)phenyl]-2-(ethylamino)-1,3-thiazol-5-yl]-2-pyrimidinamine

Ac₂O (0.2 g, 0.2 mmol) was added to a chilled (0° C.) solution of 4-[4-[3,5-bis(methyloxy)phenyl]-2-(ethylamino)-1,3-thiazol-5-yl]-N-[3-fluoro-4-(4-piperidinyloxy)phenyl]-2-pyrimidinamine in DCM (2 mL) and the reaction was stirred for 0.5 h. The reaction was diluted further with DCM and washed with saturated NaHCO₃ solution, as well as brine and water. The organic phase was filtered through Whatman 1 PS paper and concentrated in vacuo to a crude residue. Purification by chromatography (0-100%) DCM:MeOH:NH₄OH/84:15:1 to EtOAc still gave an impure product. Purification by reverse phase HPLC gave the desired product of Step E as a yellow solid (0.053 g; 50%). ¹H NMR (400 MHz, DMSO-d₆) δ 9.54 (s, 1 H), 8.28 (t, J=5.3 Hz, 1H), 8.12 (d, J=5.5 Hz, 1H), 7.85 (dd, J=14.7, 2.6 Hz, 1H), 7.35 (d, J=10.3 Hz, 1H), 7.14 (t, J=9.3 Hz, 1H), 6.60-6.65 (m, J=2.6 Hz, 2H), 6.58 (t, J=2.4 Hz, 1H), 6.34 (d, J=5.1 Hz, 1H), 4.39-4.51 (m, 1H), 3.77-3.87 (m, 1H), 3.74 (s, 6H), 3.62-3.72 (m, 1H), 3.19-3.30 (m, 4H), 2.01 (s, 3H), 1.90-1.98 (m, 1H), 1.81-1.89 (m, J=12.8, 12.8 Hz, 1H), 1.57-1.70 (m, 1H), 1.46-1.58 (m, 1H), 1.20 (t, J=7.1 Hz, 3H). MS (ESI): 593.32 [M+H].

### Example 13

### 4-[4-[3,5-Bis(methyloxy)phenyl]-2-(ethylamino)-1,3-thiazol-5-yl]-N-(3-fluoro-4-{[2-(4-morpholinyl)ethyl]oxy}phenyl)-2-pyrimidinamine

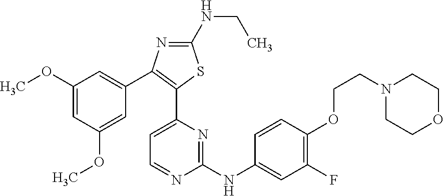

### Step A: (1,1-Dimethylethyl)({2-[(2-fluoro-4-nitrophenyl)oxy]ethyl}oxy)-dimethylsilane

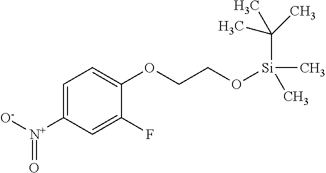

2-{[(1,1-Dimethylethyl)(dimethyl)silyl]oxy}ethanol (5.0 g, 28.3 mmol) was injected into a 0° C. stirred solution of 1,2-difluoro-4-nitrobenzene (2.39 mL, 21.8 mmol), 60% w/w suspension of NaH in oil (1.05 g, 26.2 mmol), and DMF (30 mL). The ice bath was removed and the reaction was allowed to warm to rt. The reaction was stirred for 15 h at rt. TLC confirmed consumption of the starting material and the DMF was removed under vacuum. The resulting residue was partitioned between EtOAc and water. The organic fraction was washed with brine and dried over MgSO₄. The organic fraction was concentrated and purified by silica gel chromatography (gradient: 0-10% EtOAc/hexanes). Purification provided 3 g (43%) of (1,1-dimethylethyl)({2-[(2-fluoro-4-nitrophenyl)oxy]ethyl}oxy)dimethylsilane. ¹H NMR (400 MHz, DMSO-d₆) δ ppm 8.03-8.14 (m, 2H), 7.38 (t, J=8.79 Hz, 1H), 4.21-4.26 (m, 2H), 3.89-3.95 (m, 2H), 0.80 (s, 9H), 0.01 (s, 6H).

### Step B: {4-[(2-{[(1,1-Dimethylethyl)(dimethyl)silyl]oxy}ethyl)oxy]-3-fluorophenyl}amine

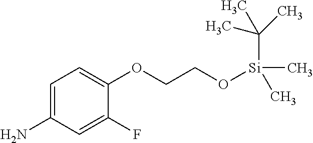

(1,1-Dimethylethyl)({2-[(2-fluoro-4-nitrophenyl)oxy]ethyl}oxy)dimethylsilane (3.0 g, 9.5 mmol) was stirred vigorously with 5% palladium on carbon (300 mg) and EtOAc (30 mL) under a H₂ atmosphere (balloon pressure) for 40 h at rt. The reaction was filtered through a pad of Celite and the filtrate was concentrated under vacuum to yield 2.71 g (100%) of the title compound of Step B. ¹H NMR (400 MHz, DMSO-d₆) δ ppm 6.77 (td, J=9.20, 2.11 Hz, 1H), 6.33 (dt, J=13.60, 2.36 Hz, 1H), 6.22 (d, J=8.61 Hz, 1H), 4.86 (s, 2H), 3.86 (s, 2H), 3.80 (s, 2H), 0.82 (s, 9H), 0.00 (s, 6H).

### Step C: 2-{[4-({4-[4-[3,5-Bis(methyloxy)phenyl]-2-(ethylamino)-1,3-thiazol-5-yl]-2-pyrimidinyl}amino)-2-fluorophenyl]oxy}ethanol

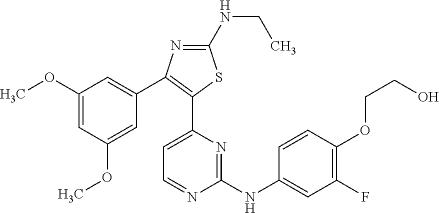

4-[3,5-Bis(methyloxy)phenyl]-5-(2-chloro-4-pyrimidinyl)-N-ethyl-1,3-thiazol-2-amine (1.5 g, 4.0 mmol), prepared by a procedure analogous to Example 1, Step F, {4-[(2-{[(1,1-dimethylethyl)(dimethyl)silyl]oxy}ethyl)oxy]-3-fluorophenyl}amine (1.03 g, 3.6 mmol), an HCl solution (4 M) in dioxane (1.8 mL, 7.2 mmol), and 2,2,2-trifluoroethanol (10 mL) were combined in a sealed vessel. The reaction was heated for 30 min at 170° C. by microwave radiation. The reaction was cooled to rt and concentrated to a residue under vacuum. The residue was purified by silica gel chromatography (gradient: 10-100% (90% CH₂Cl₂:9% MeOH:1% NH₄OH)/CH₂Cl₂). Purification yielded 1.16 g (64%) of 2-{[4-({4-[4-[3,5-bis(methyloxy)phenyl]-2-(ethylamino)-1,3-thiazol-5-yl]-2-pyrimidinyl}amino)-2-fluorophenyl]oxy}ethanol. m/z (ESI): 512.26 [M+H]⁺.

### Step D: 4-[4-[3,5-Bis(methyloxy)phenyl]-2-(ethylamino)-1,3-thiazol-5-yl]-N-{4-[(2-bromoethyl)oxy]-3-fluorophenyl}-2-pyrimidinamine

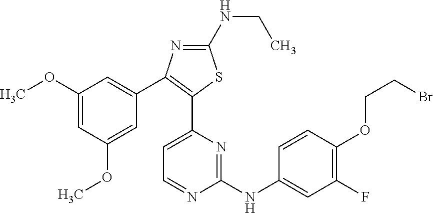

2-{[4-({4-[4-[3,5-bis(Methyloxy)phenyl]-2-(ethylamino)-1,3-thiazol-5-yl]-2-pyrimidinyl}amino)-2-fluorophenyl]oxy}ethanol (1.16 g, 2.3 mmol) was combined with phosphorous tribromide solution (1 M) in DCM (4.5 mL, 4.5 mmol) and 1,2-dichloroethane (30 mL) in a sealed vessel. The reaction was heated for 15 h at 100° C. The reaction was concentrated under vacuum and the residue was partitioned between EtOAc and 1 N NaOH. The organic fraction was washed with brine and dried over MgSO₄. The solvent was removed under vacuum and the residue was purified by silica gel chromatography (gradient: 30-100% EtOAc/hexanes). Purification yielded 225 mg (17%) of 4-[4-[3,5-bis(methyloxy)phenyl]-2-(ethylamino)-1,3-thiazol-5-yl]-N-{4-[(2-bromoethyl)oxy]-3-fluorophenyl}-2-pyrimidinamine as a yellow powder. m/z (ESI): 576.28 [M+H]⁺.

### Step E: 4-[4-[3,5-Bis(methyloxy)phenyl]-2-(ethylamino)-1,3-thiazol-5-yl]-N-(3-fluoro-4-{[2-(4-morpholinyl)ethyl]oxy}phenyl)-2-pyrimidinamine

4-[4-[3,5-Bis(methyloxy)phenyl]-2-(ethylamino)-1,3-thiazol-5-yl]-N-{4-[(2-bromoethyl)oxy]-3-fluorophenyl}-2-pyrimidinamine (33 mg, 0.09 mmol) and morpholine (neat) were combined in a sealed vessel and heated for 10 min at 100° C. by microwave radiation. The reaction was cooled to rt and the morpholine was removed under vacuum. The residue was purified by silica gel chromatography (gradient: 10-100% (90% CH₂Cl₂:9% MeOH:1% NH₄OH)/CH₂Cl₂). Purification provided 14 mg (27%) of the title compound of Example 13. ¹H NMR (400 MHz, DMSO-d₆) δ ppm 9.45 (s, 1H), 8.22 (t, J=5.31 Hz, 1H), 8.07 (d, J=5.31 Hz, 1H), 7.77 (dd, J=14.65, 2.56 Hz, 1H), 7.31 (d, J=9.16 Hz, 1H), 7.04 (t, J=9.43 Hz, 1H), 6.58 (s, 2H), 6.53 (s, 1H), 6.29 (d, J=5.31 Hz, 1H), 4.07 (t, J=5.86 Hz, 2H), 3.70 (s, 6H), 3.49-3.58 (m, 4H), 3.19-3.26 (m, 2H), 2.60-2.67 (m, 2H), 2.45 (s, 4H), and 1.15 (t, J=7.23 Hz, 3H); m/z (ESI): 581.34 [M+H]⁺.

### Example 14

### N-(2,2-Dioxido-1,3-dihydro-2-benzothien-5-yl)-4-{2-(ethylamino)-4-[3-methyl-5-(methyloxy)phenyl]-1,3-thiazol-5-yl}-2-pyrimidinamine

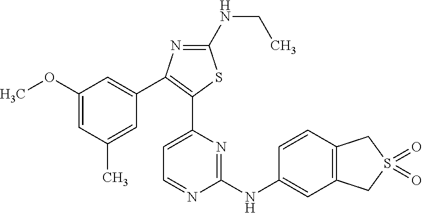

5-(2-Chloro-4-pyrimidinyl)-N-ethyl-4-[3-methyl-5-(methyloxy)phenyl]-1,3-thiazol-2-amine (100 mg, 0.27 mmol), prepared by a procedure analogous to Example 1, Step F, was combined with 1,3-dihydro-2-benzothiophen-5-amine 2,2-dioxide (46 mg, 0.25 mmol), HCl solution (4 M) in dioxane (63 μL, 0.25 mmol) and 2,2,2-trifluoroethanol (3 mL) in a sealed vessel. The reaction was heated for 30 min at 170° C. by microwave radiation. The reaction was cooled, concentrated to a residue and purified by silica gel chromatography (gradient: 5-100% (90% CH₂Cl₂:9% MeOH:1% NH₄OH)/CH₂Cl₂). The fractions with coupled adduct were combined and concentrated under vacuum. The residue was triturated with MeOH and filtered. Filtration yielded 43 mg (34%) of the title compound of Example 14 as a yellow powder. ¹H NMR (400 MHz, DMSO-d₆) δ ppm 9.61 (s, 1H), 8.18 (s, 1H), 8.06 (d, J=4.03 Hz, 1H), 7.90 (s, 1H), 7.58 (d, J=9.52 Hz, 1H), 7.21 (d, J=7.51 Hz, 1H), 6.82 (d, J=16.30 Hz, 2H), 6.78 (s, 1 H), 6.28 (d, J=4.21 Hz, 1H), 4.43 (s, 2H), 4.37 (s, 2H), 3.69 (s, 3H), 2.37-2.45 (m, 2H), 2.27 (s, 3H), 1.10-1.21 (m, 3H); m/z (ESI): 508.23 [M+H]⁺.

### Example 15

### N-{4-[4-[3,5-Bis(methyloxy)phenyl]-2-(cyclopropylamino)-1,3-thiazol-5-yl]-2-pyrimidinyl}-2-methyl-1,2,3,4-tetrahydro-7-isoquinolinamine

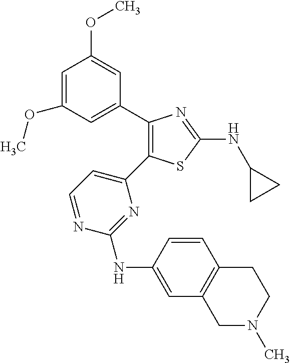

### Step A: 4-[3,5-Bis(methyloxy)phenyl]-5-(2-chloro-4-pyrimidinyl)-N-cyclopropyl-1,3-thiazol-2-amine

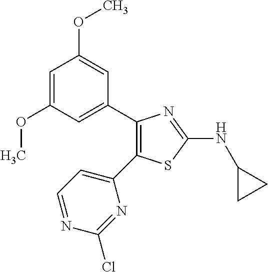

To obtain the desired compound of Step A, (E)-1-[3,5-bis(methyloxy)phenyl]-2-(2-chloro-4-pyrimidinyl)ethanone (4 g, 14 mmol), prepared by a procedure analogous to Example 1, Step E, and DCM (15 mL) were placed in a round bottom flask with stirring. NBS (2.45 g, 14 mmol) was added in one portion and the resulting mixture was allowed to stir at rt for 10 min. Next the reaction was concentrated to dryness. Acetonitrile (15 mL), N-cyclopropylthiourea (3.2 g, 17 mmol) and TEA (2.7 mL, 21 mmol) were added to the α-bromoketone. The reaction was then allowed to heat 5 h at 50° C. and stirred at rt overnight. DCM was added to the reaction mixture and the resulting crude reaction was then concentrated onto silica gel. This was then purified via column chromatography using EtOAc and DCM to yield fractions which were concentrated to dryness. The resulting material was then sonicated in ether and the solid that persisted was filtered off to yield 1.1 g of the target compound of Step A as a yellow powder (20%). ¹H NMR (400 MHz, DMSO-d₆) δ 8.92 (s, 1H), 8.30 (d, J=5.5 Hz, 1H), 6.84 (d, J=5.5 Hz, 1H), 6.63-6.61 (m, 2H), 6.59 (m, 1H), 3.72 (s, 6 H), 2.61 (m, 1H), 0.81 (m, 2H), 0.60 (m, 2H). MS (ESI) m/z 388.99 and 390.97 (M+H)⁺.

### Step B: N-{4-[4-[3,5-Bis(methyloxy)phenyl]-2-(cyclopropylamino)-1,3-thiazol-5-yl]-2-pyrimidinyl}-2-methyl-1,2,3,4-tetrahydro-7-isoquinolinamine

To obtain the title compound of Step B, 4-[3,5-bis(methyloxy)phenyl]-5-(2-chloro-4-pyrimidinyl)-N-cyclopropyl-1,3-thiazol-2-amine (0.08 g, 0.206 mmol) and 2-methyl-1,2,3,4-tetrahydro-7-isoquinolinamine (0.033 g, 0.206 mmol) were combined with iPrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness and subsequently adhered to the silica gel. Column chromatography using EtOAc, MeOH, and NH₄OH yielded fractions which were concentrated to dryness. The resulting material was then sonicated in ether and the solid that persisted was filtered off to yield 55 mg of the title compound of Example 15 (52%) as a yellow powder. ¹H NMR (400 MHz, DMSO-d₆) δ 9.38 (s, 1H), 8.62 (s, 1H), 8.10 (d, J=5.6 Hz, 1H), 7.76 (s, 1H), 7.29 (d, J=8.2 Hz, 1H), 6.97 (d, J=8.1 Hz, 1H), 6.61 (d, J=2.5 Hz, 2H), 6.56 (m, 1H), 6.32 (d, J=5.4 Hz, 1H), 3.73 (s, 6H), 3.49 (s, 2H), 2.74 (m, 2H), 2.61 (m, 1H), 2.57 (m, 2H), 2.31 (s, 3H), 0.79 (m, 2 H), 0.62 (m, 2H). HRMS C₂₈H₃₁N₆O₂S (M+H)⁺ calcd 515.2229. found 515.2238.

### Example 16

### 1-(2-{[4-({4-[4-[3,5-Bis(methyloxy)phenyl]-2-(cyclopropylamino)-1,3-thiazol-5-yl]-2-pyrimidinyl}amino)-2-fluorophenyl]oxy}ethyl)-2-pyrrolidinone trifluoroacetate

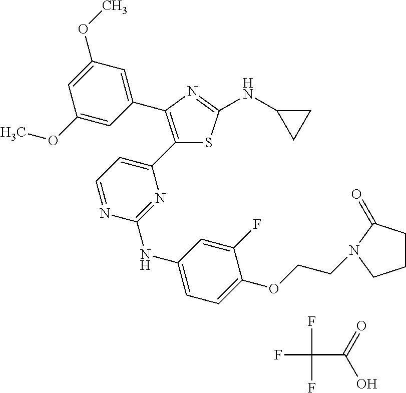

The title compound was synthesized using standard microwave displacement conditions analogous to Example 1, Step G, in trifluoroethanol using 4-[3,5-bis(methyloxy)phenyl]-5-(2-chloro-4-pyrimidinyl)-N-cyclopropyl-1,3-thiazol-2-amine (0.085 g, 0.22 mmol), prepared in a procedure analogous to Example 15, Step A, 1-{2-[(4-amino-2-fluorophenyl)oxy]ethyl}-2-pyrrolidinone (0.057 g, 0.24 mmol). Solvent was removed and the residue was taken up in DMSO/MeOH (2:1) and purified via HPLC to give 0.059 g, 38% yield, of desired as pale yellow solid. ¹H NMR (400 MHz, DMSO-d₆) δ ppm 9.58 (s, 1H), 8.10 (dd, J=5.5, 0.7 Hz, 1H) 8.71 (s, 1H), 7.85 (d, J=14.7 Hz, 1H), 7.31 (dd, J=9.0, 1.3 Hz, 1H), 7.07 (t, J=9.4 Hz, 1H), 6.60 (d, J=2.2 Hz, 2H), 6.55 (t, J=2.3 Hz, 1H), 6.33 (d, J=5.5 Hz, 1H), 4.07 (t, J=5.5 Hz, 2 H), 3.70-3.73 (m, 6H), 3.50 (t, J=5.4 Hz, 2H), 3.41-3.46 (m, 2H), 2.57-2.62 (m, 1H), 2.19 (t, J=8.1 Hz, 2H), 1.89 (qd, J=7.6, 7.42 Hz, 2H), 0.77 (td, J=6.73, 5.0 Hz, 2H), 0.57-0.61 (m, 2H). ES-LCMS m/z 591 (M+H).

### Example 17

### 4-[4-[3,5-Bis(methyloxy)phenyl]-2-(cyclopropylamino)-1,3-thiazol-5-yl]-N-[3-fluoro-4-({1-[2-(methylsulfonyl)ethyl]-4-piperidinyl}oxy)phenyl]-2-pyrimidinamine

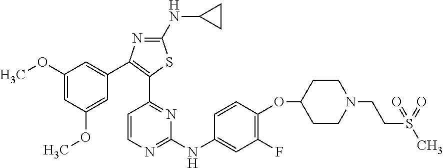

The general procedure (iPrOH (4 mL), 180° C.; 0.25 h; microwave, with 5 drops of conc. HCl), analogous to Example 1, Step G, was used for the reaction of 4-[3,5-bis(methyloxy)phenyl]-5-(2-chloro-4-pyrimidinyl)-N-cyclopropyl-1,3-thiazol-2-amine (0.07 g, 0.18 mmol), prepared by a procedure analogous to Example 15, Step A, and [3-fluoro-4-({1-[2-(methylsulfonyl)ethyl]-4-piperidinyl}oxy)phenyl]amine (0.07 g, 0.22 mmol), prepared by a procedure analogous to Example 10, Step D, to give a crude product. Purification by reverse phase HPLC gave the salt of the desired title compound of Example 17. This crude product was dissolved in DCM, washed with 50% saturated NaHCO₃ solution, filtered through Whatman 1 PS paper and concentrated in vacuo to a mustard yellow solid (0.020 g, 17%). ¹H NMR (400 MHz, DMSO-d₆) δ 9.55 (s, 1H), 8.64 (d, J=1.1 Hz, 1H), 8.14 (d, J=5.3 Hz, 1H), 7.91 (d, J=14.5 Hz, 1H), 7.33 (d, J=9.0 Hz, 1H), 7.10 (t, J=9.3 Hz, 1H), 6.62 (dd, J=2.0 Hz, 2H), 6.57 (t, J=2.2 Hz, 1H), 6.36 (d, J=5.5 Hz, 1H), 4.18-4.29 (m, 1H), 3.74 (s, 6H), 3.24-3.28 (m, 2H), 3.03 (s, 3H), 2.68-2.78 (m, J=6.7, 6.7 Hz, 4H), 2.56-2.64 (m, 1H), 2.26 (t, J=8.7 Hz, 2H), 1.85-1.95 (m, 2H), 1.56-1.68 (m, 2H), 0.74-0.83 (m, 2H), 0.56-0.65 (m, 2H). MS (ESI): 669.29 [M+H].

### Example 18

### 4-[4-[3,5-Bis(methyloxy)phenyl]-2-(cyclopropylamino)-1,3-thiazol-5-yl]-N-(6-{4-[2-(methyloxy)ethyl]-1-piperazinyl}-3-pyridinyl)-2-pyrimidinamine

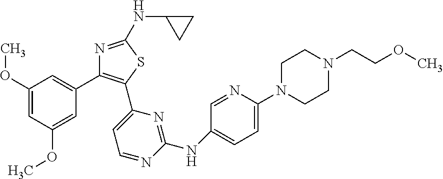

### Step A: 1-[2-(Methyloxy)ethyl]-4-(5-nitro-2-pyridinyl)piperazine

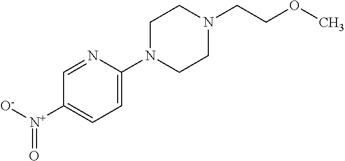

To a solution containing 2.7 g (18.9 mmol) of 1-[2-(methyloxy)ethyl]piperazine and 20 mL of THF at 0° C. was added 0.9 g (23 mmol) of a 60% suspension of NaH in mineral oil. The reaction mixture was allowed to stir for 15 min and 3.0 g (18.9 mmol) of 2-chloro-5-nitropyridine was added. The reaction mixture was heated at 60° C. overnight and then quenched by the addition of water and extracted with EtOAc. The combined organic layers were dried over MgSO₄ and the solvents were removed under reduced pressure. The residue was subjected to silica gel chromatography to give 2.7 g (54%) of 1-[2-(methyloxy)ethyl]-4-(5-nitro-2-pyridinyl)piperazine as a yellow solid: ¹H NMR (400 MHz, DMSO-d₆) δ 2.50-2.53 (m, 6H), 3.24 (s, 3H), 3.46 (t, J=5.7 Hz, 2H), 3.71-3.78 (m, 4H), 6.94 (d, J=9.7 Hz, 2H), 8.21 (dd, J=9.6 and 2.8 Hz, 1H), and 8.95 (d, J=2.75 Hz, 1H).

### Step B: 6-{4-[2-(Methyloxy)ethyl]-1-piperazinyl}-3-pyridinamine hydrochloride

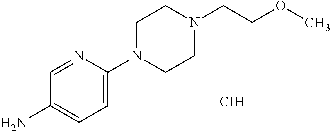

A suspension containing 2.7 g (10.1 mmol) of 1-[2-(methyloxy)ethyl]-4-(5-nitro-2-pyridinyl)piperazine, 0.27 g of 5% Pt on carbon, and 30 mL of EtOH was treated under a 55 psi atm of H₂ for 13 h, then filtered over a pad of Celite. The solvents were removed under reduced pressure to yield 2.4 g (100%) of 6-{4-[2-(methyloxy)ethyl]-1-piperazinyl}-3-pyridinamine. This material was converted to its corresponding hydrochloride salt by treatment of a EtOAc solution of the freebase with 4.0 M HCl in ether. Filtration of the resulting mixture gave 6-{4-[2-(methyloxy)ethyl]-1-piperazinyl}-3-pyridinamine hydrochloride as an off white solid: ¹H NMR (400 MHz, CD₃OD) δ 8.05 (brs, 1H), 7.66 (dd, J=9.3 and 2.8 Hz, 1H), 7.13 (d, J=9.2 Hz, 1H), 4.41 (brs, 2H), 3.75-3.80 (m, 2H), 3.66-3.75 (m, 2H), 3.41-3.46 (m, 7H), 3.33-3.41 (m, 2H), 3.18-3.29 (m, 2H).

### Step C: 4-[4-[3,5-Bis(methyloxy)phenyl]-2-(cyclopropylamino)-1,3-thiazol-5-yl]-N-(6-{4-[2-(methyloxy)ethyl]-1-piperazinyl}-3-pyridinyl)-2-pyrimidinamine

To a solution containing 0.1 g (0.26 mmol) of 4-[3,5-bis(methyloxy)phenyl]-5-(2-chloro-4-pyrimidinyl)-N-cyclopropyl-1,3-thiazol-2-amine, prepared by a procedure analogous to Example 15, Step A, 77 mg (0.28 mmol) of 6-{4-[2-(methyloxy)ethyl]-1-piperazinyl}-3-pyridinamine hydrochloride, and 2 mL of iPrOH was added 0.1 mL of a 4.0 M solution of HCl in dioxane. The reaction mixture was heated to 90° C. in a sealed tube overnight. The solvents were removed under reduced pressure and the residue was subjected to silica gel chromatography and trituration from EtOAc/ether to give 40 mg (26%) of the title compound of Example 18 as a yellow solid: ¹H NMR (400 MHz, DMSO-d₆) δ 9.24 (s, 1H), 8.59 (s, 1H), 8.41-8.45 (m, 1H), 8.07 (d, J=5.5 Hz, 1H), 7.89 (ddd, J=9.2, 2.6, and 2.5 Hz, 1H), 6.78 (d, J=9.2 Hz, 1H), 6.62 (d, J=2.2 Hz, 2H), 6.56-6.59 (m, 1H), 6.29 (d, J=5.49 Hz, 1H), 3.47 (t, J=5.8 Hz, 2H), 3.74 (s, 6H), 3.36-3.41 (m, 4H), 3.33 (brs, 2H), 3.30 (brs, 4H), 3.25 (s, 3

H), 2.57-2.65 (m, 1H), 0.76-0.82 (m, 2H), 0.57-0.66 (m, 2H), and. HRMS calcd for C₃₀H₃₇N₈O₃S: 589.2709 (M+H⁺). Found: 589.2706.

### Example 19

### 4-[4-[3,5-Bis(methyloxy)phenyl]-2-(cyclopropylamino)-1,3-thiazol-5-yl]-N-(6-{[(3R)-1-(2-fluoroethyl)-3-pyrrolidinyl]oxy}-3-pyridinyl)-2-pyrimidinamine

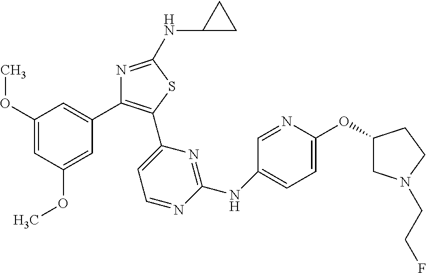

### Step A: 1,1-Dimethylethyl (3R)-3-[(5-nitro-2-pyridinyl)oxy]-1-pyrrolidinecarboxylate

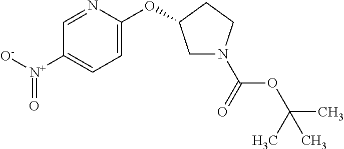

To a solution containing 3.5 g (18.9 mmol) of 1,1-dimethylethyl (3R)-3-hydroxy-1-pyrrolidinecarboxylate and 20 mL of THF was slowly added 0.9 g (23 mmol) of a 60% dispersion of NaH in mineral oil. The reaction mixture was allowed to stir at rt for 15 min, then 3.0 g of 2-chloro-5-nitropyridine was added. The reaction mixture was heated at 50° C. overnight, quenched by the addition of water, and extracted with EtOAc. The combined organic layers were dried over MgSO₄ and the solvents were removed under reduced pressure. The residue was subjected to silica gel chromatography to give 1.3 g (22%) of 1-dimethylethyl (3R)-3-[(5-nitro-2-pyridinyl)oxy]-1-pyrrolidinecarboxylate as a brown oil. An additional 1.7 g of impure material was also collected. Additional purification of this second material by silica gel chromatography gave an additional 1.0 g (17%) of clean product with identical characterization data: ¹H NMR (400 MHz, DMSO-d₆) δ 9.10 (d, J=2.9 Hz, 1H), 8.49 (dd, J=9.2 and 2.9 Hz, 1H), 7.05 (d, J=9.2 Hz, 1H), 5.62 (brs, 1H), 3.63 (td, J=13.2 and 4.1 Hz, 1H), 3.39-3.49 (m, 2H), 3.33-3.36 (m, 1H), 2.15-2.27 (m, 1H), 2.05-2.15 (m, 1H), and 1.39 (s, 9H).

### Step B: 5-Nitro-2-[(3R)-3-pyrrolidinyloxy]pyridine bis(trifluoroacetate)

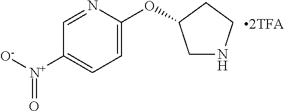

To a solution containing 1.3 g (4.2 mmol) of 1-dimethylethyl (3R)-3-[(5-nitro-2-pyridinyl)oxy]-1-pyrrolidinecarboxylate and 10 mL of DCM was added 0.5 mL of TFA. The reaction mixture was allowed to stir at rt overnight and the solvents were removed under reduced pressure to give 1.75 g (96%) of 5-nitro-2-[(3R)-3-pyrrolidinyloxy]pyridine bis(trifluoroacetate) as a sticky brown solid: ¹H NMR (400 MHz, DMSO-d₆) δ 9.18 (brs, 1H), 9.11 (d, J=2.8 Hz, 1H), 9.06 (brs, 1H), 8.53 (dd, J=9.2 and 2.9 Hz, 1H), 7.04 (d, J=9.2 Hz, 1H), 5.71 (ddd, J=7.0, 5.1, and 1.8 Hz, 1H), 3.55 (td, J=12.1 and 6.2 Hz, 1H), 3.40-3.48 (m, 1H), 3.30-3.40 (m, 2 H), 2.27-2.37 (m, 1H), 2.16-2.23 (m, 1H).

### Step C: 2-{[(3R)-1-(2-Fluoroethyl)-3-pyrrolidinyl]oxy}-5-nitropyridine

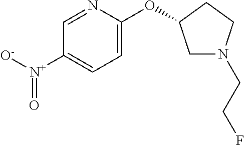

To a solution containing 0.6 g (1.37 mmol) of 5-nitro-2-[(3R)-3-pyrrolidinyloxy]pyridine bis(trifluoroacetate) and 10 mL of DMF was added 0.2 mL (2.8 mmol) of 1-bromo-2-fluoroethane and 0.58 g (5.5 mmol) of K₂CO₃. The reaction mixture was heated at 50° C. overnight and partitioned between EtOAc and water. The aqueous layer was further extracted with EtOAc and the combined organic layers were dried over MgSO₄. The solvents were removed under reduced pressure and the residue was subjected to silica gel chromatography to give 2-{[(3R)-1-(2-fluoroethyl)-3-pyrrolidinyl]oxy}-5-nitropyridine in a quantitative yield: ¹H NMR (400 MHz, DMSO-d₆) δ 9.07 (d, J=2.9 Hz, 1H), 8.46 (dd, J=9.0 and 2.8 Hz, 1H), 7.02 (d, J=9.17 Hz, 1H), 5.45-5.51 (m, 1H), 4.58 (t, J=5.0 Hz, 1H), 4.46 (t, J=5.0 Hz, 1H), 2.80-2.89 (m, 3H), 2.75-2.79 (m, 1H), 2.68-2.74 (m, 1H), 2.41-2.48 (m, 1H), 2.26-2.36 (m, 1H), and 1.79-1.89 (m, 1H); LC/MS: 256.32 (M+H⁺).

### Step D: 6-{[(3R)-1-(2-Fluoroethyl)-3-pyrrolidinyl]oxy}-3-pyridinamine

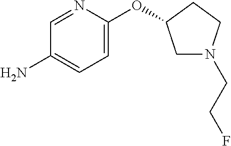

A solution containing 0.35 g (1.4 mmol) of 2-{[(3R)-1-(2-fluoroethyl)-3-pyrrolidinyl]oxy}-5-nitropyridine, 35 mg of 10% Pt on carbon, and 20 mL of EtOH was treated with a 60 psi atm of H₂ overnight. The reaction mixture was filtered through a pad of Celite, eluting with EtOH and EtOAc to give 0.3 g (99%) of 6-{[(3R)-1-(2-fluoroethyl)-3-pyrrolidinyl]oxy}-3-pyridinamine as a brown oil: ¹H NMR (400 MHz, DMSO-d₆) δ 7.47 (d, J=2.9 Hz, 1H), 6.98 (dd, J=8.4 and 2.6 Hz, 1H), 6.50 (d, J=8.4 Hz, 1H), 5.13-5.19 (m, 1H), 4.71 (brs, 2H), 4.56 (t, J=4.6 Hz, 1H), 4.44 (t, J=5.0 Hz, 1H), 2.84 (dd, J=10.6 and 6.2 Hz, 1H), 2.69-2.76 (m, 2H), 2.66 (t, J=5.1 Hz, 1H), 2.61 (dd, J=10.3 and 2.9 Hz, 1H), 2.41-2.47 (m, 1H), 2.17 (td, J=13.7 and 7.5 Hz, 1H), and 1.68-1.76 (m, 1H); LC/MS: 226.11 (M+H⁺).

### Step E: 4-[4-[3,5-Bis(methyloxy)phenyl]-2-(cyclopropylamino)-1,3-thiazol-5-yl]-N-(6-{[(3R)-1-(2-fluoroethyl)-3-pyrrolidinyl]oxy}-3-pyridinyl)-2-pyrimidinamine

A solution containing 0.1 g (0.26 mmol) of 4-[3,5-bis(methyloxy)phenyl]-5-(2-chloro-4-pyrimidinyl)-N-cyclopropyl-1,3-thiazol-2-amine, prepared by a procedure analogous to Example 15, Step A, 0.07 g (0.31 mmol) of 2-{[(3R)-1-(2-fluoroethyl)-3-pyrrolidinyl]oxy}-5-nitropyridine, 2 mL of iPrOH, and 0.1 mL of a 4.0 M solution of HCl in dioxane was heated to 90° C. in a sealed tube overnight. The solvents were removed under reduced pressure and the residue was subjected to silica gel chromatography, then further purified by HPLC to give 51 mg (34%) of 4-[4-[3,5-bis(methyloxy)phenyl]-2-(cyclopropylamino)-1,3-thiazol-5-yl]-N-(6-{[(3R)-1-(2-fluoroethyl)-3-pyrrolidinyl]oxy}-3-pyridinyl)-2-pyrimidinamine as a yellow solid: ¹H NMR (400 MHz, DMSO-d₆) δ 9.40 (s, 1H), 8.61 (d, J=1.7 Hz, 1H), 8.45 (d, J=2.8 Hz, 1H), 8.10 (d, J=5.5 Hz, 1H), 8.02 (dd, J=8.8 and 3.1 Hz, 1H), 6.74 (d, J=9.0 Hz, 1H), 6.62 (d, J=2.4 Hz, 2H), 6.57 (t, J=2.2 Hz, 1H), 6.34 (d, J=5.5 Hz, 1H), 5.25-5.32 (m, 1H), 4.56-4.61 (m, 1H), 4.46 (t, J=4.9 Hz, 1H), 3.73 (s, 6H), 3.29 (s, 1H), 2.89 (td, J=6.9 and, 4.5 Hz, 1H), 2.72-2.80 (m, 2H), 2.65-2.72 (m, 2H), 2.58-2.65 (m, 1H), 2.21-2.31 (m, 1H), 1.74-1.84 (m, 1H), 0.79 (td, J=6.7 and 4.7 Hz, 2H), and 0.58-0.65 (m, 2H); HRMS calcd for C₂₉H₃₃N₇O₃FS: 578.2350. Found: 578.2342.

### Example 20

### 4-[4-[3,5-Bis(methyloxy)phenyl]-2-(methylamino)-1,3-thiazol-5-yl]-N-(3-chloro-4-{[2-(1-pyrrolidinyl)ethyl]oxy}phenyl)-2-pyrimidinamine

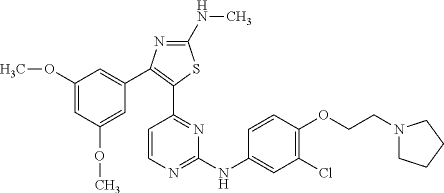

### Step A: 4-[3,5-Bis(methyloxy)phenyl]-5-(2-chloro-4-pyrimidinyl)-N-methyl-1,3-thiazol-2-amine

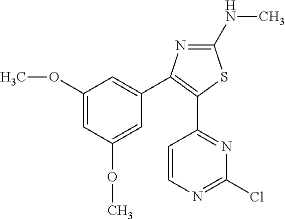

To a solution of 1-[3,5-bis(methyloxy)phenyl]-2-(2-chloro-4-pyrimidinyl)ethanone (1.00 g, 3.42 mmol), prepared by a procedure analogous to Example 1, Step E, in DCM (17 mL) was added NBS (609 mg, 3.42 mmol). The reaction was stirred for 30 min at rt and was then concentrated. The residue was redissolved in 1,4-dioxane (17 mL) and N-methylthiourea (370 mg, 4.10 mmol) and MgCO₃ (1.00 g) were added. The reaction was heated to 50° C. for 16 h and then was partitioned between water (100 mL) and EtOAc (100 mL), resulting in the precipitation of a solid. The solid was collected by filtration and washed with 0.2 N aqueous HCl (2×100 mL), water (1×100 mL), and MeOH (2×50 mL) to give 880 mg (71%) of the title compound of Step A. ¹H NMR (400 MHz, DMSO-d₆): δ 8.49 (q, 1H, J=4.5 Hz), 8.30 (d, 1H, J=5.7 Hz), 6.83 (d, 1H, J=5.7 Hz), 6.62 (m, 3H), 3.74 (s, 6H), 2.90 (d, 3H, J=4.7 Hz); MS (ESI): 363.05 [M+H]⁺.

### Step B: 1-{2-[(2-Chloro-4-nitrophenyl)oxy]ethyl}pyrrolidine

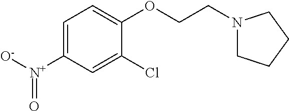

2-Chloro-1-fluoro-4-nitrobenzene (2.5 g, 14.2 mmol), 2-(1-pyrrolidinyl)ethanol (3.3 mL, 28.5 mmol), cesium carbonate (23 g, 71.2 mmol) and DMF (10 mL) were stirred for 15 h at 90° C. The reaction product was partitioned between EtOAc and water. The organic fraction was washed with brine and dried over MgSO₄. The crude oil was purified by silica gel column chromatography (0-100% gradient, (90 CH₂Cl₂: 9 MeOH: 1 NH₄OH)—CH₂Cl₂)). Purification yielded 2.68 g (70%) of a yellow oil. MS (ESI): M+H=271.02.

### Step C: (3-Chloro-4-{[2-(1-pyrrolidinyl)ethyl]oxy}phenyl)amine hydrochloride

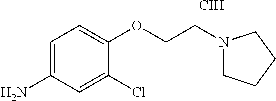

1-{2-[(2-chloro-4-nitrophenyl)oxy]ethyl}pyrrolidine (2.68 g, 9.9 mmol) was combined with 5% platinum on carbon (200 mg) and EtOH (50 mL). The reaction was stirred vigorously for 3 h at rt under a H₂ balloon. The platinum was removed by filtration through Celite and the crude filtrate was concentrated to an oil under vacuum. The residual crude material was dissolved in MeOH and treated with 6.5 mL (1 equivalent) of hydrochloric acid solution (1 N HCl in Et₂O). The solvent was removed under vacuum providing 1.56 g (57%) of (3-chloro-4-{[2-(1-pyrrolidinyl)ethyl]oxy}phenyl)amine hydrochloride, a tan powder. ¹H NMR (400 MHz, DMSO-d₆) δ 10.90 (brs, 1H), 6.95 (d, 1H), 6.75 (s, 1H), 6.59 (d, 1H), 6.10 (brs, 1H), 4.24 (t, 2H), 3.57 (brs, 2H), 3.51 (t, 2H), 3.09 (brs, 2H), 1.98 (brs, 2H) 1.85 (brs, 2H).

### Step D: 4-[4-[3,5-Bis(methyloxy)phenyl]-2-(methylamino)-1,3-thiazol-5-yl]-N-(3-chloro-4-{[2-(1-pyrrolidinyl)ethyl]oxy}phenyl)-2-pyrimidinamine (Title Compound)

A solution of 4-[3,5-bis(methyloxy)phenyl]-5-(2-chloro-4-pyrimidinyl)-N-methyl-1,3-thiazol-2-amine (100 mg, 0.287 mmol), (3-chloro-4-{[2-(1-pyrrolidinyl)ethyl]oxy}phenyl)amine hydrochloride (91 mg, 0.330 mmol) and 4 N hydrogen chloride in 1,4-dioxane (144 μL, 0.574 mmol) in 2,2,2-trifluoroethanol (2.9 mL) was heated in a microwave at 170° C. for 20 min. The reaction was then concentrated and purified by preparative HPLC (20 to 70% acetonitrile:water w/0.1% TFA). The material obtained was redissolved in EtOAc (30 mL) and washed with saturated aqueous NaHCO₃ (2×40 mL). The organic fraction was dried over Na₂SO₄, filtered, and concentrated to afford 61 mg (39%) of the title compound of Example 20. ¹H NMR (400 MHz, DMSO-d₆): δ 9.48 (br s, 1H), 8.21 (q, 1H, J=4.6 Hz), 8.11 (d, 1H, J=5.3 Hz), 8.05 (m, 1H), 7.52 (dd, 1H, J=2.3, 8.9 Hz), 7.08 (d, 1H, J=9.2 Hz), 6.63 (d, 2H, J=2.2 Hz), 6.58 (t, 1H, J=2.2 Hz), 6.32 (d, 1H, J=5.5 Hz), 4.10 (t, 2H, J=5.8 Hz), 3.75 (s, 6H), 2.89 (m, 3H), 2.81 (t, 2H, J=5.7 Hz), 2.55 (m, 4H), 1.68 (m, 4H); MS (ESI): 567.32 [M+H]⁺.

### Example 21

### 4-{4-[3,5-Bis(methyloxy)phenyl]-2-[(2-fluoroethyl)amino]-1,3-thiazol-5-yl}-N-(3-chloro-4-{[2-(1-pyrrolidinyl)ethyl]oxy}phenyl)-2-pyrimidinamine

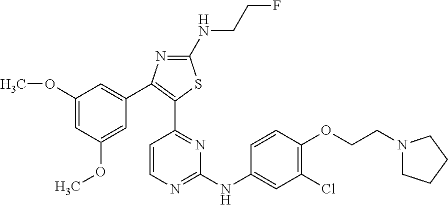

### Step A: N-(2-Fluoroethyl)thiourea

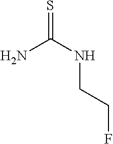

A mixture of 2-fluoroethylamine hydrochloride (2.00 g, 20.1 mmol) and potassium thiocyanate (1.95 g, 20.1 mmol) in 1:1 THF:1,4-dioxane (13.4 mL) was heated to 85° C. for 2.5 h, then stirred at rt for 17 h. The reaction mixture was concentrated and triturated with MeOH (20 mL), causing precipitation of a white solid. The mixture was filtered through Celite to remove the solid, washing with MeOH (2×7 mL), and concentrated. The residue was triturated with DCM (7 mL), filtered, and concentrated to afford 2.33 g (95%) of the title compound of Step A. ¹H NMR (400 MHz, DMSO-d₆): δ 7.94 (br s, 3H), 4.62 (dt, 2H, J=4.7, 47.1 Hz), 3.21-3.11 (m, 2H).

### Step B: 4-[3,5-Bis(methyloxy)phenyl]-5-(2-chloro-4-pyrimidinyl)-N-(2-fluoroethyl)-1,3-thiazol-2-amine

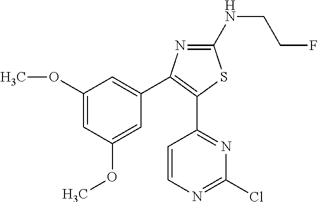

The title compound of Step B was prepared from 1-[3,5-bis(methyloxy)phenyl]-2-(2-chloro-4-pyrimidinyl)ethanone (1.00 g, 3.42 mmol), prepared by a procedure analogous to Example 1, Step E, and N-(2-fluoroethyl)thiourea (501 mg, 4.10 mmol), by a procedure analogous to Example 1, Step F, except that no MgCO₃ was used. The crude reaction mixture was concentrated onto silica gel. Purification by flash column chromatography (0 to 40% EtOAc:DCM) afforded 440 mg of the title compound of Step B in 70% purity, used without further purification. MS (ESI): 395.26 [M+H]⁺.

### Step C: 4-{4-[3,5-Bis(methyloxy)phenyl]-2-[(2-fluoroethyl)amino]-1,3-thiazol-5-yl}-N-(3-chloro-4-{[2-(1-pyrrolidinyl)ethyl]oxy}phenyl)-2-pyrimidinamine

The title compound of Example 21 was prepared from 4-[3,5-bis(methyloxy)phenyl]-5-(2-chloro-4-pyrimidinyl)-N-(2-fluoroethyl)-1,3-thiazol-2-amine (110 mg, 0.279 mmol) and (3-chloro-4-{[2-(1-pyrrolidinyl)ethyl]oxy}phenyl)amine hydrochloride (85 mg, 0.306 mmol), prepared by a procedure analogous to Example 20, Step C, in 24% yield by a procedure analogous to Example 1, Step G. ¹H NMR (400 MHz, DMSO-d₆): δ 9.50 (s, 1H), 8.50 (m, 1H), 8.12 (m, 1H), 8.01 (m, 1H), 7.53 (m, 1H), 7.15-7.00 (m, 1H), 6.71-6.58 (m, 3H), 6.34 (m, 1H), 4.60 (d, 2H, J=47.1 Hz), 4.10 (m, 2H), 3.80-3.58 (m, 8H), 2.82 (m, 2H), 2.56 (m, 4H), 1.68 (m, 4H); MS (APCI): 601.08 [M+H]⁺.

### Example 22

### 4-[4-[3,5-Bis(methyloxy)phenyl]-2-(cyclobutylamino)-1,3-thiazol-5-yl]-N-[3-fluoro-4-({1-[2-(methylsulfonyl)ethyl]-4-piperidinyl}oxy)phenyl]-2-pyrimidinamine

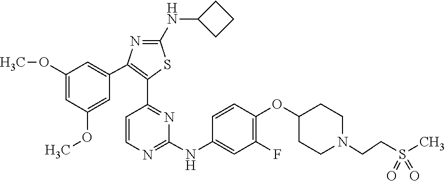

### Step A: N-Cyclobutylthiourea

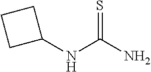

To a solution of cyclobutylamine (1.89 g, 26.6 mmol) in THF (9 mL) was added HCl (4 N solution in 1,4-dioxane, 6.7 mL, 27 mmol). The reaction stirred 30 min at rt, and then potassium thiocyanate (2.58 g, 26.6 mmol) was added. The reaction was heated to 85° C. for 2.5 h, then stirred at rt for 14 h. The reaction mixture was concentrated and triturated with MeOH (25 mL), causing precipitation of a white solid. The mixture was filtered through Celite to remove the solid, washing with MeOH (2×8 mL), and concentrated. The residue was triturated with DCM (8 mL), filtered, and concentrated to afford 3.46 g (100%) of the title compound of Step A. ¹H NMR (400 MHz, CDCl₃): δ 7.76 (br s, 3H), 3.92 (m, 1H), 2.49-2.32 (m, 4H), 2.03 (m, 1H), 1.97-1.85 (m, 1H).

### Step B: 4-[3,5-Bis(methyloxy)phenyl]-5-(2-chloro-4-pyrimidinyl)-N-cyclobutyl-1,3-thiazol-2-amine

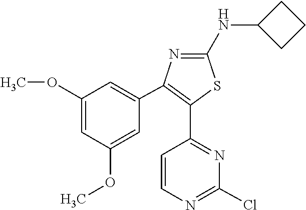

The title compound of Step B was prepared from 1-[3,5-bis(methyloxy)phenyl]-2-(2-chloro-4-pyrimidinyl)ethanone (1.00 g, 3.42 mmol), prepared by a procedure analogous to Example 1, Step E, and N-cyclobutylthiourea (534 mg, 4.10 mmol) by a procedure analogous to Example 1, Step F. Upon completion of the reaction, the reaction mixture was filtered through a fritted funnel to remove solids, washing with EtOAc (2×20 mL). The filtrate was washed with water (1×50 mL). The aqueous fraction was back-extracted with EtOAc (1×40 mL). The combined organic fractions were then washed with saturated aqueous NaCl (1×50 mL), dried over Na₂SO₄, filtered, and concentrated onto Celite. Purification by flash column chromatography (20 to 100% EtOAc:hexanes) afforded 750 mg (54%) of the title compound of Step B. ¹H NMR (400 MHz, CDCl₃): δ 8.13 (d, 1H, J=5.5 Hz), 6.84 (d, 1H, J=5.5 Hz), 6.62 (d, 2H, J=2.4 Hz), 6.55 (t, 1H, J=2.3 Hz), 6.18 (d, 1H, J=7.1 Hz), 3.97 (m, 1H), 3.79 (s, 6H), 2.52-2.44 (m, 2H), 1.92 (m, 2H), 1.77 (m, 2H); MS (ESI): 403.11 [M+H]⁺.

### Step C: 4-[4-[3,5-Bis(methyloxy)phenyl]-2-(cyclobutylamino)-1,3-thiazol-5-yl]-N-[3-fluoro-4-({1-[2-(methylsulfonyl)ethyl]-4-piperidinyl}oxy)phenyl]-2-pyrimidinamine

The title compound of Example 22 was prepared from 4-[3,5-bis(methyloxy)phenyl]-5-(2-chloro-4-pyrimidinyl)-N-cyclobutyl-1,3-thiazol-2-amine (100 mg, 0.248 mmol) and [3-fluoro-4-({1-[2-(methylsulfonyl)ethyl]-4-piperidinyl}oxy)phenyl]amine (82 mg, 0.260 mmol), prepared by a procedure analogous to Example 10, Step D, in 41% yield by a procedure analogous to Example 1, Step G. ¹H NMR (400 MHz, DMSO-d₆): δ 9.53 (s, 1H), 8.62 (d, 1H, J=7.0 Hz), 8.12 (d, 1H, J=5.5 Hz), 7.85 (dd, 1H, J=2.2, 14.5 Hz), 7.32 (d, 1H, J=8.8 Hz), 7.10 (t, 1H, J=9.3 Hz), 6.62 (d, 2H, J=2.2 Hz), 6.58 (t, 1H, J=2.1 Hz, 6.34 (d, 1H, J=5.3 Hz), 4.24 (m, 1H), 4.06 (m, 1H), 3.74 (s, 6H), 3.28 (m, 2H), 3.03 (s, 3H), 2.72 (m, 4H), 2.36-2.24 (m, 4H), 2.04-1.89 (m, 4H), 1.74-1.60 (m, 4H); MS (ESI): 683.37 [M+H]⁺.

### Example 23

### 4-[4-[3,5-Bis(methyloxy)phenyl]-2-(phenylamino)-1,3-thiazol-5-yl]-N-[3-fluoro-4-({1-[2-(methylsulfonyl)ethyl]-4-piperidinyl}oxy)phenyl]-2-pyrimidinamine hydrochloride

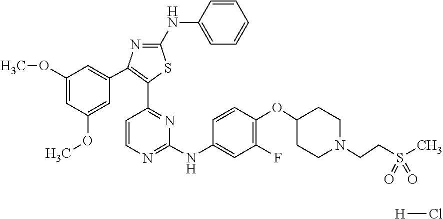

### Step A: 4-[3,5-Bis(methyloxy)phenyl]-5-(2-chloro-4-pyrimidinyl)-N-phenyl-1,3-thiazol-2-amine

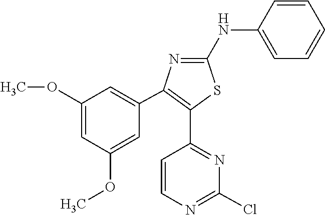

To a solution of 1-[3,5-bis(methyloxy)phenyl]-2-(2-chloro-4-pyrimidinyl)ethanone (1.00 g, 3.42 mmol), prepared by a procedure analogous to Example 1, Step E, in DCM (17 mL) was added NBS (609 mg, 3.42 mmol). The reaction was stirred for 30 min at rt and was then concentrated. The residue was redissolved in 1,4-dioxane (17 mL) and N-phenylthiourea (624 mg, 4.10 mmol) and MgCO₃ (1.00 g) were added. The reaction was heated to 50° C. for 18 h and then was concentrated onto silica gel. Purification by flash column chromatography (0 to 10% EtOAc:hexanes) afforded 1.20 g (83%) of the title compound of Step A. ¹H NMR (400 MHz, CDCl₃): δ 8.58 (br s, 1H), 8.17 (d, 1H, J=5.5 Hz), 7.35 (m, 2H), 7.22 (m, 2H), 7.17 (m, 1H), 6.89 (d, 1H, J=5.5 Hz), 6.62 (d, 2H, J=2.4 Hz), 6.51 (t, 1H, J=2.3 Hz), 3.76 (s, 6H); MS (APCI): 425.09 [M+H]⁺.

### Step B: 4-[4-[3,5-Bis(methyloxy)phenyl]-2-(phenylamino)-1,3-thiazol-5-yl]-N-[3-fluoro-4-({1-[2-(methylsulfonyl)ethyl]-4-piperidinyl}oxy)phenyl]-2-pyrimidinamine hydrochloride

A suspension of 4-[3,5-bis(methyloxy)phenyl]-5-(2-chloro-4-pyrimidinyl)-N-phenyl-1,3-thiazol-2-amine (100 mg, 0.235 mmol) and [3-fluoro-4-({1-[2-(methylsulfonyl)ethyl]-4-piperidinyl}oxy)phenyl]amine (82 mg, 0.259 mmol), prepared by a procedure analogous to Example 10, Step D, in iPrOH (2.4 mL) with 3 drops of concentrated HCl was heated in a microwave at 180° C. for 15 min. The crude reaction mixture was triturated with MeOH (10 mL), and the resultant solid was collected by vacuum filtration, washing with MeOH (2×5 mL) and ether (2×5 mL), to afford 126 mg (72%) of the title compound of Example 23. ¹H NMR (400 MHz, DMSO-d₆): δ 10.88 (br s, 1H), 10.74 (s, 1H), 9.69 (d, 1H, J=3.9 Hz), 8.22 (d, 1H, J=5.3 Hz), 7.89 (d, 1H, J=14.3 Hz), 7.64 (d, 1H, J=7.9 Hz), 7.37 (m, 3H), 7.19 (m, 1H), 7.03 (t, 1H, J=7.3 Hz), 6.71 (d, 1H, J=2.2 Hz), 6.62 (t, 1H, J=2.3 Hz), 6.47 (d, 1H, J=5.3 Hz), 4.58 (m, 1H), 4.38 (m, 1H), 3.76 (s, 6H), 3.59 (m, 2H), 3.48 (m, 3H), 3.25 (m, 2H), 3.13 (s, 3H), 2.26 (m, 1H), 2.11 (m, 2H), 1.93 (m, 1H); MS (ESI): 705.38 [M+H]⁺.

### Example 24

### 4-[4-[3,5-bis(methyloxy)phenyl]-2-(dimethylamino)-1,3-thiazol-5-yl]-N-[3-fluoro-4-({1-[2-(methylsulfonyl)ethyl]-4-piperidinyl}oxy)phenyl]-2-pyrimidinamine

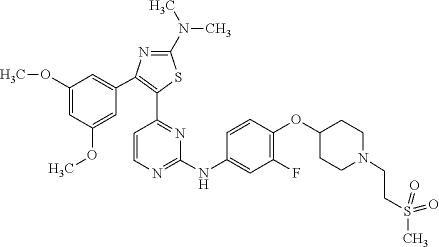

### Step A: N,N-Dimethylthiourea

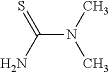

To obtain the title compound of Step A, 2 M dimethyl amine in THF (5 mL) was placed in a round bottom flask and 4 N HCl in dioxane (2.5 mL, 10 mmol) was added drop-wise over 15 min. Potassium thiocyanate (0.97 g, 10 mmol) dissolved in 1 mL water was then added in one portion to the stirring solution of dimethyl amine hydrochloride. This mixture was then allowed to stir 16 h at rt and concentrated to dryness. MeOH (50 mL) was added to the concentrated reaction and solids that persisted were filtered away and subsequent concentration of the MeOH solution yielded 1.1 g of the crude thiourea. ¹H NMR (400 MHz, DMSO-d₆) δ 8.18 (br, 2H), 2.51 (t, J=5.9 Hz, 6H).

### Step B: 4-[3,5-Bis(methyloxy)phenyl]-5-(2-chloro-4-pyrimidinyl)-N,N-dimethyl-1,3-thiazol-2-amine

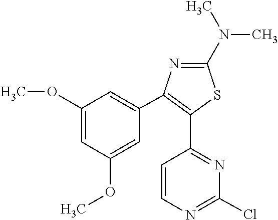

The title compound of Step B was prepared from 1-[3,5-bis(methyloxy)phenyl]-2-(2-chloro-4-pyrimidinyl)ethanone (1.00 g, 3.42 mmol), prepared by a procedure analogous to Example 1, Step E, and N,N-dimethylthiourea (428 mg, 4.10 mmol), by a procedure analogous to Example 1, Step F. The crude reaction mixture was concentrated onto silica. Purification by flash column chromatography (0 to 40% EtOAc:DCM) and subsequent trituration of the chromatographed material with 1:1 ether:hexanes (˜25 mL) afforded 520 mg of the title compound of Step A of 91% purity, used without further purification. MS (ESI): 395.26 [M+H]⁺.

### Step C: 4-[4-[3,5-Bis(methyloxy)phenyl]-2-(dimethylamino)-1,3-thiazol-5-yl]-N-[3-fluoro-4-({1-[2-(methylsulfonyl)ethyl]-4-piperidinyl}oxy)phenyl]-2-pyrimidinamine

To a mixture 4-[3,5-bis(methyloxy)phenyl]-5-(2-chloro-4-pyrimidinyl)-N,N-dimethyl-1,3-thiazol-2-amine (100 mg, 0.265 mmol) and [3-fluoro-4-({1-[2-(methylsulfonyl)ethyl]-4-piperidinyl}oxy)phenyl]amine (88 mg, 0.279 mmol), prepared by a procedure analogous to Example 10, Step D, in iPrOH (2.7 mL) with 3 drops of concentrated HCl added was heated in the microwave at 180° C. for 10 min. The reaction mixture was concentrated and purified by preparative HPLC (20 to 70% acetonitrile:water w/0.1% TFA). The material obtained was redissolved in EtOAc (30 mL) and washed with saturated aqueous NaHCO₃ (2×40 mL). The organic fraction was dried over Na₂SO₄, filtered, and concentrated to afford 59 mg (34%) of the title compound of Example 24. ¹H NMR (400 MHz, DMSO-d₆): δ 9.54 (br s, 1H), 8.13 (d, 1H, J=5.3 Hz), 7.84 (d, 1H, J=14.3 Hz), 7.36 (d, 1H, J=8.2 Hz), 7.12 (t, 1H, J=9.2 Hz), 6.61 (m, 3H), 6.31 (d, 1H, J=4.4 Hz), 4.24 (m, 1H), 3.78 (m, 8H), 3.14 (s, 6H), 3.03 (s, 3H), 2.72 (m, 4H), 2.27 (m, 2H), 1.90 (m, 2H), 1.63 (m, 2H); MS (ESI): 657.47 [M+H]⁺.

### Example 25

### 4-{2-(Ethylamino)-4-[3-methyl-5-(methyloxy)phenyl]-1,3-thiazol-5-yl}-N-[4-(4-morpholinyl)-3-(trifluoromethyl)phenyl]-2-pyrimidinamine

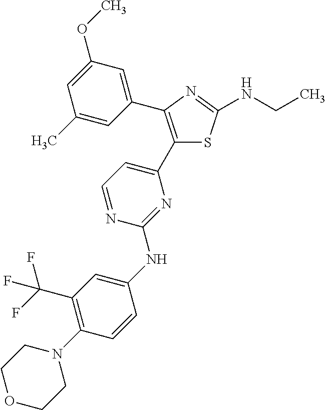

### Step A: Ethyl 3-acetyl-4-hydroxy-5-oxo-2-(2-oxopropyl)-2,5-dihydro-2-furancarboxylate

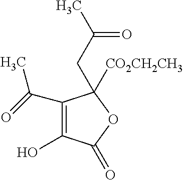

A 1:1 solution of acetic acid and water (680 mL) was added alternately with sodium acetate (311 g, 3.8 moles) in small increments to a flask with ethyl acetopyruvate (300 g, 1.9 moles). The reaction stirred for 15 h at rt. The dark-colored solution was poured into a slurry of ice (500 mL) and concentrated sulfuric acid (100 mL) and the resulting slurry was filtered. The white solid collected was washed rigorously with water. Filtration yielded a wet white powder which was carried directly to the next step without drying or purification. R_{f}=0.1 1:1 EtOAc/hexanes.

### Step B: 3-Hydroxy-5-methylbenzoic acid

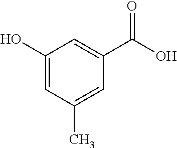

The Ethyl 3-acetyl-4-hydroxy-5-oxo-2-(2-oxopropyl)-2,5-dihydro-2-furancarboxylate from Step A was combined with magnesium oxide (115 g, 2.8 moles) and water (1 L) and heated to 80° C. for 2 h. The reaction was cooled and filtered through a pad of Celite. The filtrate was collected and the pH was adjusted to pH 5 by careful addition of 1 N HCl. The reaction solution was extracted with ether. The organic phase was separated, washed with brine and dried over MgSO₄. The reaction solution was concentrated under vacuum to provide 65 g (45%) of a mixture of the carboxylic acid and ethyl ester as a tan solid. The crude mixture was used directly in the next step without further purification.

### Step C: Methyl 3-hydroxy-5-methylbenzoate

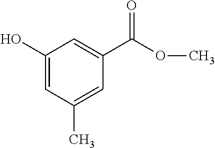

Acetyl chloride (152 mL, 2.1 moles) was added drop-wise to MeOH (200 mL) at 0° C. 3-Hydroxy-5-methylbenzoic acid (65 g, 0.427 moles) from step B was dissolved in MeOH (200 mL) and added drop-wise to the 0° C. acetyl chloride/MeOH solution with stirring. The reaction was brought to rt and stirred for 15 h. The solvent was removed under vacuum and the resulting solid was dissolved in ether. The ether solution was washed with a 10% w/w aqueous solution of NH₄OH, brine and dried over MgSO₄. The organic solution was concentrated under vacuum to yield 54 g (68%) of a brown solid. ¹H NMR (400 MHz, DMSO-d₆) δ ppm 9.68 (s, 1H), 7.20 (s, 1 H), 7.13 (s, 1H), 6.83 (s, 1H), 3.79 (s, 3H), 2.26 (s, 3H).

### Step D: Methyl 3-methyl-5-(methyloxy)benzoate

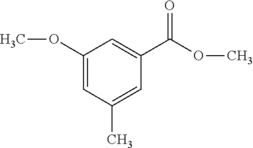

Methyl 3-hydroxy-5-methylbenzoate (54 g, 0.325 moles) from step C was combined methyl iodide (40 mL, 0.650 moles), K₂CO₃ (59 g, 0.422 moles) and DMF (50 mL) and heated to 55° C. for 15 h. LCMS suggested only partial conversion so one additional equivalent of methyl iodide (20 mL, 0.325 moles) and K₂CO₃ (45 g, 0.325 moles) were added and the reaction was stirred at rt for 4 h. The DMF was removed under vacuum and the residue was partitioned between EtOAc and water. The organic layer was washed with brine and dried over MgSO₄. The crude product was purified by silica gel chromatography (gradient: 5-30% EtOAc/hexanes) and produced a yield of 45 g (71%) of a clear oil. ¹H NMR (400 MHz, DMSO-d₆) δ ppm 7.33 (s, 1H), 7.20 (s, 1H), 7.01 (s, 1H), 3.79 (s, 3H), 3.74 (s, 3H), 2.29 (s, 3H).

### Step E: 2-(2-Chloro-4-pyrimidinyl)-1-[3-methyl-5-(methyloxy)phenyl]ethanone

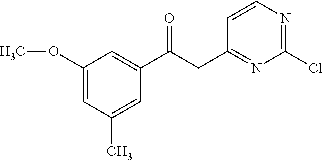

A solution of LHMDS (1 M) in THF (568 mL), was added dropwise to a 0° C. solution of methyl 3-methyl-5-(methyloxy)benzoate (45 g, 0.271 moles) in THF (200 mL). The reaction was stirred for 10 min and a solution of 2-chloro-4-methylpyrimidine (41.8 g, 0.325 moles) in THF (200 mL) was added drop-wise over 30 min to the 0° C. solution of ester and base. TLC suggested all starting material was consumed immediately after addition of pyrimidine. The reaction was quenched with 10% w/w aqueous sodium hydrogen sulfate. The reaction was concentrated to a residue under vacuum. The residue was partitioned between EtOAc and 10% w/w aqueous sodium hydrogen sulfate. The organic layer was separated, washed with brine, and dried over MgSO₄. The crude was purified by silica gel chromatography (gradient: 10-30% EtOAc/hexanes). Purification produced 59 g (80%) of a mixture of tautomers as a yellow powder. m/z (ESI): 277.02 [M+H]⁺.

### Step F: 5-(2-Chloro-4-pyrimidinyl)-N-ethyl-4-[3-methyl-5-(methyloxy)phenyl]-1,3-thiazol-2-amine

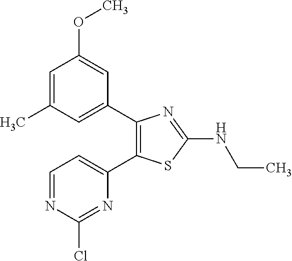

Using a procedure analogous to that described for Example 1, Step F, 122 g of the title compound of Step F was made from 123 g of 2-(2-chloro-4-pyrimidinyl)-1-[3-methyl-5-(methyloxy)phenyl]ethanone (prepared from multiple batches). ¹H NMR (400 MHz, CDCl₃) δ 8.10 (d, J=5.5 Hz, 1H), 7.33 (br. s., 1H), 6.89 (s, 1H), 6.83 (s, 1H), 6.80 (s, 1H), 6.76 (d, J=5.5 Hz, 1H), 3.80 (s, 3H), 3.11 (q, 2H), 2.37 (s, 3H), 1.12 (t, J=7.1 Hz, 3H). MS (ESI): 361 [M+H]⁺.

### Step G: 4-{2-(Ethylamino)-4-[3-methyl-5-(methyloxy)phenyl]-1,3-thiazol-5-yl}-N-[4-(4-morpholinyl)-3-(trifluoromethyl)phenyl]-2-pyrimidinamine

The title compound of Example 25 was synthesized using standard microwave displacement conditions analogous to Example 1, Step G, in trifluoroethanol using 5-(2-chloro-4-pyrimidinyl)-N-ethyl-4-[3-methyl-5-(methyloxy)phenyl]-1,3-thiazol-2-amine (0.100 g, 0.28 mmol) and [4-(4-morpholinyl)-3-(trifluoromethyl)phenyl]amine (0.075 g, 0.31 mmol). Solvent was removed and the residue was taken up in DMSO/MeOH (2:1) and purified via HPLC. The desired fractions were combined, diluted with EtOAc, and washed with NaHCO₃x₂. The organic layer was dried over MgSO₄ and the solvent was removed to give 0.034 g, 22% yield, of the title compound of Example 25 as a pale yellow solid. ¹H NMR (400 MHz, DMSO-d₆) δ ppm 9.65-9.75 (m, 1H), 8.28 (s, 1H), 8.02-8.11 (m, 2H), 7.40-7.51 (m, 1H), 6.88 (s, 1H), 6.73-6.84 (m, 2H), 6.26-6.38 (m, 2H), 4.59 (d, J=4.8 Hz, 1H), 4.40-4.49 (m, 1H), 3.93 (d, J=6.6 Hz, 1H), 3.64-3.73 (m, 5H), 3.16 (d, J=7.7 Hz, 2H), 2.80 (d, J=3.7 Hz, 3H), 2.62-2.70 (m, 1H), 2.52 (s, 1H), 2.29 (d, J=3.3 Hz, 3H), 1.19 (t, J=7.15 Hz, 1H). ES-LCMS m/z 571 (M+H).

### Example 26

### 5-[(4-{2-(Ethylamino)-4-[3-methyl-5-(methyloxy)phenyl]-1,3-thiazol-5-yl}-2-pyrimidinyl)amino]-2-(4-morpholinyl)benzoic acid

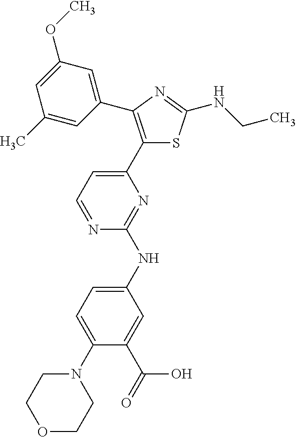

The title compound of Example 26 was synthesized using standard microwave displacement conditions analogous to Example 1, Step G in trifluoroethanol using 5-(2-chloro-4-pyrimidinyl)-N-ethyl-4-[3-methyl-5-(methyloxy)phenyl]-1,3-thiazol-2-amine (1.000 g, 2.78 mmol), prepared in a procedure analogous to Example 25, Step F, and methyl 5-amino-2-(4-morpholinyl)benzoate (0.653 g, 3.33 mmol). Solvent was removed and the residue was taken up in DMSO/MeOH (2:1) and purified via HPLC. Desired fractions were combined diluted with EtOAc and washed twice with NaHCO₃. Organic layer dried over MgSO₄ and solvent removed to give 0.619 g, 43% yield, of desired product as a yellow solid. ¹H NMR (400 MHz, DMSO-d₆) δ ppm 9.73 (s, 1 H), 8.46 (d, J=2.93 Hz, 1H), 8.23 (t, J=5.50 Hz, 1H), 8.10 (d, J=5.50 Hz, 1H), 8.01 (dd, J=8.80, 2.93 Hz, 1H), 7.62 (d, J=8.80 Hz, 1H), 6.88 (s, 1H), 6.82 (d, J=9.90 Hz, 2H), 6.33 (d, J=5.50 Hz, 1H), 3.94-3.98 (m, 2H), 3.76-3.82 (m, 3H), 3.72 (s, 3H), 2.99-3.07 (m, 4H), 2.30 (s, 3H), 1.44-1.55 (m, 1H), 1.30 (dd, J=15.03, 7.70 Hz, 1H), 0.86 (t, J=7.33 Hz, 3H). ES-LCMS m/z 547 (M+H).

### Example 27

### 4-{2-(Ethylamino)-4-[3-methyl-5-(methyloxy)phenyl]-1,3-thiazol-5-yl}-N-(3-fluoro-4-{4-[2-(methylsulfonyl)ethyl]-1-piperazinyl}phenyl)-2-pyrimidinamine

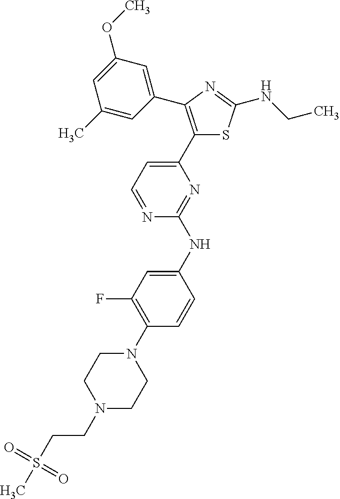

The title compound of Example 28 was synthesized using standard microwave displacement conditions analogous to Example 1, Step G, in trifluoroethanol using 5-(2-chloro-4-pyrimidinyl)-N-ethyl-4-[3-methyl-5-(methyloxy)phenyl]-1,3-thiazol-2-amine (0.085 g, 0.24 mmol), prepared by a procedure analogous to Example 25, Step F and (3-fluoro-4-{4-[2-(methylsulfonyl)ethyl]-1-piperazinyl}phenyl)amine (0.078 g, 0.26 mmol), prepared by a procedure analogous to Example 6, Step B. The solvent was removed and the residue was taken up in DMSO/MeOH (2:1) and purified via HPLC.

Desired fractions were combined, diluted with EtOAc, and washed twice with NaHCO₃. The organic layer was dried over MgSO₄ and the solvent was removed to give 0.055 g, 37% yield, of desired product as a yellow solid. ¹H NMR (400 MHz, DMSO-d₆) δ ppm 9.46 (s, 1H), 8.20 (s, 1H), 8.04 (d, J=5.5 Hz, 1H), 7.70 (dd, J=15.8, 2.38 Hz, 1H), 7.33 (dd, J=8.7, 2.11 Hz, 1H), 6.87-6.96 (m, 1H), 6.75-6.85 (m, 2H), 6.24 (d, J=5.5 Hz, 1H), 3.69 (s, 3H), 3.21-3.31 (m, 6H), 2.95-3.02 (m, 3H), 2.92 (s, 3H), 2.67-2.75 (m, 2H), 2.55 (s, 4H), 2.27 (s, 3H), 1.15 (t, J=7.23 Hz, 3H). ES-LCMS m/z 626 (M+H).

### Example 28

### 4-{2-(Ethylamino)-4-[3-methyl-5-(methyloxy)phenyl]-1,3-thiazol-5-yl}-N-[3-fluoro-4-(4-morpholinyl)phenyl]-2-pyrimidinamine

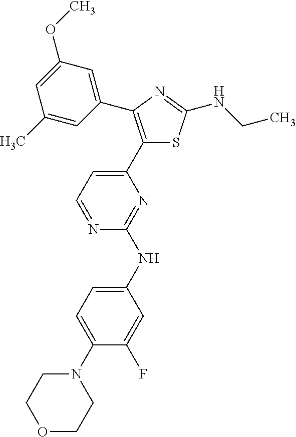

The title compound of Example 29 was synthesized using standard microwave displacement conditions in trifluoroethanol analogous to Example 1, Step G, using 5-(2-chloro-4-pyrimidinyl)-N-ethyl-4-[3-methyl-5-(methyloxy)phenyl]-1,3-thiazol-2-amine (0.085 g, 0.24 mmol) prepared by a procedure analogous to Example 25, Step F, and [3-fluoro-4-(4-morpholinyl)phenyl]amine (0.051 g, 0.26 mmol) prepared by a procedure analogous to Example 7, Step B. The solvent was removed and the residue was taken up in DMSO/MeOH (2:1) and purified via HPLC. Desired fractions were combined, diluted with EtOAc, and washed twice with saturated aqueous NaHCO₃. The organic layer was dried over MgSO₄ and the solvent was removed to give 0.064 g, 52% yield, of desired product as a solid. ¹H NMR (400 MHz, DMSO-d₆) δ ppm 9.49 (s, 1H), 8.24 (s, 1H), 8.03-8.07 (m, 1H), 7.73 (dd, J=15.75, 2.38 Hz, 1H), 7.36 (dd, J=8.79, 1.83 Hz, 1H), 6.89-6.96 (m, 1H), 6.86 (s, 1H), 6.80 (d, J=11.54 Hz, 2H), 6.26 (d, J=5.31 Hz, 1H), 3.65-3.73 (m, 7H), 3.26 (br. s., 2H), 2.85-2.94 (m, 4H), 2.28 (s, 3H), and 1.17 (t, J=7.23 Hz, 3H). ES-LCMS m/z 521 (M+H).

### Example 29

### 4-{2-(Ethylamino)-4-[3-methyl-5-(methyloxy)phenyl]-1,3-thiazol-5-yl}-N-{3-fluoro-4-[4-(2-fluoroethyl)-1-piperazinyl]phenyl}-2-pyrimidinamine

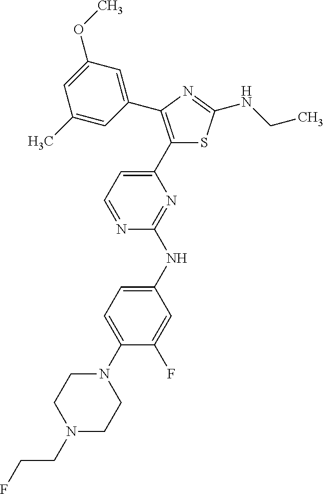

The title compound of Example 29 was synthesized using standard microwave displacement conditions analogous to Example 1, Step G in trifluoroethanol using 5-(2-chloro-4-pyrimidinyl)-N-ethyl-4-[3-methyl-5-(methyloxy)phenyl]-1,3-thiazol-2-amine (0.085 g, 0.24 mmol), prepared by a procedure analogous to Example 25, Step F, and {3-fluoro-4-[4-(2-fluoroethyl)-1-piperazinyl]phenyl}amine (0.060 g, 0.25 mmol), prepared by a procedure analogous to Example 8, Step B. The solvent was removed and the residue was taken up in DMSO/MeOH (2:1) and purified via HPLC. Desired fractions were combined, diluted with EtOAc, and washed twice with NaHCO₃. The organic layer was dried over MgSO₄ and the solvent was removed to give 0.036 g, 27% yield, of desired product as a solid. ¹H NMR (400 MHz, DMSO-d₆) δ ppm 9.48 (s, 1H), 8.23 (t, J=5.5 Hz, 1H), 8.07 (d, J=5.5 Hz, 1H), 7.73 (dd, J=15.8, 2.6 Hz, 1H), 7.31-7.38 (m, 1H), 6.90-6.97 (m, 1H), 6.87 (s, 1H), 6.82 (d, J=11.4 Hz, 2H), 4.61 (t, J=4.8 Hz, 1H), 4.47-4.51 (m, 1H), 3.72 (s, 3H), 3.28 (dd, J=7.3, 5.5 Hz, 2H), 2.92-2.96 (m, 3H), 2.69 (t, J=5.0 Hz, 1H), 2.55-2.64 (m, 5 H), 2.50-2.54 (m, 1H), 2.30 (s, 3H), 1.18 (t, J=7.2 Hz, 3H). ES-LCMS m/z 566 (M+H).

### Example 30

### 1-Acetyl-N-(4-{2-(ethylamino)-4-[3-methyl-5-(methyloxy)phenyl]-1,3-thiazol-5-yl}-2-pyrimidinyl)-2,3-dihydro-1H-indol-5-amine

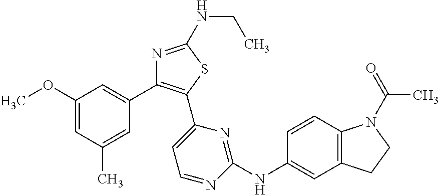

5-(2-Chloro-4-pyrimidinyl)-N-ethyl-4-[3-methyl-5-(methyloxy)phenyl]-1,3-thiazol-2-amine (500 mg, 1.4 mmol), prepared in a procedure analogous to Example 25, Step F, was combined with 1-acetyl-2,3-dihydro-1H-indol-5-amine (224 mg, 1.3 mmol), HCl solution (4M) in dioxane (30 μL, 0.13 mmol) and 2,2,2-trifluoroethanol (10 mL) in a sealed vessel. The reaction was heated for 30 min at 170° C. by microwave radiation. The reaction was cooled, concentrated to a residue and purified by silica gel chromatography (gradient: 5-100% (90% CH₂Cl₂:9% MeOH:1% NH₄OH)/CH₂Cl₂). The fractions with coupled adduct were combined and concentrated under vacuum. The residue was triturated with methanol and filtered. Filtration yielded 400 mg (63%) of the title compound of Example 30 as a yellow powder. ¹H NMR (400 MHz, DMSO-d₆) δ ppm 9.36 (s, 1H), 8.18 (t, J=5.4 Hz, 1H), 8.02 (d, J=5.5 Hz, 1H), 7.89 (d, J=8.8 Hz, 1H), 7.59 (s, 1H), 7.50 (d, J=8.4 Hz, 1H), 6.84 (s, 1H), 6.79 (d, J=9.7 Hz, 2H), 6.22 (d, J=5.5 Hz, 1H), 4.04 (t, J=8.4 Hz, 2H), 3.69 (s, 3H), 3.21-3.27 (m, 2H), 3.09 (t, J=8.3 Hz, 2H), 2.27 (s, 3H), 2.09 (s, 3H), 1.15 (t, J=7.2 Hz, 3H); m/z (ESI): 501.38 [M+H]⁺.

### Example 31

### N-[3-Chloro-4-(4-methyl-1-piperazinyl)phenyl]-4-{2-(ethylamino)-4-[3-methyl-5-(methyloxy)phenyl]-1,3-thiazol-5-yl}-2-pyrimidinamine

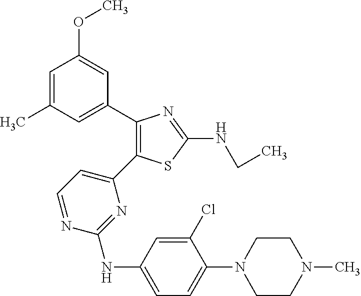

To obtain the title compound of Example 31, 5-(2-chloro-4-pyrimidinyl)-N-ethyl-4-[3-methyl-5-(methyloxy)phenyl]-1,3-thiazol-2-amine (0.066 g, 0.183 mmol), prepared by a procedure analogous to Example 25, Step F, and [3-chloro-4-(4-methyl-1-piperazinyl)phenyl]amine (0.041 g, 0.183 mmol) were combined with iPrOH (2 mL) and concentrated HCl (2 drops) in a microwave vial. The reaction was heated to 180° C. for 20 min in the microwave then cooled to rt. TEA (approx. 0.1 mL) and silica gel were combined with the reaction and the resulting mixture was concentrated to dryness and subsequently adhered to silica gel. Column chromatography using EtOAc, MeOH, and NH₄OH yielded fractions which were concentrated to dryness. This material was then further purified on a reverse phase acidic HPLC. The resulting fractions were free-based via extraction and concentrated to dryness to yield 35 mg of the title compound of Example 31 (35% Y). ¹H NMR (400 MHz, DMSO-d₆) δ 9.51 (s, 1H), 8.25 (t, J=5.4 Hz, 1H), 8.07 (d, J=5.6 Hz, 1H), 8.03 (d, J=2.5 Hz, 1H), 7.54 (dd, J=8.8, 2.6 Hz, 1H), 7.06 (d, J=8.9 Hz, 1H), 6.88 (s, 1 H), 6.82 (d, J=9.7 Hz, 2H), 6.29 (d, J=5.5 Hz, 1H), 3.72 (s, 3H), 3.28 (m, 2H), 2.91 (br, 4H), 2.48 (m, 4H), 2.30 (s, 3H), 2.23 (s, 3H), 1.19 (t, J=7.0 Hz, 3H). HRMS C₂₈H₃₃N₇OSCl (M+H)⁺ calcd 550.2156. found 550.2167.

### Example 32

### 4-{2-(Ethylamino)-4-[3-methyl-5-(methyloxy)phenyl]-1,3-thiazol-5-yl}-N-[3-fluoro-4-({1-[2-(methylsulfonyl)ethyl]-4-piperidinyl}oxy)phenyl]-2-pyrimidinamine

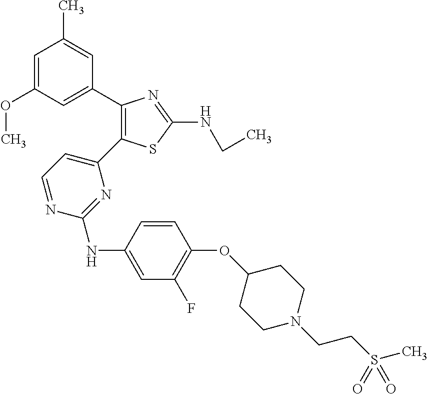

To obtain the title compound of Example 32, 5-(2-chloro-4-pyrimidinyl)-N-ethyl-4-[3-methyl-5-(methyloxy)phenyl]-1,3-thiazol-2-amine (0.08 g, 0.222 mmol), prepared by a procedure analogous to Example 25, Step F, and [3-fluoro-4-({1-[2-(methylsulfonyl)ethyl]-4-piperidinyl}oxy)phenyl]amine (0.07 g, 0.222 mmol), prepared by a procedure analogous to Example 10, Step D, were combined with iPrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness and subsequently adhered to silica gel. Column chromatography using EtOAc, MeOH, and NH₄OH yielded fractions which were concentrated to dryness. This material was then further purified on a reverse phase acidic HPLC. The resulting fractions were free-based via extraction and concentrated to dryness to yield 47 mg of the title compound of Example 32 (27% Y). ¹H NMR (400 MHz, DMSO-d₆) δ 9.50 (s, 1H), 8.23 (t, J=5.4 Hz, 1H), 8.07 (d, J=5.5 Hz, 1H), 7.82 (dd, J=14.4, 2.5 Hz, 1H), 7.32 (d, J=9.1 Hz, 1H), 7.08 (t, J=9.3 Hz, 1H), 6.87 (s, 1H), 6.82 (d, J=11.3 Hz, 2H), 6.28 (d, J=5.4 Hz, 1H), 4.22 (m, 1H), 3.72 (s, 3H), 3.27 (m, 4H), 3.02 (s, 3H), 2.70 (m, 4H), 2.30 (s, 3H), 2.25 (m, 2H), 1.89 (m, 2H), 1.63 (m, 2H), 1.18 (t, J=7.5 Hz, 3H); HRMS C₃₁H₃₈N₆O₄FS₂ (M+H)⁺ calcd 641.2380, found 641.2390.

### Example 33

### 4-{2-(Ethylamino)-4-[3-methyl-5-(methyloxy)phenyl]-1,3-thiazol-5-yl}-N-[3-(methyloxy)-4-(4-methyl-1-piperazinyl)phenyl]-2-pyrimidinamine

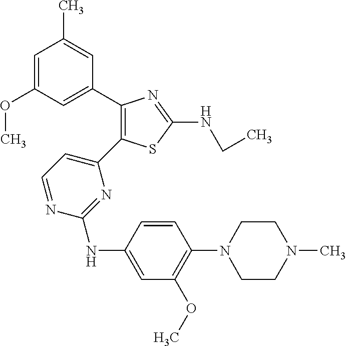

To obtain the title compound of Example 33, 5-(2-chloro-4-pyrimidinyl)-N-ethyl-4-[3-methyl-5-(methyloxy)phenyl]-1,3-thiazol-2-amine (0.07 g, 0.194 mmol), prepared by a procedure analogous to Example 25, Step F, and [3-(methyloxy)-4-(4-methyl-1-piperazinyl)phenyl]amine (0.043 g, 0.194 mmol) were combined with iPrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness and subsequently adhered to the silica gel. Column chromatography using EtOAc, MeOH, and NH₄OH yielded fractions which were concentrated to dryness. The resulting material was then sonicated in ether and the solid that persisted was filtered off to yield 57 mg of the title compound of Example 33 (54% Y). ¹H NMR (400 MHz, DMSO-d₆) δ 9.17 (s, 1H), 8.20 (t, J=5.4 Hz, 1H), 8.02 (d, J=5.3 Hz, 1H), 7.35 (s, 1H), 7.26 (d, J=8.3 Hz, 1H), 6.89-6.78 (m, 4H), 6.22 (d, J=5.5 Hz, 1H), 3.73 (s, 3H), 3.72 (s, 3H), 3.26 (m, 2H), 2.99 (br, 4H), 2.45 (br, 4H), 2.29 (s, 3H), 2.21 (s, 3H), 1.18 (t, J=7.1 Hz, 3H). HRMS C₂₉H₃₆N₇O₂S (M+H)⁺ calcd 546.2651. found 546.2662.

### Example 34

### N-[6-(1,1-Dioxido-4-thiomorpholinyl)-3-pyridinyl]-4-{2-(ethylamino)-4-[3-methyl-5-(methyloxy)phenyl]-1,3-thiazol-5-yl}-2-pyrimidinamine

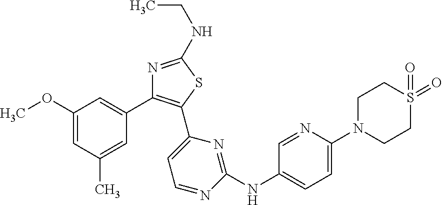

### Step A: 4-(5-Nitro-2-pyridinyl)thiomorpholine 1,1-dioxide

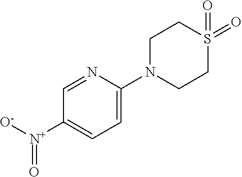

To a solution containing 2.8 g (20.8 mmol) of thiomorpholine 1,1-dioxide and 30 mL of THF was added 0.98 g (24.6 mmol) of a 60% dispersion of NaH in mineral oil. The reaction mixture was allowed to stir for 20 min and 3.0 g (18.9 mmol) of 2-chloro-5-nitropyridine was added slowly. The reaction mixture was heated at 50° C. overnight, quenched by the addition of water, and extracted with EtOAc and DCM. The combined organic layers were dried over MgSO₄ and the solvents were removed under reduced pressure. The residue was subjected to silica gel chromatography to give 2.0 g (38%) of 4-(5-nitro-2-pyridinyl)thiomorpholine 1,1-dioxide: ¹H NMR (400 MHz, DMSO-d₆) δ 9.01 (d, J=2.8 Hz), 8.31 (dd, J=9.5 and 2.8 Hz, 1H), 7.16 (d, J=9.7 Hz, 1H), 4.18-4.25 (m, 4H), 3.20-3.25 (m, 4H).

### Step B: 6-(1,1-Dioxido-4-thiomorpholinyl)-3-pyridinamine

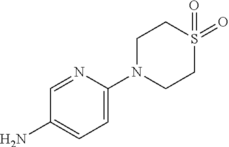

A mixture containing 2.0 g (7.9 mmol) of 4-(5-nitro-2-pyridinyl)thiomorpholine 1,1-dioxide, 0.2 g of 5% Pt on carbon, and 30 mL of EtOH was subjected to a 60 psi H₂ atmosphere for 18 h. The reaction mixture was filtered through a pad of Celite, eluting with EtOH and EtOAc and the solvent was removed under reduced pressure to give 0.9 g (51%) of 6-(1,1-dioxido-4-thiomorpholinyl)-3-pyridinamine: ¹H NMR (400 MHz, DMSO-d₆) δ 7.61 (d, J=3.30 Hz, 1H), 6.96 (dd, J=8.8 and 2.9 Hz, 1H), 6.80 (d, J=8.2 Hz, 1H), 4.68 (brs, 2H), 3.82-3.88 (m, 4H), 3.00-3.05 (m, 4H).

### Step C: N-[6-(1,1-Dioxido-4-thiomorpholinyl)-3-pyridinyl]-4-{2-(ethylamino)-4-[3-methyl-5-(methyloxy)phenyl]-1,3-thiazol-5-yl}-2-pyrimidinamine

To a suspension containing 0.1 g (0.28 mmol) of 5-(2-chloro-4-pyrimidinyl)-N-ethyl-4-[3-methyl-5-(methyloxy)phenyl]-1,3-thiazol-2-amine, prepared by a procedure analogous to Example 25, Step F, 0.08 g (0.33 mmol) of 6-(1,1-dioxido-4-thiomorpholinyl)-3-pyridinamine and 2 mL of iPrOH was added 0.1 mL of a 4.0 M solution of HCl in dioxane. The reaction mixture was heated at 90° C. in a sealed tube for 12 h and the solvent was removed under reduced pressure. The residue was subjected to silica gel chromatography and further purified by HPLC to give 92 mg (61%) of N-[6-(1,1-dioxido-4-thiomorpholinyl)-3-pyridinyl]-4-{2-(ethylamino)-4-[3-methyl-5-(methyloxy)phenyl]-1,3-thiazol-5-yl}-2-pyrimidinamine as a yellow solid: ¹H NMR (400 MHz, DMSO-d₆) δ 9.31 (s, 1H), 8.53 (d, J=2.4 Hz, 1H), 8.22 (t, J=5.3 Hz, 1H), 8.04 (d, J=5.5 Hz, 1H), 7.94 (dd, J=9.2 and 2.8 Hz, 1H), 6.99 (d, J=9.0 Hz, 1H), 6.88 (s, 1H), 6.84 (s, 1H), 6.81 (s, 1H), 6.26 (d, J=5.3 Hz, 1H), 3.97-4.04 (m, 4H), 3.73 (s, 3H), 3.28-3.31 (m, 2H), 3.06-3.12 (m, 4H), 2.31 (s, 3H), 1.18 (t, J=7.2 Hz, 3H). HRMS calcd for C₂₆H₃₀N₇O₃S₂ (M+H⁺): 552.1852. Found: 552.1860.

### Example 35

### 4-{2-(Ethylamino)-4-[3-methyl-5-(methyloxy)phenyl]-1,3-thiazol-5-yl}-N-[6-(4-morpholinyl)-3-pyridinyl]-2-pyrimidinamine

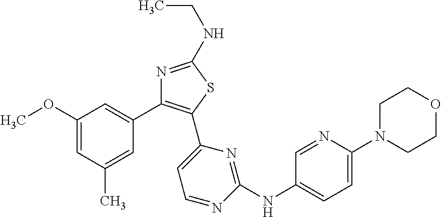

### Step A: 4-(5-Nitro-2-pyridinyl)morpholine

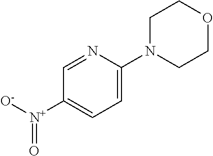

To a solution containing 1.2 mL (13.9 mmol) of morpholine and 20 mL of THF was added 0.66 g (16.4 mmol) of a 60% dispersion of NaH in mineral oil. The reaction was allowed to stir for 15 min and 2.0 g (12.6 mmol) of 2-chloro-5-nitropyridine was added. The reaction mixture was heated at 50° C. overnight, then quenched by the addition of water and extracted with DCM. The combined organic layers were dried over MgSO₄ and the solvents were removed under reduced pressure. The residue was subjected to silica gel chromatography to give 1.65 g (63%) of 4-(5-nitro-2-pyridinyl)morpholine as a yellow solid: ¹H NMR (400 MHz, DMSO-d₆) δ 8.97 (d, J=2.9 Hz, 1H), 8.25 (dd, J=9.5, 2.9 Hz, 1H), 6.95 (d, J=9.5 Hz, 1H), 3.72-3.75 (m, 4H), 3.68-3.71 (m, 4H). MS (ESI): 210.28 (M+H⁺).

### Step B: 6-(4-Morpholinyl)-3-pyridinamine

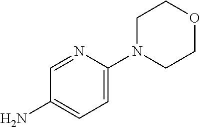

A mixture containing 1.65 g (7.8 mmol) of 4-(5-nitro-2-pyridinyl)morpholine, 160 mg of 5% Pt on carbon, and 20 mL of EtOH was subjected to a 50 psi H₂ atm for 5 h. The reaction mixture was filtered through a pad of Celite and the solvents were removed under reduced pressure to give 1.4 g (100%) of 6-(4-morpholinyl)-3-pyridinamine as a purple solid: ¹H NMR (400 MHz, DMSO-d₆) δ 7.60 (d, J=2.9 Hz, 1H), 6.92 (dd, J=8.8 and 2.9 Hz, 1H), 6.62 (d, J=8.8 Hz, 1H), 4.59 (brs, 2H), 3.65-3.72 (m, 4H), and 3.17 (dt, J=4.9 and 2.4 Hz, 4H).

### Step C: 4-{2-(Ethylamino)-4-[3-methyl-5-(methyloxy)phenyl]-1,3-thiazol-5-yl}-N-[6-(4-morpholinyl)-3-pyridinyl]-2-pyrimidinamine

To a suspension containing 0.1 g (0.28 mmol) of 5-(2-chloro-4-pyrimidinyl)-N-ethyl-4-[3-methyl-5-(methyloxy)phenyl]-1,3-thiazol-2-amine, prepared by a procedure analogous to Example 25, Step F, 0.06 g (0.33 mmol) of 6-(4-morpholinyl)-3-pyridinamine and 1 mL of iPrOH was added 0.1 mL of a 4.0 M solution of HCl in dioxane. The reaction mixture was heated at 180° C. in a microwave reactor for 20 min and the solvent was removed under reduced pressure. The residue was subjected to silica gel chromatography and further purified by HPLC to give 10 mg (7%) of 4-{2-(ethylamino)-4-[3-methyl-5-(methyloxy)phenyl]-1,3-thiazol-5-yl}-N-[6-(4-morpholinyl)-3-pyridinyl]-2-pyrimidinamine as a yellow solid: ¹H NMR (400 MHz, DMSO-d₆) δ 9.26 (s, 1H), 8.49 (d, J=2.6 Hz, 1H), 8.21 (t, J=5.4 Hz, 1H), 8.03 (d, J=5.1 Hz, 1H), 7.89 (dd, J=9.3, 2.7 Hz, 1H), 6.88 (s, 1H), 6.84 (s, 1H), 6.79-6.82 (m, 2H), 6.24 (d, J=5.3 Hz, 1H), 3.73 (s, 3H), 3.70-3.73 (m, 4H), 3.33-3.37 (m, 6H), 3.30 (dd, J=5.2 and 1.7 Hz, 2H), 2.55 (dt, J=3.8 and, 1.8 Hz, 2H), 2.31 (s, 3H), and 1.18 (t, J=7.1 Hz, 3H). HRMS Calcd for C₂₆H₃₀N₇O₂S (M+H⁺): 504.2182. Found: 504.2192.

### Example 36

### N-[6-(4-Acetyl-1-piperazinyl)-3-pyridinyl]-4-{2-(ethylamino)-4-[3-methyl-5-(methyloxy)phenyl]-1,3-thiazol-5-yl}-2-pyrimidinamine

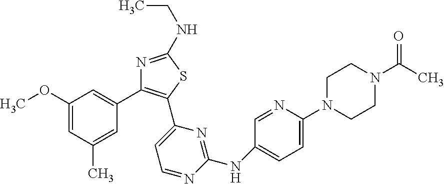

To a suspension containing 0.1 g (0.28 mmol) of 5-(2-chloro-4-pyrimidinyl)-N-ethyl-4-[3-methyl-5-(methyloxy)phenyl]-1,3-thiazol-2-amine, prepared by a procedure analogous to Example 25, Step F, 0.07 g (0.33 mmol) of 6-(4-acetyl-1-piperazinyl)-3-pyridinamine, prepared by a procedure analogous to Example 4, Step B, and 2 mL of iPrOH was added 0.1 mL of a 4.0 M solution of HCl in dioxane. The reaction mixture was heated at 90° C. in sealed tube overnight and 0.5 mL of TEA was added. The solvent was removed under reduced pressure and the residue was subjected to silica gel chromatography and further purified by trituration from EtOAc to give 85 mg (56%) of N-[6-(4-acetyl-1-piperazinyl)-3-pyridinyl]-4-{2-(ethylamino)-4-[3-methyl-5-(methyloxy)phenyl]-1,3-thiazol-5-yl}-2-pyrimidinamine as an orange solid: ¹H NMR (400 MHz, DMSO-d₆) δ 9.26 (s, 1H), 8.49 (d, J=2.4 Hz, 1H), 8.21 (t, J=5.2 Hz, 1H), 8.03 (d, J=5.5 Hz, 1H), 7.89 (dd, J=9.2 and 2.6 Hz, 1H), 6.78-6.89 (m, 3H), 6.24 (d, J=5.5 Hz, 1H), 3.74 (s, 3H), 3.51-3.58 (m, 4H), 3.41-3.49 (m, 2H), 3.35-3.41 (m, 2H), 3.25-3.31 (m, 3H), 2.31 (s, 3H), 2.05 (s, 3H), and 1.18 (t, J=7.2 Hz, 3H). HRMS calcd for C₂₈H₃₃N₈O₂S (M+H⁺): 545.2447. Found: 545.2457.

### Example 37

### 4-{2-(Ethylamino)-4-[3-methyl-5-(methyloxy)phenyl]-1,3-thiazol-5-yl}-N-(6-{4-[(methyloxy)acetyl]-1-piperazinyl}-3-pyridinyl)-2-pyrimidinamine

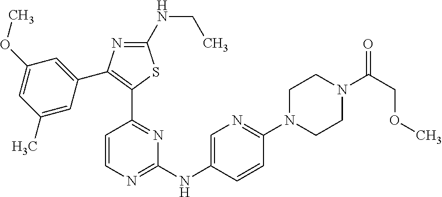

### Step A; 1,1-Dimethylethyl 4-(5-nitro-2-pyridinyl)-1-piperazinecarboxylate

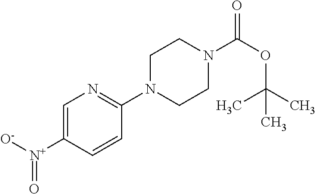

To a solution of tert-butyl piperazine-1-carboxylate (64 g, 346 mmol) in 600 mL of THF at 0° C. was added NaH (16.4 g, 409 mmol, 60% in mineral oil) portionwise. The reaction mixture was stirred for 15 min and 2-chloro-5-nitropyridine (50 g, 314 mmol) was added. The reaction mixture was allowed to warm to rt and then heated to 50° C. for 4 h. The reaction was quenched by water (30 mL) and extracted with DCM (1.5 L×3). The combined organic layers were dried over Na₂SO₄ and the solvent was removed under reduced pressure. The residue was subjected to wash with petroleum ether to give the desired product of Step A (80 g, yield 83%). ¹H NMR (CDCl₃, 400 MHz) δ 8.95 (d, J=2.4 Hz, 1H), 8.24 (d, J=12 Hz, 1H), 6.92 (d, J=6.0 Hz, 1H), 3.75 (s, 4H), 3.44 (s, 4H), and 1.41 (s, 9H).

### Step B: 1-(5-Nitro-2-pyridinyl)piperazine hydrochloride

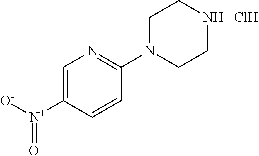

1,1-Dimethylethyl 4-(5-nitro-2-pyridinyl)-1-piperazinecarboxylate (75 g, 244 mmol) was dissolved in DCM (1 L) and excess HCl in MeOH was added dropwise to the solution. The mixture was stirred for about 24 h and then concentrated under vacuum to give the crude product. The crude product was washed with petroleum ether to give the desired product (50 g, yield 70%). ¹H NMR (CDCl₃, 400 MHz) δ 9.56 (s, 2H), 8.98 (d, J=2.8 Hz, 1H), 8.28 (d, J=12 Hz, 1H), 7.01 (d, J=9.2 Hz, 1H), 3.98 (s, 4 H), and 3.17 (s, 4H).

### Step C: 1-[(Methyloxy)acetyl]-4-(5-nitro-2-pyridinyl)piperazine

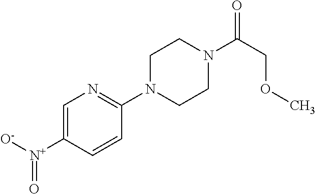

To a solution of 1-(5-nitro-2-pyridinyl)piperazine hydrochloride (2.44 g, 10 mmol) in 50 mL of dry DCM was added Et₃N (7.07 g, 70 mmol). The mixture was stirred for 30 min at rt. 2-Methoxyacetic acid (1.08 g, 12 mmol) was added, followed by HOBT (0.27 g, 2 mmol) and EDCl (5.74 g, 30 mmol). The mixture was stirred at rt for 2 h. The solution was washed with saturated aqueous NaHCO₃ and brine and dried over Na₂SO₄. After evaporation of the solvent, the crude product was purified by chromatography on silica gel (EtOAc/petroleum ether=1:3) to give the desired product of Step C (1.2 g, 55%). ¹H NMR (CDCl₃, 400 MHz) δ 9.05 (s, 1H), 8.26 (dd, J=9.6, 2.0 Hz, 1H), 6.60 (d, J=9.6 Hz, 1H), 4.16 (s, 2H), 3.91-3.82 (brs, 2H), 3.81-3.71 (brs, 4H), 3.72-3.62 (brs, 2H), and 3.45 (s, 3H)

### Step D: 6-{4-[(Methyloxy)acetyl]-1-piperazinyl}-3-pyridinamine hydrochloride

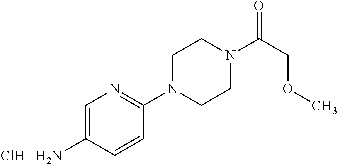

To a solution of 1-[(methyloxy)acetyl]-4-(5-nitro-2-pyridinyl)piperazine (1.2 g, 5.4 mmol) in MeOH (50 mL) was added Raney Ni (0.15 g). The mixture was stirred under hydrogen atmosphere (50 psi/25° C.) for 3.5 h. The reaction mixture was filtered and the filtrate was concentrated under the reduced pressure to dryness to give the crude product, which was purified by recrystallization in EtOAc to afford the desired product of Step D (1.2 g, yield 78%). ¹H NMR (CDCl₃, 400 MHz) δ 7.79 (dd, J=10, 2.8 Hz, 1H), 7.73 (d, J=4.2 Hz, 1H), 7.20 (d, J=9.6 Hz, 1H), 4.23 (s, 2H), 3.80-3.60 (m, 8H), and 3.34 (s, 3H)

### Step E: 4-{2-(Ethylamino)-4-[3-methyl-5-(methyloxy)phenyl]-1,3-thiazol-5-yl}-N-(6-{4-[(methyloxy)acetyl]-1-piperazinyl}-3-pyridinyl)-2-pyrimidinamine

To a solution of 6-{4-[(methyloxy)acetyl]-1-piperazinyl}-3-pyridinamine hydrochloride (119 mg, 0.42 mmol) in BuOH (10 mL) and MeOH (1 mL) was added 5-(2-chloro-4-pyrimidinyl)-N-ethyl-4-[3-methyl-5-(methyloxy)phenyl]-1,3-thiazol-2-amine (150 mg, 0.42 mmol), prepared by a procedure analogous to Example 25, Step F. The mixture was stirred at 140° C. for 4 h. The solvent was removed in vacuo to give the crude product. Further purification by preparative TLC gave the desired product (18 mg, yield 7.5%). ¹H-NMR (CDCl₃, 400 MHz) δ 8.31 (d, J=2.8 Hz, 1H), 7.96 (d, J=5.2 Hz, 1H), 7.91 (dd, J=2.4 and 9.2 Hz, 1H), 7.08 (brs, 1H), 6.92 (s, 1H), 6.83 (s, 1H), 6.78 (s, 1H), 6.68 (d, J=8.8 Hz, 1H), 6.36 (d, J=5.6 Hz, 1H), 6.24 (brs, 1H), 4.16 (s, 2H), 3.77 (brs, 5H), 3.62-3.48 (m, 6H), 3.28 (s, 3H), 3.28 (brs, 2H), 2.34 (s, 3H), and 1.25 (t, J=7.2 Hz, 3H). MS (ESI) m/e (M+H⁺) 575.3.

### Example 38

### 4-{2-(Ethylamino)-4-[3-methyl-5-(methyloxy)phenyl]-1,3-thiazol-5-yl}-N-(3-fluoro-4-{[2-(methyloxy)ethyl]oxy}phenyl)-2-pyrimidinamine

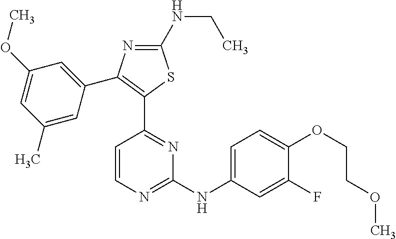

A suspension containing 157 mg (0.44 mmol) of 5-(2-chloro-4-pyrimidinyl)-N-ethyl-4-[3-methyl-5-(methyloxy)phenyl]-1,3-thiazol-2-amine, prepared by a procedure analogous to Example 25, Step F, 97 mg (0.52 mmol) of (3-fluoro-4-{[2-(methyloxy)ethyl]oxy}phenyl)amine, 2 mL of iPrOH, and 0.1 mL of a 4.0 M solution of HCl in dioxane were heated at 90° C. in a sealed tube for 50 h. The solvents were removed under reduced pressure and the residue was subjected to HPLC purification. Desired fractions were taken up in EtOAc, washed with saturated aqueous NaHCO₃, and filtered. The solvent was removed under reduced pressure to give 156 mg (70%) of 4-{2-(ethylamino)-4-[3-methyl-5-(methyloxy)phenyl]-1,3-thiazol-5-yl}-N-(3-fluoro-4-{[2-(methyloxy)ethyl]oxy}phenyl)-2-pyrimidinamine as a yellow solid. ¹H NMR (400 MHz, DMSO-d₆) δ 9.49 (s, 1H), 8.25 (t, J=8.1 Hz, 1H), 8.08 (d, J=5.5 Hz, 1H), 7.81 (dd, J=14.6 and 2.1 Hz, 1H), 7.36 (d, J=8.2 Hz, 1H), 7.07 (t, J=9.5 Hz, 1H), 6.89 (s, 1H), 6.85 (s, 1H), 6.82 (s, 1H), 6.29 (d, J=5.5 Hz, 1H), 4.10-4.14 (m, 2H), 3.74 (s, 3H), 3.64-3.67 (m, 2H), 3.31 (s, 3H), 3.26-3.30 (m, 2H), 2.31 (s, 3H), and 1.19 (t, J=7.1 Hz, 3H). ESIMS: 510.25 (M+H)⁺.

### Example 39

### 4-{2-(Ethylamino)-4-[3-(ethyloxy)-5-methylphenyl]-1,3-thiazol-5-yl}-N-[3-fluoro-4-(4-methyl-1-piperazinyl)phenyl]-2-pyrimidinamine

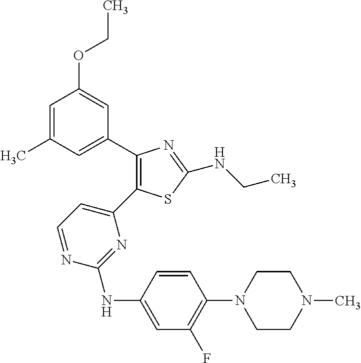

### Step A: Ethyl 3-(ethyloxy)-5-methylbenzoate

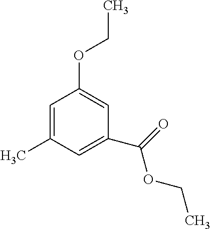

To obtain the desired compound of Step A, NaH (0.122 g, 3.05 mmol) was placed in a 100 mL round bottom flask which was then cooled to 0° C. under a blanket of N₂. THF (2.5 mL) was added to the stirring NaH solids. Ethyl 3-hydroxy-5-methylbenzoate (0.5 g, 2.77 mmol) was then added portion-wise to the stirring reaction over 15 min. The reaction was then allowed to stir at 0° C. for 10 min followed by the addition of ethyl iodide (0.433 g, 2.77 mmol). The resulting mixture was allowed to warm and stir at rt for 1 h. By LC/MS no desired product is seen. DMF was added (2 mL) to the reaction, followed by K₂CO₃ (0.958 g, 6.94 mmol) and more ethyl iodide (0.433 g, 2.77 mmol). The resulting mixture was heated at 60° C. for 30 min. EtOAc and water were added to the reaction mixture and the desired was extracted into the organic phase which was then concentrated to dryness to yield 0.57 g (quant.) of the target compound of Step A. ¹H NMR (400 MHz, DMSO-d₆) δ 7.34 (s, 1H), 7.21 (s, 1H), 7.01 (s, 1H), 4.27 (q, J=7.1 Hz, 2H), 4.04 (q, J=7.0 Hz, 2H), 2.31 (s, 3H), 1.30 (q, J=6.9 Hz, 6H).

### Step B: 2-(2-Chloro-4-pyrimidinyl)-1-[3-(ethyloxy)-5-methylphenyl]ethanone

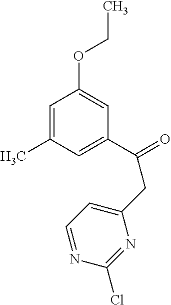

To obtain the title compound of Step B, ethyl 3-(ethyloxy)-5-methylbenzoate (0.57 g, 2.77 mmol) and LHMDS (6.2 mL, 6.2 mmol, 1 M in THF) were placed in a round bottom flask and cooled to 0° C. 2-Chloro-4-methylpyrimidine (0.433 g, 3.38 mmol) was added in one portion and the resulting mixture was allowed to stir and warm to rt overnight. EtOAc and water were added to the reaction mixture and the desired was extracted into the organic phase which was then concentrated onto silica gel and purified via column chromatography to yield 0.438 g of ketone/enolate mixture of the desired target compound of Step B. MS (ESI) m/z 291.34 (M+H)⁺.

### Step C: 5-(2-Chloro-4-pyrimidinyl)-N-ethyl-4-[3-(ethyloxy)-5-methylphenyl]-1,3-thiazol-2-amine

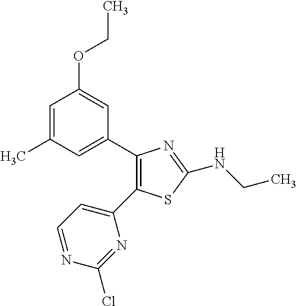

To obtain the desired compound of Step C, 2-(2-chloro-4-pyrimidinyl)-1-[3-(ethyloxy)-5-methylphenyl]ethanone (0.438 g, 1.5 mmol) and DCM (5 mL) were placed in a round bottom flask with stirring. NBS (0.268 g, 1.5 mmol) was added in one portion and the resulting mixture was allowed to stir at rt for 10 min. The reaction was concentrated to dryness. MgCO₃ (0.188 g), ethylthiourea (0.188 g, 1.8 mmol) and 1, 4 dioxane (5 mL) were then added to the α-bromoketone. The reaction was then allowed to heat 5 h at 50° C. and stirred at rt overnight. EtOAc and water were added to the reaction mixture and the desired product was extracted into the organic phase which was then concentrated onto silica gel and purified via column chromatography to yield 0.4 g of the title compound of Step C. ¹H NMR (400 MHz, DMSO-d₆) δ 8.52 (t, J=5.2 Hz, 1H), 8.25 (d, J=5.8 Hz, 1H), 6.86-6.84 (m, 2H), 6.79-6.77 (m, 2H), 3.98 (q, J=6.9 Hz, 2H), 3.29 (m, 2H), 2.29 (s, 3H), 1.28 (t, J=7.0 Hz, 3H), 1.18 (t, J=7.5 Hz, 3H). HRMS calcd for C₂₉H₃₆N₇OFS (M+H)⁺: 548.2608. found: 548.2607.

### Step D: 4-{2-(Ethylamino)-4-[3-(ethyloxy)-5-methylphenyl]-1,3-thiazol-5-yl}-N-[3-fluoro-4-(4-methyl-1-piperazinyl)phenyl]-2-pyrimidinamine

To obtain the title compound of Step D, 5-(2-chloro-4-pyrimidinyl)-N-ethyl-4-[3-(ethyloxy)-5-methylphenyl]-1,3-thiazol-2-amine (0.08 g, 0.219 mmol) and [3-fluoro-4-(4-methyl-1-piperazinyl)phenyl]amine (0.046 g, 0.219 mmol) were combined with iPrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness and subsequently adhered to the silica gel. Column chromatography using EtOAc, MeOH, and NH₄OH yielded fractions which were concentrated to dryness. The resulting material was then sonicated in ether and the solid that persisted was filtered off to yield 41 mg of the title compound of Example 39 (34% Y). ¹H NMR (400 MHz, DMSO-d₆) δ 9.48 (s, 1H), 8.22 (t, J=5.4 Hz, 1H), 8.07 (d, J=5.4 Hz, 1H), 7.72 (dd, J=15.7, 2.4 Hz, 1H), 7.36 (dd, J=8.8, 2.5 Hz, 1H), 6.93 (t, J=9.4 Hz, 1H), 6.86 (s, 1H), 6.79 (d, J=13.4 Hz, 2H), 6.27 (d, J=5.7 Hz, 1H), 3.99 (m, 2H), 3.28 (m, 2H), 2.94 (m, 4H), 2.45 (br, 4H), 2.29 (s, 3H), 2.21 (s, 3H), 1.28 (t, J=7.2 Hz, 3H), 1.18 (t, J=7.6 Hz, 3H). HRMS C₂₉H₃₅N₇OS (M+H)⁺.

### Example 40

### 4-{2-(Ethylamino)-4-[3-(methyloxy)phenyl]-1,3-thiazol-5-yl}-N-(6-{2-[4-(methylsulfonyl)-1-piperazinyl]ethyl}-3-pyridinyl)-2-pyrimidinamine

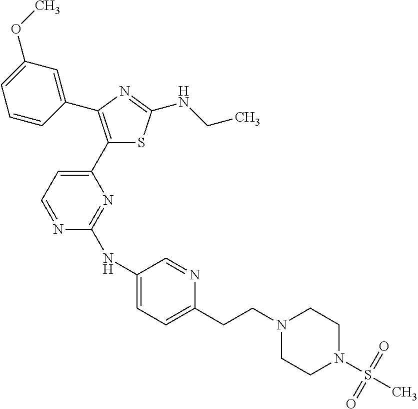

### Step A: 2-(2-Chloro-4-pyrimidinyl)-1-[3-(methyloxy)phenyl]ethanone

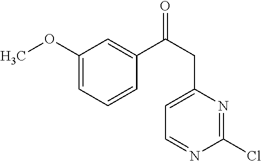

To ethyl 3-methoxybenzoate (20.0 g, 111 mmol) in THF (150 mL) at 0° C. was added 1 N LHMDS in THF (150 mL, 150 mmol) and 2-chloro-4-methylpyrimidine (14.3 g, 111 mmol) in THF (50 mL) was added slowly via addition funnel. The reaction mixture was stirred for 0.5 h then the volatiles were removed under reduced pressure. EtOAc, water and 6 N HCl were added and the layers were separated. The EtOAc layer was dried over Na₂SO₄, filtered and concentrated onto silica. The residue was purified by silica gel chromatography eluting with EtOAc:hexane (10% to 80%) to yield 17 g of the title compound of Step A. MS (ESI): 263 [M+H]⁺.

### Step B: 5-(2-Chloro-4-pyrimidinyl)-N-ethyl-4-[3-(methyloxy)phenyl]-1,3-thiazol-2-amine

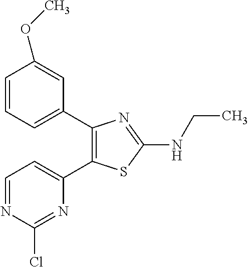

To a solution of 2-(2-chloro-4-pyrimidinyl)-1-[3-(methyloxy)phenyl]ethanone (10.0 g, 38.1 mmol) and 60 mL of DCM was added NBS (6.8 g, 38.1 mmol). The reaction mixture was allowed to stir at rt for 30 min and the solvents were removed under reduced pressure. The residue was diluted with 60 mL of DMF and ethylthiourea (4.4 g, 42 mmol) was added. The reaction mixture was allowed to stir for 30 min at rt and the solvent was removed under reduced pressure. A portion of EtOAc was added and the resulting mixture was filtered to give 10.2 g (77%) of 5-(2-Chloro-4-pyrimidinyl)-N-ethyl-4-[3-(methyloxy)phenyl]-1,3-thiazol-2-amine: ESI MS m/z=347.26 (M+H⁺).

### Step C: 2-Ethenyl-5-nitropyridine

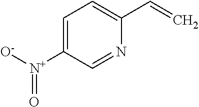

Under nitrogen, tributyl(vinyl)tin (13.196 g, 41.63 mmol) and palladium tetrakis triphenyl phosphine (2.187 g, 1.89 mmol) were added to 2-chloro-5-nitropyridine (6.00 g, 37.84 mmol) and cresol (2.044 g, 18.92 mmol) in 200 mL THF. The reaction mixture was heated to reflux overnight, cooled to rt, and EtOAc and 5 g of sodium fluoride were added. The mixture was stirred at rt for 7 h then filtered. The organic layer was washed with water and brine, and dried over MgSO₄ The solvents were removed and the residue was loaded onto silica and purified via flash chromatography EtOAc/Hexane to give 5.20 g, 89% yield, of the desired product of Step C. ¹H NMR (400 MHz, DMSO-d₆) δ ppm 9.30 (d, J=2.75 Hz, 1H), 8.55 (dd, J=8.70, 2.66 Hz, 1H), 7.76 (d, J=8.79 Hz, 1H), 6.96 (dd, J=17.40, 10.62 Hz, 1H), 6.45 (dd, J=17.40, 1.28 Hz, 1H), 5.71-5.75 (m, 1H).

### Step D: 1-(Methylsulfonyl)-4-[2-(5-nitro-2-pyridinyl)ethyl]piperazine

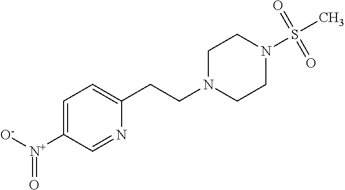

2-Ethenyl-5-nitropyridine (0.550 g, 3.67 mmol) was dissolved in iPrOH and 1-(methylsulfonyl)piperazine (0.662 g, 4.03 mmol) was added. The reaction mixture was heated in microwave to 140° C. for 10 min then loaded onto silica and purified via flash chromatography, EtOAc/MeOH 0-20% gradient. Desired fractions were combined to give 0.990 g, 86% yield of the title compound of Step D. ¹H NMR (400 MHz, DMSO-d₆) δ ppm 9.26 (d, J=2.20 Hz, 1H), 8.49 (dd, J=8.43, 2.93 Hz, 1H), 7.62 (d, J=8.80 Hz, 1H), 3.28 (s, 1H), 3.02-3.08 (m, 5H), 2.83 (s, 3H), 2.76 (t, J=7.33 Hz, 2H), and 2.50-2.53 (m, 4H).

### Step E: 6-{2-[4-(Methylsulfonyl)-1-piperazinyl]ethyl}-3-pyridinamine

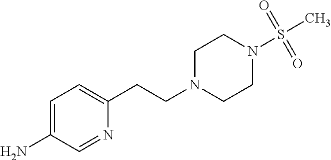

1-(Methylsulfonyl)-4-[2-(5-nitro-2-pyridinyl)ethyl]piperazine (0.990 g, 3.25 mmol) was taken up in MeOH (20 mL) and 10% palladium/carbon (0.050 g) was added. The mixture was stirred under H₂, 1 atm, for 18 h, and filtered through a celite plug. The solvent was removed to give 0.920 g, 99% yield, of a solid. ¹H NMR (400 MHz, DMSO-d₆) δ ppm 7.81 (d, J=2.57 Hz, 1H), 6.87-6.92 (m, 1H), 6.78-6.84 (m, 1 H), 5.03 (d, J=8.07 Hz, 2H), 3.29 (s, 1H), 3.04-3.08 (m, 5H), 2.84 (s, 3H), 2.64-2.70 (m, 3H), and 2.55-2.60 (m, 3H).

### Step F: 4-{2-(Ethylamino)-4-[3-(methyloxy)phenyl]-1,3-thiazol-5-yl}-N-(6-{2-[4-(methylsulfonyl)-1-piperazinyl]ethyl}-3-pyridinyl)-2-pyrimidinamine

5-(2-chloro-4-pyrimidinyl)-N-ethyl-4-[3-(methyloxy)phenyl]-1,3-thiazol-2-amine (0.050 g, 0.14 mmol) and 6-{2-[4-(methylsulfonyl)-1-piperazinyl]ethyl}-3-pyridinamine (0.041 g, 0.14 mmol) were dissolved in 5 mL of toluene. Pd(OAc)₂(0.002 g, cat), Xantphos (0.002 g, cat) and CsCO₃ (0.094 g, 0.29 mmol) were added and the mixture was heated in the microwave to 140° C. for 30 min. The solvent was removed and the residue was taken up in DMSO/MeOH (2:1) and purified via HPLC. Desired fractions were combined, diluted with EtOAc, and washed twice with NaHCO₃. The organic layer was dried over MgSO₄ and the solvent was removed to give 0.015 g, 17% yield of the title compound of Example 40 as a solid. ¹H NMR (400 MHz, DMSO-d₆) δ ppm 9.55 (s, 1H), 8.81 (d, J=2.0 Hz, 1H), 8.22-8.29 (m, 1H), 8.04-8.12 (m, 1H), 8.00 (d, J=11.0 Hz, 1H), 7.31-7.40 (m, 1H), 7.18 (t, J=7.8 Hz, 1H), 6.98-7.07 (m, 2 H), 6.25-6.33 (m, 1H), 3.74 (s, 3H), 3.24-3.32 (m, 6H), 3.07 (s, 3H), 2.79-2.86 (m, 4H), 2.63-2.70 (m, 2H), 2.51 (s, 3H), and 1.14-1.22 (m, 3H). ES-LCMS m/z 595 (M+H).

### Example 41

### 4-{2-(Ethylamino)-4-[3-(methyloxy)phenyl]-1,3-thiazol-5-yl}-N-[3-fluoro-4-({1-[2-(methylsulfonyl)ethyl]-4-piperidinyl}oxy)phenyl]-2-pyrimidinamine

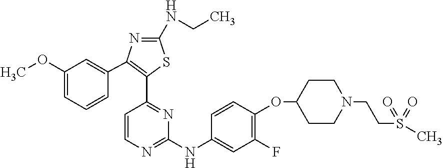

The general procedure (iPrOH (4 mL), 180° C.; 0.25 h; microwave, with 5 drops of conc. HCl) analogous to Example 1, Step G, was used for the reaction of 4-[3-(methyloxy)phenyl]-5-(2-chloro-4-pyrimidinyl)-N-ethyl-1,3-thiazol-2-amine (0.1 g, 0.29 mmol), prepared by a procedure analogous to Example 40, Step B, and [3-fluoro-4-({1-[2-(methylsulfonyl)ethyl]-4-piperidinyl}oxy)phenyl]amine (0.10 g, 0.32 mmol), prepared by a procedure analogous to Example 10, Step D, to give a crude product. Purification by reverse phase HPLC gave the salt of the desired title compound of Example 39. This crude product was dissolved in DCM, washed with 50% saturated NaHCO₃ solution, filtered through Whatman 1 PS paper and concentrated in vacuo to a mustard yellow solid (0.040 g, 22%). ¹H NMR (400 MHz, DMSO-d₆) δ 9.53 (s, 1H), 8.27 (t, J=4.9 Hz, 1H), 8.09 (d, J=5.1 Hz, 1H), 7.83 (d, J=13.6 Hz, 1H), 7.28-7.41 (m, 2H), 6.97-7.15 (m, 4H), 6.28 (d, J=5.1 Hz, 1H), 4.18-4.29 (m, 1H), 3.77 (s, 3H), 3.25-3.30 (m, 4H), 3.04 (s, 3H), 2.68-2.79 (m, 4H), 2.27 (t, J=10.2 Hz, 2H), 1.84-1.96 (m, J=14.1 Hz, 2H), 1.58-1.69 (m, 2H), 1.20 (t, J=7.1 Hz, 3 H). MS (ESI): 627.34 [M+H].

### Example 42

### 4-{2-(Ethylamino)-4-[3-(methyloxy)phenyl]-1,3-thiazol-5-yl}-N-[6-(4-morpholinyl)-3-pyridinyl]-2-pyrimidinamine

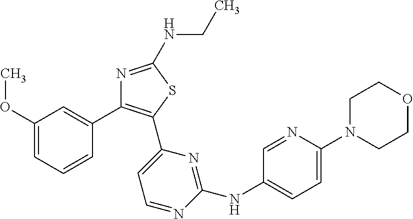

To a solution containing 100 mg (0.29 mmol) of 5-(2-chloro-4-pyrimidinyl)-N-ethyl-4-[3-(methyloxy)phenyl]-1,3-thiazol-2-amine, prepared by a procedure analogous to Example 40, Step B, 63 mg (0.35 mmol) of 6-(4-morpholinyl)-3-pyridinamine, prepared by a procedure analogous to Example 35, Step B, and 2 mL of iPrOH was added 0.1 mL of a 4.0 M solution of HCl in dioxane. The reaction mixture was heated at 90° C. overnight in a sealed tube and the solvents were removed under reduced pressure. The residue was subjected to silica gel chromatography and HPLC purification to give 25 mg of the title compound as a yellow solid: ¹H NMR (400 MHz, DMSO-d₆) δ 9.26 (s, 1H), 8.49 (d, J=2.4 Hz, 1H), 8.22 (t, J=5.2 Hz, 1H), 8.03 (d, J=5.3 Hz, 1H), 7.89 (dd, J=9.2 and 2.8 Hz, 1H), 7.34-7.40 (m, 1H), 7.00-7.07 (m, 3H), 6.80 (d, J=9.2 Hz, 1H), 6.23 (d, J=5.5 Hz, 1H), 3.76 (s, 3H), 3.69-3.74 (m, 4H), 3.33-3.37 (m, 4H), 3.26-3.30 (m, 2H) and 1.19 (t, J=7.2 Hz, 3H). HRMS Calcd for C₂₅H₂₈N₇O₂: 490.2025 (M+H⁺). Found: 490.2017.

### Example 43

### N-[3-Chloro-4-(4-morpholinyl)phenyl]-4-{2-{[2-(methyloxy)ethyl]amino}-4-[3-(methyloxy)phenyl]-1,3-thiazol-5-yl}-2-pyrimidinamine hydrochloride

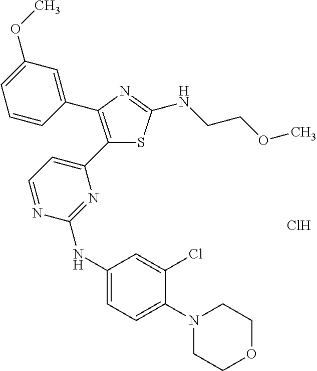

### Step A: 5-(2-Chloro-4-pyrimidinyl)-N-[2-(methyloxy)ethyl]-4-[3-(methyloxy)phenyl]-1,3-thiazol-2-amine

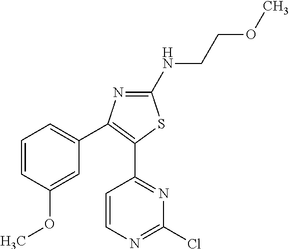

2-(2-Chloro-4-pyrimidinyl)-1-[3-(methyloxy)phenyl]ethanone (700 mg, 2.7 mmol), prepared by a procedure analogous to Example 40, Step A, was dissolved in DCM (15 mL) and treated with NBS (474 mg; 2.7 mmol). The reaction was allowed to stir for 10 min and the solvent was removed. The residual solid was dissolved in DMF (10 mL) and 1-(2-methoxyethyl)-2-thiourea (464 mg, 3.5 mmol) was added. The reaction mixture was allowed to stir overnight and was partitioned between EtOAc and water. The layers were separated and the organic layer was dried over Na₂SO₄, filtered, concentrated onto added silica gel, and purified by column chromatography (95:5 to 1:4 hexanes:EtOAc. The title compound of Step A was obtained as a yellow solid (799 mg, 80% yield) MS (ESI): 377.04 [M+H].

### Step B: N-[3-Chloro-4-(4-morpholinyl)phenyl]-4-{2-{[2-(methyloxy)ethyl]amino}-4-[3-(methyloxy)phenyl]-1,3-thiazol-5-yl}-2-pyrimidinamine hydrochloride

The title compound of Example 41 was synthesized using standard microwave displacement conditions analogous to Example 1, Step G in trifluoroethanol using 5-(2-chloro-4-pyrimidinyl)-N-[2-(methyloxy)ethyl]-4-[3-(methyloxy)phenyl]-1,3-thiazol-2-amine (0.087 g, 0.23 mmol) and [3-chloro-4-(4-morpholinyl)phenyl]amine (0.054 g, 0.25 mmol). Precipitate formed during reaction and was filtered, washed with EtOAc ×3 and ether ×3. The HCl salt form was isolated as 0.078 g, 57% yield, of a light brown solid. ¹H NMR (400 MHz, DMSO-d₆) δ ppm 9.54 (s, 1H), 8.35 (s, 1H), 8.08 (d, J=5.5 Hz, 1H), 8.01 (s, 1H), 7.54-7.60 (m, 1H), 7.36 (t, J=7.7 Hz, 1H), 7.00-7.09 (m, 3H), 6.27 (d, J=5.5 Hz, 1H), 3.75 (s, 3H), 3.70-3.74 (m, 3H), 3.51 (t, J=5.1 Hz, 2H), 3.45 (d, J=4.0 Hz, 2H), 3.28 (s, 3H), 3.08 (dd, J=7.3, 3.3 Hz, 1H), 2.87-2.93 (m, 3H), and 1.16 (t, J=7.33 Hz, 2H). ES-LCMS m/z 553 (M+H).

### Example 44

### 3-[5-{2-[3-Chloro-4-{[2-(1-pyrrolidinyl)ethyl]oxy}phenyl)amino]-4-pyrimidinyl}-2-(ethylamino)-1,3-thiazol-4-yl]phenol

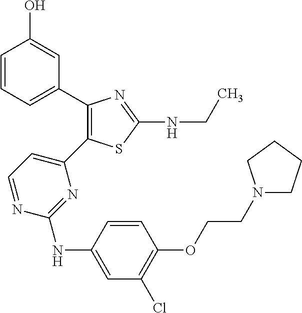

### Step A: Methyl 3-({[4-(methyloxy)phenyl]methyl}oxy)benzoate

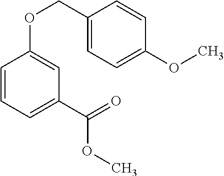

A solution of methyl 3-hydroxybenzoate (10.0 g, 65.7 mmol), 1-(chloromethyl)-4-(methyloxy)benzene (11.3 g, 72.3 mmol), K₂CO₃ (17.8 g, 131 mmol) and tetrabutylammonium iodide (10 mg, 0.03 mmol) in acetone (100 mL) was heated to 70° C. for 24 h. Acetone was removed in vacuo and the reaction mixture was subsequently diluted with EtOAc and washed with water. The organic layer was dried over MgSO₄ and filtered through 1 inch of silica gel, rinsing with 1:1 EtOAc:Hexanes, followed by chromatography, 1:1 EtOAc:Hexanes. The title compound of Step A was obtained as a solid in 35% yield (6.3 g). ¹H NMR (400 MHz, DMSO-d₆): δ 7.52-7.49 (m, 2H), 7.45-7.30 (m, 3H), 7.25 (d, 1H, J=8.0 Hz), 6.91 (d, 2H, J=8 Hz), 5.04 (s, 2H), 3.81 (s, 3H), 3.72 (s, 3H).

### Step B: 2-(2-Chloro-4-pyrimidinyl)-1-[3-({[4-(methyloxy)phenyl]methyl}oxy)phenyl]-ethanone

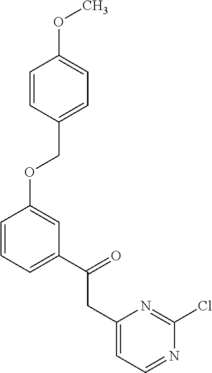

The title compound of Step B was prepared from methyl 3-({[4-(methyloxy)phenyl]methyl}oxy)benzoate (5.0 g, 18.4 mmol) and 2-chloro-4-methylpyrimidine (2.8 g, 22.1 mmol) by a procedure analogous to Example 1, Step E. The title compound of Step B was isolated after chromatography, 40-100% EtOAc in hexanes, in 53% yield as a solid (3.6 g). MS (ESI): 269.3 [M+H]⁺.

### Step C: 5-(2-Chloro-4-pyrimidinyl)-N-ethyl-4-[3-({[4-(methyloxy)phenyl]methyl}oxy)-phenyl]-1,3-thiazol-2-amine

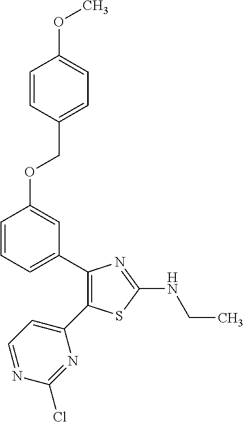

The title compound of Step C was prepared from 2-(2-chloro-4-pyrimidinyl)-1-[3-({[4-(methyloxy)phenyl]methyl}oxy)phenyl]ethanone (3.6 g, 9.8 mmol) and ethyl thiourea (1.22 g, 11.7 mmol) by a procedure analogous to Example 1, Step F. The title compound of Step B was isolated in 55% yield as a solid (2.44 g). MS (ESI): 453.3 [M+H]⁺.

### Step D: 3-[5-{2-[(3-Chloro-4-{[2-(1-pyrrolidinyl)ethyl]oxy}phenyl)amino]-4-pyrimidinyl}-2-(ethylamino)-1,3-thiazol-4-yl]phenol

The title compound of Example 44 was prepared by a procedure analogous to Example 1, Step G, from 5-(2-chloro-4-pyrimidinyl)-N-ethyl-4-[3-({[4-(methyloxy)phenyl]methyl}-oxy)phenyl]-1,3-thiazol-2-amine (200 mg, 0.44 mmol) and (3-chloro-4-{[2-(1-pyrrolidinyl)ethyl]oxy}phenyl)amine (123 mg, 0.44 mmol), prepared by a procedure analogous to Example 20, Step C. The title compound of Example 44 was isolated in 16% yield as an orange solid (38 mg). ¹H NMR (400 MHz, DMSO-d₆) δ ppm 9.56 (s, 1H), 9.50 (s, 1H), 8.23 (t, J=5.13 Hz, 1H), 7.99-8.11 (m, 2H), 7.53 (d, J=9.34 Hz, 1H), 7.20 (t, J=7.69 Hz, 1H), 7.11 (d, J=8.97 Hz, 1H), 6.74-6.89 (m, 3H), 6.27 (d, J=5.68 Hz, 1H), 4.33 (br s, 2H), 3.58 (br s, 4H), 3.19-3.26 (m, 2H), 3.12 (br s, 2H), 1.99 (br s, 2H), 1.85 (br s, 2H), 1.16 (t, J=7.05 Hz, 3H); MS (ESI): 537.3 [M+H]⁺.

### Example 45

### N-(3-Chloro-4-{[2-(1-pyrrolidinyl)ethyl]oxy}phenyl)-4-[4-[3-(dimethylamino)phenyl]-2-(ethylamino)-1,3-thiazol-5-yl]-2-pyrimidinamine

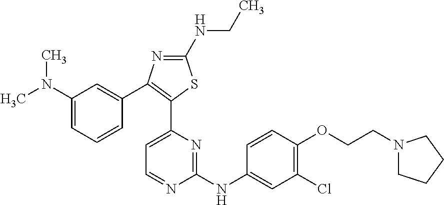

### Step A: Methyl 3-(dimethylamino)benzoate

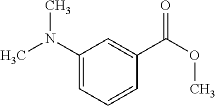

A suspension of 3-(dimethylamino)benzoic acid (2.0 g, 12.1 mmol) in DCM (20 mL) and MeOH (2 mL) was cooled to 0° C. A 2 M solution of TMS-diazomethane in ether (6.4 mL, 12.7 mmol) was added dropwise over 10 min, and the reaction was stirred at 0° C. After 30 min, the reaction mixture was washed with saturated aqueous NaHCO₃, concentrated, adsorbed onto silica gel, and purified by column chromatography (eluting with 0-50% EtOAc/DCM) to generate the desired product in 94% yield (2.04 g, 11.3 mmol). MS (ESI) m/z=180 [M+H]⁺.

### Step B: 2-(2-Chloro-4-pyrimidinyl)-1-[3-(dimethylamino)phenyl]ethanone

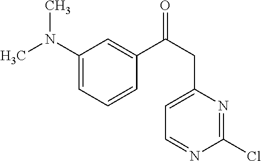

To a solution of methyl 3-(dimethylamino)benzoate (2.04 g, 11.3 mmol) in anhydrous THF (40 mL), cooled to 0° C., was added a 1M solution of LHMDS in THF (14.8 mL, 14.8 mmol). A solution of 2-chloro-4-methylpyrimidine (1.46 g, 11.4 mmol) in anhydrous THF (10 mL) was added dropwise over 10 min. The icebath was removed, and the reaction was allowed to warm to rt. After 16 h, the reaction was concentrated on the rotovap, and the residue was redissolved in EtOAc and adsorbed onto silica gel. The crude product was purified by column chromatography (eluting with 0-100% EtOAc/hexanes) to generate the desired product of Step B in 56% yield (1.77 g, 6.4 mmol). MS (ESI) m/z=276 [M+H]⁺.

### Step C: 5-(2-Chloro-4-pyrimidinyl)-4-[3-(dimethylamino)phenyl]-N-ethyl-1,3-thiazol-2-amine

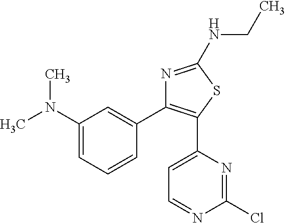

The title compound of Step C was made using a procedure analogous to Example 1, Step F, using 2-(2-chloro-4-pyrimidinyl)-1-[3-(dimethylamino)phenyl]ethanone in place of the 2-(2-chloro-4-pyrimidinyl)-1-[4-(methyloxy)-2-pyridinyl]ethanone. Yield: 0.52 g (1.4 mmol, 80% yield). MS (ESI) m/z=360 [M+H]⁺.

### Step D: N-(3-Chloro-4-{[2-(1-pyrrolidinyl)ethyl]oxy}phenyl)-4-[4-[3-(dimethylamino)phenyl]-2-(ethylamino)-1,3-thiazol-5-yl]-2-pyrimidinamine

The title compound of Example 43 was synthesized using a procedure analogous to Example 1, Step G, using 5-(2-chloro-4-pyrimidinyl)-4-[3-(dimethylamino)phenyl]-N-ethyl-1,3-thiazol-2-amine and (3-chloro-4-{[2-(1-pyrrolidinyl)ethyl]oxy}phenyl)ammonium chloride, prepared by a procedure analogous to Example 20, Step C. The desired product of Step D was obtained in 46% yield (47 mg, 0.08 mmol). ¹H NMR (400 MHz, DMSO-d₆) δ 9.47 (s, 1H), 8.28 (bs, 1H), 8.04 (d, 1H, J=5.3 Hz), 7.84 (d, 1H, J=4.3 Hz), 7.32 (d, 1H, J=9.2 Hz), 7.09 (t, 1H, J=9.2 Hz), 6.63 (s, 1H), 6.39 (s, 1H), 5.92 (d, 1H, J=5.5 Hz), 3.82 (s, 3H), 3.72 (s, 3H), 3.27 (m, 2H), 2.70 (bs, 4H), 2.26 (bs, 2H), 1.87 (m, 4H), 1.69 (bs, 4H), 1.19 (t, 3H, J=7.2 Hz). MS (ESI) m/z=564 [M+H]⁺.

### Example 46

### N-(3-Chloro-4-{[2-(dimethylamino)ethyl]oxy}phenyl)-4-[4-(3-chlorophenyl)-2-(ethylamino)-1,3-thiazol-5-yl]-2-pyrimidinamine

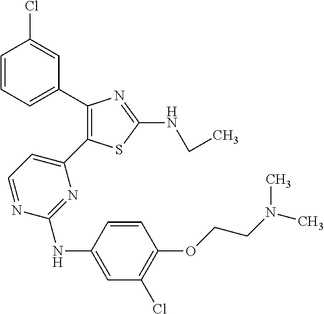

### Step A: (E)-1-(3-Chlorophenyl)-2-(2-chloro-4-pyrimidinyl)ethanol

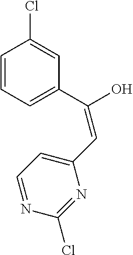

To obtain the desired compound of Step A, ethyl 3-chlorobenzoate (3.0 g, 16 mmol) and LHMDS (36 mL, 36 mmol, 1 M in THF) were placed in a round bottom flask and cooled to 0° C. 2-Chloro-4-methylpyrimidine (2.1 g, 16 mmol) was added in one portion and the resulting mixture was allowed to stir and warm to rt overnight. EtOAc and water were added to the reaction mixture and the desired product was extracted into the organic phase which was then concentrated onto silica gel and purified via column chromatography to yield 2.25 g of ketone/enolate mixture of the desired target compound of Step A (53% Y). MS (ESI) m/z 266.96 and 268.93 (M+H)⁺.

### Step B: 4-(3-Chlorophenyl)-5-(2-chloro-4-pyrimidinyl)-N-ethyl-1,3-thiazol-2-amine

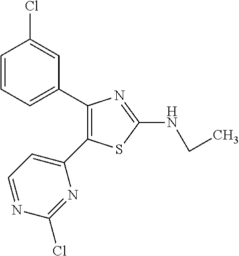

To obtain the desired compound of Step B, (E)-1-(3-chlorophenyl)-2-(2-chloro-4-pyrimidinyl)ethanol (1.0 g, 3.76 mmol) was dissolved in DMF (10 mL) in a round bottom flask with stirring. NBS (0.670 g, 3.76 mmol) was added in one portion and the resulting mixture was stirred at rt for 10 min. Ethylthiourea (0.430 g, 4.1 mmol) was then added and the reaction was stirred about 1 h at which time it was complete. EtOAc and water were added to the reaction mixture and the desired product was extracted into the organic phase which was then concentrated onto silica gel and purified via column chromatography. Pure fractions were combined and concentrated to yield 500 mg of the desired compound of Step B (38% Y). ¹H NMR (400 MHz, DMSO-d₆) δ 8.57 (t, J=5.3 Hz, 1H), 8.30 (d, J=5.6 Hz, 1H), 7.57 (s, 1

H), 7.57-7.52 (m, 1H), 7.48 (m, 2H), 6.79 (d, J=5.5 Hz, 1H), 3.30 (m, 2H), 1.18 (t, J=7.3 Hz, 3H).

### Step C: N-(3-Chloro-4-{[2-(dimethylamino)ethyl]oxy}phenyl)-4-[4-(3-chlorophenyl)-2-(ethylamino)-1,3-thiazol-5-yl]-2-pyrimidinamine

To obtain the title compound of Example 46, 4-(3-chlorophenyl)-5-(2-chloro-4-pyrimidinyl)-N-ethyl-1,3-thiazol-2-amine (0.098 g, 0.280 mmol) and {2-[(4-amino-2-chlorophenyl)oxy]ethyl}dimethylamine (0.070 g, 0.280 mmol) were combined with iPrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness and subsequently adhered to the silica gel. Column chromatography using EtOAc, MeOH, and NH₄OH yielded fractions which were concentrated to dryness. The resulting material was then sonicated in ether and the solid that persisted was filtered off to yield 86 mg of the title compound of Example 46 (58% Y). ¹H NMR (400 MHz, DMSO-d₆) δ 9.49 (s, 1H), 8.28 (t, J=5.5 Hz, 1H), 8.09 (d, J=5.7 Hz, 1H), 7.98 (d, J=2.6 Hz, 1H), 7.54-7.42 (m, 5H), 7.04 (d, J=8.8 Hz, 1H), 6.22 (d, J=5.5 Hz, 1H), 4.05 (t, J=5.9 Hz, 2H), 3.26 (m, 2H), 2.62 (t, J=5.9 Hz, 2H), 2.21 (s, 6H), 1.17 (t, J=7.2 Hz, 3H); HRMS C₂₆H₂₇N₆OSCl₂ (M+H)⁺ calcd 529.1344. found 529.1346.

### Example 47

### 4-[4-[3-(Dimethylamino)phenyl]-2-(ethylamino)-1,3-thiazol-5-yl]-N-[6-(4-morpholinyl)-3-pyridinyl]-2-pyrimidinamine

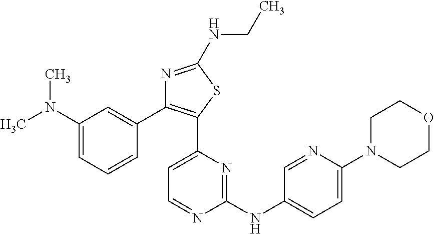

The title compound of Example 47 was prepared in a similar procedure to that described in Example 1, Step G, using 6-(4-morpholinyl)-3-pyridinamine (prepared by a procedure analogous to Example 35, Step B) and 5-(2-chloro-4-pyrimidinyl)-4-[3-(dimethylamino)phenyl]-N-ethyl-1,3-thiazol-2-amine (prepared by a procedure analogous to Example 45, Step C). The desired product was obtained in 20% yield (27 mg, 0.05 mmol). ¹H NMR (400 MHz, DMSO-d₆) δ 9.22 (s, 1H), 8.48 (d, 1H, J=2.0 Hz), 8.19 (t, 1H, J=5.3 Hz), 8.00 (d, 1H, J=5.5 Hz), 7.88 (dd, 1H, J=2.6 Hz, 9.2 Hz), 7.24 (t, 1H, J=8.1 Hz), 6.77 (m, 2H), 6.26 (d, 1H, J=5.5 Hz), 3.70 (t, 4H, J=4.8 Hz), 3.34 (m, 4H), 3.27 (m, 2H), 2.89 (s, 6H), 1.18 (t, 3H, J=7.3 Hz). MS (ESI) m/z=503 [M+H]⁺.

### Example 48

### 4-{2-(Ethylamino)-4-[2-fluoro-3-(methyloxy)phenyl]-1,3-thiazol-5-yl}-N-[3-fluoro-4-({1-[2-(methylsulfonyl)ethyl]-4-piperidinyl}oxy)phenyl]-2-pyrimidinamine

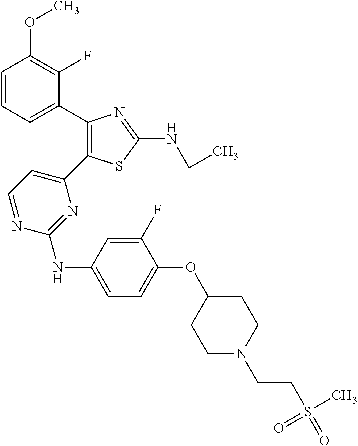

### Step A: Methyl 2-fluoro-3-(methyloxy)benzoate

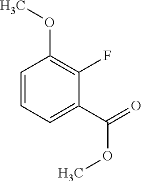

To a suspension of 2-fluoro-3-(methyloxy)benzoic acid (2.0 g, 11.8 mmol) in DCM (20 mL) and MeOH (2 mL) at 0° C., trimethylsilyl diazomethane (2 M in ether, 12.3 mmol, 6.2 mL) was added dropwise. The reaction mixture was allowed to stir for 30 min at 0° C. and the solvents were removed under reduced pressure. The residue was diluted with EtOAc, adsorbed onto silica gel, and subjected to silica gel chromatography in two batches to give 2.0 g (92%) of methyl 2-fluoro-3-(methyloxy)benzoate: ¹H NMR (CDCl₃, 400 MHz) δ 7.48-7.44 (m, 1H), 7.15-7.09 (m, 2H), 3.93 (s, 3H), 3.91 (s, 3H).

### Step B: 2-(2-Chloro-4-pyrimidinyl)-1-[2-fluoro-3-(methyloxy)phenyl]ethanone

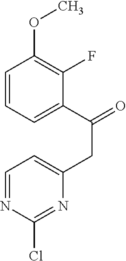

To a solution of methyl 2-fluoro-3-(methyloxy)benzoate (2.0 g, 10.8 mmol) in dry THF (30 mL) at 0° C., LHMDS (1 M in THF, 21.2 mmol, 21.2 mL) was added and the solution was allowed to stir for 10 min at 0° C. A solution of 2-chloro-4-methylpyrimidine (13.0 mmol, 1.7 g) in THF was then added dropwise to the solution of ester and base at 0° C. over 15 min. The solution was allowed to stir 30 minutes at 0° C., quenched at 0° C. by the addition of MeOH and the solvents were removed under reduced pressure. The residue was diluted with EtOAc and washed with water. The water layer was extracted twice with EtOAc, dried with MgSO₄, and filtered through a short plug of silica gel, eluting with EtOAc. The solvents were removed under reduced pressure to obtain 2.6 g (86%) of 2-(2-chloro-4-pyrimidinyl)-1-[2-fluoro-3-(methyloxy)phenyl]ethanone: ¹H NMR (CDCl₃, 300 MHz) δ 13.75 (s, 0.7H), 8.59 (d, J=5.1 Hz, 0.3H), 8.40 (d, J=5.4 Hz, 0.7H), 7.48-7.40 (m, 1H), 7.28 (d, J=5.1 Hz, 0.3H), 7.19-7.13 (m, 1H), 7.08-7.02 (m, 0.7H), 6.92 (d, J=5.4 Hz, 0.7H), 6.22 (s, 0.7H), 4.48 (d, J=3.0 Hz, 0.7H), 3.92 (d, J=2.7 Hz, 3H).

### Step C: 5-(2-Chloro-4-pyrimidinyl)-N-ethyl-4-[2-fluoro-3-(methyloxy)phenyl]-1,3-thiazol-2-amine

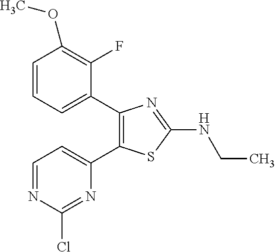

To a solution of 2-(2-chloro-4-pyrimidinyl)-1-[2-fluoro-3-(methyloxy)phenyl]ethanone (2.1 g, 7.5 mmol) in 60 mL of DME, NBS (1.3 g, 7.5 mmol) was added and the solution was allowed to stir at rt for 30 min. The reaction mixture was then concentrated and the resulting oil was diluted with DMF (20 mL) and ethyl thiourea (0.94 g, 9 mmol) was added at once. The reaction was allowed to stir for 1 h at rt and diluted with EtOAc and partitioned between EtOAc and water. The combined organic layers were washed with water and brine and the solvents were removed under reduced pressure. The residue was washed with ether to give 1.49 g (51%) of 5-(2-chloro-4-pyrimidinyl)-N-ethyl-4-[2-fluoro-3-(methyloxy)phenyl]-1,3-thiazol-2-amine: ¹H NMR (CDCl₃, 300 MHz) δ 8.14 (d, J=5.7 Hz, 1H), 7.22-7.16 (m, 1H), 7.12-7.06 (m, 1H), 7.02-6.97 (m, 1H), 6.67 (d, J=5.7 Hz, 1H), 6.31 (brs, 1H), 3.93 (s, 3H), 3.32-3.23 (m, 2H), 1.27-1.22 (m, 3H).

### Step D: 4-{2-(Ethylamino)-4-[2-fluoro-3-(methyloxy)phenyl]-1,3-thiazol-5-yl}-N-[3-fluoro-4-({1-[2-(methylsulfonyl)ethyl])-4-piperidinyl}oxy)phenyl]-2-pyrimidinamine

To obtain the title compound of Example 48, 5-(2-chloro-4-pyrimidinyl)-N-ethyl-4-[2-fluoro-3-(methyloxy)phenyl]-1,3-thiazol-2-amine (0.1 g, 0.274 mmol) and [3-fluoro-4-({1-[2-(methylsulfonyl)ethyl]-4-piperidinyl}oxy)phenyl]amine (0.086 g, 0.274 mmol), prepared by a procedure analogous to Example 10, Step D, were combined with iPrOH (2 mL) and concentrated HCl (2 drops) in a microwave vial. The reaction was heated to 180° C. for 20 min in the microwave then cooled to rt. TEA (approx. 0.1 mL) and silica gel were combined with the reaction and the resulting mixture was concentrated to dryness and subsequently adhered to silica gel. Column chromatography using EtOAc, MeOH, and NH₄OH yielded fractions which were concentrated to dryness. This material was then further purified on a reverse phase acidic HPLC. The resulting fractions were free-based via extraction and concentrated to dryness to yield 47 mg of the title compound of Example 48 (27% Y). ¹H NMR (400 MHz, DMSO-d₆) δ 9.51 (s, 1H), 8.29 (t, J=5.3 Hz, 1H), 8.08 (d, J=5.9 Hz, 1H), 7.78 (dd, J=14.5, 2.7 Hz, 1H), 7.30 (d, J=8.9 Hz, 1H), 7.24 (m, 2H), 7.07 (t, J=9.3 Hz, 1H), 6.98 (m, 1H), 6.06 (d, J=5.1 Hz, 1H), 4.21 (m, 1H), 3.85 (s, 3H), 3.25 (m, 4H), 3.00 (s, 3H), 2.68 (m, 4H), 2.24 (m, 2H), 1.87 (br, 2H), 1.60 (m, 2H), 1.16 (t, J=7.7 Hz, 3H); HRMS C₃₀H₃₅N₆O₄F₂S₂ (M+H)⁺ calcd 645.2129. found 645.2127.

### Example 49

### 4-[4-[4-Chloro-3-(methyloxy)phenyl]-2-(ethylamino)-1,3-thiazol-5-yl]-N-(3-chloro-4-{[2-(1-pyrrolidinyl)ethyl]oxy}phenyl)-2-pyrimidinamine

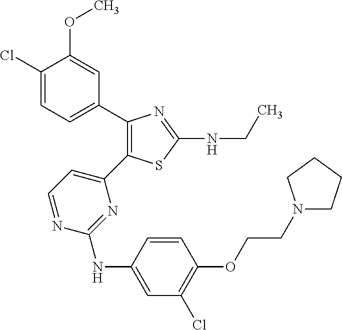

### Step A: 1-[4-Chloro-3-(methyloxy)phenyl]-2-(2-chloro-4-pyrimidinyl)ethanone

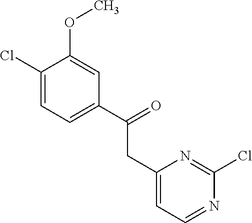

The title compound of Step A was prepared from methyl 4-chloro-3-(methyloxy)benzoate (2.17 g, 10.9 mmol) and 2-chloro-4-methylpyrimidine (1.4 g, 10.9 mmol) by a procedure analogous to Example 1, Step E. The title compound of Step A was isolated in 77% yield as a tan solid (2.51 g). MS (APCI): 297.1 [M+H]⁺.

### Step B: 4-[4-Chloro-3-(methyloxy)phenyl]-5-(2-chloro-4-pyrimidinyl)-N-ethyl-1,3-thiazol-2-amine

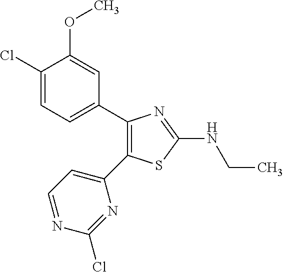

The title compound of Step B was prepared from 1-[4-chloro-3-(methyloxy)phenyl]-2-(2-chloro-4-pyrimidinyl)ethanone (0.50 g, 1.68 mmol) and ethyl thiourea (0.21 g, 2.02 mmol) by a procedure analogous to Example 1, Step F. The title compound of Step B was isolated in 93% yield as a yellow solid (0.60 g). MS (APCI): 381.1 [M+H]⁺.

### Step C: 4-[4-[4-Chloro-3-(methyloxy)phenyl]-2-(ethylamino)-1,3-thiazol-5-yl]-N-(3-chloro-4-{[2-(1-pyrrolidinyl)ethyl]oxy}phenyl)-2-pyrimidinamine

The title compound of Example 49 was prepared from 4-[4-chloro-3-(methyloxy)phenyl]-5-(2-chloro-4-pyrimidinyl)-N-ethyl-1,3-thiazol-2-amine (100 mg, 0.26 mmol) and (3-chloro-4-{[2-(1-pyrrolidinyl)ethyl]oxy}phenyl)amine (73 mg, 0.26 mmol), prepared in a procedure analogous to Example 20, Step C, by a procedure analogous to Example 1, Step G. The title compound of Example 49 was isolated in 54% yield as a yellow solid (82 mg). ¹H NMR (400 MHz, DMSO-d₆) δ ppm 9.47 (s, 1 H), 8.28 (t, J=5.13 Hz, 1H), 8.10 (d, J=6.41 Hz, 1H), 8.00 (s, 1H), 7.47 (d, J=8.06 Hz, 2H), 7.23 (s, 1H), 7.07 (d, J=8.1 Hz, 1H), 7.03 (d, J=9.0 Hz, 1H), 6.30 (d, J=6.4 Hz, 1H), 4.07 (t, J=5.3 Hz, 2H), 3.80 (s, 3H), 3.19-3.27 (m, 2H), 2.77 (t, J=5.7 Hz, 2H), 2.52 (br s, 4H), 1.65 (br s, 4H), 1.18 (t, J=7.1 Hz, 3H); MS (APCI): 586.1 [M+H]⁺.

### Example 50

### 5-[(4-{2-(Ethylamino)-4-[4-methyl-3-(methyloxy)phenyl]-1,3-thiazol-5-yl}-2-pyrimidinyl)amino]-2-{[2-(1-pyrrolidinyl)ethyl]oxy}benzonitrile

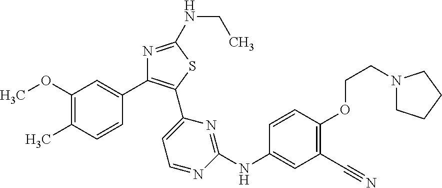

### Step A: 5-Nitro-2-{[2-(1-pyrrolidinyl)ethyl]oxy}benzonitrile

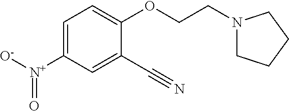

2-(1-Pyrrolidinyl)ethanol (770 μL, 12.0 mmol) was injected into a 0° C. stirred suspension of 2-fluoro-5-nitrobenzonitrile (1.0 g, 6.0 mmol), 60% w/w NaH as a suspension in oil (361 mg, 9.0 mmol) and DMF (10 mL). The ice bath was removed and the reaction was allowed to warm to rt. TLC confirmed consumption of the starting material and the DMF was removed under vacuum. The resulting residue was partitioned between EtOAc and water. The organic fraction was washed with brine and dried over MgSO₄. The organic fraction was concentrated and purified by silica gel chromatography (gradient: 0-100% (90% CH₂Cl₂:9% MeOH:1% NH₄OH)/CH₂Cl₂). Purification provided 330 mg (22%) of 5-nitro-2-{[2-(1-pyrrolidinyl)ethyl]oxy}benzonitrile. ¹H NMR (400 MHz, DMSO-d₆) δ ppm 8.62-8.69 (m, 1H), 8.47 (ddd, J=9.3, 2.9, 0.9 Hz, 1H), 7.46 (d, J=9.3 Hz, 1H), 4.37 (t, J=5.5 Hz, 2H), 2.84 (t, J=5.7 Hz, 2H), 2.53 (s, 4H), 1.65 (s, 4H).

### Step B: 5-Amino-2-{[2-(1-pyrrolidinyl)ethyl]oxy}benzonitrile

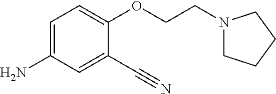

5-Nitro-2-{[2-(1-pyrrolidinyl)ethyl]oxy}benzonitrile (330 mg, 1.3 mmol) was stirred vigorously with 5% platinum on carbon (75 mg) in EtOH (10 mL) under a H₂ atm (balloon pressure) for 15 h at rt. The reaction was filtered through a pad of Celite and concentrated to provide 230 mg (79%) of the title compound of Step B as a clear oil. ¹H NMR (400 MHz, DMSO-d₆) δ ppm 6.95 (d, J=9.2 Hz, 1H), 6.82 (dd, J=9.0, 2.75 Hz, 1H), 6.76 (d, J=2.6 Hz, 1H), 5.05 (s, 2H), 4.04 (t, J=5.9 Hz, 2H), 2.74 (t, J=5.9 Hz, 2H), 2.47-2.53 (m, 4H), and 1.65 (s, 4H).

### Step C: 2-(2-Chloro-4-pyrimidinyl)-1-[4-methyl-3-(methyloxy)phenyl]ethanone

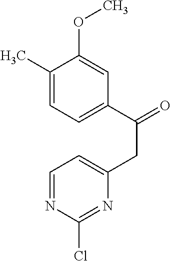

To a solution of methyl 4-methyl-3-(methyloxy)benzoate (2.0 g, 11.1 mmol) in dry THF (20 mL) at 0° C., LHMDS (1 M in THF, 2.1 eq, 23.3 mmol, 23.3 mL) was added and the solution was allowed to stir for 10 min at 0° C. A solution of 2-chloro-4-methylpyrimidine (1.2 eq, 13.3 mmol, 1.7 g) in 5 mL of THF was then added dropwise to the reaction mixture at 0° C. over 10 min. The solution was allowed to stir 30 min at 0° C. The reaction mixture was then quenched at 0° C. with MeOH and the solvent was removed in vacuo. The residue was diluted with EtOAc and washed with water. The water layer was extracted twice with EtOAc, dried with MgSO₄, and the solvent was removed to produce a crude solid. The crude solid was triturated in EtOAc to give 1.12 g of the title product of Step C as a solid (36%). MS (AFCI): 277.2 [M+H]⁺.

### Step D: 5-(2-chloro-4-pyrimidinyl)-N-ethyl-4-[4-methyl-3-(methyloxy)phenyl]-1,3-thiazol-2-amine

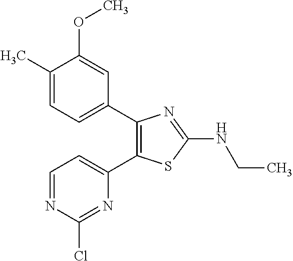

The title compound of Step D was prepared from 2-(2-chloro-4-pyrimidinyl)-1-[4-methyl-3-(methyloxy)phenyl]ethanone (512 mg, 1.85 mmol) and N-ethylthiourea (1.2 eq., 231 mg, 2.22 mmol) by a procedure analogous to Example 1, Step F. The crude solid was triturated in ether to give 400 mg of the title product of Step D as a beige solid (60%). MS (AFCI): 361.1 [M+H]⁺.

### Step E: 5-[(4-{2-(Ethylamino)-4-[4-methyl-3-(methyloxy)phenyl]-1,3-thiazol-5-yl}-2-pyrimidinyl)amino]-2-{[2-(1-pyrrolidinyl)ethyl]oxy}benzonitrile

5-(2-Chloro-4-pyrimidinyl)-N-ethyl-4-[4-methyl-3-(methyloxy)phenyl]-1,3-thiazol-2-amine (123 mg, 0.34 mmol) was combined with 5-amino-2-{[2-(1-pyrrolidinyl)ethyl]oxy}benzonitrile (66 mg, 0.28 mmol), HCl solution (4 M) in dioxane (142 μL, 0.60 mmol) and 2,2,2-trifluoroethanol (3 mL) in a sealed vessel. The reaction was heated for 30 min at 170° C. by microwave radiation. The reaction was cooled to rt and concentrated to a residue under vacuum. The residue was purified by silica gel chromatography (gradient: 0-100% (90% CH₂Cl₂:9% MeOH:1% NH₄OH)/CH₂Cl₂). The fractions containing the coupled adduct were combined and concentrated. The purified oil was triturated with MeOH and filtered. Filtration yielded 68 mg (43%) of the title compound of Example 50 as a yellow powder. ¹H NMR (400 MHz, DMSO-d₆) δ ppm 9.58 (s, 1H), 8.27 (t, J=4.9 Hz, 1H), 8.22 (s, 1H), 8.08 (d, J=5.3 Hz, 1H), 7.78 (d, J=8.6 Hz, 1H), 7.12-7.21 (m, 2H), 7.01 (s, 1H), 6.96 (d, J=7.7 Hz, 1H), 6.33 (d, J=5.3 Hz, 1H), 4.16 (t, J=5.5 Hz, 2H), 3.72 (s, 3H), 3.19-3.29 (m, 2H), 2.79 (t, J=5.2 Hz, 2H), 2.53 (br s, 4 H), 2.12-2.21 (m, 3H), 1.65 (br. s., 4H), and 1.17 (t, J=7.1 Hz, 3H); m/z (AFCI): 556.19 [M+H]⁺.

### Example 51

### 4-[4-[4-Chloro-3-(methyloxy)phenyl]-2-(cyclopropylamino)-1,3-thiazol-5-yl]-N-(3-chloro-4-{[2-(1-pyrrolidinyl)ethyl]oxy}phenyl)-2-pyrimidinamine

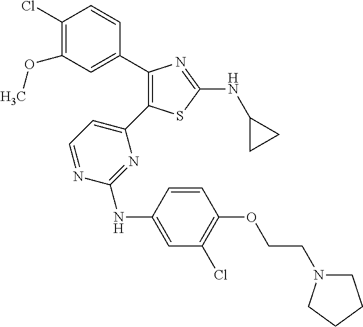

### Step A: 1-[4-Chloro-3-(methyloxy)phenyl]-2-(2-chloro-4-pyrimidinyl)ethanone

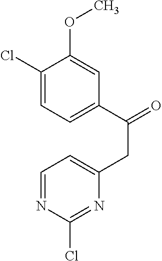

To a solution of methyl 4-chloro-3-(methyloxy)benzoate (2.17 g, 10.9 mmol) in dry THF (20 mL) at 0° C., LHMDS (1 M in THF, 2.1 eq, 22.8 mmol, 22.8 mL) was added and the solution was allowed to stir for 10 min at 0° C. A solution of 2-chloro-4-methylpyrimidine (1.0 eq, 10.9 mmol, 1.4 g) in 5 mL of THF was then added dropwise to the reaction mixture at 0° C. over 10 min. The solution was allowed to stir 30 min at 0° C. The reaction mixture was then quenched at 0° C. with MeOH and the solvent was removed in vacuo. The residue was diluted with EtOAc and washed with water. The water layer was extracted twice with EtOAc, dried with MgSO₄, and the solvent was removed to produce a crude solid. The crude solid was triturated in EtOAc to give 2.51 g of the title compound of Step A as a tan solid (77%). MS (APCI): 297.1 [M+H]⁺.

### Step B: 4-[4-Chloro-3-(methyloxy)phenyl]-5-(2-chloro-4-pyrimidinyl)-N-cyclopropyl-1,3-thiazol-2-amine

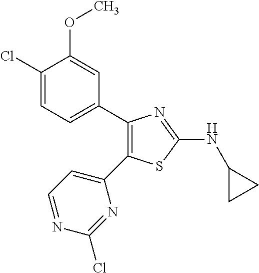

To obtain the desired compound of Step B, 1-[4-chloro-3-(methyloxy)phenyl]-2-(2-chloro-4-pyrimidinyl)ethanone (0.4 g, 1.35 mmol) and DMF (4 mL) were placed in a round bottom flask with stirring. NBS (0.24 g, 1.35 mmol) was added in one portion and the resulting mixture was allowed to stir at rt for 5 min. N-Cyclopropylthiourea (0.2 g, 1.75 mmol) was then added to the α-bromoketone. The reaction was then allowed to stir at rt overnight. EtOAc and water were added to the reaction mixture and the desired product was extracted into the organic phase, which was then concentrated onto silica gel and purified via column chromatography to yield 0.18 g of the target compound of Step B as a yellow oily solid. MS (ESI) m/z 392.96 (M+H)⁺.

### Step C: 4-[4-[4-Chloro-3-(methyloxy)phenyl]-2-(cyclopropylamino)-1,3-thiazol-5-yl]-N-(3-chloro-4-{[2-(1-pyrrolidinyl)ethyl]oxy}phenyl)-2-pyrimidinamine

To obtain the title compound of Example 51, 4-[4-chloro-3-(methyloxy)phenyl]-5-(2-chloro-4-pyrimidinyl)-N-cyclopropyl-1,3-thiazol-2-amine (0.075 g, 0.19 mmol) and (3-chloro-4-{[2-(1-pyrrolidinyl)ethyl]oxy}phenyl)amine (0.053 g, 0.19 mmol), prepared by a procedure analogous to Example 20, Step C, were combined with iPrOH (2 mL) and concentrated HCl (2 drops) in a microwave vial. The reaction was heated to 180° C. for 20 min in the microwave then cooled to rt. TEA (approx. 0.1 mL) and silica gel were combined with the reaction and the resulting mixture was concentrated to dryness and subsequently adhered to silica gel. Column chromatography using EtOAc, MeOH, and NH₄OH yielded fractions which were concentrated to dryness. This material was then further purified on a reverse phase acidic HPLC. The resulting fractions were free-based via extraction and concentrated to dryness. This material was then sonicated in ether and the solid that persisted was filtered off to yield 32 mg of the title compound of Example 51 (28% Y). ¹H NMR (400 MHz, DMSO-d₆) δ 9.52 (s, 1H), 8.67 (s, 1H), 8.12 (d, J=5.2 Hz, 2H), 7.47 (d, J=8.2 Hz, 1H), 7.43 (d, J=8.3 Hz, 1H), 7.23 (d, J=1.9 Hz, 1H), 7.05 (t, J=8.4 Hz, 2H), 6.32 (d, J=5.6 Hz, 1H), 4.08 (t, J=6.0 Hz, 2H), 3.80 (s, 3H), 2.81 (br, 2H), 2.57 (br, 5 H), 1.67 (br, 4H), 0.78 (m, 2H), and 0.59 (m, 2H); HRMS C₂₉H₃₁N₆O₂SCl₂ (M+H)⁺ calcd 597.1606. found 597.1616.

### Example 52

### 4-[4-(1,3-Benzodioxol-5-yl)-2-(ethylamino)-1,3-thiazol-5-yl]-N-[3-fluoro-4-({1-[2-(methylsulfonyl)ethyl]-4-piperidinyl}oxy)phenyl]-2-pyrimidinamine

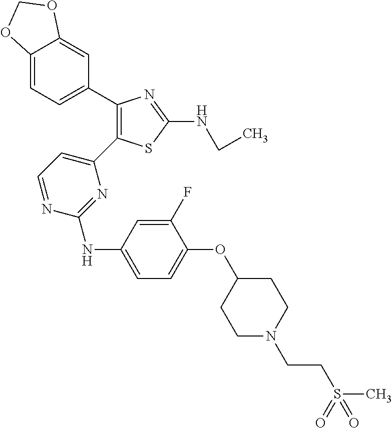

### Step A: Methyl 1,3-benzodioxole-5-carboxylate

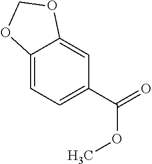

To a suspension of 1,3-benzodioxole-5-carboxylic acid (8.3 g, 50 mmol) in MeOH (100 mL) was added para-toluenesulfonic acid (0.8 g, 5%). The reaction mixture was heated to reflux overnight and the solvent was removed under reduced pressure. The residue was taken up in EtOAc and washed with a saturated aqueous solution of NaHCO₃, washed with brine, dried over Na₂SO₄ and filtered. The solvents were removed under reduced pressure to give 7.1 g (79%) of methyl 1,3-benzodioxole-5-carboxylate: ¹H-NMR (CDCl₃, 400 MHz) δ 7.63 (dd, J=1.6, 8.1 Hz, 1H), 7.44 (d, J=1.6 Hz, 1H), 6.01 (s, 2H), 3.86 (s, 3H).

### Step B: 1-(1,3-Benzodioxol-5-yl)-2-(2-chloro-4-pyrimidinyl)ethanone

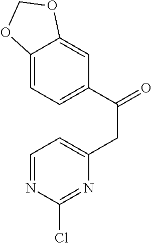

To a solution of methyl 1,3-benzodioxole-5-carboxylate (3.6 g, 20 mmol) in dry THF (40 mL) at 0° C., LHMDS (1 M in THF, 42 mmol, 42 mL) was added and the solution was allowed to stir for 10 min at 0° C. A solution of 2-chloro-4-methylpyrimidine (24 mmol, 3.1 g) in THF was then added dropwise to the solution of ester and base at 0° C. over 15 min. The solution was allowed to stir 30 minutes at 0° C., quenched at 0° C. by the addition of MeOH, and the solvents were removed under reduced pressure. The residue was diluted with EtOAc and washed with water. The water layer was extracted twice with EtOAc, dried with MgSO₄, and filtered through a short plug of silica gel, eluting with EtOAc. The solvents were removed under reduced pressure to obtain 1.5 g (27%) of 1-(1,3-benzodioxol-5-yl)-2-(2-chloro-4-pyrimidinyl)ethanone: ¹H NMR (CDCl₃, 300 MHz) δ 13.81 (s, 1H), 8.57 (d, J=5.1 Hz, 1H), 7.63 (dd, J=8.1, 1.8 Hz, 1H), 7.47 (d, J=1.8 Hz, 1H), 7.31 (d, J=5.1 Hz, 1H), 6.88 (d, J=8.1 Hz, 2H), 6.85 (s, 2H), and 4.39 (s, 2H).

### Step C: 4-(1,3-Benzodioxol-5-yl)-5-(2-chloro-4-pyrimidinyl)-N-ethyl-1,3-thiazol-2-amine

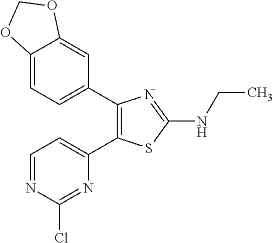

To a solution 1-(1,3-benzodioxol-5-yl)-2-(2-chloro-4-pyrimidinyl)ethanone (1.5 g, 5.6 mmol) in DME, NBS (0.99 g, 5.6 mmol) was added and the solution was allowed to stir at rt for 30 min. The reaction mixture was then concentrated and the resulting oil is diluted with DMF (20 mL) and ethyl thiourea (0.7 g, 6.7 mmol) was added at once. The reaction was allowed to stir for 1 h at rt and diluted with EtOAc and partitioned between EtOAc and water. The combined organic layers were washed with water and brine and the solvents were removed under reduced pressure. The residue was washed with ether to give 1.1 g (55%) of 4-(1,3-benzodioxol-5-yl)-5-(2-chloro-4-pyrimidinyl)-N-ethyl-1,3-thiazol-2-amine: ¹H NMR (CDCl₃, 400 MHz) δ 8.14 (d, J=5.6 Hz, 1H), 7.00-6.97 (m, 2H), 6.90-6.87 (m, 2H), 6.04 (s, 2H), 5.99 (br, 1H), 3.49-3.29 (m, 2H), and 1.27 (t, J=5.6 Hz, 3H).

### Step D: 4-[4-(1,3-Benzodioxol-5-yl)-2-(ethylamino)-1,3-thiazol-5-yl]-N-[3-fluoro-4-({1-[2-(methylsulfonyl)ethyl]-4-piperidinyl}oxy)phenyl]-2-pyrimidinamine

To obtain the title compound of Example 52, 4-(1,3-benzodioxol-5-yl)-5-(2-chloro-4-pyrimidinyl)-N-ethyl-1,3-thiazol-2-amine (0.1 g, 0.277 mmol) and [3-fluoro-4-({1-[2-(methylsulfonyl)ethyl]-4-piperidinyl}oxy)phenyl]amine (0.088 g, 0.277 mmol), prepared by a procedure analogous to Example 10, Step D, were combined with iPrOH (2 mL) and concentrated HCl (2 drops) in a microwave vial. The reaction was heated to 180° C. for 20 min in the microwave then cooled to rt. TEA (approx. 0.1 mL) and silica gel were combined with the reaction and the resulting mixture was concentrated to dryness and subsequently adhered to the silica gel. Column chromatography using EtOAc, MeOH, and NH₄OH yielded fractions which were concentrated to dryness. The resulting material was then sonicated in ether and the solid that persisted was filtered off to yield 58 mg of the title compound of Example 52 (33% yield) as a yellow powder. ¹H NMR (400 MHz, DMSO-d₆) δ 9.49 (s, 1H), 8.21 (t, J=5.5 Hz, 1H), 8.08 (d, J=5.5 Hz, 1H), 7.80 (dd, J=14.4, 2.6 Hz, 1H), 7.31 (d, J=8.9 Hz, 1H), 7.07 (t, J=9.4 Hz, 1H), 7.01-6.94 (m, 3H), 6.33 (d, J=5.0 Hz, 1H), 6.06 (s, 2H), 4.21 (m, 1H), 3.25 (m, 4H), 3.00 (s, 3H), 2.69 (m, 4H), 2.24 (m, 2H), 1.88 (m, 2H), 1.61 (m, 2H), and 1.16 (t, J=7.7 Hz, 3H). HRMS C₃₀H₃₄N₆O₅FS₂ (M+H)⁺ calcd 641.2016. found 641.2027.

### Example 53

### N-[6-(4-Acetyl-1-piperazinyl)-3-pyridinyl]-4-[4-(1,3-benzodioxol-5-yl)-2-(ethylamino)-1,3-thiazol-5-yl]-2-pyrimidinamine

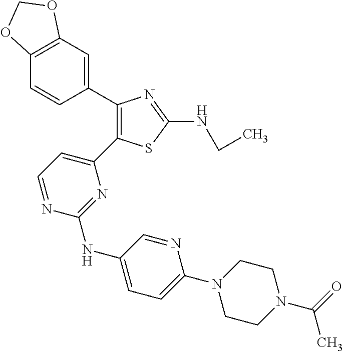

To obtain the title compound, 4-(1,3-benzodioxol-5-yl)-5-(2-chloro-4-pyrimidinyl)-N-ethyl-1,3-thiazol-2-amine (0.1 g, 0.277 mmol), prepared by a procedure analogous to Example 50, Step C, and 6-(4-acetyl-1-piperazinyl)-3-pyridinamine (0.06 g, 0.277 mmol), prepared by a procedure analogous to Example 4, Step B, were combined with iPrOH (2 mL) and concentrated HCl (2 drops) in a microwave vial. The reaction was heated to 180° C. for 15 min in the microwave then cooled to rt. TEA (approx. 0.1 mL) was added and the reaction was concentrated to dryness then purified on a reverse phase acidic HPLC. The resulting fractions were freebased via extraction and concentrated to dryness to yield 26 mg of the title compound of Example 53 (17% yield). ¹H NMR (400 MHz, DMSO-d₆) δ 9.24 (s, 1H), 8.47 (d, J=2.5 Hz, 1H), 8.18 (t, J=5.4 Hz, 1H), 8.04 (d, J=5.3 Hz, 1H), 7.87 (dd, J=9.1, 2.7 Hz, 1H), 6.98 (d, J=13.0 Hz, 2H), 6.97 (s, 1H), 6.81 (d, J=9.0 Hz, 1H), 6.29 (d, J=5.5 Hz, 1H), 6.07 (s, 2H), 3.53 (m, 4H), 3.43 (m, 2H), 3.36 (m, 2H), 3.28 (m, 2H), 2.03 (s, 3H), and 1.16 (t, J=7.6 Hz, 3H). HRMS C₂₇H₂₉N₈O₃S (M+H)⁺ calcd 545.2083. found 545.2092.

### Example 54

### 4-[4-(4-Chloro-3-methylphenyl)-2-(ethylamino)-1,3-thiazol-5-yl]-N-(3-chloro-4-{[2-(1-pyrrolidinyl)ethyl]oxy}phenyl)-2-pyrimidinamine

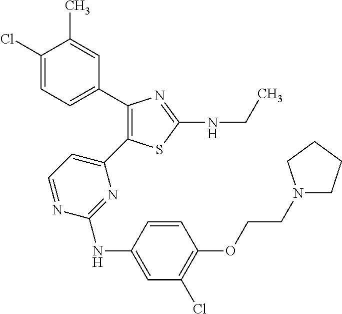

### Step A: 1-(4-Chloro-3-methylphenyl)-2-(2-chloro-4-pyrimidinyl)ethanone

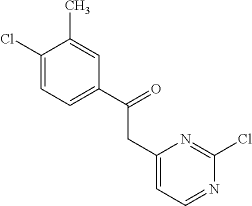

To a solution of methyl 4-chloro-3-methylbenzoate (5.0 g, 27.0 mmol) in THF (30 mL) at 0° C., LHMDS (1.0 M in THF, 57 mL, 57 mmol) was added and the solution was allowed to stir for 5 min. A solution of 2-chloro-4-methylpyrimidine (3.47 g, 27.0 mmol) dissolved in THF (8 mL) was added dropwise at 0° C. and the reaction mixture was allowed to stir for 30 min. The reaction mixture was quenched at 0° C. with MeOH (50 mL) and the solvent was removed in vacuo. The residue was diluted with EtOAc and washed with water. The organic layer was dried over MgSO₄, filtered and evaporated. The title compound of Step A was purified by trituration with EtOAc. The title compound of Step A was obtained as a solid in 20% yield (1.5 g). MS (AFCI): 281.1 [M+H]⁺.

### Step B: 4-(4-Chloro-3-methylphenyl)-5-(2-chloro-4-pyrimidinyl)-N-ethyl-1,3-thiazol-2-amine

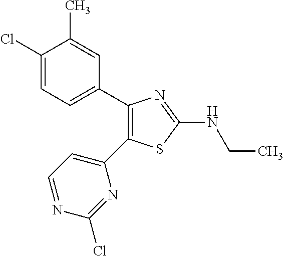

To a solution of 1-(4-chloro-3-methylphenyl)-2-(2-chloro-4-pyrimidinyl)ethanone (300 mg, 1.1 mmol) in DCM (10 mL), NBS (190 mg, 1.1 mmol) was added and the reaction mixture was allowed to stir for 30 min at rt. The solvent was removed and the residue was taken up in DMF (3 mL). Ethyl thiourea (133 mg, 1.28 mmol) was then added and the reaction was allowed to stir for one hour at rt. The reaction mixture was evaporated onto silica gel and chromatographed, 0-50% EtOAc in hexanes followed by 1:9:90 NH₄OH:MeOH:DCM. The title compound of Step B was isolated in 64% yield as a solid after ether trituration (248 mg). MS (APCI): 365.1 [M+H]⁺.

### Step C: 4-[4-(4-Chloro-3-methylphenyl)-2-(ethylamino)-1,3-thiazol-5-yl]-N-(3-chloro-4-{[2-(1-pyrrolidinyl)ethyl]oxy}phenyl)-2-pyrimidinamine

To a mixture of 4-(4-chloro-3-methylphenyl)-5-(2-chloro-4-pyrimidinyl)-N-ethyl-1,3-thiazol-2-amine (100 mg, 0.27 mmol) and (3-chloro-4-{[2-(1-pyrrolidinyl)ethyl]oxy}phenyl)amine (76 mg, 0.27 mmol), prepared in a procedure analogous to Example 20, Step C, in trifluoroethanol (1.5 mL), HCl (4 N in dioxane, 0.2 mL) was added and the solution was heated in a microwave reactor at 170° C. for 10 min. The reaction mixture was evaporated onto silica gel and chromatographed, 10-90% 1:9:90 NH₄OH:MeOH:DCM in DCM. The desired product was obtained as a yellow solid in 74% yield after MeOH:Et₂O trituration (115 mg). ¹H NMR (400 MHz, DMSO-d₆): δ 9.46 (s, 1H), 8.24 (s, 1H), 8.08 (d, J=4.0 Hz, 1H), 7.99 (s, 1H), 7.49-7.44 (m, 3H), 7.33 (d, J=4.0 Hz, 1H), 7.03 (d, J=8.0 Hz, 1H), 6.25 (d, J=8.0 Hz, 1H), 4.07 (t, J=4.0 Hz, 2H), 3.28-3.20 (m, 2H), 2.78 (t, J=4.0 Hz, 2H), 2.50 (br s, 4H), 2.33 (s, 3H), 1.65 (br s, 4H), and 1.17 (t, J=6.0 Hz, 3H); MS (APCI): 571.1 [M+H]⁺.

### Example 55

### 4-[5-(2-{[6-(4-Acetyl-1-piperazinyl)-3-pyridinyl]amino}-4-pyrimidinyl)-2-ethyl-1,3-thiazol-4-yl]-2-(methyloxy)phenyl]methanol

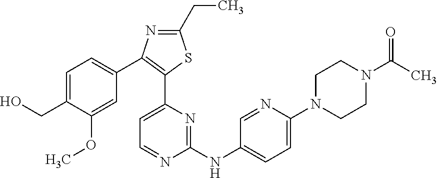

### Step A: Methyl 4-(bromomethyl)-3-(methyloxy)benzoate

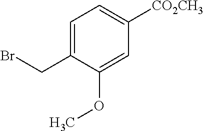

To a solution of methyl 3-methoxy-4-methylbenzoate (10 g, 55.6 mmol) and NBS (11.8 g, 66.7 mmol) in CCl₄ (200 mL) was added AIBN (900 mg, 5.56 mmol). The reaction was stirred for 5 hours at 80° C. TLC showed the reaction was completed. The mixture was filtered and the solvent was removed under the reduced pressure to give the desired product of Step A (11 g, 76.9%). ¹H NMR (CDCl₃, 300 MHz) δ 7.63 (dd, J=1.2 and 6.0 Hz, 1H), 7.55 (s, 1H), 7.39 (d, J=6 Hz, 1H), 4.56 (s, 2H), 3.96 (s, 3H) and 3.91 (s, 3H).

### Step B: Methyl 4-(hydroxymethyl)-3-(methyloxy)benzoate

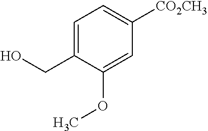

To a suspension of methyl 4-(bromomethyl)-3-methoxybenzoate (1 g, 3.9 mmol) in water (35 mL) was added n-Bu₄NBr (0.2 g, 0.624 mmol) and NaHCO₃ (3.5 g, 42 mmol) at rt. The reaction mixture was heated to 70° C. and stirred for 5 h. The resulting solution was acidified with aqueous HCl (2 mol/L) and extracted with EtOAc. The organic layer was washed with brine, dried over anhydrous Na₂SO₄ and evaporated under vacuum to give the product of Step B (0.4 g, 52.3%): ¹H NMR (CDCl₃, 300 MHz) δ 7.62 (dd, J=11.7 and 12.6 Hz, 1H), 7.57 (s, 1H), 7.46 (dd, J=0.9 and 4.5 Hz, 1H), 4.66 (s, 2H), and 3.85 (s, 6H).

### Step C: Methyl 4-({[(1,1-dimethylethyl)(dimethyl)silyl]oxy}methyl)-3-(methyloxy)benzoate

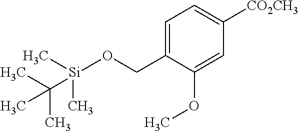

To a solution of methyl 4-(hydroxymethyl)-3-(methyloxy)benzoate (5 g, 25.5 mmol) and imidazole (5.2 g, 76.5 mmol) in dry DCM (100 mL) was added TBSCI (4.6 g, 30.6 mmol) in portions at 0° C. The reaction was stirred for 2 h at rt and then washed by brine and water. The organic layer was separated, and then the solvent was removed under the reduced pressure. The residue was purified by flash column chromatography on silica gel (hexane eluent) to give the desired product of Step C (5 g, 63.3%): ¹H NMR (CDCl₃, 400 MHz) δ 7.56 (d, J=8.8 Hz, 1H), 7.23 (d, J=8.0 Hz, 1H), 7.36 (s, 1H), 4.66 (s, 2H), 3.76 (s, 3H), 3.75 (s, 3H), 0.82 (s, 9H), and 0.01 (s, 6H).

### Step D: 2-(2-Chloro-4-pyrimidinyl)-1-[4-({[(1,1-dimethylethyl)(dimethyl)silyl]oxy}methyl)-3-(methyloxy)phenyl]ethanone

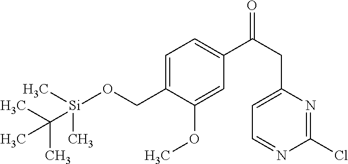

To a solution of methyl 4-({[(1,1-dimethylethyl)(dimethyl)silyl]oxy}methyl)-3-(methyloxy)benzoate (5 g, 16.1 mmol) in dry THF (60 mL) at 0° C., LHMDS (1 M in THF, 2.1 eq, 34.2 mmol, 34.2 mL) was added and the solution was allowed to stir for 10 min at 0° C. A solution of 4-methyl-2-pyrimidinylchloride (1.2 eq, 19.35 mmol, 2.5 g) in 10 mL of THF was then added dropwise to the solution of ester and base at 0° C. over 10 min. Reaction mixture turns black. The solution was allowed to stir 30 min at 0° C. LC-MS showed the reaction was complete. The reaction mixture was quenched at 0° C. with 10 mL of MeOH and the mixture was concentrated to dryness. The residue was diluted with EtOAc and washed with water. The water layer was extracted twice with EtOAc and then dried with Na₂SO₄. The solvent was removed under the reduced pressure, the residue was purified by flash column chromatography on silica gel (10 to 20% EtOAc:hexanes) to give the desired product of Step D as an approximately 1:1 mixture of ketone and enol tautomers (4.9 g, 76%). The reported data is for the observed mixture: ¹H NMR (CDCl₃, 300 MHz) δ 13.85 (s, 0.5H), 8.59 (d, J=3.9 Hz, 0.5H), 8.37 (d, J=3.9 Hz, 0.5H), 7.65-7.63 (m, 1H), 7.54-7.53 (m, 0.5H), 7.48-7.47 (m, 1H), 7.33-7.27 (m, 1H), 6.90 (d, J=3.9 Hz), 6.06 (s, 0.5H), 4.79 (s, 1H), 4.48 (s, 1H), 3.40 (s, 1.5H), 3.89 (s, 1.5H), 0.98 (s, 9H), and 0.14 (s, 6H).

### Step E: 2-Chloro-4-{4-[4-({[(1,1-dimethylethyl)(dimethyl)silyl]oxy}methyl)-3-(methyloxy)phenyl]-2-ethyl-1,3-thiazol-5-yl}pyrimidine

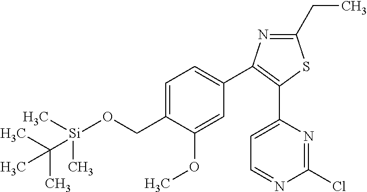

To a solution of 2-(2-chloro-4-pyrimidinyl)-1-[4-({[(1,1-dimethylethyl)(dimethyl)silyl]oxy}methyl)-3-(methyloxy)phenyl]ethanone (6.6 g, 16.3 mmol) in 40 mL of DCM, NBS was added and the solution was allowed to stir at rt for 30 min. The reaction mixture was then concentrated on the rotovap and the resulting oil was diluted with DMSO (40 mL) and propanethioamide (1.5 equivalents, 2.2 g, 24.4 mmol) was added. The reaction was complete after stirring one h at rt. The mixture was washed with water and extracted with DCM, repeated until DMSO was removed completely. The organic layer was separated and dried with anhydrous Na₂SO₄. Solvent was removed under the reduced pressure and the residue was dissolved in dry DCM (40 mL). Imidazole (19.6 mmol, 1.33 g) was added, followed by TBSCI (16.3 mmol, 2.45 g). The reaction was stirred for 1 h at rt, and then washed by brine. The organic layer was separated, and then the solvent was removed under the reduced pressure. The residue was purified by column chromatography on silica gel (5% to 20% EtOAc: hexanes) to give the desired product of Step E (3 g, 38.7%). ¹H NMR (CDCl₃, 300 MHz) δ 8.31 (d, J=0.9 Hz, 1H), 7.54 (d, J=6.0 Hz, 1H), 7.11 (d, J=2.7 Hz, 1H), 7.06 (d, J=3.9 Hz, 1H), 7.00 (s, 1H), 4.82 (s, 3H), 3.10 (dd, J=5.7 and 11.1 Hz, 2H), 1.46 (t, J=5.7 Hz, 3H), 0.97 (s, 9H) and 0.14 (s, 6H). MS (ESI) m/e (M+H⁺) 476.1

### Step F: (u24334/131/5) [4-[5-(2-{[6-(4-Acetyl-1-piperazinyl)-3-pyridinyl]amino}-4-pyrimidinyl)-2-ethyl-1,3-thiazol-4-yl]-2-(methyloxy)phenyl]methanol

To a stirring suspension of 2-chloro-4-{4-[4-({[(1,1-dimethylethyl)(dimethyl)silyl]oxy}methyl)-3-(methyloxy)phenyl]-2-ethyl-1,3-thiazol-5-yl}pyrimidine (0.150 g, 1.00 eq) in 10:1 n-BuOH/MeOH (3.3 mL) in a microwave vial was added 6-(4-acetylpiperazin-1-yl)pyridin-3-amine (0.075 g, 1.08 eq), prepared by a procedure analogous to Example 4, Step B. The suspension was heated in the microwave at 170° C. for 25 min. The cooled reaction mixture was diluted with DCM (3.0 mL) and stirred with HCl (4 M solution in dioxane, 0.3 mL). After 10 min the reaction solution was quenched with TEA (0.5 mL), concentrated onto silica gel, and purified by column chromatography (gradient elution: 0 to 40% DCM/solution of 15% 2M NH₃ in MeOH and 85% DCM). The appropriate fractions were combined and concentrated until a solid precipitated from solution. The solids were filtered off and the resultant solution concentrated to give a solid. This solid was dissolved in DCM and washed 2× with NaHCO₃ (aq). The organic extracts were dried over MgSO₄, filtered, and concentrated to provide 0.016 g (11%) of the title compound of Example 55: ¹H NMR (400 MHz, DMSO-d₆) δ 9.50 (s, 1H), 8.44 (d, J=2.4 Hz, 1H), 8.26 (d, J=5.1 Hz, 1H), 7.87 (dd, J=9.0, 2.6 Hz, 1H), 7.45 (d, J=7.7 Hz, 1H), 7.08-7.14 (m, 2H), 6.84 (d, J=9.2 Hz, 1H), 6.48 (d, J=5.1 Hz, 1H), 5.12 (t, J=5.6 Hz, 1H), 4.55 (d, J=5.5 Hz, 2H), 3.75 (s, 3H), 3.50-3.57 (m, 4H), 3.44-3.50 (m, 2H), 3.36-3.42 (m, 2H), 3.05 (q, J=7.5 Hz, 2H), 2.05 (s, 3H), and 1.36 (t, J=7.5 Hz, 3H); MS (ESI): 546.05 [M+H]⁺.

### Example 56

### 4-{2-(Ethylamino)-4-[2-fluoro-5-(methyloxy)phenyl]-1,3-thiazol-5-yl}-N-[3-fluoro-4-(4-methyl-1-piperazinyl)phenyl]-2-pyrimidinamine

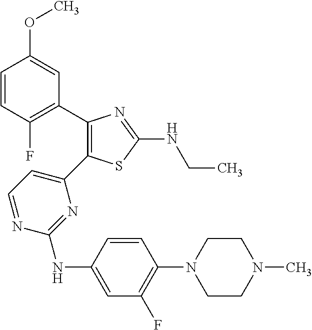

### Step A: Methyl 2-fluoro-5-(methyloxy)benzoate

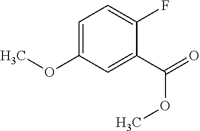

To a suspension of 2-fluoro-5-(methyloxy)benzoic acid (2.0 g, 11.8 mmol) in DCM (20 mL) and MeOH (2 mL) at 0° C., trimethylsilyl diazomethane (2 M in ether, 12.3 mmol, 6.2 mL) was added dropwise. The reaction mixture was allowed to stir for 30 min at 0° C. and the solvents were removed under reduced pressure. The residue was diluted with EtOAc, adsorbed onto silica gel, and subjected to silica gel chromatography to give 2.1 g (97%) of the title compound of Step A: ¹H NMR (CDCl₃, 300 MHz) δ 7.41-7.39 (m, 1H), 7.07-7.02 (m, 2H), 3.93 (s, 3H), and 3.82 (s, 3H).

### Step B: 2-(2-Chloro-4-pyrimidinyl)-1-[2-fluoro-5-(methyloxy)phenyl]ethanone

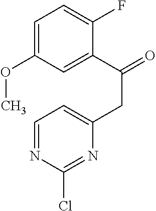

To a solution of methyl 2-fluoro-5-(methyloxy)benzoate (2.1 g, 1.3 mmol) in dry THF (30 mL) at 0° C., LHMDS (1 M in THF, 24 mmol, 24 mL) was added and the solution was allowed to stir for 10 min at 0° C. A solution of 2-chloro-4-methylpyrimidine (13.5 mmol, 1.7 g) in THF was then added dropwise to the solution of ester and base at 0° C. over 15 min. The solution was allowed to stir 30 min at 0° C., quenched at 0° C. by the addition of MeOH and the solvents were removed under reduced pressure. The residue was diluted with EtOAc and washed with water. The water layer was extracted with EtOAc. Crystallization from EtOAc and ether gave 2.5 g (79%) of 2-(2-chloro-4-pyrimidinyl)-1-[2-fluoro-5-(methyloxy)phenyl]ethanone: ¹H NMR (CDCl₃, 400 MHz) δ 13.83 (s, 0.7H), 8.60 (d, J=5.2 Hz, 0.3H), 8.41 (d, J=5.2 Hz, 0.7H), 7.43 (dd, J=3.2, 6.0 Hz, 0.7H), 7.36 (t, J=3.2 Hz, 0.3H), 7.28 (d, J=4.8 Hz, 0.3H), 7.12-7.04 (m, 1.4H), 6.96-6.92 (m, 1.4H), 6.29 (s, 0.7H), 4.48 (d, J=2.8 Hz, 0.7H), 3.84 (s, 2.1H), and 3.82 (s, 0.9H).

### Step C: 5-(2-Chloro-4-pyrimidinyl)-N-ethyl-4-[2-fluoro-5-(methyloxy)phenyl]-1,3-thiazol-2-amine

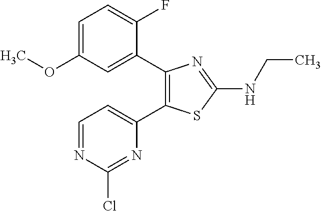

In a procedure analogous to Example 1, Step F, 1.1 g (42% yield) of 5-(2-chloro-4-pyrimidinyl)-N-ethyl-4-[2-fluoro-5-(methyloxy)phenyl]-1,3-thiazol-2-amine was produced from 2.2 g (7.8 mmol) of 2-(2-chloro-4-pyrimidinyl)-1-[2-fluoro-5-(methyloxy)phenyl]ethanone: ¹H NMR (CDCl₃, 400 MHz) δ 8.18 (d, J=5.6 Hz, 1H), 7.10 (t, J=8.4 Hz, 1H), 7.00-6.95 (m, 2H), 6.72 (d, J=5.6 Hz, 1H), 6.11 (brs, 1H), 3.81 (s, 3H), 3.36-3.30 (m, 2H), and 1.30 (t, J=7.2 Hz, 3H).

### Step D: 4-{2-(Ethylamino)-4-[2-fluoro-5-(methyloxy)phenyl]-1,3-thiazol-5-yl}-N-[3-fluoro-4-(4-methyl-1-piperazinyl)phenyl]-2-pyrimidinamine

To obtain the title compound of Example 56, 5-(2-chloro-4-pyrimidinyl)-N-ethyl-4-[2-fluoro-5-(methyloxy)phenyl]-1,3-thiazol-2-amine (0.100 g, 0.274 mmol) and [3-fluoro-4-(4-methyl-1-piperazinyl)phenyl]amine (0.057 g, 0.274 mmol) were combined with iPrOH (2 mL) and concentrated HCl (2 drops) in a microwave vial. The reaction was heated to 180° C. for 20 min in the microwave then cooled to rt. TEA (approx. 0.1 mL) and silica gel were combined with the reaction and the resulting mixture was concentrated to dryness and subsequently adhered to silica gel. Column chromatography using EtOAc, MeOH, and ammonium hydroxide yielded fractions which were concentrated to dryness. This material was then sonicated in ether and the solid that persisted was filtered off to yield 41 mg of the title compound of Example 56 (63% Y). ¹H NMR (400 MHz, DMSO-d₆) δ 9.51 (s, 1H), 8.31 (t, J=5.3 Hz, 1H), 8.11 (d, J=5.5 Hz, 1H), 7.70 (dd, J=15.7, 2.4 Hz, 1H), 7.34 (dd, J=8.9, 2.3 Hz, 1H), 7.23 (t, J=9.0 Hz, 1H), 7.05 (m, 1H), 7.01 (m, 1H), 6.92 (t, J=9.3 Hz, 1H), 6.12 (d, J=5.2 Hz, 1H), 3.75 (s, 3H), 3.28 (m, 2H), 2.93 (m, 4H), 2.45 (br, 4H), 2.21 (s, 3H), 1.18 (t, J=7.6 Hz, 3H). HRMS C₂₇H₃₀N₇OF₂S (M+H)⁺ calcd 538.2201. found 538.2211.

### Example 57

### N-(3-Chloro-4-{[2-(1-pyrrolidinyl)ethyl]oxy}phenyl)-4-{2-(ethylamino)-4-[4-(methyloxy)-2-pyridinyl]-1,3-thiazol-5-yl}-2-pyrimidinamine

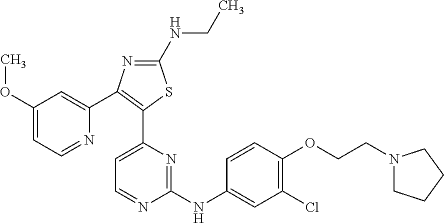

### Step A: Methyl 4-(methyloxy)-2-pyridinecarboxylate

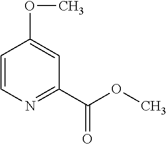

A solution of methyl 4-chloro-2-pyridinecarboxylate hydrochloride (2.0 g, 9.7 mmol) in MeOH (25 mL) was stirred at 60° C. for 36 h. The MeOH was removed on the rotovap, and the residue was partitioned between EtOAc and saturated aqueous NaHCO₃. The aqueous layer was extracted with EtOAc, and the combined organic layers were dried over anhydrous Na₂SO₄, filtered, and concentrated to generate the title compound of Step A as a white crystalline solid in quantitative yield (1.6 g, 9.7 mmol). ¹H NMR (400 MHz, DMSO-d₆): δ 8.48 (d, 1H, J=5.7 Hz), 7.51 (d, 1H, J=2.8 Hz), 7.19 (dd, 1H, J=2.6 Hz, 5.7 Hz), 3.87 (s, 3H), and 3.84 (s, 3H).

### Step B: 2-(2-Chloro-4-pyrimidinyl)-1-[4-(methyloxy)-2-pyridinyl]ethanone

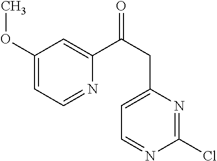

To a solution of methyl 4-(methyloxy)-2-pyridinecarboxylate (0.8 g, 4.8 mmol) in anhydrous THF (10 mL) cooled to 0° C. was added a 1.0 M solution of LHMDS in THF (6.2 mL, 6.2 mmol). A solution of 2-chloro-4-methylpyrimidine (0.61 g, 4.8 mmol) in anhydrous THF (5 mL) was added, and the reaction was stirred at 0° C. After 30 min, the reaction was quenched with MeOH (1 mL) and concentrated. The residue was partitioned between EtOAc and water. The aqueous layer was extracted with EtOAc, and the combined organic layers were washed with brine, concentrated, and absorbed onto silica gel. The crude product was purified by column chromatography to generate the title compound of Step B in 16% yield (0.20 g, 0.76 mmol). MS (ESI) m/z=264 [M+H]⁺.

### Step C: 5-(2-Chloro-4-pyrimidinyl)-N-ethyl-4-[4-(methyloxy)-2-pyridinyl]-1,3-thiazol-2-amine

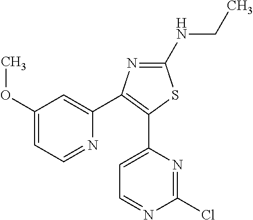

To a suspension of 2-(2-chloro-4-pyrimidinyl)-1-[4-(methyloxy)-2-pyridinyl]ethanone (0.20 g, 0.76 mmol) in DCM (5 mL) was added NBS (0.14 g, 0.80 mmol). After stirring for 30 min at rt, the solvent was removed on the rotovap. The residue was redissolved in DMF (5 mL), and 1-ethyl-2-thiourea (95 mg, 0.91 mmol) was added. After 3 h at rt, the reaction mixture was partitioned between EtOAc and water. The pH of the aqueous layer was raised to >9 with 1 M aqueous Na₂CO₃, and the layers were separated. The aqueous layer was extracted with EtOAc and DCM. The combined organic layers were concentrated to a brown oil that slowly solidified. The crude title compound of Step C (320 mg, 0.92 mmol) was used in the next reaction without further purification. MS (ESI) m/z=348 [M+H]⁺.

### Step D: N-(3-chloro-4-{[2-(1-pyrrolidinyl)ethyl]oxy}phenyl)-4-{2-(ethylamino)-4-[4-(methyloxy)-2-pyridinyl]-1,3-thiazol-5-yl}-2-pyrimidinamine

To a suspension of 5-(2-chloro-4-pyrimidinyl)-N-ethyl-4-[4-(methyloxy)-2-pyridinyl]-1,3-thiazol-2-amine (132 mg, 0.38 mmol) in iPrOH (2 mL) was added (3-chloro-4-{[2-(1-pyrrolidinyl)ethyl]oxy}phenyl)amine hydrochloride (105 mg, 3.8 mmol), prepared in a procedure analogous to Example 20, Step C. The reaction was heated to 170° C. in a microwave reactor for 15 min. The reaction mixture was concentrated, redissolved in 3 mL 1:1 MeOH/DMSO, and purified by RP-HPLC (eluting with 5-50% acetonitrile/water/0.1% TFA). The product fractions were combined, basified to pH>14 with 1 N NaOH, and extracted with EtOAc. The organic layer was concentrated, redissolved with 10:1 water/MeOH, frozen with dry ice/acetone, and lyophilized to generate the title compound of Example 54 as a yellow solid in 24% yield (50 mg, 0.09 mmol). ¹H NMR (400 MHz, DMSO-d₆, mixture of rotamers): δ 9.46 (s, 0.5H), 8.61 (d, 0.5H, J=5.2 Hz), 8.43 (d, 1H, J=5.7 Hz), 8.31-8.35 (m, 0.5H), 8.27 (t, 0.5H, J=5.4 Hz), 8.18 (m, 0.5H), 8.12 (d, 0.5H, J=5.5 Hz), 8.03 (dd, 1H, J=2.3 Hz, 7.3 Hz), 7.80 (m, 0.5H), 7.75 (dd, 0.5H, J=2.2 Hz, 5.3 Hz), 7.52 (dd, 0.5H, J=2.6 Hz, 9.2 Hz), 7.24 (d, 0.5H, J=2.4 Hz), 7.08 (m, 1.5H), 6.55 (d, 0.5H, J=5.5 Hz), 4.11 (t, 2H, J=5.7 Hz), 3.87 (s, 3H), 2.84 (bs, 2H), 2.58 (bs, 4H), 1.69 (bs, 4H), and 1.20 (t, 3H, J=7.2 Hz). MS (ESI) m/z=552 [M+H]⁺.

### Example 58

### N-(3-Chloro-4-{[2-(1-pyrrolidinyl)ethyl]oxy}phenyl)-4-{2-(ethylamino)-4-[2-(methyloxy)-4-pyridinyl]-1,3-thiazol-5-yl}-2-pyrimidinamine

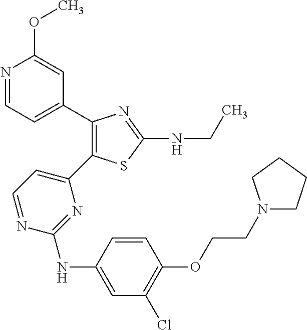

### Step A: 1-(2-Chloro-4-pyridinyl)-2-(2-chloro-4-pyrimidinyl)ethanone

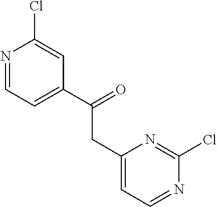

To a solution of 2-chloro-4-pyridinecarboxylic acid (5.0 g, 31.7 mmol) in DCM (20 mL) and MeOH (2 mL) at 0° C., (trimethylsilyl)diazomethane (2.0 M in ether, 16 mL) was added dropwise. After stirring 15 min the reaction mixture was evaporated onto silica gel and chromatographed, 20-80% EtOAc in hexanes. The intermediate material, methyl 2-chloro-4-pyridinecarboxylate, was dissolved in THF (30 mL) and cooled to 0° C., LHMDS (1.0 M in THF, 57 mL, 57 mmol) was added. A solution of 2-chloro-4-methylpyrimidine (3.47 g, 27.0 mmol) dissolved in THF (8 mL) was added dropwise at 0° C. and the reaction mixture was allowed to stir for 30 min. The reaction mixture was quenched at 0° C. with MeOH (50 mL). Solvent was removed in vacuo. The residue was diluted with EtOAc and washed with water. The organic layer was dried over MgSO₄, filtered and evaporated. The title compound of Step A was purified by trituration with EtOAc. The title compound of Step A was obtained as a solid in 22% yield (1.75 g). MS (ESI): 268.2 [M+H]⁺.

### Step B: 4-(2-Chloro-4-pyridinyl)-5-(2-chloro-4-pyrimidinyl)-N-ethyl-1,3-thiazol-2-amine

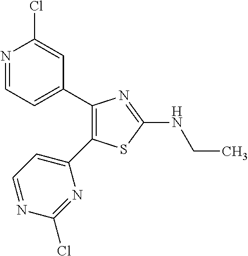

The title compound of Step B was prepared from 1-(2-chloro-4-pyridinyl)-2-(2-chloro-4-pyrimidinyl)ethanone (1.0 g, 3.72 mmol) and ethyl thiourea (0.47 g, 4.48 mmol) by a procedure analogous to Example 1, Step F. The title compound of Step B was isolated in 39% yield as a beige solid (0.51 g). MS (ESI): 354.2 [M+H]⁺.

### Step C: 4-[4-(2-Chloro-4-pyridinyl)-2-(ethylamino)-1,3-thiazol-5-yl]-N-(3-chloro-4-{[2-(1-pyrrolidinyl)ethyl]oxy}phenyl)-2-pyrimidinamine

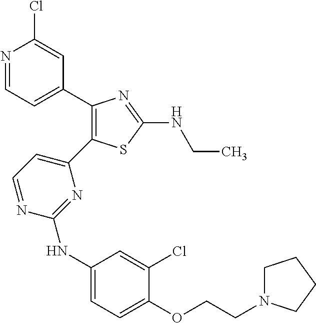

The title compound of Step C was prepared from 4-(2-chloro-4-pyridinyl)-5-(2-chloro-4-pyrimidinyl)-N-ethyl-1,3-thiazol-2-amine (250 mg, 0.71 mmol) and (3-chloro-4-{[2-(1-pyrrolidinyl)ethyl]oxy}phenyl)amine hydrochloride (197 mg, 0.71 mmol), prepared by a procedure analogous to Example 20, Step C, by a procedure analogous to Example 1, Step G. The title compound of Step C was isolated in 39% yield as a solid (156 mg). MS (APCI): 556.1 [M+H]⁺.

### Step D: N-(3-Chloro-4-{[2-(1-pyrrolidinyl)ethyl]oxy}phenyl)-4-{2-(ethylamino)-4-[2-(methyloxy)-4-pyridinyl]-1,3-thiazol-5-yl}-2-pyrimidinamine (title compound)

A mixture of 4-[4-(2-chloro-4-pyridinyl)-2-(ethylamino)-1,3-thiazol-5-yl]-N-(3-chloro-4-{[2-(1-pyrrolidinyl)ethyl]oxy}phenyl)-2-pyrimidinamine (112 mg, 0.20 mmol) in sodium methoxide (25% wt in MeOH, 2 mL) was heated in a microwave reactor at 150° C. for 10 min. The reaction mixture was then evaporated onto silica gel and chromatographed, 10-90% 1:9:90 NH₄OH:MeOH:DCM in DCM. The desired product of Step D was obtained as a solid in 77% yield after MeOH trituration (85 mg). ¹H NMR (400 MHz, DMSO-d₆) δ ppm 9.49 (s, 1H), 8.29 (s, 1H), 8.19 (d, J=5.1 Hz, 1H), 8.12 (d, J=5.3 Hz, 1H), 7.96 (s, 1H), 7.45 (d, J=9.5 Hz, 1H), 6.96-7.10 (m, 2 H), 6.88 (s, 1H), 6.32 (d, J=5.5 Hz, 1H), 3.99-4.11 (m, 2H), 3.84 (s, 3H), 3.19-3.26 (m, 2H), 2.69-2.80 (m, J=5.6, 5.6 Hz, 2H), 2.51 (br s, 4H), 1.64 (br s, 4H), and 1.16 (t, J=7.2 Hz, 3H); MS (APCI): 552.1 [M+H]⁺.

### Example 59

### N-(3-Fluoro-4-{[1-(1-methylethyl)-4-piperidinyl]oxy}phenyl)-4-{2-(1-methylethyl)-4-[3-(methyloxy)phenyl]-1,3-thiazol-5-yl}-2-pyrimidinamine

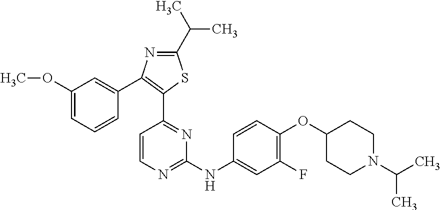

### Step A: 2-Methylpropanethioamide

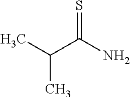

A solution of 2-methylpropanamide (6.53 g, 75.0 mmol) and 2,4-bis(4-methoxyphenyl)-1,3-dithia-2,4-diphosphetane-2,4-disulfide (15.17 g, 37.51 mmol) in THF (100 mL) was heated to reflux for 4 h. The reaction mixture was then cooled to rt and poured into saturated aqueous NaHCO₃ (200 mL). The mixture was extracted with ether (4×100 mL). The organic fractions were combined, dried over Na₂SO₄, filtered, and concentrated. Purification by flash column chromatography (20% EtOAc:hexanes) afforded 4.77 g (62%) of the title compound of Step A. ¹H NMR (400 MHz, CDCl₃) δ 7.63 (br s, 1H), 6.90 (br s, 1H), 2.88 (m, 1H), and 1.27 (d, 6H, J=6.8 Hz).

### Step B: 2-Chloro-4-{2-(1-methylethyl)-4-[3-(methyloxy)phenyl]-1,3-thiazol-5-yl}pyrimidine

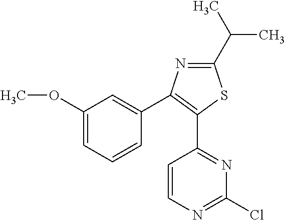

To a solution of 2-(2-chloro-4-pyrimidinyl)-1-[3-(methyloxy)phenyl]ethanone (1.50 g, 5.72 mmol), prepared by a procedure analogous to Example 40, Step A, in DMF (19 mL) was added NBS (1.02 g, 5.72 mmol). After stirring 30 min at rt, 2-methylpropanethioamide (885 mg, 8.58 mmol) was added and the reaction was stirred a further 1 h at rt. The reaction mixture was then poured into EtOAc (100 mL) and washed with water (3×100 mL). The organic fraction was dried over Na₂SO₄, filtered, and concentrated onto silica gel. Purification by flash column chromatography (10 to 50% EtOAc:hexanes) afforded 920 mg (46%) of the title compound of Step B. ¹H NMR (400 MHz, CDCl₃): δ 8.31 (d, 1H, J=5.3 Hz), 7.41-7.29 (m, 1H), 7.15-7.03 (m, 2H), 7.00 (m, 2H), 3.82 (s, 3H), 3.57-3.12 (m, 1H), and 1.47 (d, 6H, J=6.8 Hz); MS (ESI): 346.24 [M+H]⁺.

### Step C: (3-Fluoro-4-{[1-(1-methylethyl)-4-piperidinyl]oxy}phenyl)amine

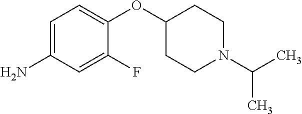

To a suspension containing 2.0 g (5.65 mmol) of 4-[(2-fluoro-4-nitrophenyl)oxy]-1-(1-methylethyl)piperidine hydrobromide, prepared by a procedure analogous to Example 10, Step B, and 150 mL acetone was added 20 mL of MeOH and 20 mL of DCM. To this mixture was added 20 g (94.4 mmol) of sodium triacetoxy borohydride. The reaction was allowed to stir overnight at rt and the reaction was partitioned between EtOAc and water. The organic phase was washed with brine, dried, and filtered, and the solvents were removed under reduced pressure. The residue was dissolved in DCM and washed with water. The organic layer was dried and filtered and the solvent was removed under reduced pressure to give 1.5 g of a crude material that was used without further purification. The crude compound was dissolved in MeOH and 0.7 g (2.94 mmol) of nickel (II) chloride hexahydrate was added. The mixture was cooled to 0° C. and 0.5 g (13.2 mmol) of sodium borohydride was added. The reaction mixture was allowed to stir for 30 min and a small amount more of sodium borohydride was added and the reaction was allowed to stir for an additional 15 min. The solvents were removed under reduced pressure and the reaction was quenched by the addition of 2 N aqueous NaOH and extracted with EtOAc. The organic layer was washed with brine and dried, and the solvent was removed under reduced pressure to give 1.05 g (73%) of a crude residue that was used without further purification. ¹H NMR (400 MHz, DMSO-d₆) δ 6.81 (t, J=9.2 Hz, 1H), 6.36 (d, J=13.6 Hz, 1H), 6.27 (dd, J=8.7 and 1.4 Hz, 1H), 4.97 (s, 2H), 3.90 (ddd, J=12.4, 8.2 and 3.7 Hz, 1H), 2.62-2.72 (m, 3H), 2.20 (t, J=10.0 Hz, 2H), 1.84 (d, J=2.4 Hz, 1H), 1.81 (s, 1H), 1.47-1.58 (m, 2H), and 0.94 (d, J=6.6 Hz, 6H).

### Step D: N-(3-Fluoro-4-{[1-(1-methylethyl)-4-piperidinyl]oxy}phenyl)-4-{2-(1-methylethyl)-4-[3-(methyloxy)phenyl]-1,3-thiazol-5-yl}-2-pyrimidinamine

A suspension of 2-chloro-4-{2-(1-methylethyl)-4-[3-(methyloxy)phenyl]-1,3-thiazol-5-yl}pyrimidine (100 mg, 0.289 mmol) and (3-fluoro-4-{[1-(1-methylethyl)-4-piperidinyl]oxy}phenyl)amine (104 mg, 0.361 mmol) in iPrOH (2.9 mL) with 3 drops of concentrated HCl added was heated in the microwave at 180° C. for 10 min. The reaction mixture was partitioned between EtOAc (30 mL) and saturated aqueous NaHCO₃ (30 mL). The organic fraction was dried over Na₂SO₄, filtered, and concentrated. The crude material was purified by flash column chromatography (0 to 50% (15% MeOH/1% NH₄OH/DCM):DCM) and then preparative HPLC (10 to 70% acetonitrile:water w/0.1% TFA). The material obtained was redissolved in EtOAc (30 mL) and washed with saturated aqueous NaHCO₃ (2×40 mL). The organic fraction was dried over Na₂SO₄, filtered, and concentrated to afford 63 mg (39%) of the title compound of Example 59. ¹H NMR (400 MHz, CDCl₃): δ 8.16 (d, 1H, J=5.3 Hz), 7.69 (dd, 1H, J=2.6, 13.4 Hz), 7.33 (t, 1H, J=7.9 Hz), 7.12 (m, 2H), 7.07 (m, 2H), 6.96 (m, 2H), 6.56 (d, 1H, J=5.3 Hz), 4.20 (m, 1H), 3.81 (s, 3H), 3.37 (m, 1H), 2.84-2.72 (m, 3H), 2.36 (t, 2H, J=8.7 Hz), 2.01 (m, 2H), 1.85 (m, 2H), 1.47 (d, 6H, J=7.0 Hz), and 1.06 (d, 6H, J=6.6 Hz); MS (ESI): 562.26 [M+H]⁺.

### Example 60

### {5-{2-[(3-Fluoro-4-{[2-(1-pyrrolidinyl)ethyl]oxy}phenyl)amino]-4-pyrimidinyl}-4-[3-(methyloxy)phenyl]-1,3-thiazol-2-yl}methanol

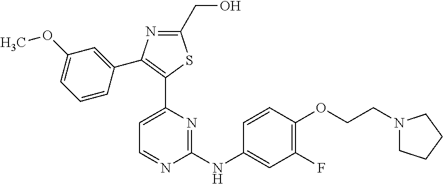

### Step A: {5-(2-Chloro-4-pyrimidinyl)-4-[3-(methyloxy)phenyl]-1,3-thiazol-2-yl}methyl 2,2-dimethylpropanoate

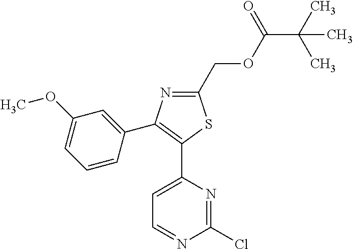

To 2-(2-chloro-4-pyrimidinyl)-1-[3-(methyloxy)phenyl]ethanone (0.44 g, 1.68 mmol) in DCM (50 mL) was added NBS (0.45 g, 2.52 mmol) and stirred for 15 min. The volatiles were removed under reduced pressure and DMF (20 mL) was added. The mixture was cooled in ice bath and 2-amino-2-thioxoethyl 2,2-dimethylpropanoate (0.44 g, 2.52 mmol) was added. The ice bath was removed and the reaction mixture was stirred at rt for 3 h. EtOAc was added and the organic layer was separated, washed with water (4×50 mL), dried over Na₂SO₄, absorbed onto silica and purified with EtOAc:hexane (0% to 50%) to afford 0.18 g of the title compound of Step A. ¹H NMR (400 MHz, CDCl₃) δ 8.35 (d, J=5.3 Hz, 1H), 7.36 (t, J=8.1 Hz, 1H), 6.93-7.16 (m, 4H), 5.42 (s, 2H), 3.82 (s, 3H), 1.32 (s, 9H). MS (ESI): 418 [M+H]⁺.

### Step B: 1-{2-[(2-Fluoro-4-nitrophenyl)oxy]ethyl}pyrrolidine

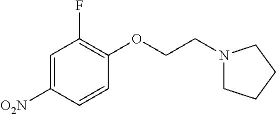

To obtain the target compound, NaH (2.5 g, 63 mmol, 60% dispersion) was placed in a round bottom flask with THF (25 mL) at 0° C. 2-(1-Pyrrolidinyl)ethanol (7.5 mL, 68 mmol) was added portion-wise to the stirring NaH solution over 15 min. This reaction mixture was stirred at 0° C. for 45 min. 3,4-Difluoronitrobenzene (10 g, 63 mmol) was then added portion-wise to the reaction at 0° C. The reaction was allowed to stir and warm rt over several hours. A few drops of MeOH were added to the reaction. EtOAc and water were then added to the reaction mixture and the desired product was extracted into the organic phase which was then concentrated onto silica gel and purified via column chromatography to yield 8.5 g of the title compound of Step B. (53%) ¹H NMR (400 MHz, DMSO-d₆) δ ppm 8.06-8.15 (m, 2H), 7.40 (t, J=8.79 Hz, 1H), 4.28 (t, J=5.68 Hz, 2H), 2.82 (t, J=5.68 Hz, 2H), 2.49 (m, 4H), 1.65 (m, 4H).

### Step C: (3-Fluoro-4-{[2-(1-pyrrolidinyl)ethyl]oxy}phenyl)amine hydrochloride

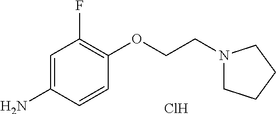

To obtain the title compound of Step C, 1-{2-[(2-fluoro-4-nitrophenyl)oxy]ethyl}pyrrolidine (8.5 g, 33 mmol) and EtOH (50 mL) were placed in a pressure reaction vessel under N₂. Platinum on carbon (500 mg, 5% by wt) was added followed by 40 psi of H₂. The reaction was stirred overnight at rt. The reaction was then filtered through celite and the subsequent filtrate was concentrated to dryness. A small amount of EtOAc was added followed by 4 N HCl in dioxane (8.25 mL, 33 mmol). This was then concentrated to dryness to yield a sticky solid which was then sonicated in ether and filtered off as a beige powder to yield 8.0 g of the title compound of Step C as a hydrochloride salt. ¹H NMR (400 MHz, DMSO-d₆) δ ppm 6.92 (m, 1H), 6.41 (m, 1H), 6.31 (m, 1H), 5.28 (s, 2H), 4.20 (m, 2H), 3.54 (s, 2H), 3.47 (m, 2H), 3.06 (s, 2H), 1.97 (s, 2H), 1.86 (s, 2H).

### Step D: {5-{2-[(3-Fluoro-4-{[2-(1-pyrrolidinyl)ethyl]oxy}phenyl)amino]-4-pyrimidinyl}-4-[3-(methyloxy)phenyl]-1,3-thiazol-2-yl}methanol

The general procedure analogous to Example 1, Step G was followed to make the intermediate, {5-{2-[(3-fluoro-4-{[2-(1-pyrrolidinyl)ethyl]oxy}phenyl)amino]-4-pyrimidinyl}-4-[3-(methyloxy)phenyl]-1,3-thiazol-2-yl}methyl 2,2-dimethylpropanoate from {5-(2-chloro-4-pyrimidinyl)-4-[3-(methyloxy)phenyl]-1,3-thiazol-2-yl}methyl 2,2-dimethylpropanoate (0.095 g, 0.21 mmol) and (3-fluoro-4-{[2-(1-pyrrolidinyl)ethyl]oxy}phenyl)amine (0.066 g, 0.26 mmol). To this intermediate was added MeOH (5 mL) and 0.5 N sodium methoxide in MeOH (1.0 mL). The reaction mixture was stirred at rt for 2 h, absorbed onto silica and purified by column chromatography using 25% to 100% DCM: (84% DCM, 15% MeOH, and 1% NH₄OH) to afford 0.042 g of the title compound of Example 60. ¹H NMR (400 MHz, DMSO-d₆) δ 9.74 (s, 1H), 8.32 (d, J=5.1 Hz, 1H), 7.75 (dd, J=14.5, 2.4 Hz, 1H), 7.37 (t, J=8.1 Hz, 2H), 7.06-7.13 (m, 2H), 7.02 (dd, J=8.1, 2.2 Hz, 1H), 6.53 (d, J=5.1 Hz, 1H), 6.26 (t, J=5.7 Hz, 1H), 4.79 (d, J=5.9 Hz, 2H), 4.10 (t, J=5.9 Hz, 2H), 3.76 (s, 3H), 2.82 (t, J=5.3 Hz, 2H), 2.56 (s, 4H), and 1.69 (s, 4H). MS (ESI): 522 [M+H]⁺.

### Example 61

### 4-{2-(1-Methylethyl)-4-[3-methyl-5-(methyloxy)phenyl]-1,3-thiazol-5-yl}-N-[6-(4-morpholinyl)-3-pyridinyl]-2-pyrimidinamine

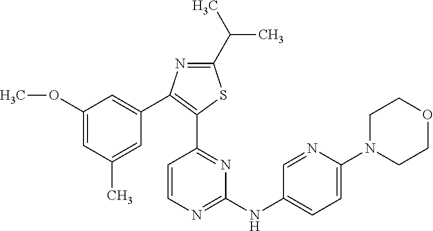

### Step A: 2-Chloro-4-{2-(1-methylethyl)-4-[3-methyl-5-(methyloxy)phenyl]-1,3-thiazol-5-yl}pyrimidine

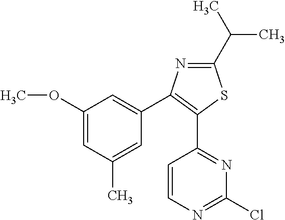

The title compound of Step A was prepared from 2-(2-chloro-4-pyrimidinyl)-1-[3-methyl-5-(methyloxy)phenyl]ethanone (3.00 g, 10.8 mmol), prepared by a procedure analogous to Example 25, Step E, and 2-methylpropanethioamide (1.68 g, 16.3 mmol), prepared by a procedure analogous to Example 59, Step A, by a procedure analogous to Example 59, Step B. Upon completion of the reaction, the reaction mixture was diluted with water (50 mL), causing the formation of a sticky red gum. The solvent mixture was decanted off and the gum was dissolved in a mixture of ether (50 mL) and EtOAc (25 mL). The organic fraction was washed with aqueous 1 N NaOH (2×100 mL) and saturated aqueous NaCl (1×100 mL), dried over Na₂SO₄, filtered, and concentrated. Purification by flash column chromatography (10 to 70% EtOAc:hexanes) afforded 1.40 g (36%) of the title compound of Step A. ¹H NMR (400 MHz, CDCl₃): δ 8.31 (d, 1H, J=5.5 Hz), 7.03 (d, 1H, J=5.5 Hz), 6.94 (s, 1H), 6.83 (d, 1H, J=5.1 Hz), 3.79 (s, 3H), 3.37 (m, 1H), 2.36 (s, 3H), and 1.47 (d, 6H, J=7.0 Hz); MS (ESI): 360.03 [M+H]⁺.

### Step B: 4-{2-(1-Methylethyl)-4-[3-methyl-5-(methyloxy)phenyl]-1,3-thiazol-5-yl}-N-[6-(4-morpholinyl)-3-pyridinyl]-2-pyrimidinamine

A solution of 2-chloro-4-{2-(1-methylethyl)-4-[3-methyl-5-(methyloxy)phenyl]-1,3-thiazol-5-yl}pyrimidine (250 mg, 0.695 mmol), 6-(4-morpholinyl)-3-pyridinamine (149 mg, 0.834 mmol), prepared by a procedure analogous to Example 35, Step B, and p-toluenesulfonic acid hydrate (264 mg, 1.39 mmol) in trifluoroethanol (2.8 mL) was heated in a microwave at 160° C. for 1 h. The reaction mixture was concentrated onto silica gel. Purification by flash column chromatography (0 to 100% (15% MeOH/1% NH₄OH/DCM):EtOAc) afforded 157 mg (45%) of the title compound of Example 61. ¹H NMR (400 MHz, DMSO-d₆): δ 9.49 (s, 1H), 8.44 (d, 1H, J=2.8 Hz), 8.26 (d, 1H, J=5.3 Hz), 7.88 (dd, 1H, J=2.8, 9.0 Hz), 6.94 (s, 1H), 6.85 (s, 2H), 6.81 (d, 1H, J=9.2 Hz), 6.47 (d, 1H, J=5.3 Hz), 3.71 (m, 7H), 3.36 (m, 4H), 3.29 (m, 1H), 2.31 (s, 3H), and 1.38 (d, 6H, J=6.8 Hz); MS (ESI): 503.21 [M+H]⁺.

### Example 62

### N-(3-Fluoro-4-{4-[2-(methylsulfonyl)ethyl]-1-piperazinyl}phenyl)-4-{2-(1-methylethyl)-4-[3-methyl-5-(methyloxy)phenyl]-1,3-thiazol-5-yl}-2-pyrimidinamine

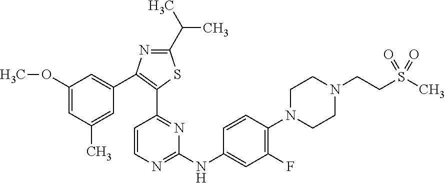

A solution of 2-chloro-4-{2-(1-methylethyl)-4-[3-methyl-5-(methyloxy)phenyl]-1,3-thiazol-5-yl}pyrimidine (100 mg, 0.278 mmol), prepared by a procedure analogous to Example 61, Step A, and (3-fluoro-4-{4-[2-(methylsulfonyl)ethyl]-1-piperazinyl}phenyl)amine (92 mg, 0.306 mmol), prepared by a procedure analogous to Example 6, Step B, in trifluoroethanol (2.8 mL) with 3 drops of concentrated HCl was heated in a microwave at 170° C. for 15 min. The reaction mixture was then concentrated. Purification by flash column chromatography (0 to 100% (15% MeOH/1% NH₄OH/DCM):EtOAc) afforded 148 mg (85%) of the title compound of Example 62. ¹H NMR (400 MHz, DMSO-d₆): δ 9.74 (s, 1H), 8.32 (d, 1H, J=5.1 Hz), 7.71 (dd, 1H, J=2.4, 15.6 Hz), 7.37 (dd, 1H, J=2.1, 8.9 Hz), 6.97 (m, 2H), 6.86 (s, 2H), 6.52 (d, 1H, J=5.3 Hz), 3.72 (s, 3H), 3.33 (m, 3H), 3.05 (s, 3H), 2.96 (m, 4H), 2.77 (t, 2H, J=6.8 Hz), 2.60 (m, 4H), 2.31 (s, 3H), and 1.39 (d, 6H, J=7.0 Hz); MS (ESI): 625.12 [M+H]⁺.

### Example 63

### N-[6-(4-Acetyl-1-piperazinyl)-3-pyridinyl]-4-{2-(1-methylethyl)-4-[3-methyl-5-(methyloxy)phenyl]-1,3-thiazol-5-yl}-2-pyrimidinamine

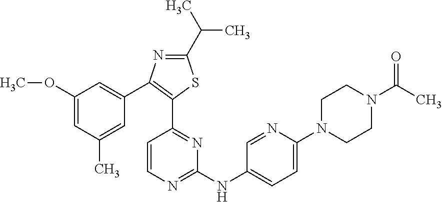

The title compound of Example 63 was prepared from 2-chloro-4-{2-(1-methylethyl)-4-[3-methyl-5-(methyloxy)phenyl]-1,3-thiazol-5-yl}pyrimidine (100 mg, 0.278 mmol), prepared by a procedure analogous to Example 61, Step A, and 6-(4-acetyl-1-piperazinyl)-3-pyridinamine (67 mg, 0.306 mmol), prepared by a procedure analogous to Example 4, Step B in 36% yield by a procedure analogous to Example 1, Step G, except the reaction was run in a microwave for a total of 45 min. ¹H NMR (400 MHz, DMSO-d₆): δ 9.49 (s, 1H), 8.43 (m, 1H), 8.25 (d, 1H, J=5.1 Hz), 7.88 (dd, 1H, J=1.9, 9.4 Hz), 6.94 (s, 1H), 6.84 (m, 3H), 6.47 (d, 1H, J=4.9 Hz), 3.72 (s, 3H), 3.55 (m, 4H), 3.46 (m, 2H), 3.38 (m, 2H), 3.34 (m, 1H), 3.57 (s, 3H), 2.05 (s, 3H), 1.39 (d, 6H, J=6.8 Hz); MS (ESI): 544.41 [M+H]⁺.

### Example 64

### N-({5-(2-{[6-(4-Acetyl-1-piperazinyl)-3-pyridinyl]amino}-4-pyrimidinyl)-4-[3-methyl-5-(methyloxy)phenyl]-1,3-thiazol-2-yl}methyl)methanesulfonamide

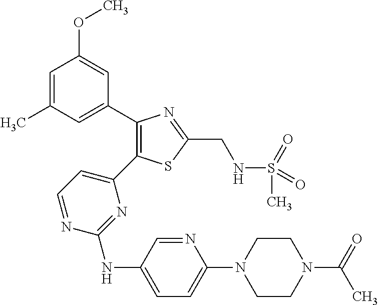

### Step A: Phenylmethyl ({5-(2-chloro-4-pyrimidinyl)-4-[3-methyl-5-(methyloxy)phenyl]-1,3-thiazol-2-yl}methyl)carbamate

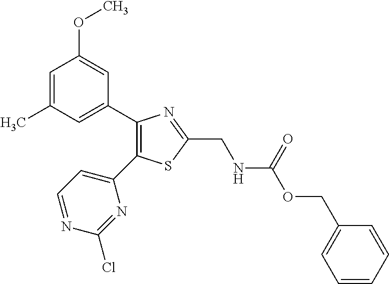

To a solution of (Z)-2-(2-Chloro-4-pyrimidinyl)-1-[3-methyl-5-(methyloxy)phenyl]ethanol (1.0 g, 3.6 mmol), prepared by a procedure analogous to Example 25, Step E, in DCM (25 mL), NBS (0.6 g, 3.6 mmol) was added and the reaction mixture was allowed to stir for 30 min at rt. The solvent was removed and the residue was taken up in DMF (10 mL). Phenylmethyl (2-amino-2-thioxoethyl)carbamate (0.9 g, 4.0 mmol) was then added and the reaction was allowed to stir for 2 h at rt. The reaction mixture was diluted with DCM and washed with water. The aqueous layer was extracted twice with DCM and the combined extracts were washed again with water. The combined organics were dried over MgSO₄, filtered and evaporated. The title compound of Step A was isolated in 59% yield as a solid after MeOH trituration (0.98 g). MS (ESI): 497.3 [M+H]⁺.

### Step B: N-({5-(2-{[6-(4-Acetyl-1-piperazinyl)-3-pyridinyl]amino}-4-pyrimidinyl)-4-[3-methyl-5-(methyloxy)phenyl]-1,3-thiazol-2-yl}methyl)methanesulfonamide

To a mixture of phenylmethyl ({5-(2-chloro-4-pyrimidinyl)-4-[3-methyl-5-(methyloxy)phenyl]-1,3-thiazol-2-yl}methyl)carbamate (150 mg, 0.31 mmol) and 6-(4-acetyl-1-piperazinyl)-3-pyridinamine (76 mg, 0.34 mmol), prepared by a procedure analogous to Example 4, Step B, in trifluoroethanol (2.0 mL), HCl (4 N in dioxane, 0.2 mL) was added and the solution was heated in a microwave reactor at 170° C. for 10 min. The reaction mixture was evaporated onto silica gel and chromatographed, 10-90% 1:9:90 NH₄OH:MeOH:DCM in DCM. The intermediate product, N-[6-(4-acetyl-1-piperazinyl)-3-pyridinyl]-4-{2-(aminomethyl)-4-[3-methyl-5-(methyloxy)phenyl]-1,3-thiazol-5-yl}-2-pyrimidinamine, was dissolved in DCM (2 mL) and treated with TEA (0.09 mL, 0.62 mmol) and methanesulfonyl chloride (0.024 mL, 0.031 mmol). After stirring 30 min at rt, the reaction mixture was evaporated onto silica gel and chromatographed, 10-90% 1:9:90 NH₄OH:MeOH:DCM in DCM. The title compound of Example 64 was isolated in 22% yield as an orange solid (42 mg). ¹H NMR (400 MHz, DMSO-d₆) δ ppm 9.48 (s, 1H), 8.42 (d, J=2.4 Hz, 1H), 8.23 (d, J=4.9 Hz, 1H), 8.12 (t, J=6.6 Hz, 1H), 7.80 (dd, J=9.3, 2.6 Hz, 1H), 6.90 (s, 1H), 6.82 (s, 2H), 6.78 (d, J=9.2 Hz, 1H), 6.44 (d, J=5.1 Hz, 1H), 4.47 (d, J=6.4 Hz, 2H), 3.68 (s, 3H), 3.45-3.54 (m, 4H), 3.38-3.45 (m, 2H), 3.28-3.38 (m, 2H), 3.01 (s, 3H), 2.26 (s, 3H), 2.00 (s, 3H); MS (ESI): 609.2 [M+H]⁺.

### Example 65

### [3-[5-(2-{[6-(4-Acetyl-1-piperazinyl)-3-pyridinyl]amino}-4-pyrimidinyl)-2-ethyl-1,3-thiazol-4-yl]-5-(methyloxy)phenyl]methanol

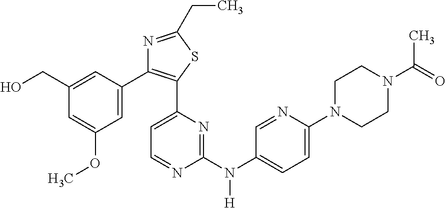

### Step A: Methyl 3-(hydroxymethyl)-5-(methyloxy)benzoate

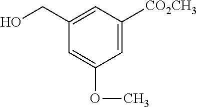

To a stirring suspension of 3-(methyloxy)-5-[(methyloxy)carbonyl]benzoic acid (21.30 g, 1.0 eq) in THF (100 mL) at 0° C. was added BH₃.THF (170 mL, 1.9 eq) by addition funnel over 1.0 h. The reaction mixture was warmed to rt and stirred overnight. The reaction was quenched by dropwise addition of 1:1 water:acetic acid (20 mL) over 0.25 h (until the evolution of gas ceased) and concentrated. The slurry was taken up in EtOAc and washed with NaHCO₃ (aq). The aqueous extracts were washed with EtOAc and the combined organic extracts were dried over MgSO₄, filtered, and concentrated to give 20.77 g (104%) of the title compound of Step A which was taken on without any further purification. ¹H NMR (400 MHz, CDCl₃) δ 7.62 (s, 1H), 7.48 (s, 1H), 7.14 (s, 1H), 4.72 (s, 2H), 3.92 (s, 3H), 3.86 (s, 3H).

### Step B: Methyl 3-({[(1,1-dimethylethyl)(dimethyl)silyl]oxy}methyl)-5-(methyloxy)benzoate

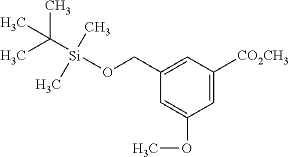

To a stirring solution of methyl 3-(hydroxymethyl)-5-(methyloxy)benzoate (5.0 g, 1.0 eq) in DCM (130 mL) was added imidazole (4.34 g, 2.5 eq) followed by TBSCI (5.76 g, 1.5 eq). After stirring 1 h, the reaction mixture was quenched with water and extracted 2× with DCM. The combined organic extracts were dried over MgSO₄, filtered, concentrated onto silica gel, and purified by column chromatography (gradient elution: 0 to 20% Hex/EtOAc) to provide 5.76 g (73%) of the title compound of Step B. ¹H NMR (400 MHz, DMSO-d₆) δ 7.53 (s, 1H), 7.32 (s, 1H), 7.14 (s, 1H), 4.75 (s, 2H), 3.84 (s, 3H), 3.80 (s, 3H), 3.32 (s, 1H), 0.91 (s, 9H), and 0.08 (s, 6H).

### Step C: 2-(2-Chloro-4-pyrimidinyl)-1-[3-({[(1,1-dimethylethyl)(dimethyl)silyl]oxy}methyl)-5-(methyloxy)phenyl]ethanone

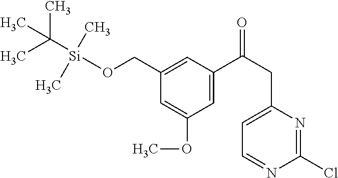

To a stirring solution of methyl 3-({[(1,1-dimethylethyl)(dimethyl)silyl]oxy}methyl)-5-(methyloxy)benzoate (5.76 g, 1.0 eq) in THF (30 mL) at 0° C. was added LHMDS (39.0 mL of a 1.0 M solution in THF, 2.1 eq) over a few min. After stirring 5 min, 2-chloro-4-methyl-pryimidine (2.62 g, 1.1 eq) was added in portions over 10 min. After stirring 0.5 h, the reaction was quenched with MeOH (7 mL) and concentrated. The residue was dissolved in EtOAc and washed with water. The organic extracts were dried over MgSO₄, filtered, concentrated onto silica gel, and purified by column chromatography (gradient elution: 0 to 25% hexane/EtOAc) to provide 5.13 g (68%) of an oil as a mixture of keto/enol tautomers. MS (ESI): 407.12 [M+H]⁺.

### Step D: 2-(2-Chloro-4-pyrimidinyl)-1-[3-({[(1,1-dimethylethyl)(dimethyl)silyl]-oxy}methyl)-5-(methyloxy)phenyl]ethanone

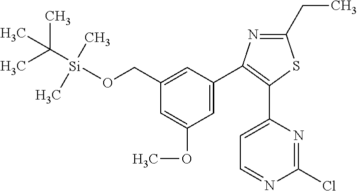

To a stirring solution of 2-(2-chloro-4-pyrimidinyl)-1-[3-({[(1,1-dimethylethyl)(dimethyl)silyl]oxy}methyl)-5-(methyloxy)phenyl]ethanone (5.13 g, 1.0 eq) in DCM (126 mL) was added NBS (2.36 g, 1.05 eq). After stirring 0.25 h, the reaction mixture was quenched with water and extracted 2× with DCM. The organic extracts were dried over MgSO₄, filtered, and concentrated. The resultant reside was taken up in DMF (30 mL) and treated with thiopropionamide (1.23 g, 1.1 eq). After stirring 0.25 h, the reaction mixture was diluted with 1:1 water/EtOAc. The mixture was extracted 2×EtOAc, dried over MgSO₄, filtered, concentrated onto silica gel, and purified by column chromatography (gradient elution: 0 to 50% hexane/EtOAc) to provide 1.68 g (28%) of the title compound of Step D. ¹H NMR (400 MHz, DMSO-d₆) δ 8.56 (d, J=5.3 Hz, 2H), 7.14 (d, J=5.5 Hz, 2H), 7.04 (s, 1H), 7.02 (s, 1H), 6.98 (s, 1H), 4.71 (s, 2H), 3.75 (s, 3H), 3.06 (q, J=7.6 Hz, 2H), 1.36 (t, J=7.5 Hz, 3H), 0.81-0.88 (m, 9H), and 0.04 (s, 6H); MS (ESI): 475.23 [M−H]⁻.

### Step E: [3-[5-(2-{[6-(4-Acetyl-1-piperazinyl)-3-pyridinyl]amino}-4-pyrimidinyl)-2-ethyl-1,3-thiazol-4-yl]-5-(methyloxy)phenyl]methanol

To a stirring solution of 2-(2-chloro-4-pyrimidinyl)-1-[3-({[(1,1-dimethylethyl)(dimethyl)silyl]oxy}methyl)-5-(methyloxy)phenyl]ethanone (0.100 g, 1.0 eq) in 2,2,2-trifluoroethanol (3 mL) was added 6-(4-acetylpiperazin-1-yl)pyridin-3-amine (0.055 g, 1.0 eq), prepared by a procedure analogous to Example 4, Step B, and p-toluenesulfonic acid monohydrate (0.080 g, 2.0 eq). The vial was heated in the microwave at 180° C. for 1.25 h. The reaction mixture was quenched with TEA (0.5 mL), concentrated onto silica gel, and purified by column chromatography (gradient elution: 0 to 25% DCM/30% MeOH in DCM) followed by purification by preparative HPLC (10 to 70% acetonitrile:water w/0.1% TFA). The resultant TFA salt was dissolved in DCM and washed 2× with NaHCO₃ (aq). The organic extracts were dried over MgSO₄, filtered, and concentrated to provide 0.032 g (28%) of the title compound of Example 65. ¹H NMR (400 MHz, DMSO-d₆) δ 9.50 (s, 1H), 8.44 (d, J=2.6 Hz, 1H), 8.25 (d, J=5.1 Hz, 1H), 7.87 (dd, J=5.9 Hz, 1H), 7.09 (s, 1H), 6.97 (s, 1H), 6.92 (s, 1H), 6.86 (s, 1H), 6.84 (s, 1H), 6.47 (d, J=5.3 Hz, 1H), 5.27 (t, J=5.9 Hz, 1H), 4.51 (d, J=5.9 Hz, 2H), 3.74 (s, 3H), 3.50-3.60 (m, 4H), 3.42-3.50 (m, 2H), 3.35-3.42 (m, 2H), 3.04 (q, J=7.5 Hz, 2H), 2.05 (s, 3H), 1.35 (t, J=7.51 Hz, 3H); MS (ESI): 446.17 [M+H]⁺.

### Example 66

### (3-{2-Ethyl-5-[2-({6-[4-(methylsulfonyl)piperazin-1-yl]pyridin-3-yl}amino)pyrimidin-4-yl]-1,3-thiazol-4-yl}-5-methylphenyl)methanol

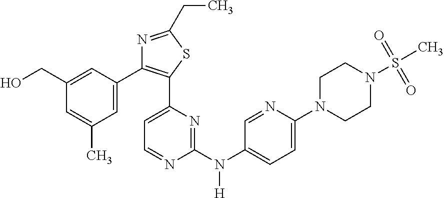

### Step A: Methyl 3-methyl-5-{[(triisopropylsilyl)oxy]methyl}benzoate

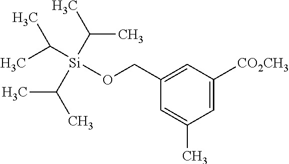

To a stirring suspension of 3-(methoxycarbonyl)-5-methylbenzoic acid (5.0 g, 1.0 eq) in THF (15 mL) at 0° C. was added BH₃.THF (28 mL, 1.5 eq) by addition funnel over 0.5 h. The reaction mixture was warmed to rt and stirred overnight. The reaction was quenched by dropwise addition of acetic acid (3 mL) over 0.25 h (until the evolution of gas ceased) and concentrated. The slurry was taken up in EtOAc and washed with NaHCO₃ (aq). The aqueous extracts were washed with EtOAc and the combined organic extracts were dried over MgSO₄, filtered, and concentrated to give 3.7 g (88%) of the alcohol which was taken on without any further purification. The alcohol (3.7 g, 1.0 eq) was dissolved in DCM (30 mL) and treated sequentially with imidazole (3.5 g, 2.3) and triisopropylsilyl chloride (6.27 mL, 1.3 eq). After stirring 1 h, the reaction mixture was quenched with saturated aqueous NaHCO₃ and extracted 2× with DCM. The combined organic extracts were dried over MgSO₄, filtered, concentrated onto silica gel, and purified by column chromatography (gradient elution: 0 to 40% hexane/EtOAc) to provide 5.29 g (80%) of the title compound of Step A. ¹H NMR (400 MHz, CDCl₃) δ 7.82 (s, 1H), 7.72-7.76 (m, 1H), 7.39-7.42 (m, 1H), 4.85 (s, 2H), 3.91 (s, 3H), 2.41 (s, 3H), 1.01-1.14 (m, 21H).

### Step B: 2-(2-Chloropyrimidin-4-yl)-1-(3-methyl-5-{[(triisopropylsilyl)oxy]methyl}-phenyl)ethanone

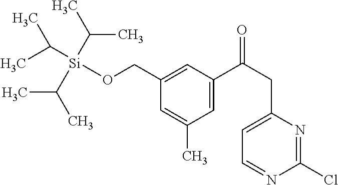

To a stirring solution of methyl 3-methyl-5-{[(tri isopropylsilyl)oxy]methyl}benzoate (4.17 g, 1.0 eq) in THF (23 mL) at 0° C. was added LHMDS (30 mL of a 1.0 M solution in THF, 2.1 eq) over a few min. After stirring 5 min, 2-chloro-4-methyl-pryimidine (2.18 g, 1.1 eq) was added in portions over 10 min. After stirring 0.5 h, the reaction was quenched with MeOH (5 mL) and concentrated. The residue was dissolved in EtOAc and washed with water. The organic extracts were dried over MgSO₄, filtered, concentrated onto silica gel, and purified by column chromatography (gradient elution: 0 to 60% hexane/EtOAc) to provide 3.93 g (64%) of an oil as a mixture of keto/enol tautomers. MS (ESI): 433.48 [M+H]⁺.

### Step C: {3-[5-(2-Chloropyrimidin-4-yl)-2-ethyl-1,3-thiazol-4-yl]-5-methylphenyl}methanol

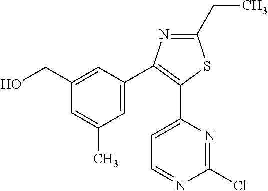

To a stirring solution of 2-(2-chloropyrimidin-4-yl)-1-(3-methyl-5-{[(triisopropylsilyl)oxy]methyl}phenyl)ethanone (2.47 g, 1.0 eq) in DCM (85 mL) was added NBS (1.07 g, 1.05 eq). After stirring 0.25 h, the reaction mixture was concentrated and taken up in DMF (40 mL) and treated with thiopropionamide (0.75 g, 1.5 eq). After stirring 1 h, the reaction mixture was quenched with NaHCO₃ (aq) and extracted 2× with EtOAc. The organic extracts were dried over MgSO₄, filtered, concentrated onto silica gel, and purified by column chromatography (gradient elution: 0 to 60% hexane/EtOAc) to provide 0.51 g (26%) of the title compound of Step C; ¹H NMR (400 MHz, CDCl₃) δ 8.36 (d, J=5.5 Hz, 1H), 7.41 (s, 1H), 7.32 (s, 1H), 7.28 (s, 1H), 7.06 (d, J=5.3 Hz, 1H), 4.73 (s, 2H), 3.20 (q, J=7.5 Hz, 2H), 2.37 (s, 3H), and 1.50 (t, J=7.6 Hz, 3H).

### Step D: (3-{2-Ethyl-5-[2-({6-[4-(methylsulfonyl)piperazin-1-yl]pyridin-3-yl}amino)pyrimidin-4-yl]-1,3-thiazol-4-yl}-5-methylphenyl)methanol

To a stirring solution of {3-[5-(2-chloropyrimidin-4-yl)-2-ethyl-1,3-thiazol-4-yl]-5-methylphenyl}methanol (0.10 g, 1.0 eq) in 2,2,2-trifluoroethanol (3 mL) was added 6-[4-(methylsulfonyl)piperazin-1-yl]pyridin-3-amine (0.089 g, 1.0 eq), prepared by a procedure analogous to Example 2, Step B, and 4 N HCl in dioxane (0.289 mL, 4 eq). The reaction was heated in the microwave at 185° C. for 0.5 h. The reaction mixture was quenched with TEA (0.5 mL), concentrated onto silica gel, and purified by column chromatography (gradient elution: 0 to 40% DCM/30% MeOH in DCM) to provide 0.016 g (10%) of the title compound of Step D. ¹H NMR (400 MHz, CDCl₃) δ 8.34 (d, J=2.6 Hz, 1H), 8.12 (d, J=5.3 Hz, 1H), 7.91 (m, 1H), 7.38 (s, 2H), 7.28 (s, 1H), 7.24 (s, 1H), 6.73 (d, J=9.2 Hz, 1H), 6.56 (d, J=5.3 Hz, 1H), 4.68 (s, 2H), 3.61-3.71 (m, 4H), 3.31-3.41 (m, 4H), 3.08 (q, J=7.6 Hz, 2H), 2.82 (s, 3H), 2.36 (s, 3H), and 1.46 (t, J=7.6 Hz, 3H).

### Example 67

### {3-[2-Ethyl-5-(2-{[3-fluoro-4-(4-morpholinyl)phenyl]amino}-4-pyrimidinyl)-1,3-thiazol-4-yl]phenyl}methanol

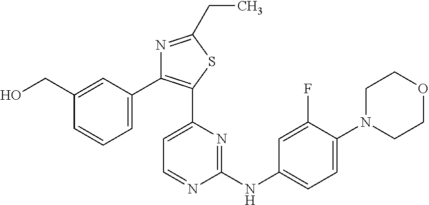

### Step A: Methyl 3-[(2-chloro-4-pyrimidinyl)acetyl]benzoate

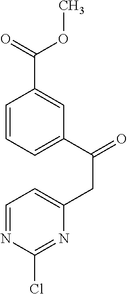

To a solution of dimethyl 1,3-benzenedicarboxylate (3.1 eq, 14.6 g, 75.2 mmol) in dry THF (75 mL) at 0° C., LHMDS (1 M in THF, 5.0 eq, 120 mmol, 120 mL) was added and the solution was allowed to stir for 10 min at 0° C. A solution of 2-chloro-4-methylpyrimidine (1.0 eq, 24.1 mmol, 3.1 g) in 10 mL of THF was then added dropwise to the reaction mixture at 0° C. over 10 min. The mixture was allowed to stir 30 minutes at 0° C. The reaction mixture was then quenched at 0° C. with MeOH and the solvent was removed in vacuo. The residue was diluted with EtOAc and washed with water. The water layer was extracted twice with EtOAc, dried with MgSO₄, and evaporated onto silica gel. Purification by column chromatography (10-70% EtOAc in hexanes) provided 6.1 g of the title product of Step A as a light yellow solid (87%). MS (ESI): 291.1 [M+H]⁺.

### Step B: Methyl 3-[5-(2-chloro-4-pyrimidinyl)-2-ethyl-1,3-thiazol-4-yl]benzoate

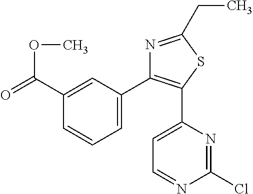

To a stirring solution of methyl 3-[(2-chloropyrimidin-4-yl)acetyl]benzoate (5.0 g, 1.0 eq) in DCM (250 mL) was added NBS (3.21 g, 1.05 eq). After stirring 0.25 h, the reaction mixture was concentrated, taken up in DMF (50 mL), and treated with thiopropionamide (1.84 g, 1.05 eq). After stirring 0.25 h, the reaction mixture was quenched with 1:1 water/EtOAc. The mixture was extracted 2×EtOAc, dried over MgSO₄, filtered, concentrated onto silica gel, and purified by column chromatography (gradient elution: 0 to 40% hexane/EtOAc) to provide 0.89 g (14%) of the title compound of Step A. ¹H NMR (400 MHz, CDCl₃) δ 8.34 (d, J=5.3 Hz, 1H), 8.25 (ddd, J=1.6 Hz, 1H), 8.14-8.18 (m, 1H), 7.72-7.78 (m, 1H), 7.56 (t, J=7.8 Hz, 1H), 6.95 (d, J=5.3 Hz, 1H), 3.93 (s, 3H), 3.13 (q, J=7.6 Hz, 2H), and 1.48 (t, J=7.6 Hz, 3H); MS (ESI): 360.30 [M+H]⁺.

### Step C: Methyl 3-[2-ethyl-5-(2-{[3-fluoro-4-(4-morpholinyl)phenyl]amino}-4-pyrimidinyl)-1,3-thiazol-4-yl]benzoate

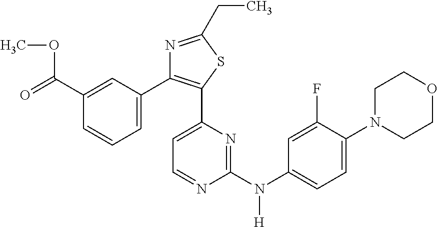

To a stirring suspension of methyl 3-[5-(2-chloro-4-pyrimidinyl)-2-ethyl-1,3-thiazol-4-yl]benzoate (0.20 g, 1.0 eq) and [3-fluoro-4-(4-morpholinyl)phenyl]amine (0.12 g, 1.1 eq), prepared by a procedure analogous to Example 7, Step B, in 2,2,2-trifluoroethanol (6 mL) was added 4 N HCl in dioxane (0.46 mL, 4 eq) and heated in the microwave at 170° C. for 20 min. The reaction mixture was quenched with TEA (0.5 mL), concentrated onto silica gel, and purified by column chromatography (gradient elution: 0 to 20% DCM/MeOH) to provide 0.21 g (14%) of the title compound of Step B. ¹H NMR (400 MHz, CDCl₃) δ 8.31 (t, J=1.6 Hz, 1H), 8.14 (m, 1H), 8.05 (d, J=5.3 Hz, 1H), 7.78 (m, 1H), 7.64 (d, J=15.0 Hz, 1H), 7.56 (t, J=7.7 Hz, 1H), 7.20 (d, J=6.8 Hz, 1H), 6.54 (d, J=5.9 Hz, 1H), 3.90-4.02 (m, 7H), 3.07-3.23 (m, 6H), and 1.49 (t, J=7.6 Hz, 3H); MS (ESI): 520.40 [M+H]⁺.

### Step D: {3-[2-Ethyl-5-(2-{[3-fluoro-4-(4-morpholinyl)phenyl]amino}-4-pyrimidinyl)-1,3-thiazol-4-yl]phenyl}methanol

To a stirring solution of methyl 3-[2-ethyl-5-(2-{[3-fluoro-4-(4-morpholinyl)phenyl]amino}-4-pyrimidinyl)-1,3-thiazol-4-yl]benzoate (0.102 g, 1.0 eq) in THF (2 mL) at −78° C. was added LAH (0.39 mL of a 1 M solution in THF). The reaction vial was warmed to rt, sealed, and heated overnight at 50° C. The reaction was quenched by the sequential addition of water (1 mL) and 1 M MeOH/NaOH (1 mL). The reaction mixture was diluted with water and extracted with EtOAc and Et₂O. The organic extracts were dried over MgSO₄, filtered, concentrated onto silica gel, and purified by column chromatography (gradient elution: 0 to 80% hexane/EtOAc) to provide 0.024 g (25%) of the title compound of Example 67. ¹H NMR (400 MHz, CDCl₃) δ 8.07 (s, 1H), 7.54-7.71 (m, 2H), 7.37-7.53 (m, 3H), 7.10-7.23 (m, 1H), 6.88-7.09 (m, 1H), 6.50-6.65 (m, 1H), 4.68-4.84 (m, 4H), 3.74-4.04 (m, 4H), 2.84-3.34 (m, 6H), and 1.33-1.64 (m, 3H); MS (ESI): 492.50 [M+H]⁺.

### Example 68

### 4-[2-Ethyl-4-(1H-indol-6-yl)-1,3-thiazol-5-yl]-N-[3-fluoro-4-({1-[2-(methylsulfonyl)ethyl]-4-piperidinyl}oxy)phenyl]-2-pyrimidinamine

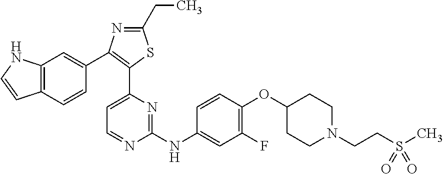

### Step A: Methyl 1-(phenylsulfonyl)-1H-indole-6-carboxylate

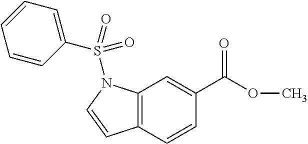

To a solution of methyl 1H-indole-6-carboxylate (1.88 g, 10.7 mmol) in 2-butanone (30 mL) was added K₂CO₃ (5.9 g, 43 mmol) and benzenesulfonyl chloride (2.7 mL, 21.5 mmol). The reaction mixture was heated to reflux overnight. After 16 h, the reaction mixture was filtered, concentrated on the rotovap, and absorbed onto silica gel. The crude product was purified by column chromatography (eluting with 0-100% EtOAc/DCM) to generate the desired product of Step A in 75% yield (2.54 g, 8.1 mmol). MS (ESI) m/z=315 [M+H]⁺.

### Step B: 2-(2-Chloro-4-pyrimidinyl)-1-[1-(phenylsulfonyl)-1H-indol-6-yl]ethanone

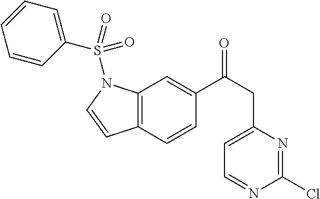

To a solution of methyl 1-(phenylsulfonyl)-1H-indole-6-carboxylate (2.38 g, 7.6 mmol) in anhydrous THF (20 mL), cooled to 0° C., was added a 1 M solution of LHMDS in THF (13.6 mL, 13.6 mmol). A solution of 2-chloro-4-methylpyrimidine (1.06 g, 8.3 mmol) in anhydrous THF (20 mL) was added dropwise over 15 min. The icebath was removed, and the reaction was allowed to warm to rt. After 3 h, the reaction was concentrated on the rotovap, and the residue was redissolved in EtOAc and adsorbed onto silica gel. The crude product was purified by column chromatography (eluting with 0-50% EtOAc/DCM), but the product fractions were not clean. The product fractions were combined and concentrated on the rotovap, during which a yellow precipitate formed. The precipitate was collected by vacuum filtration, washed with EtOAc, and dried under vacuum to afford the desired product of Step B in 33% yield (1.04 g, 2.5 mmol). MS (ESI) m/z=412 [M+H]⁺.

### Step C: 6-[5-(2-Chloro-4-pyrimidinyl)-2-ethyl-1,3-thiazol-4-yl]-1-(phenylsulfonyl)-1H-indole

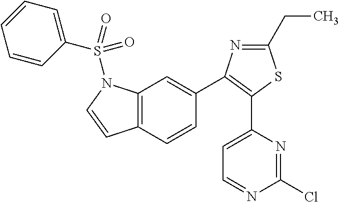

To a solution of 2-(2-chloro-4-pyrimidinyl)-1-[1-(phenylsulfonyl)-1H-indol-6-yl]ethanone (0.25 g, 0.61 mmol) in DCM (15 mL) was added NBS (0.11 g, 0.64 mmol). After stirring for 30 min at rt, the solvent was removed on the rotovap. The residue was redissolved in DMF (5 mL), and thiopropionamide (81 mg, 0.91 mmol) was added. After 2 h at rt, the reaction mixture was partitioned between EtOAc and water. The pH of the aqueous layer was raised to >7 with saturated aqueous NaHCO₃, and the layers were separated. The aqueous layer was extracted with EtOAc. The combined organic layers were washed with brine, dried over anhydrous Na₂SO₄, filtered, and adsorbed onto silica gel. The crude product was purified by column chromatography (eluting with 0-50% EtOAc/DCM) to generate the desired product of Step C in 51% yield (150 mg, 0.31 mmol). MS (ESI) m/z=481 [M+H]⁺.

### Step D: 4-{2-Ethyl-4-[1-(phenylsulfonyl)-1H-indol-6-yl]-1,3-thiazol-5-yl}-N-[3-fluoro-4-({1-[2-(methylsulfonyl)ethyl]-4-piperidinyl}oxy)phenyl]-2-pyrimidinamine

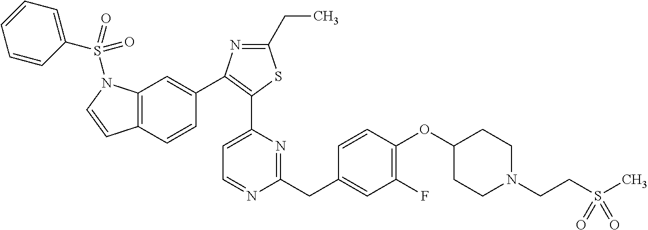

To a suspension of 6-[5-(2-chloro-4-pyrimidinyl)-2-ethyl-1,3-thiazol-4-yl]-1-(phenylsulfonyl)-1H-indole (53 mg, 0.11 mmol) in iPrOH (2 mL) was added [3-fluoro-4-({1-[2-(methylsulfonyl)ethyl]-4-piperidinyl}oxy)phenyl]amine (50 mg, 0.16 mmol), prepared by a procedure analogous to Example 10, Step D, and concentrated HCl (1 drop). The mixture was heated to 170° C. for 35 min in a microwave reactor. The reaction mixture was concentrated on the rotovap, redissolved in 2 mL 1:1 DMSO/MeOH, and purified by reverse-phase HPLC (eluting with 10-60% acetonitrile/0.1% TFA) to generate the desired product of Step D in 73% yield (61 mg, 0.08 mmol). MS (ESI) m/z=761 [M+H]⁺.

### Step E: 4-[2-Ethyl-4-(1H-indol-6-yl)-1,3-thiazol-5-yl]-N-[3-fluoro-4-({1-[2-(methylsulfonyl)ethyl]-4-piperidinyl}oxy)phenyl]-2-pyrimidinamine

To a solution of 6-[5-(2-chloro-4-pyrimidinyl)-2-ethyl-1,3-thiazol-4-yl]-1-(phenylsulfonyl)-1H-indole (61 mg, 0.08 mmol) in MeOH (6 mL) was added 5 M aqueous NaOH (1 mL, 5 mmol). The reaction was stirred for 2 h at 50° C., and then partitioned between water and EtOAc. The layers were separated, and the aqueous layer was extracted with EtOAc. The combined organic layers were dried over anhydrous Na₂SO₄, filtered, and concentrated to generate the title compound of Example 68 as a yellow powder (46 mg, 0.07 mmol, 93% yield). ¹H NMR (400 MHz, DMSO-d₆) δ 11.24 (s, 1H), 9.73 (s, 1H), 8.23 (d, 1H, J=5.1 Hz), 7.75 (d, 1H, J=14.3 Hz), 7.59 (m, 2H), 7.44 (t, 1H, J=2.8 Hz), 7.34 (d, 1H, J=7.2 Hz), 7.15 (d, 1H, J=9.5 Hz), 7.05 (t, 1H, J=9.4 Hz), 6.51 (d, 1H, J=4.5 Hz), 6.47 (m, 1H), 3.27 (m, 2H), 3.06 (m, 2H), 3.04 (s, 3H), 2.71 (m, 4H), 2.26 (m, 2H), 1.90 (m, 2H), 1.62 (m, 2H), and 1.36 (t, 3H, J=7.6 Hz). MS (ESI) m/z=621 [M+H]⁺.

### Example 69

### 4-[4-[3,5-Bis(methyloxy)phenyl]-2-(ethylamino)-1,3-oxazol-5-yl]-N-[3-chloro-4-(4-methyl-1-piperazinyl)phenyl]-2-pyrimidinamine

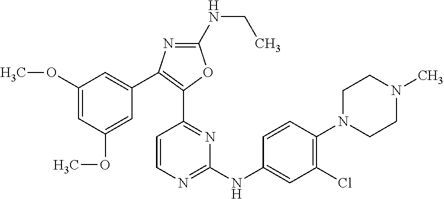

### Step A: 4-[3,5-Bis(methyloxy)phenyl]-5-(2-chloro-4-pyrimidinyl)-N-ethyl-1,3-oxazol-2-amine

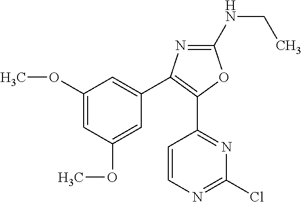

To a solution of 1-[3,5-bis(methyloxy)phenyl]-2-(2-chloro-4-pyrimidinyl)ethanone (500 mg, 1.71 mmol), prepared by a procedure analogous to Example 25, Step E, in DCM (8.6 mL) was added NBS (304 mg, 1.71 mmol). The reaction was stirred at rt for 30 min and then concentrated. The residue was redissolved in 1,4-dioxane (5.2 mL) and N-ethylurea (978 mg, 11.1 mmol) was added. The reaction was heated in a microwave at 150° C. for 40 min, then diluted with DCM (30 mL) and concentrated onto silica gel. Purification by flash column chromatography (20 to 80% EtOAc:hexanes) afforded 340 mg (55%) of the title compound of Step A. ¹H NMR (400 MHz, DMSO-d₆): δ 8.59 (d, 1H, J=5.5 Hz), 8.27 (t, 1H, J=5.6 Hz), 7.35 (d, 1H, J=5.3 Hz), 7.27 (d, 2H, J=2.4 Hz), 6.57 (t, 1H, J=2.3 Hz), 3.80 (s, 6H), 3.37 (m, 2H), and 1.20 (t, 3H); MS (ESI): 361.17 [M+H]⁺.

### Step B: 4-[4-[3,5-bis(methyloxy)phenyl]-2-(ethylamino)-1,3-oxazol-5-yl]-N-[3-chloro-4-(4-methyl-1-piperazinyl)phenyl]-2-pyrimidinamine

The title compound of Example 69 was prepared from 4-[3,5-bis(methyloxy)phenyl]-5-(2-chloro-4-pyrimidinyl)-N-ethyl-1,3-oxazol-2-amine (100 mg, 0.277 mmol) and [3-chloro-4-(4-methyl-1-piperazinyl)phenyl]amine (69 mg, 0.305 mmol) in 22% yield by a procedure analogous to Example 1, Step G, except that the reaction was run 15 min in the microwave. ¹H NMR (400 MHz, DMSO-d₆): δ 9.51 (s, 1H), 8.38 (d, 1H, J=5.3 Hz), 7.89 (t, 1H, J=5.3 Hz), 7.69 (s, 1H), 7.50 (d, 1H, J=7.0 Hz), 6.91 (s, 2H), 6.81 (m, 2H), 6.52 (s, 1H), 3.66 (s, 6H), 3.36 (m, 2H), 2.88 (m, 4H), 2.48 (m, 4H), 2.23 (s, 3H), and 1.20 (t, 3H, J=7.1 Hz); MS (ESI): 550.38 [M+H]⁺.

### Example 70

### N-[6-(4-Acetyl-1-piperazinyl)-3-pyridinyl]-4-{2-(ethylamino)-4-[3-methyl-5-(methyloxy)phenyl]-1,3-oxazol-5-yl}-2-pyrimidinamine

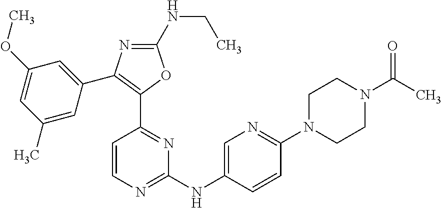

### Step A: 5-(2-Chloro-4-pyrimidinyl)-N-ethyl-4-[3-methyl-5-(methyloxy)phenyl]-1,3-oxazol-2-amine

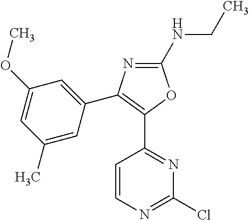

To a solution of 2-(2-chloro-4-pyrimidinyl)-1-[3-methyl-5-(methyloxy)phenyl]ethanone (1 g, 3.42 mmol), prepared by a procedure analogous to Example 25, Step E, in DCM (20 mL) was added NBS (0.61 g, 3.42 mmol). The reaction was stirred for 30 min at rt, and then the solvent was removed under reduced pressure. The residue was dissolved in iPrOH (20 mL) and ethylurea (1.95 g, 22.2 mmol) was added. The mixture was heated in a microwave at 150° C. for 40 min and then the solvent was removed under the reduced pressure. The residue was purified by flash column chromatography on silica gel (20 to 80% EtOAc:hexanes) to give the desired oxazole pyrimidyl chloride of Step A (0.68 mg, 55%). ¹H NMR (CDCl₃, 400 MHz) δ 8.40 (d, J=6.0 Hz, 1H), 7.26-7.21 (m, 3H), 6.56 (t, J=2.4 Hz, 1H), 5.14 (t, J=6.0 Hz, 1H), 3.93 (s, 6H), 3.58-3.52 (m, 2H), and 1.50 (t, J=7.2 Hz, 3H).

### Step B: N-[6-(4-Acetyl-1-piperazinyl)-3-pyridinyl]-4-{2-(ethylamino)-4-[3-methyl-5-(methyloxy)phenyl]-1,3-oxazol-5-yl}-2-pyrimidinamine

To a suspension of 150 mg (0.44 mmol) of 5-(2-chloro-4-pyrimidinyl)-N-ethyl-4-[3-methyl-5-(methyloxy)phenyl]-1,3-oxazol-2-amine, in 50 mL of THF was added 96 mg (0.436 mmol) of 6-(4-acetyl-1-piperazinyl)-3-pyridinamine and 2 drops of concentrated HCl. The reaction mixture was heated at 150° C. in a microwave reactor for 45 min and then the solvent was removed under reduced pressure. The residue was taken up in EtOAc and washed with water and the combined organic layers were dried over Na₂SO₄ and filtered. The residue was subjected to silica gel chromatography to give 59 mg (26%) of N-[6-(4-acetyl-1-piperazinyl)-3-pyridinyl]-4-{2-(ethylamino)-4-[3-methyl-5-(methyloxy)phenyl]-1,3-oxazol-5-yl}-2-pyrimidinamine: ¹H NMR (CDCl₃, 400 MHz) δ 8.15-8.12 (m, 2H), 7.68 (dd, J=2.0 and 8.8 Hz, 1H), 7.14 (s, 1H), 7.07 (s, 1H), 7.02 (s, 1H), 6.69-6.66 (m, 2H), 6.40 (d, J=9.2 Hz, 1H), 5.35 (brs, 1H), 3.70-3.66 (m, 5H), 3.52-3.33 (m, 8H), 2.22 (s, 3H), 2.08 (s, 3H), and 1.21 (t, J=7.2 Hz, 3H). MS (ESI) m/e (M+H⁺) 529.3.

### Example 71

### 4-{2-(Ethylamino)-4-[3-methyl-5-(methyloxy)phenyl]-1,3-thiazol-5-yl}-N-{6-[(9aR)-hexahydropyrazino[2,1-c][1,4]oxazin-8(1H)-yl]-3-pyridinyl}-2-pyrimidinamine

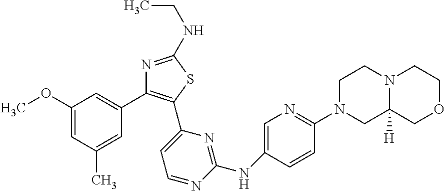

### Step A: (9aR)-Octahydropyrazino[2,1-c][1,4]oxazine hydrochloride

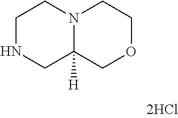

To a solution containing 2.8 g (12.1 mmol) of (9aR)-8-(phenylmethyl)octahydro-pyrazino[2,1-c][1,4]oxazine and 30 mL of MeOH was added 4.05 mL of 6 N aqueous HCl and 300 mg of 10% palladium on carbon. The reaction mixture was subjected to a hydrogen atmosphere overnight and filtered through Celite, eluting with MeOH. The solvents were removed under reduced pressure to give 1.6 g (62%) of (9aR)-octahydropyrazino[2,1-c][1,4]oxazine hydrochloride as a white foam. ¹H NMR (400 MHz, DMSO-d₆) δ 9.99 (brs, 2H), 3.84-4.01 (m, 4H), and 3.00-3.68 (m, 10H).

### Step B: (9aR)-8-(5-Nitro-2-pyridinyl)octahydropyrazino[2,1-c][1,4]oxazine

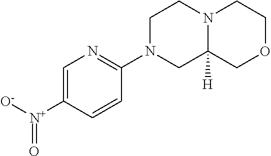

To a solution containing 0.63 g (3.97 mmol) of 2-chloro-5-nitropyridine and 5 mL of acetonitrile was added 0.85 g of (9aR)-octahydropyrazino[2,1-c][1,4]oxazine hydrochloride and 1.64 g (11.9 mmol) of potassium carbonate. The reaction mixture was allowed to stir at rt overnight, then partitioned between EtOAc and water. The water layer was further extracted with EtOAc and the combined organic layers were dried over MgSO₄ and filtered. The solvents were removed under reduced pressure and the residue was subjected to silica gel chromatography to give 0.77 g (73%) of (9aR)-8-(5-Nitro-2-pyridinyl)octahydropyrazino[2,1-c][1,4]oxazine as a yellow oil. ¹H-NMR (400 MHz, DMSO-d₆) δ 8.96 (d, J=2.8 Hz, 1H), 8.23 (dd, J=9.6 and 2.8 Hz, 1H), 6.97 (d, J=9.5 Hz, 1H), 4.49 (d, J=10.4 Hz, 1H), 4.38 (d, J=11.5 Hz, 1H), 3.77 (dt, J=11.1 and 2.7 Hz, 2H), 3.54 (td, J=11.4 and 2.3 Hz, 1H), 3.07-3.19 (m, 2H), 2.84 (ddd, J=11.2, 2.7, and 2.5 Hz, 1H), 2.61-2.71 (m, 2H), and 2.10-2.21 (m, 3H); ESIMS: 265.20 (M+H)⁺.

### Step C: 6-[(9aR)-Hexahydropyrazino[2,1-c][1,4]oxazin-8(1H)-yl]-3-pyridinamine

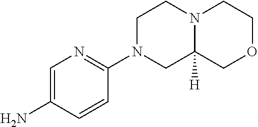

To a solution containing 0.77 g (2.9 mmol) of (9aR)-8-(5-nitro-2-pyridinyl)octahydro-pyrazino[2,1-c][1,4]oxazine, 5 mL of MeOH, and 10 mL of EtOH was added 70 mg of 5% platinum on carbon. The reaction mixture was subjected to a 40 psi atmosphere of hydrogen for 13 h, and then filtered over Celite. The solvents were removed under reduced pressure to give 0.6 g (88%) of dark solid: ¹H-NMR (400 MHz, DMSO-d₆) δ 7.58 (d, J=2.8 Hz, 1H), 6.90 (dd, J=8.8 and 2.93 Hz, 1H), 6.62 (d, J=8.8 Hz, 1H), 4.57 (s, 2H), 3.83 (d, J=12.3 Hz, 1H), 3.67-3.77 (m, 2H), 3.48-3.56 (m, 1H), 3.14 (t, J=10.2 Hz, 1H), 2.71-2.79 (m, 1H), 2.61-2.69 (m, 2H), 2.52-2.59 (m, 1 H), 2.12-2.22 (m, 3H), and 1.95-2.06 (m, 1H).

### Step D: 4-{2-(Ethylamino)-4-[3-methyl-5-(methyloxy)phenyl]-1,3-thiazol-5-yl}-N-{6-[(9aR)-hexahydropyrazino[2,1-c][1,4]oxazin-8(1H)-yl]-3-pyridinyl}-2-pyrimidinamine

To a solution containing 100 mg (0.28 mmol) of 5-(2-chloro-4-pyrimidinyl)-N-ethyl-4-[3-methyl-5-(methyloxy)phenyl]-1,3-thiazol-2-amine, prepared by a procedure similar to Example 25, Step F, 78 mg (0.33 mmol) of 6-[(9aR)-hexahydropyrazino[2,1-c][1,4]oxazin-8(1H)-yl]-3-pyridinamine, and 2 mL of IPA was added 0.14 mL of a 4.0M solution of HCl in dioxane. The reaction mixture was heated at 90° C. in a sealed tube overnight, then 0.5 mL of TEA was added and the solvents were removed under reduced pressure. The residue was subjected to HPLC purification to give 54 mg of 4-{2-(ethylamino)-4-[3-methyl-5-(methyloxy)phenyl]-1,3-thiazol-5-yl}-N-{6-[(9aR)-hexahydropyrazino[2,1-c][1,4]oxazin-8(1H)-yl]-3-pyridinyl}-2-pyrimidinamine as a yellow powder: ¹H NMR (400 MHz, DMSO-d₆) δ ppm 9.24 (s, 1H), 8.46 (d, J=1.8 Hz, 1H), 8.21 (t, J=5.3 Hz, 1H), 8.03 (d, J=5.3 Hz, 1H), 7.87 (dd, J=9.1 and 2.7 Hz, 1H), 6.76-6.91 (m, 4H), 6.24 (d, J=5.3 Hz, 1H), 4.06 (d, J=11.9 Hz, 1H), 3.97 (d, J=11.4 Hz, 1H), 3.74-3.82 (m, 2H), 3.73 (s, 3H), 3.54 (td, J=11.4, 2.1 Hz, 1H), 3.25-3.31 (m, 2H), 3.17 (t, J=10.44 Hz, 1H), 2.75-2.85 (m, 3H), 2.68 (d, J=11.4 Hz, 1H), 2.31 (s, 3H), 2.10-2.27 (m, 3H), and 1.18 (t, J=7.1 Hz, 3H). HRMS calcd for C₂₉H₃₅N₈O₂S (M+H⁺): 559.2604. found: 559.2611.

### Example 72

### 4-{2-(Ethylamino)-4-[3-(methyloxy)phenyl]-1,3-thiazol-5-yl}-N-(3-fluoro-4-{[2-(1-pyrrolidinyl)ethyl]oxy}phenyl)-2-pyrimidinamine

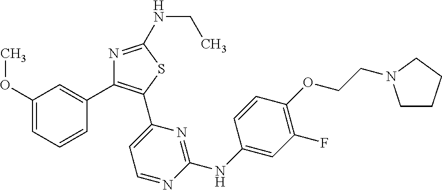

5-(2-Chloro-4-pyrimidinyl)-N-ethyl-4-[3-(methyloxy)phenyl]-1,3-thiazol-2-amine (120 mg, 0.35 mmol), prepared by a procedure analogous to Example 40, Step B, was mixed with (3-fluoro-4-{[2-(1 pyrrolidinyl)ethyl]oxy}phenyl)amine (88 mg, 0.34 mmol), prepared by a procedure analogous to Example 60, Step C, 2,2,2-trifluoroethanol (2 mL) and 4 N HCl in dioxane (170 μL, 0.68 mmol) and placed in the microwave at high absorption at 170° C. for 10 min. Silica gel and EtOAc were added and the mixture was concentrated, then purified on silica gel, eluting with 9:1 DCM:(90:9:1 DCM:MeOH:ammonium hydroxide) to 100% 90:9:1 DCM:MeOH:ammonium hydroxide. The clean fractions were concentrated, triturated with ether, and filtered to obtain the title compound as a yellow solid (86 mg, 47%): ¹H NMR (400 MHz, DMSO-d₆) δ ppm 9.47 (s, 1H), 8.24 (t, J=5.3 Hz, 1H), 8.05 (d, J=5.5 Hz, 1H), 7.78 (dd, J=14.6 and 2.5 Hz, 1H), 7.14-7.40 (m, 2H), 6.81-7.14 (m, 4H), 6.24 (d, J=5.4 Hz, 1H), 4.06 (t, J=5.9 Hz, 2H), 3.73 (s, 3H), 3.14-3.28 (m, 2H), 2.76 (brs, 2H), 2.49-2.55 (m, 4H), 1.66 (brs, 4H), and 1.17 (t, J=7.2 Hz, 3H). HRMS calcd for C₂₈H₃₂N₆O₂FS: [M+H]⁺535.2291. found 535.2302.

### Example 73

### 4-{2-(Fluoromethyl)-4-[3-(methyloxy)phenyl]-1,3-thiazol-5-yl}-N-(3-fluoro-4-{[2-(1-pyrrolidinyl)ethyl]oxy}phenyl)-2-pyrimidinamine

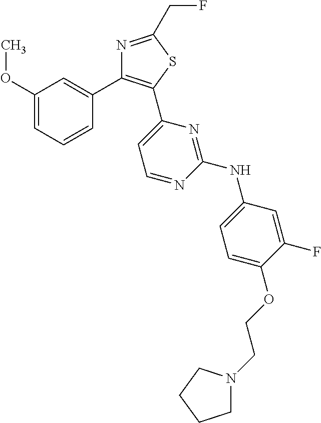

### Step A: 2-Chloro-4-{2-(fluoromethyl)-4-[3-(methyloxy)phenyl]-1,3-thiazol-5-yl}pyrimidine

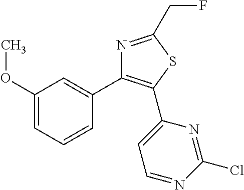

2-(2-Chloro-4-pyrimidinyl)-1-[3-(methyloxy)phenyl]ethanone (940 mg, 3.58 mmol), prepared by a procedure similar to Example 40, Step A, was partially dissolved in DCM (25 mL), treated with NBS (637 mg, 3.58 mmol) and stirred for 10 min. The solvent was removed and the residue was dissolved in DMF (15 mL). 2-Fluoroethanethioamide (500 mg, 5.37 mmol) was added and the mixture was stirred for 1.5 h. The reaction mixture was partitioned between EtOAc and water. The organic layer was dried over sodium sulfate and filtered. Silica gel was added and the mixture was concentrated and purified by silica gel chromatography, eluting with 95:5 Hexane:EtOAc to 100% EtOAc. The clean fractions were concentrated and triturated with IPA. The unclean fractions were combined and purified on silica gel with 100% DCM to 9:1 DCM:EtOAc. Clean product from both columns was combined to yield the title compound as a yellow solid (453 mg, 38%) MS (ESI): 336.2[M+H]⁺.

### Step B: 4-{2-(Fluoromethyl)-4-[3-(methyloxy)phenyl]-1,3-thiazol-5-yl}-N-(3-fluoro-4-{[2-(1-pyrrolidinyl)ethyl]oxy}phenyl)-2-pyrimidinamine

2-Chloro-4-{2-(fluoromethyl)-4-[3-(methyloxy)phenyl]-1,3-thiazol-5-yl}pyrimidine (70 mg, 0.21 mmol) was mixed with (3-fluoro-4-{[2-(1 pyrrolidinyl)ethyl]oxy}phenyl)amine (53 mg, 0.20 mmol), prepared by a procedure analogous to Example 60, Step C, 2,2,2-trifluoroethanol (3 mL) and 4 N HCl in dioxane (105 μL, 0.42 mmol) and placed in the microwave at high absorption at 170° C. for 16 min. Silica gel and EtOAc were added and the mixture was concentrated and purified on silica gel, eluting with 9:1 DCM:(90:9:1 DCM:MeOH:ammonium hydroxide) to 100% 90:9:1 DCM:MeOH:ammonium hydroxide. The clean fractions were concentrated and triturated with ether. The product was repurified with silica gel chromatography, eluting with EtOAc:9:1 EtOAc with 0.5% triethylamine:MeOH to give the title compound as a yellow foam (33 mg, 32%): ¹H NMR (400 MHz, DMSO-d₆) δ ppm 9.77 (s, 1H), 8.35 (d, J=5.2 Hz, 1H), 7.69 (dd, J=14.2 and 2.6 Hz, 1H), 7.30-7.47 (m, 2H), 7.07-7.13 (m, 3H), 6.99-7.09 (m, 1H), 6.54 (d, J=5.1 Hz, 1H), 5.83 (s, 1H), 5.72 (s, 1H), 4.08 (t, J=5.9 Hz, 2H), 3.75 (s, 3H), 2.77 (t, J=5.9 Hz, 2H), 2.50-2.54 (m, 4H), and 1.67 (ddd, J=6.8, 3.3, and 3.2 Hz, 4H). HRMS calcd for C₂₇H₂₈N₅O₂F₂S [M+H]⁺: 524.1932. found 524.1932.

### Example 74

### [2-Ethyl-5-(2-{[3-fluoro-4-({1-[2-(methylsulfonyl)ethyl]-4-piperidinyl}oxy)phenyl]amino}-4-pyrimidinyl)-1,3-thiazol-4-yl]-5-(methyloxy)phenol trifluoroacetic acid salt

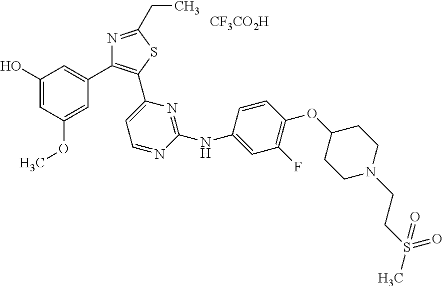

### Step A: Methyl 3-(methyloxy)-5-({[4-(methyloxy)phenyl]methyl}oxy)benzoate

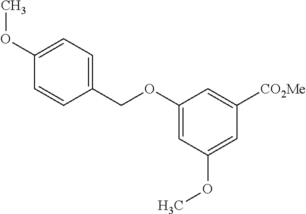

Methyl 3-hydroxy-5-(methyloxy)benzoate (397 mg, 2.18 mmol), 1-(chloromethyl)-4-(methyloxy)benzene (360 μL, 2.66 mmol) tetrabutylammonium iodide (5 mg, 0.01 mmol) and potassium carbonate (901 mg, 6.54 mmol) were suspended in acetone (7 mL) and heated to reflux for 5 days. The reaction mixture was partitioned between water and EtOAc. The organic layer was dried over sodium sulfate and filtered. Silica gel was added and the mixture was concentrated and purified on silica gel, eluting with 95:5 to 7:3 Hexane:EtOAc. The title compound of Step A was obtained as a colorless oil (583 mg, 88%): ¹H NMR (400 MHz, DMSO-d₆) δ ppm 7.35 (d, J=8.6 Hz, 2H), 7.11 (dd, J=2.1 and 1.3 Hz, 1H), 7.00-7.07 (m, 1H), 6.87-6.96 (m, 2H), 6.82 (t, J=2.3 Hz, 1H), 5.04 (s, 2H), 3.81 (s, 3H), 3.76 (s, 3H), and 3.73 (s, 3 H).

### Step B: 2-(2-Chloro-4-pyrimidinyl)-1-[3-(methyloxy)-5-({[4-(methyloxy)phenyl]methyl}oxy)phenyl]ethanone

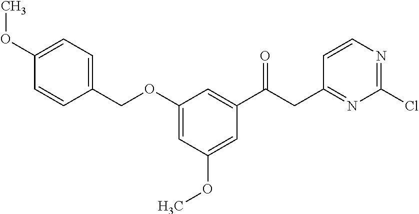

Methyl 3-(methyloxy)-5-({[4-(methyloxy)phenyl]methyl}oxy)benzoate (287 mg, 0.95 mmol) was dissolved in THF (7 mL), cooled to 0° C., and treated with 1.0 M LHMDS in THF (2.85 mL, 2.85 mmol). The reaction mixture was stirred for 30 min. 2-Chloro-4-methylpyrimidine (123 mg, 0.95 mmol) was dissolved in THF (3 mL) and added to the reaction. The reaction mixture was stirred for 1 h and water was added. The THF was removed under reduced pressure and the water layer was extracted three times with EtOAc. The EtOAc layers were combined, dried over sodium sulfate, filtered, and concentrated. The residue was dissolved in DCM and silica gel was added. The mixture was concentrated and purified on silica gel, eluting with 95: 5 to 8:2 Hexane:EtOAc. The product fractions were combined to yield the title compound of Step B as an orange solid (201 mg, 53%). MS (ESI): 399.2[M+H]⁺.

### Step C: 2-Chloro-4-{2-ethyl-4-[3-(methyloxy)-5-({[4-(methyloxy)phenyl]methyl}oxy)phenyl]-1,3-thiazol-5-yl}pyrimidine

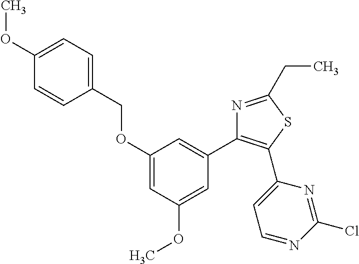

2-Chloro-4-{2-ethyl-4-[3-(methyloxy)-5-({[4-(methyloxy)phenyl]methyl}oxy)phenyl]-1,3-thiazol-5-yl}pyrimidine (375 mg, 0.94 mmol) (combined batch from Step B and an earlier batch of material prepared in a similar way) was dissolved in DCM (15 mL), treated with NBS (167 mg, 0.94 mmol), and stirred for 10 min. The solvent was removed and the residue was dissolved in DMF (15 mL). Propanethioamide (168 mg, 1.88 mmol) was added and the reaction mixture was stirred over the weekend, then partitioned between EtOAc and water. The organic layer was dried over sodium sulfate and filtered. Silica gel was added and the mixture was concentrated and purified by silica gel chromatography, eluting with 95:5 Hexane:EtOAc to 1:1 Hexane:EtOAc. The clean fractions were concentrated to obtain the title compound of Step C as a yellow oil (343 mg, 78%) MS (ESI): 468.3[M+H]⁺.

### Step D: [2-Ethyl-5-(2-{[3-fluoro-4-({1-[2-(methylsulfonyl)ethyl]-4-piperidinyl}oxy)phenyl]amino}-4-pyrimidinyl)-1,3-thiazol-4-yl]-5-(methyloxy)phenol trifluoroacetic acid salt

2-Chloro-4-{2-ethyl-4-[3-(methyloxy)-5-({[4-(methyloxy)phenyl]methyl}oxy)phenyl]-1,3-thiazol-5-yl}pyrimidine (221 mg, 0.47 mmol), [3-fluoro-4-({1-[2-(methylsulfonyl)ethyl]-4-piperidinyl}oxy)phenyl]amine (148 mg, 0.47 mmol), prepared by a procedure analogous to Example 10, Step D, 4 N HCl in dioxane (470 μL, 1.88 mmol) and 2,2,2-trifluoroethanol (4 mL) were combined and placed in the microwave at high absorption at 170° C. for 15 min. A DCM:MeOH solvent mixture and silica gel were added and the mixture was concentrated and purified on silica gel, eluting with 100% EtOAc to 9:1 EtOAc:MeOH. Product fractions were concentrated, redissolved in MeOH, and purified by reverse phase LC (solvent system: acetonitrile:water with 0.1% TFA in both). Clean fractions were combined to obtain the title compound as a yellow solid (40 mg, 11%): ¹H NMR (400 MHz, DMSO-d₆) δ ppm 9.75-9.82 (m, 1H), 9.60 (td, J=6.3 and 2.0 Hz, 1H), 8.32 (d, J=5.2 Hz, 1H), 7.71-7.86 (m, 1H), 7.37 (s, 1H), 7.02-7.23 (m, 1H), 6.56 (d, J=5.2 Hz, 1H), 6.47 (d, J=2.0 Hz, 2H), 6.37 (d, J=1.9 Hz, 1H), 4.26-4.62 (m, 2H), 3.65 (s, 3H), 3.37-3.49 (m, 2H), 3.16-3.29 (m, 1H), 3.12 (s, 1H), 3.09 (s, 3H), 3.00 (q, 2H), 2.17-2.34 (m, 1H), 2.02-2.13 (m, 1H), 1.86-2.00 (m, 1H), 1.65-1.81 (m, 1H), and 1.32 (t, J=7.5 Hz, 3H). HRMS calcd for C₃₀H₃₆N₆O₆FS₂ [M+H]⁺: 628.2064. found 628.2061.

### Example 75

### 4-{2-Amino-4-[3,5-bis(methyloxy)phenyl]-1,3-oxazol-5-yl}-N-[3-fluoro-4-({1-[2-(methylsulfonyl)ethyl]-4-piperidinyl}oxy)phenyl]-2-pyrimidinamine

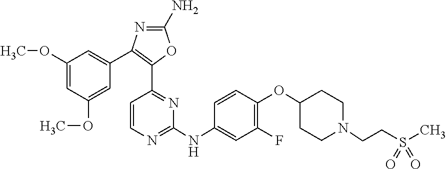

### Step A: 4-[3,5-Bis(methyloxy)phenyl]-5-(2-chloro-4-pyrimidinyl)-1,3-oxazol-2-amine

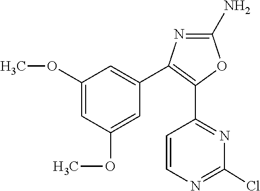

To a solution of 1-[3,5-bis(methyloxy)phenyl]-2-(2-chloro-4-pyrimidinyl)ethanone (500 mg, 1.71 mmol), prepared by a procedure analogous to Example 25, Step E, in DCM (8.6 mL) was added NBS (304 mg, 1.71 mmol). The reaction was stirred at rt for 30 min and then concentrated. The residue was redissolved in 1,4-dioxane (5.2 mL) and urea (668 mg, 11.1 mmol) was added. The reaction was heated in a microwave at 150° C. for 40 min, then diluted with DCM (50 mL) and water (50 mL) and stirred for 5 min. The layers were separated, and the organic fraction was concentrated onto silica gel. Purification by flash column chromatography (10 to 100% EtOAc:hexanes) afforded 140 mg (25%) of the title compound of Step A. ¹H NMR (400 MHz, DMSO-d₆) δ ppm 8.59 (d, J=5.5 Hz, 1H), 7.74 (s, 1H), 7.32 (d, J=5.5 Hz, 1H), 7.26 (d, J=2.2 Hz, 2H), 7.03 (d, J=2.4 Hz, 1H), 6.56 (t, J=2.3 Hz, 1H), and 3.80 (s, 6H); MS (ESI): 333.12 [M+H]⁺.

### Step B: 4-[4-[3,5-Bis(methyloxy)phenyl]-2-(ethylamino)-1,3-oxazol-5-yl]-N-[3-chloro-4-(4-methyl-1-piperazinyl)phenyl]-2-pyrimidinamine

The title compound of Example 75 was prepared from 4-[3,5-bis(methyloxy)phenyl]-5-(2-chloro-4-pyrimidinyl)-1,3-oxazol-2-amine (132 mg, 0.396 mmol) and [3-fluoro-4-({1-[2-(methylsulfonyl)ethyl]-4-piperidinyl}oxy)phenyl]amine (138 mg, 0.436 mmol), prepared by a procedure analogous to Example 10, Step D, in 14% yield by a procedure analogous to Example 1, Step G, except that the reaction was run 16 h at 90° C. on the benchtop. ¹H NMR (400 MHz, DMSO-d₆) δ ppm 9.52 (s, 1H), 8.40 (d, J=5.1 Hz, 1H), 7.50 (d, J=13.9 Hz, 1H), 7.37 (s, 2H), 7.27 (d, J=9.0 Hz, 1H), 6.66-7.11 (m, 4H), 6.47 (t, J=2.2 Hz, 1H), 4.08-4.25 (m, 1H), 3.65 (s, 6H), 3.18-3.31 (m, 2H), 3.04 (s, 3H), 2.59-2.84 (m, 4H), 2.14-2.37 (m, 2H), 1.78-1.96 (m, 2H), and 1.46-1.73 (m, 2H); MS (ESI): 613.39 [M+H]⁺.

### Example 76

### N-(4-{2-(1-Methylethyl)-4-[3-(methyloxy)phenyl]-1,3-thiazol-5-yl}-2-pyrimidinyl)-N′-[2-(1-pyrrolidinyl)ethyl]-1,4-benzenediamine

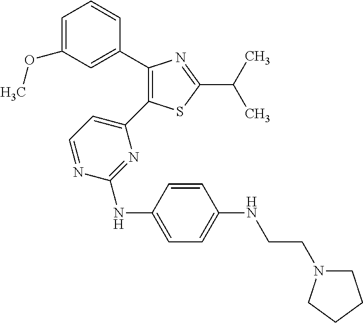

2-Chloro-4-{2-(1-methylethyl)-4-[3-(methyloxy)phenyl]-1,3-thiazol-5-yl}pyrimidine, prepared by a procedure analogous to Example 59, Step B, (80 mg, 0.23 mmol) and (4-aminophenyl)[2-(1-pyrrolidinyl)ethyl]amine (47 mg, 0.23 mmol) were combined in a microwave reaction vessel containing IPA (2 mL) and concentrated HCl (2 drops). This mixture was heated at 180° C. for 15 min in a microwave. TEA (0.1 mL) was added to the crude reaction, which was then concentrated to dryness onto silica gel. Purification via flash chromatography and concentration of desired fractions provided a solid which was then sonicated in ether and filtered to yield 41 mg of the target compound. ¹H NMR (400 MHz, DMSO-d₆) δ ppm 9.44 (1H, s), 8.26 (d, J=5.1 Hz, 1H), 7.53 (d, J=9.0 Hz, 2H), 7.38 (t, J=8.1 Hz, 1H), 7.07-7.14 (m, 2H), 6.99-7.07 (m, 1H), 6.87 (d, J=9.0 Hz, 2H), 6.45 (d, J=5.1 Hz, 1H), 3.76 (s, 3H), 3.33-3.37 (m, 1H), 3.30 (s, 4H), 3.04-3.10 (m, 4H), 2.37 (q, J=7.3 Hz, 2H), 1.40 (d, J=7.0 Hz, 6H), and 1.04 (t, J=7.1 Hz, 2H).

### Example 77

### N-(4-{[2-(Dimethylamino)ethyl]oxy}-3-methylphenyl)-4-{2-(ethylamino)-4-[3-(methyloxy)phenyl]-1,3-thiazol-5-yl}-2-pyrimidinamine

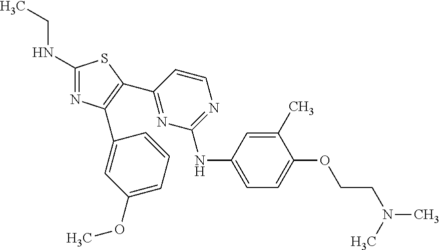

### Step A: Dimethyl{2-[(2-methyl-4-nitrophenyl)oxy]ethyl}amine

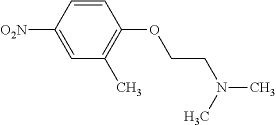

2-Methyl-4-nitrophenol (5 g, 32.6 mmol) and 1-(2-chloroethyl)pyrrolidine hydrochloride (8.4 g, 58.3 mmol) were combined in a round bottom flask with DMF and cesium carbonate (32 g, 98 mmol). The mixture was stirred at 70° C. overnight.

The completed reaction was concentrated to remove DMF and then partitioned between EtOAc and water. The organic layers was washed with water and brine, treated with magnesium sulfate, filtered and concentrated to dryness. The crude oil was chromatographed on normal phase silica gel and clean fractions were combined and concentrated to yield 3 g of the target compound of Step A. ¹H NMR (400 MHz, DMSO-d₆) δ ppm 8.00-8.11 (m, 2H), 7.13 (d, J=8.8 Hz, 1H), 4.18 (t, J=5.7 Hz, 2 H), 2.66 (t, J=5.6 Hz, 2H), and 2.14-2.24 (m, 9H).

### Step B: 4-{[2-(Dimethylamino)ethyl]oxy}-3-methylaniline hydrochloride

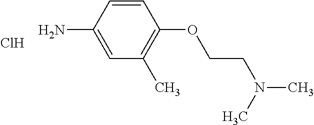

Dimethyl{2-[(2-methyl-4-nitrophenyl)oxy]ethyl}amine (3.0 g, 13.3 mmol) was combined with 5% Pd/C (300 mg) and EtOH. The mixture was placed under 55 psi of H₂ for 3 h. The reaction was purged with N₂ and filtered trough celite to remove Pd catalyst. The filtrate was concentrated to dryness to yield 2.44 g of the freebase. This material was treated with 4 M HCl in dioxane (3.13 mL and concentrated to obtain 2.74 g of the title compound of Step B. ¹H NMR (400 MHz, DMSO-d₆) δ ppm 6.63 (d, J=8.4 Hz, 1H), 6.38 (d, J=2.6 Hz, 1H), 6.33 (dd, J=8.5 and 2.6 Hz, 1H), 4.50 (brs, 2H), 3.87 (t, J=5.9 Hz, 2H), 2.57 (t, J=5.9 Hz, 2H), 2.21 (s, 6H), and 2.03 (s, 3H).

### Step C: N-(4-{[2-(Dimethylamino)ethyl]oxy}-3-methylphenyl)-4-{2-(ethylamino)-4-[3-(methyloxy)phenyl]-1,3-thiazol-5-yl}-2-pyrimidinamine

A mixture of 5-(2-chloro-4-pyrimidinyl)-N-ethyl-4-[3-(methyloxy)phenyl]-1,3-thiazol-2-amine (200 mg, 0.577 mmol) (prepared according to methods similar to that used in Example 40, Step B) and 4-{[2-(dimethylamino)ethyl]oxy}-3-methylaniline hydrochloride (148 mg, 0.577 mmol) in trifluoroethanol (2 mL) was heated at 170° C. for 10 min in a microwave apparatus. The mixture was allowed to cool to room temperature and was partitioned between sat. aq. NaHCO₃ and DCM. The organic layer was separated, dried over Na₂SO₄, filtered, and concentrated to give a residue that was absorbed onto silica and purified by silica gel chromatography, eluting with a gradient starting with 100% EtOAc and going to a 80:20 mixture of A to B (A=5:1:0.1 EtOAc/MeOH/concentrated NH₄OH, B=100% EtOAc) to give 80.8 mg of a yellow solid. ¹H NMR showed the material to be primarily product with a small amount of starting aniline. Trituration with MeOH provided 44.9 mg (15% yield) of the title compound of Step C as a bright yellow solid. MS (ESI): 505.41 (M+H⁺). ¹H NMR (400 MHz, DMSO-d₆) δ ppm 9.21 (1H, s), 8.22 (t, J=5.2 Hz, 1H), 8.04 (d, J=5.3 Hz, 1H), 7.62 (brs, 1H), 7.33-7.45 (m, 2H), 6.95-7.09 (m, 3H), 6.84 (d, J=8.8 Hz, 1H), 6.22 (d, J=5.3 Hz, 1H), 4.01 (t, J=5.8 Hz, 2H), 3.76 (s, 3H), 3.25-3.32 (m, 2H), 2.64 (t, J=5.8 Hz, 2H), 2.24 (s, 6H), 2.17 (s, 3H), and 1.20 (t, J=7.2 Hz, 3H).

### Example 78

### 4-[4-[4-Chloro-3-(methyloxy)phenyl]-2-(1-pyrrolidinyl)-1,3-thiazol-5-yl]-N-(3-fluoro-4-{[2-(1-pyrrolidinyl)ethyl]oxy}phenyl)-2-pyrimidinamine

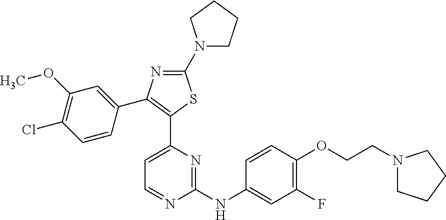

### Step A: 1-Pyrrolidinecarbothioamide

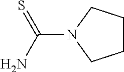

Pyrrolidine (1.5 g, 21 mmol) was placed in a round bottom flask under N₂ with stirring. THF (4 mL) was added, followed by the drop-wise addition of 4 N HCl in dioxane (5.3 mL, 21 mmol). Potassium thiocyanate (2.0 g, 21 mmol) was then added in one portion to the stirring solution of pyrrolidine hydrochloride. This mixture was then stirred at rt for 30 min followed by heating at 100° C. for 2 h. The reaction was then cooled to rt, MeOH (50 mL) was added, and solids that persisted were filtered away. Subsequent concentration of the MeOH/reaction solution yielded 3.0 g of the crude 1-pyrrolidinecarbothioamide. ¹H-NMR (400 MHz, DMSO-d₆) δ 8.60 (brs, 2H), 3.07 (m, 4H), and 1.82 (m, 4H).

### Step B: 2-Chloro-4-[4-[4-chloro-3-(methyloxy)phenyl]-2-(1-pyrrolidinyl)-1,3-thiazol-5-yl]pyrimidine

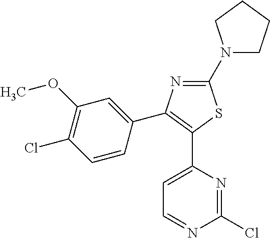

The title compound of Step B was prepared from 1-[4-chloro-3-(methyloxy)phenyl]-2-(2-chloro-4-pyrimidinyl)ethanone (174 mg, 0.59 mmol) and 1-pyrrolidinecarbothioamide (153 mg, 1.18 mmol) by a procedure analogous to Example 1, Step F. The crude reaction mixture was concentrated onto silica. Purification by flash column chromatography (0 to 20% EtOAc:Hexanes) and subsequent trituration of the chromatographed material with ether afforded 75 mg of the title compound of Step B. MS (AP): 407.0 [M+H]⁺.

### Step C: 4-[4-[4-Chloro-3-(methyloxy)phenyl]-2-(1-pyrrolidinyl)-1,3-thiazol-5-yl]-N-(3-fluoro-4-{[2-(1-pyrrolidinyl)ethyl]oxy}phenyl)-2-pyrimidinamine

The title compound was prepared from 2-chloro-4-[4-[4-chloro-3-(methyloxy)phenyl]-2-(1-pyrrolidinyl)-1,3-thiazol-5-yl]pyrimidine (75 mg, 0.18 mmol) and (3-fluoro-4-{[2-(1-pyrrolidinyl)ethyl]oxy}phenyl)amine hydrochloride (48 mg, 0.18 mmol), prepared by a procedure analogous to Example 60, Step C, by a procedure analogous to Example 1, Step G. The crude reaction mixture was concentrated onto silica. Purification by flash column chromatography (0 to 70% 1:9:90 ammonium hydroxide:MeOH:DCM I DCM) and subsequent trituration of the chromatographed material with ether afforded 49 mg of the title compound. ¹H NMR (400 MHz, METHANOL-d4) δ ppm 8.00 (d, J=5.5 Hz, 1H), 7.72 (dd, J=14.1 and 2.6 Hz, 1H), 7.45 (d, J=8.1 Hz, 1H), 7.19 (d, J=1.6 Hz, 2H), 6.96-7.10 (m, 2H), 6.26 (d, J=5.3 Hz, 1H), 4.17 (t, J=5.6 Hz, 2H), 3.48-3.58 (m, 4H), 2.97 (t, J=5.4 Hz, 2H), 2.75 (brs, 4H), 2.03-2.15 (m, 4H), and 1.76-1.91 (m, 4H). MS (AP): 407.0 [M+H]⁺.

The following compounds were also prepared by methods similar to those described and/or demonstrated above:

| Example | Structure | Analytical data |
|---|---|---|
| 79 | 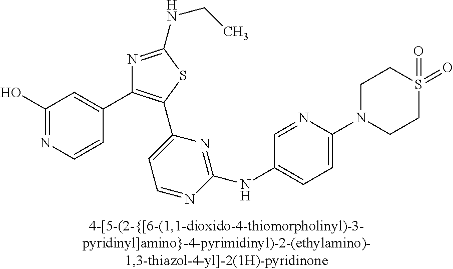 | ¹H NMR (400 MHz, DMSO-d₆) δ 11.68 (brs, 1H), 9.39 (s, 1H), 8.51 (d, J = 2.6 Hz, 1H), 8.28 (t, J = 5.3 Hz, 1H), 8.19 (d, J = 5.3 Hz, 1H), 7.92 (dd, J = 9.1 and 2.7 Hz, 1H), 7.42 (d, J = 6.6 Hz, 1H), 6.98 (d, J = 9.2 Hz, 1H), 6.56 (d, J = 5.3 Hz, 1H), 6.41 (s, 1H), 6.20 (d, J = 5.3 Hz, 1H), 4.01 (brs, 4H), 3.24- 3.31 (m, 2H), 3.09 (brs, 4H), and 1.18 (t, J = 7.2 Hz, 3H) |
| 80 | 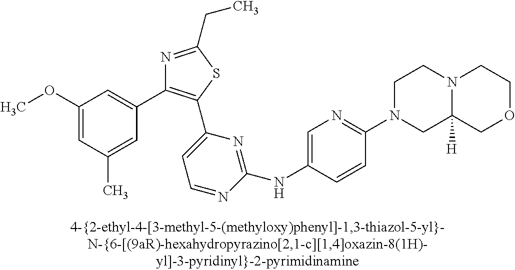 | ¹H NMR (400 MHz, DMSO-d₆) δ ppm 9.48 (s, 1H), 8.42 (d, J = 2.6 Hz, 1H), 8.25 (d, J = 5.3 Hz, 1H), 7.86 (dd, J = 9.1 and 2.7 Hz, 1H), 6.94 (s, 1H), 6.80-6.87 (m, 3H), 6.46 (d, J = 5.1 Hz, 1H), 4.08 (d, J = 11.9 Hz, 1H), 3.99 (d, J = 11.5 Hz, 1H), 3.74-3.80 (m, 3H), 3.72 (s, 3H), 3.50-3.59 (m, 1H), 3.30 (s, 1H), 3.17 (t, J = 10.4 Hz, 1H), 3.04 (q, J = 7.5 Hz, 2H), 2.77-2.86 (m, 2H), 2.66-2.70 (m, 1H), 2.29-2.34 (m, 4H), 2.11- 2.27 (m, 3H), and 1.35 (t, J = 7.5 Hz, 2H) |
| 81 | 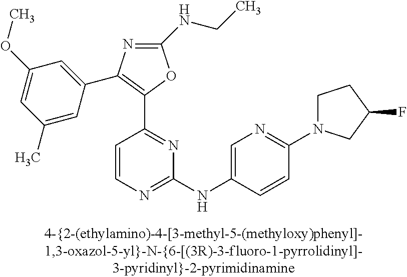 | ¹H NMR (400 MHz, DMSO-d₆) δ ppm 9.11 (s, 1H), 8.29 (d, J = 5.1 Hz, 1H), 8.18 (brs, 1H), 7.84 (t, J = 5.4 Hz, 1H), 7.66 (d, J = 8.6 Hz, 1H), 7.18 (s, 1H), 7.09 (brs, 1H), 6.75 (s, 1H), 6.68 (d, J = 5.1 Hz, 1H), 6.19 (d, J = 8.6 Hz, 1H), 3.67 (s, 3H), 3.52-3.63 (m, 5H), 3.48 (d, J = 3.3 Hz, 1H), 3.35-3.42 (m, 1H), 2.26 (d, J = 7.3 Hz, 1H), 2.20 (s, 3H), 2.06-2.17 (m, 1H), and 1.19 (t, J = 7.1 Hz, 3H) |
| 82 | 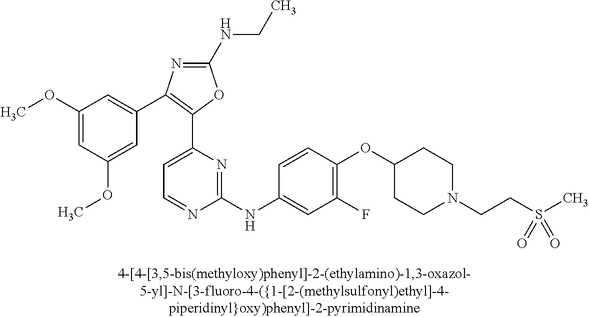 | ¹H NMR (400 MHz, DMSO-d₆) δ ppm 9.51 (s, 1H), 8.38 (d, J = 5.3 Hz, 1H), 7.90 (t, J = 5.7 Hz, 1H), 7.56 (dd, J = 14.1, 2.2 Hz, 1H), 7.15-7.39 (m, 1H), 6.82-7.03 (m, 3H), 6.79 (d, J = 5.3 Hz, 1H), 6.49 (t, J = 2.3 Hz, 1H), 4.10- 4.26 (m, 1H), 3.66 (s, 6H), 3.19- 3.42 (m, 1H), 3.04 (s, 3H), 2.59- 2.85 (m, 4H), 2.13-2.40 (m, 2H), 1.80- 1.96 (m, 2H), 1.50-1.76 (m, 2H), and 1.02-1.34 (m, 5H); MS (ESI): 641.33 [M + H]⁺. |
| 83 | 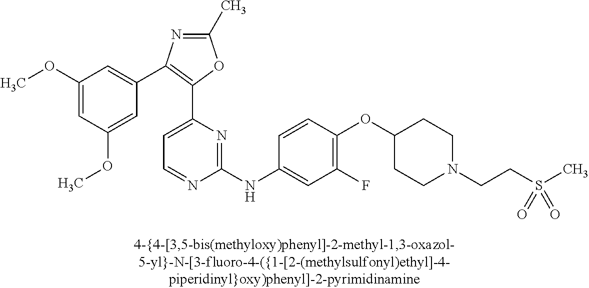 | ¹H NMR (400 MHz, DMSO-d₆) δ ppm 9.75 (s, 1H), 8.54 (d, J = 5.1 Hz, 1H), 7.56 (d, J = 13.9 Hz, 1H), 7.26 (d, J = 8.8 Hz, 1H), 7.01 (d, J = 5.3 Hz, 1H), 6.76- 6.97 (m, 3H), 6.51 (t, J = 2.2 Hz, 1H), 4.11-4.29 (m, 1H), 3.67 (s, 6H), 3.21-3.31 (m, 2H), 3.03 (s, 3H), 2.63-2.81 (m, 4H), 2.56 (s, 3H), 2.14-2.35 (m, 2H), 1.77- 1.98 (m, 2H), and 1.48-1.71 (m, 2H); MS (ESI): 612.54 [M + H]⁺. |
| 84 | 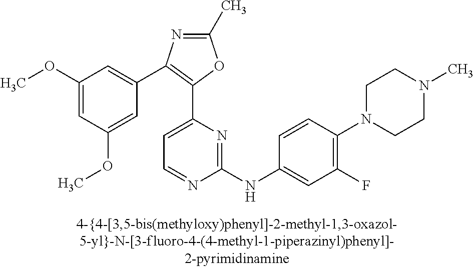 | ¹H NMR (400 MHz, DMSO-d₆) δ ppm 9.60-9.89 (m, 1H), 8.42- 8.69 (m, 1H), 7.36-7.66 (m, 1H), 7.15-7.35 (m, 1H), 6.96-7.04 (m, 1H), 6.84-6.96 (m, 2H), 6.65- 6.84 (m, 1H), 6.42-6.59 (m, 1H), 3.67 (s, 6H), 2.77-3.05 (m, 4H), 2.56 (s, 3H), 2.45 (s, 3H), and 2.08- 2.30 (m, 4H); MS (APCI): 505.23 [M + H]⁺. |
| 85 | 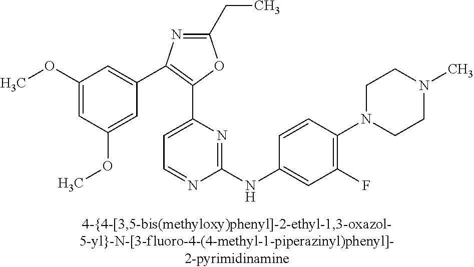 | ¹H NMR (400 MHz, DMSO-d₆) δ ppm 9.73 (s, 1H), 8.52 (d, J = 5.1 Hz, 1H), 7.52 (dd, J = 15.2 and 2.0 Hz, 1H), 7.26 (dd, J = 8.8 and 1.6 Hz, 1H), 6.99 (d, J = 5.1 Hz, 1H), 6.91 (d, J = 2.2 Hz, 2H), 6.77 (t, J = 9.4 Hz, 1H), 6.53 (t, J = 2.3 Hz, 1H), 3.67 (s, 6H), 2.79-3.00 (m, 6H), 2.38-2.48 (m, 4H), 2.22 (s, 3H), and 1.34 (t, J = 7.6 Hz, 3H); MS (ESI): 519.38 [M + H]⁺. |
| 86 | 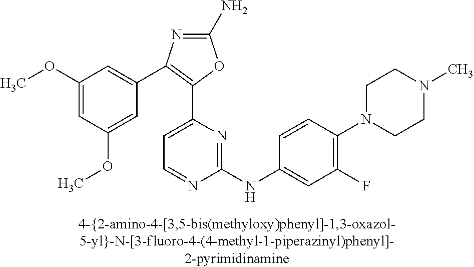 | ¹H NMR (400 MHz, DMSO-d₆) δ ppm 9.48 (s, 1H), 8.39 (d, J = 5.3 Hz, 1H), 7.16-7.56 (m, 4H), 6.94 (d, J = 1.8 Hz, 2H), 6.66-6.83 (m, 2H), 6.49 (s, 1H), 3.65 (s, 6H), 2.82-3.01 (m, 4H), 2.37-2.48 (m, 4H), and 2.22 (s, 3H); MS (ESI): 506.34 [M + H]⁺. |
| 87 | 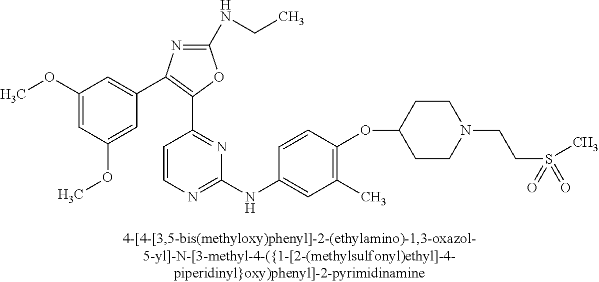 | ¹H NMR (400 MHz, DMSO-d₆) δ ppm 9.19 (s, 1H), 8.34 (d, J = 5.2 Hz, 1H), 7.85 (s, 1H), 7.33 (s, 2H), 6.96 (d, J = 2.1 Hz, 2H), 6.73 (d, J = 5.2 Hz, 1H), 6.64 (d, J = 8.6 Hz, 1H), 6.48 (t, J = 2.2 Hz, 1H), 4.17-4.25 (m, 1H), 3.65 (s, 6H), 3.24-3.38 (m, 6H), 3.04 (s, 3H), 2.66-2.75 (m, 5H), 2.27-2.35 (m, 2H), 1.85-1.93 (m, 2H), 1.58-1.69 (m, 2H), and 1.20 (t, J = 7.1 Hz, 3H) |
| 88 | 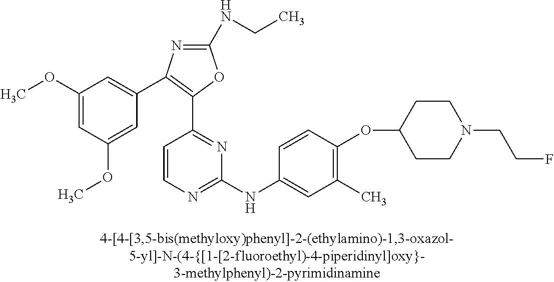 | ¹H NMR (400 MHz, DMSO-d₆) δ ppm 9.19 (s, 1H), 8.34 (d, J = 5.2 Hz, 1H), 7.85 (t, J = 5.6 Hz, 1H), 7.29-7.34 (m, 2H), 6.96 (d, J = 2.2 Hz, 2H), 6.73 (d, J = 5.2 Hz, 1H), 6.64 (d, J = 8.8 Hz, 1H), 6.48 (t, J = 2.2 Hz, 1H), 4.59 (t, J = 4.9 Hz, 1H), 4.47 (t, J = 4.9 Hz, 1H), 4.16-4.24 (m, 1H), 3.65 (s, 6H), 3.34 (d, J = 7.1 Hz, 2H), 2.71 (brs, 2H), 2.66 (t, J = 4.9 Hz, 1H), 2.59 (t, J = 4.9 Hz, 1H), 2.29-2.37 (m, 2H), 1.99 (s, 3H), 1.88 (d, J = 3.2 Hz, 2H), 1.65 (dd, J = 8.1 and 3.8 Hz, 2H), and 1.20 (t, J = 7.2 Hz, 3H) |
| 89 | 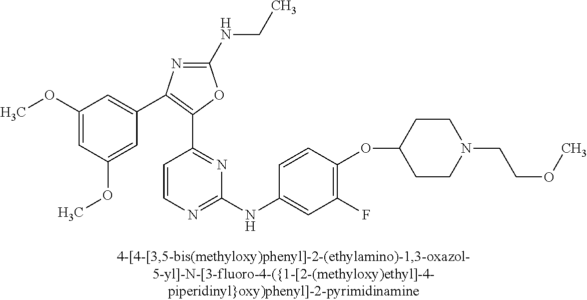 | ¹H NMR (400 MHz, DMSO-d₆) δ ppm 9.51 (s, 1H), 8.38 (d, J = 5.3 Hz, 1H), 7.90 (t, J = 5.6 Hz, 1H), 7.56 (dd, J = 14.1 and 1.7 Hz, 1H), 7.25 (d, J = 8.9 Hz, 1H), 6.92 (d, J = 2.1 Hz, 2H), 6.87 (t, J = 9.3 Hz, 1H), 6.78 (d, J = 5.2 Hz, 1H), 6.48-6.50 (m, 1H), 4.14 (brs, 1H), 3.66 (s, 6H), 3.43 (t, J = 5.8 Hz, 2H), 3.34 (d, J = 7.0 Hz, 2H), 3.23 (s, 3H), 2.73 (brs, 2H), 2.48 (brs, 2H), 2.23 (brs, 2H), 1.87 (brs, 2H), 1.61 (d, J = 9.2 Hz, 2H), and 1.20 (t , J = 7.1 Hz, 3H) |
| 90 | 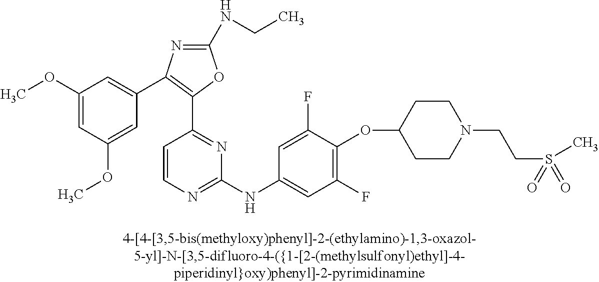 | ¹H NMR (400 MHz, DMSO-d₆) δ ppm 9.76 (s, 1H), 8.42 (d, J = 5.3 Hz, 1H), 7.93 (t, J = 5.6 Hz, 1H), 7.39 (d, J =11.1 Hz, 2H), 6.93 (d, J = 2.0 Hz, 2H), 6.84 (d, J = 5.2 Hz, 1H), 6.46-6.48 (m, 1H), 3.99 (brs, 1H), 3.65 (s, 6H), 3.32- 3.39 (m, 2H), 3.28 (t, J = 6.7 Hz, 2H), 3.03 (s, 3H), 2.75 (d, J = 5.2 Hz, 2H), 2.71 (t, J = 6.7 Hz, 2H), 2.23 (t, J = 8.8 Hz, 2H), 1.87 (d, J = 10.5 Hz, 2H), 1.61-1.70 (m, 2H), and 1.20 (t, J = 7.1 Hz, 3H) |
| 91 | 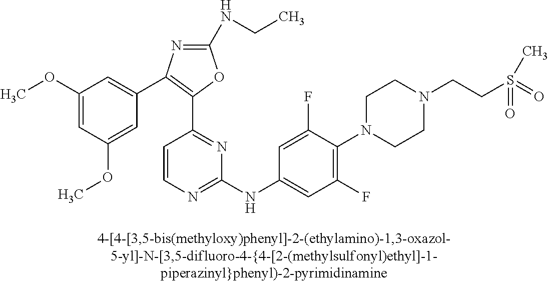 | ¹H NMR (400 MHz, DMSO-d₆) δ ppm 9.73 (s, 1H), 8.41 (d, J = 5.3 Hz, 1H), 7.92 (t, J = 5.6 Hz, 1H), 7.32 (d, J = 12.1 Hz, 2H), 6.92 (d, J = 2.1 Hz, 2H), 6.83 (d, J = 5.3 Hz, 1H), 6.49 (t, J = 2.1 Hz, 1H), 3.66 (s, 6H), 3.32-3.39 (m, 2H), 3.32 (s, 2H), 3.06 (s, 3H), 3.02 (t, 4H), 2.75 (t, J = 6.7 Hz, 2H), 2.54 (brs, 4H), and 1.20 (t, J = 7.1 Hz, 3H) |
| 92 | 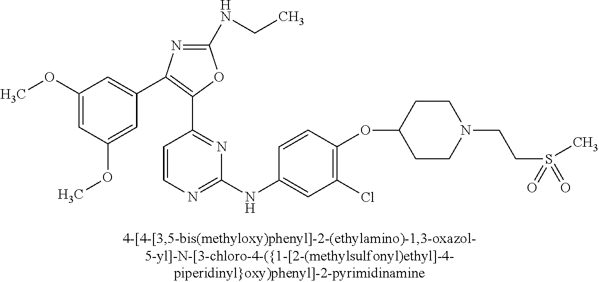 | ¹H NMR (400 MHz, DMSO-d₆) δ ppm 9.48 (s, 1H), 8.38 (d, J = 5.3 Hz, 1H), 7.88 (t, J = 5.6 Hz, 1H), 7.72 (d, J = 2.2 Hz, 1H), 7.47 (dd, J = 9.0 and 2.5 Hz, 1H), 6.93 (d, J = 2.1 Hz, 2H), 6.86 (d, J = 9.1 Hz, 1H), 6.78 (d, J = 5.2 Hz, 1H), 6.50 (t, J = 2.2 Hz, 1H), 4.28 (brs, 1H), 3.66 (s, 6H), 3.35 (d, J = 6.2 Hz, 2H), 3.29 (t, J = 7.2 Hz, 2H), 3.04 (s, 3H), 2.72 (t, J = 6.6 Hz, 4H), 2.26-2.35 (m, 2H), 1.85-1.94 (m, 2H), 1.60-1.72 (m, 2H), and 1.20 (t, J = 7.2 Hz, 3H) |
| 93 | 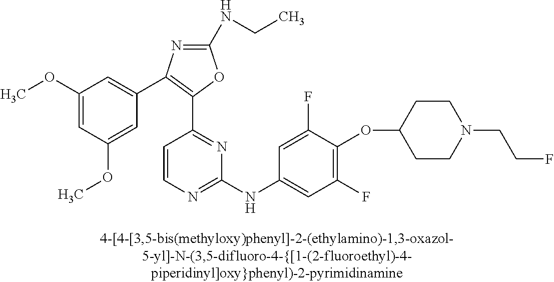 | ¹H NMR (400 MHz, DMSO-d₆) δ ppm 9.77 (s, 1H), 8.42 (d, J = 5.3 Hz, 1H ), 7.95 (brs, 1H), 7.39 (d, J = 11.0 Hz, 2H), 6.93 (d, J = 1.3 Hz, 2H), 6.84 (d, J = 5.2 Hz, 1H), 6.47 (s, 1H), 4.58 (t, J = 4.9 Hz, 1H), 4.46 (t, J = 4.9 Hz, 1H), 3.97 (d, J = 3.7 Hz, 1H), 3.65 (s, 6H), 3.36 (brs, 2H), 2.73-2.79 (m, 2H), 2.65 (t, J = 4.9 Hz, 1H), 2.58 (t , J = 4.9 Hz, 1H), 2.24 (t, J = 9.2 Hz, 2H), 1.87 (d, J = 10.3 Hz, 2H), 1.59-1.75 (m, 2H), and 1.20 (t, 3H) |
| 94 | 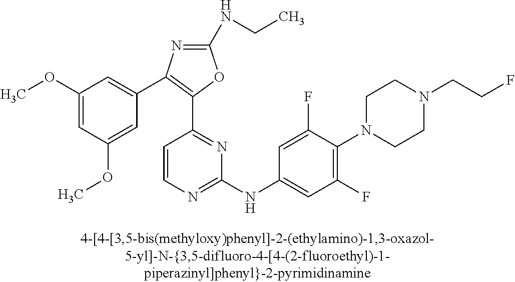 | ¹H NMR (400 MHz, DMSO-d₆) δ ppm 9.72 (brs, 1H), 8.41 (d, J = 4.9 Hz, 1H), 7.93 (brs, 2H), 7.32 (d, J = 11.8 Hz, 1H), 6.92 (brs, 2H), 6.83 (d, J = 4.9 Hz, 1H), 6.49 (brs, 1H), 4.61 (d, J = 4.2 Hz, 1H), 4.50 (brs, 1H), 3.66 (s, 6H), 3.34- 3.42 (m, 2H), 3.02 (brs, 2H), 2.94 (brs, 2H), 2.80 (brs, 2H), 2.70 (brs, 1H), 2.63 (brs, 1H), 2.54 (brs, 2H), and 1.20 (t, J = 6.8 Hz, 3H) |
| 95 | 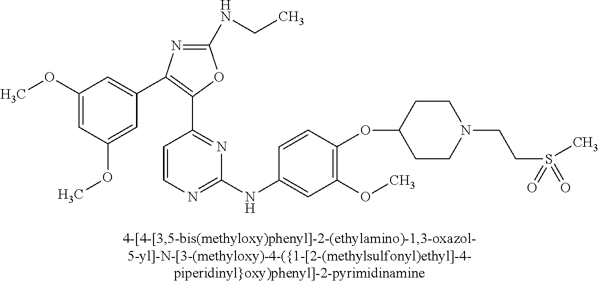 | ¹H NMR (400 MHz, DMSO-d₆) δ ppm 9.30 (s, 1H), 8.37 (d, J = 5.3 Hz, 1H), 7.86 (t, J = 5.6 Hz, 1H), 7.31 (d, J = 2.4 Hz, 1H), 7.06 (dd, J = 8.7 and 2.3 Hz, 1H), 6.97 (d, J = 2.3 Hz, 2H), 6.76 (d, J = 5.2 Hz, 1H), 6.66 (d, J = 8.7 Hz, 1H), 6.47 (t, J = 2.3 Hz, 1H), 4.04- 4.12 (m, 1H), 3.63 (s, 6H), 3.51 (s, 3H), 3.25-3.31 (m, 4H), 3.04 (s, 3H), 2.76 (brs, 2H), 2.71 (t, J = 6.7 Hz, 2H), 2.17-2.25 (m, 2H), 1.83-1.90 (m, 2H), 1.53-1.64 (m, 2H), and 1.19 (t, J = 7.2 Hz, 3H) |
| 96 | 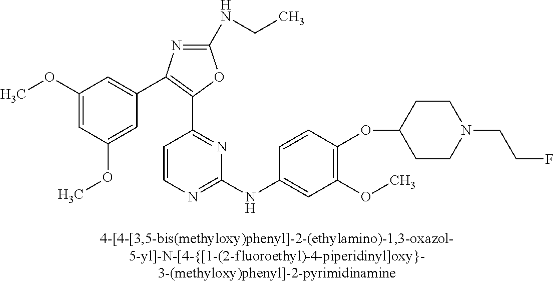 | ¹H NMR (400 MHz, DMSO-d₆) δ ppm 9.29 (s, 1H), 8.37 (d, J = 5.2 Hz, 1H), 7.85 (t, J = 5.6 Hz, 1H), 7.30 (d, J = 2.4 Hz, 1H), 7.06 (dd, J = 8.7, 2.4 Hz, 1H), 6.97 (d, J = 2.3 Hz, 2H), 6.76 (d, J = 5.2 Hz, 1H), 6.65 (d, J = 8.7 Hz, 1H), 6.46 (t, J = 2.3 Hz, 1H), 4.59 (t, J = 4.9 Hz, 1H), 4.47 (t, J = 4.9 Hz, 1H), 4.05 (brs, 1H), 3.63 (s, 6H), 3.51 (s, 3H), 3.34 (s, 2H), 2.74-2.81 (m, 2H), 2.66 (t, J = 4.9 Hz, 1H), 2.59 (t, J = 4.9 Hz, 1H), 2.20- 2.28 (m, 2H), 1.83-1.91 (m, 2H), 1.54-1.65 (m, 2H), and 1.20 (t, J = 7.2 Hz, 3H) |
| 97 | 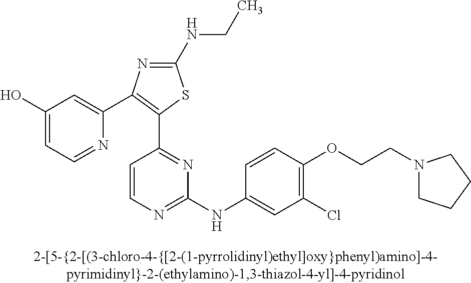 | ¹H NMR (400 MHz, DMS0-d₆) δ 9.45 (brs, 1H), 8.03-8.28 (m, 3H), 7.52 (d, J = 8.9 Hz, 1H), 7.07 (d, J = 8.8 Hz, 2H), 6.83 (brs, 1H), 6.59 (brs, 1H), 6.23 (brs, 1H), 4.11 (t, J = 5.2 Hz, 2H), 2.85 (s, 2H), 2.60 (s, 4H), 1.70 (s, 4H), and 1.20 (q, J = 7.2 Hz, 3H) |
| 98 | 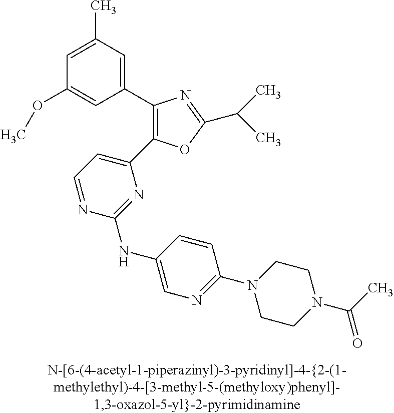 | ¹H NMR (400 MHz, DMSO-d₆) δ ppm 9.47 (s, 1H), 8.47 (d, J = 5.1 Hz, 1H), 8.28, (brs, 1H), 7.72 (d, J = 7.5 Hz, 1H), 7.20 (s, 1H), 7.10 (s, 1H), 6.95 (d, J = 5.1 Hz, 1H), 6.79 (s, 1H), 6.57 (d, J = 10.5 Hz, 1H), 3.67 (s, 3H), 3.54 (d, J = 3.1 Hz, 4H), 3.38-3.47 (m, 2H), 3.29-3.38 (m, 2H), 3.21 (dt, J = 13.9 and 6.9 Hz, 1H), 2.22 (s, 3H), 2.05 (s, 3H), and 1.37 (d, J = 7.0 Hz, 6H) |
| 99 | 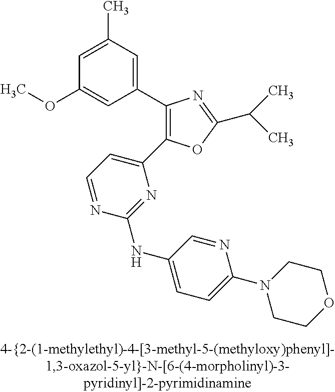 | ¹H NMR (400 MHz, DMSO-d₆) δ ppm 9.47 (s, 1H), 8.47 (d, J = 5.1 Hz, 1H), 8.28 (brs, 1H), 7.73 (d, J = 8.1 Hz, 1H), 7.20 (s, 1H), 7.10 (s, 1H), 6.95 (d, J = 5.1 Hz, 1H), 6.79 (s, 1H), 6.54 (d, J = 7.5 Hz, 1H), 3.69-3.74 (m, 4H), 3.68 (s, 3H), 3.28-3.35 (m, 4 H), 3.21 (dt, J = 13.9 and 6.9 Hz, 1H), 2.22 (s, 3H), and 1.38 (d, J = 7.0 Hz, 6H) |
| 100 | 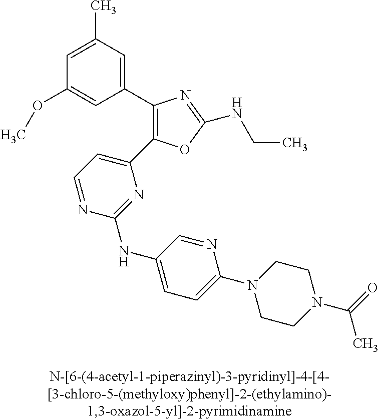 | ¹H NMR (400 MHz, DMSO-d₆) δ ppm 9.25 (s, 1H), 8.38 (d, J = 5.1 Hz,. 1H), 8.21 (s, 1H), 7.93 (t, J = 5.6 Hz, 1H), 7.65 (dd, J = 9.0 and 2.2 Hz, 1H), 7.39 (s, 1H), 7.33 (brs, 1H), 7.00 (t, J = 2.1 Hz, 1H), 6.76 (d, J = 5.1 Hz, 1H), 6.54 (d, J = 9.0 Hz, 1H), 3.68 (s, 3H), 3.50-3.60 (m, 4H), 3.39-3.46 (m, 2H), 3.24-3.39 (m, 4H), 2.06 (s, 3H), and 1.20 (t, J = 7.2 Hz, 3H) |
| 101 | 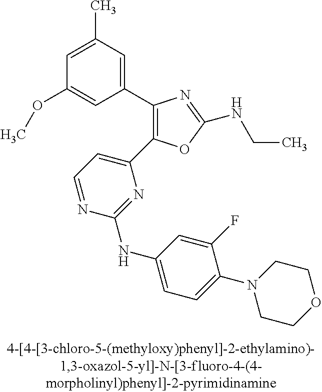 | ¹H NMR (400 MHz, DMSO-d₆) δ ppm 9.51 (s, 1H), 8.43 (d, J = 5.1 Hz, 1H), 7.95 (t, J = 5.7 Hz, 1H), 7.36-7.47 (m, 2H), 7.33 (s, 1H), 7.19 (dd, J = 8.7 and 1.9 Hz, 1H), 7.01 (t, J = 2.1 Hz, 1H), 6.81 (d, J = 5.1 Hz, 1H), 6.73 (t, J = 9.4 Hz, 1H), 3.69-3.77 (m, 4H), 3.65 (s, 3H), 3.32-3.38 (m, 2H), 2.88-2.93 (m, 4H), and 1.20 (t, J = 7.2 Hz, 3H) |
| 102 | 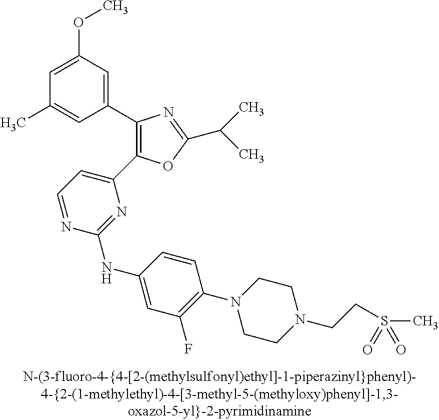 | ¹H NMR (400 MHz, DMSO-d₆) δ ppm 9.71 (s, 1H), 8.52 (d, J = 5.1 Hz, 1H), 7.55 (dd, J = 15.4 and 2.2 Hz, 1H), 7.23 (d, J = 9.0 Hz, 1H), 7.20 (s, 1H), 7.10 (s, 1H), 6.99 (d, J = 5.1 Hz, 1H), 6.71- 6.81 (m, 2H), 3.66 (s, 3H), 3.31- 3.36 (m, 2H), 3.22 (dt, J = 13.9 and 7.0 Hz, 1H), 3.06 (s, 3H), 2.93 (brs, 4H), 2.77 (t, J = 6.7 Hz, 2H), 2.60 (brs, 4H), 2.21 (s, 3H), and 1.39 (d, J = 7.0 Hz, 6H) |
| 103 | 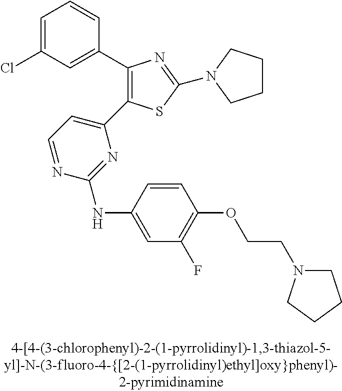 | ¹H NMR (400 MHz, DMSO-d₆) δ ppm 9.55 (s, 1H), 8.12 (d, J = 5.3 Hz, 1H), 7.78 (dd, J = 14.4 and 2.3 Hz, 1H), 7.43-7.60 (m, 4H), 7.37 (d, J = 8.6 Hz, 1H), 7.09 (t, J = 9.4 Hz, 1H), 6.23 (d, J = 5.3 Hz, 1H), 4.09 (t, J = 5.9 Hz, 2H), 3.47 (brs, 4H), 2.78 (t, J = 5.8 Hz, 2H), 2.50 (brs, 4H), 2.02 (brs, 4H), and 1.68 (brs, 4H) |
| 104 | 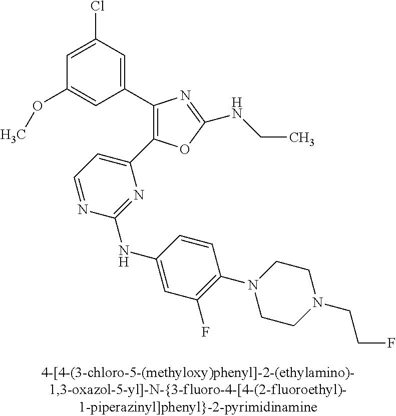 | ¹H NMR (400 MHz, DMSO-d₆) δ ppm 9.49 (s, 1H), 8.43 (d, J = 5.3 Hz, 1H), 7.95 (t, J = 5.8 Hz, 1H), 7.30-7.43 (m, 3H), 7.18 (dd, J = 8.6 and 1.8 Hz, 1H), 7.00 (t, J = 2.1 Hz, 1H), 6.75-6.84 (m, 1H), 6.71 (t, J = 9.2 Hz, 1H), 4.63 (t, J = 5.0 Hz, 1H), 4.51 (t, J = 4.9 Hz, 1H), 3.65 (s, 3H), 3.31-3.37 (m, 2H), 2.88-2.96 (m, 4H), 2.71 (t, J = 4.9 Hz, 1H), 2.61-2.66 (m,1H), 2.60 (brs, 4H), and 1.20 (t, J = 7.2 Hz, 3H) |
| 105 | 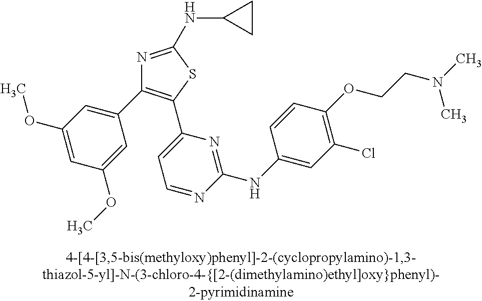 | ¹H NMR (400 MHz, DMSO-d₆) δ ppm 9.52 (s, 1H), 8.65 (s, 1H,), 8.17 (brs, 1H), 8.13 (d, J = 5.3 Hz, 1H,), 7.47 (dd, J = 9.0 and 2.2 Hz, 1H), 7.09 (d, J = 9.0 Hz, 1H), 6.63 (d, J = 2.2 Hz, 2H), 6.58 (t, J = 2.3 Hz, 1H), 6.35 (d, J = 5.5 Hz, 1H), 4.09 (t, J = 5.8 Hz, 2H), 3.74 (s, 6H), 2.65 (t, J = 5.7 Hz, 2H), 2.56-2.63 (m, 1H), 2.25 (s, 6H), 0.76-0.85 (m, 2H), and 0.56-0.65 (m, 2H) |
| 106 | 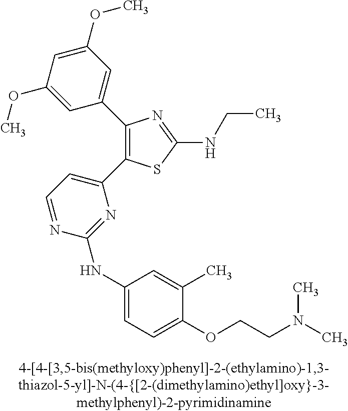 | ¹H NMR (400 MHz, DMSO-d₆) δ ppm 9.21 (s, 1H), 8.22 (t, J = 5.2 Hz, 1H), 8.07 (d, J = 5.3 Hz, 1H), 7.62 (brs, 1H), 7.42 (d, J = 11.0 Hz, 1H), 6.84 (d, J = 9.0 Hz, 1H), 6.62 (d, J = 2.2 Hz, 2H), 6.57 (t, J = 2.0 Hz, 1H), 6.28 (d, J = 5.3 Hz, 1H), 4.01 (t, J = 5.8 Hz, 2H), 3.74 (s, 6H), 3.24-3.32 (m, 2H), 2.64 (t, J = 5.8 Hz, 2H), 2.24 (s, 6H), 2.17 (s, 3H), and 1.20 (t, J =7.1 Hz, 3H,) |
| 107 | 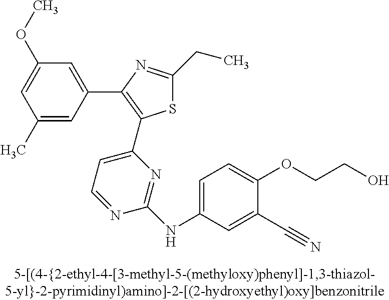 | ¹H NMR (400 MHz, DMSO-d₆) δ ppm 9.85 (s, 1H), 8.34 (d, J = 5.1 Hz, 1H), 8.19 (d, J = 2.2 Hz, 1H), 7.83 (dd, J = 9.3 and 2.3 Hz, 1H), 7.22 (d, J = 9.2 Hz, 1H), 6.95 (s, 1H), 6.86 (s, 2H), 6.55 (d, J = 5.1 Hz, 1H), 4.93 (t, J = 5.2 Hz, 1H), 4.13 (t, J = 4.9 Hz, 2H), 3.62-3.83 (m, 5H), 3.04 (q, J = 7.5 Hz, 2H), 2.31 (s, 3H), and 1.37 (t, J = 7.5 Hz, 3H) |
| 108 | 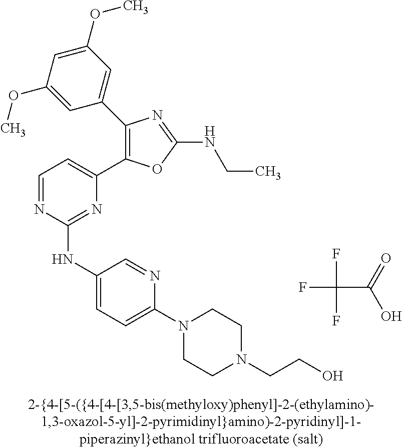 | ¹H NMR (400 MHz, METHANOL- d₄) δ ppm 8.13-8.27 (m, 2H), 7.68 (dd, J = 9.2 and 2.8 Hz, 1H), 6.97 (d, J = 6.2 Hz, 1H), 6.83 (d, J = 2.2 Hz, 2H), 6.76 (d, J = 9.2 Hz, 1H), 6.49 (t, J = 2.2 Hz, 1H), 3.89-4.00 (m, 2H), 3.70 (s, 6H), 3.54 (brs, 4H), 3.48 (q, J = 7.3 Hz, 2H), 3.35-3.42 (m, 2H), and 1.31 (t, J = 7.2 Hz, 3H) |
| 109 | 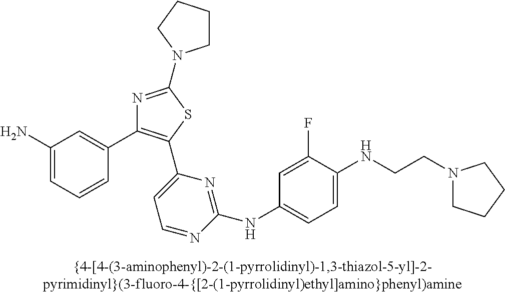 | ¹H NMR (400 MHz, DMSO-d₆) δ ppm 9.45 (s, 1H), 8.05 (d, J = 5.5 Hz, 1H), 7.78 (d, J = 2.4 Hz, 1H), 7.35 (d, 1H), 7.01-7.15 (m, 2H), 6.59-6.67 (m, 2H), 6.55 (d, J = 7.1 Hz, 1H), 6.30 (d, J = 5.5 Hz, 1H), 5.22 (s, 2H), 4.07 (s, 2H), 3.44 (brs, 4H), 2.76 (brs, 2H), 2.49-2.55 (m, 4H), 2.00 (brs, 4H), and 1.67 (brs, 4H) |
| 110 | 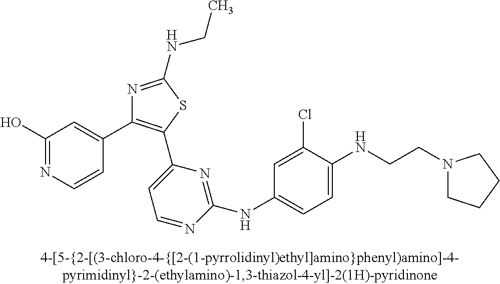 | ¹H NMR (400 MHz, DMSO-d₆) δ ppm 11.64 (brs, 1H), 9.50 (s, 1H) 8.28 (t, J = 5.2 Hz, 1H), 8.19 (d, J = 5.5 Hz, 1H), 7.96 (d, J = 2.4 Hz, 1H), 7.48 (dd, J = 9.0 and 2.6 Hz, 1H), 7.38 (d, J = 6.6 Hz, 1H), 7.03 (d, J = 9.0 Hz, 1H), 6.55 (d, J = 5.3 Hz, 1H), 6.38 (s, 1H), 6.17 (dd, J = 6.7 and 1.4 Hz, 1H), 4.06 (t, J = 5.9 Hz, 2H), 3.19-3.26 (m, 2H), 2.77 (brs, 2H), 2.49-2.56 (m, 4H), 1.64 (brs, 4H), and 1.15 (t, J = 7.1 Hz, 3H) |
| 111 | 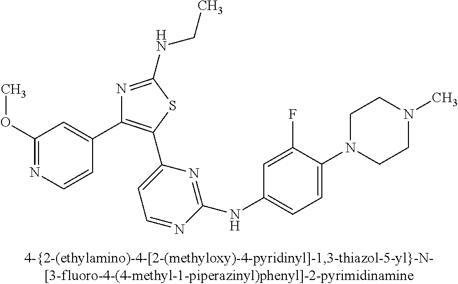 | ¹H NMR (400 MHz, DMSO-d₆) δ ppm 9.51 (s, 1H), 8.28 (t, J = 5.2 Hz, 1H), 8.19 (d, J = 5.3 Hz, 1H), 8.12 (d, J = 5.3 Hz, 1H), 7.65 (dd, J = 15.6 and 2.4 Hz, 1H), 7.30 (dd, J = 8.6 and 1.8 Hz, 1H), 7.05 (dd, J = 5.2 and 1.2 Hz, 1H), 6.79- 6.95 (m, 2H), 6.32 (d, J = 5.3 Hz, 1H), 3.84 (s, 3H) 3.20-3.26 (m, 2H), 2.90 (brs, 4H), 2.42 (brs, 4H), 2.18 (s, 3H), and 1.15 (t, J = 7.1 Hz, 3H) |
| 112 | 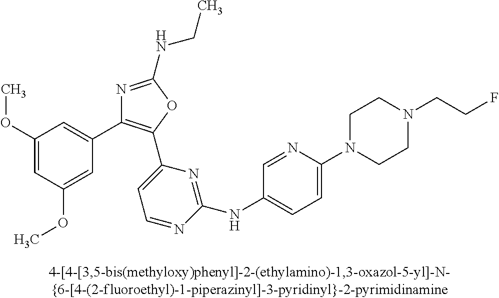 | ¹H NMR (400 MHz, DMSO-d₆) δ ppm 9.16 (s, 1H), 8.27 (d, J = 5.3 Hz, 1H), 8.21 (d, J = 2.4 Hz, 1H), 7.81 (t, J = 5.6 Hz, 1H), 7.68 (dd, J = 9.0, 2.4 Hz, 1H), 6.86 (d, J = 1.1 Hz, 2H), 6.67 (d, J = 5.3 Hz, 1H), 6.41-6.55 (m, 2H), 4.59 (t, J = 4.8 Hz, 1H), 4.47 (t, J = 4.9 Hz, 1H), 3.63 (s, 6H), 3.28-3.34 (m, 6H), 2.65 (t, J = 4.9 Hz, 1H), 2.58 (t, J = 4.9 Hz, 1H), 2.47-2.54 (m, 4H), and 1.14 (t, J = 7.1 Hz, 3H) |
| 113 | 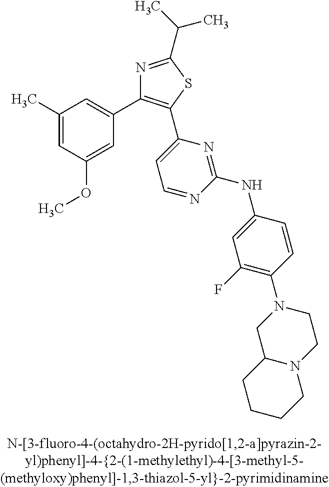 | ¹H NMR (400 MHz, DMSO-d₆) δ ppm 9.68 (s, 1H), 8.28 (d, J = 5.1 Hz, 1H), 7.66 (dd, J = 15.6 and 2.2 Hz, 1H), 7.26-7.41 (m, 1H), 6.75-6.97 (m, 4H), 6.49 (d, J = 5.1 Hz, 1H), 3.69 (s, 4H), 3.10- 3.20 (m, 1H), 3.05 (d, J = 11.2 Hz, 1H), 2.64-2.80 (m, 4H), 2.36 (t, J = 10.4 Hz, 1H), 2.19- 2.32 (m, 5H), 1.86-2.07 (m, 2H), 1.62-1.72 (m, 1H), 1.42-1.59 (m, 3H), and 1.36 (d, J = 6.8 Hz, 6H). |
| 114 | 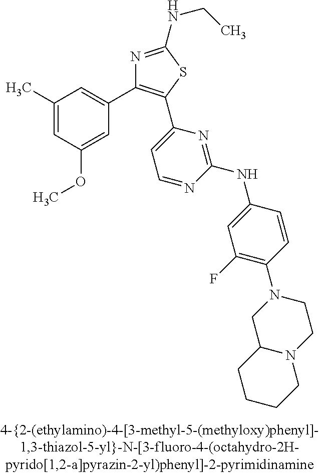 | ¹H NMR (400 MHz, DMSO-d₆) δ ppm 9.45 (s, 1H), 8.14-8.29 (m, 1H), 8.04 (d, J = 5.3 Hz, 1H), 7.69 (dd, J = 15.9 and 1.8 Hz, 1H), 7.34 (dd, J = 9.0 and 1.6 Hz, 1H), 6.70-6.99 (m, 3H), 6.25 (d, J = 5.3 Hz, 1H), 3.70 (s, 3H), 2.99- 3.19 (m, 2H), 2.58-2.79 (m, 4H), 2.32-2.41 (m, 2H), 2.18-2.31 (m, 5H), 1.86-2.07 (m, 2H), 1.37- 1.71 (m, 4H), and 1.06-1.28 (m, 5H). |
| 115 | 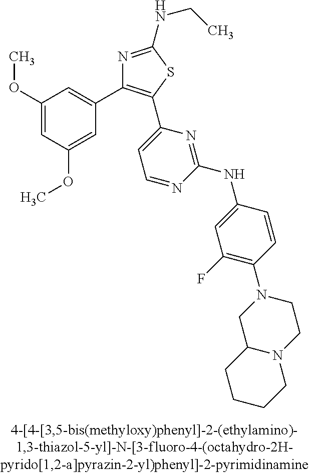 | ¹H NMR (400 MHz, DMSO-d₆) δ ppm 9.45 (s, 1H), 8.23 (t, J = 5.2 Hz, 1H), 8.07 (d, J = 5.5 Hz, 1H), 7.69 (dd, J = 15.7 and 2.2 Hz, 1H), 7.34 (dd, J = 8.9 and 1.9 Hz, 1H), 6.89 (t, J = 9.4 Hz, 1H), 6.50-6.63 (m, 3H), 6.29 (d, J = 5.5 Hz, 1H), 3.62-3.79 (m, 6H), 3.14 (d, J = 9.9 Hz,1H), 3.05 (d, J = 10.8 Hz, 1H), 2.65-2.80 (m, 4H), 2.36 (t, J = 10.5 Hz, 1H), 2.25 (td, J = 11.4 and 2.1 Hz, 1H), 1.89-2.04 (m, 3H), 1.61-1.72 (m, 1H), 1.39- 1.61 (m, 4H), and 1.16 (t, J = 7.1 Hz, 4H) |
| 116 | 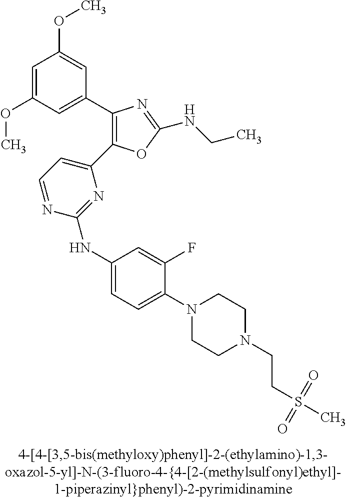 | ¹H NMR (400 MHz, DMSO-d₆) δ ppm 9.59 (s, 1H), 8.39 (d, J = 5.3 Hz, 1H), 7.93 (t, J = 5.6 Hz, 1H), 7.50-7.59 (m, 1H), 7.29 (d, J = 9.2 Hz, 1H), 6.93 (d, J = 1.8 Hz, 2H), 6.77-6.88 (m, 2H), 6.51 (s, 1H), 3.57-3.74 (m, 14H), 3.34 (dt, J = 13.2 and 6.7 Hz, 6H), 2.92- 3.01 (m, 3H), and 1.20 (t, J = 7.1 Hz, 3H) |
| 117 | 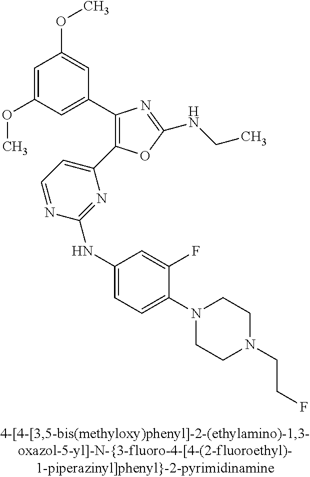 | ¹H NMR (400 MHz, DMSO-d₆) δ ppm 9.77-9.91 (m, 1H), 9.56 (s, 1H), 8.37 (d, J = 5.1 Hz, 1H), 7.90 (t, J = 5.2 Hz, 1H), 7.51 (d, J = 15.4 Hz, 1H), 7.26 (d, J = 8.8 Hz, 1H), 6.90 (s, 2H), 6.74-6.83 (m, 2H), 6.49 (brs, 1H), 4.74-4.96 (m, 3H), 3.49-3.68 (m, 9H), 3.23- 3.40 (m, 5H), 2.93-3.05 (m, 2H), and 1.17 (t , J = 7.1 Hz, 3H) |
| 118 | 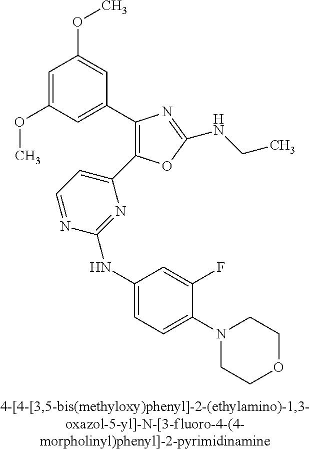 | ¹H NMR (400 MHz, DMSO-d₆) δ ppm 9.51 (s, 1H), 8.38 (d, J = 5.3 Hz, 1H), 7.90 (brs, 1H), 7.47- 7.55 (m, 1H), 7.28 (d, J = 8.6 Hz, 1H), 6.92 (d, J = 1.8 Hz, 2H), 6.71-6.82 (m, 2H), 6.52 (s, 1H), 3.70-3.77 (m, 5H), 3.66 (s, 7H), 2.87-2.95 (m, 4H), and 1.20 (t, J = 7.1 Hz, 3H) |
| 119 | 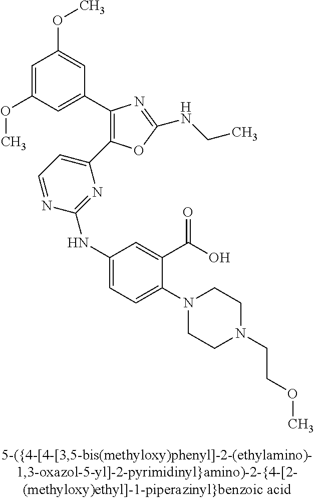 | ¹H NMR (400 MHz, DMSO-d₆) δ ppm 9.26 (s, 1H), 8.25-8.35 (m, 2H), 7.88 (d, J = 0.9 Hz, 1H), 7.76 (dd, J = 8.9 and 2.1 Hz, 1H), 6.92 (s, 2H), 6.73 (d, J = 5.3 Hz, 1H), 6.58 (d, J = 9.3 Hz, 1H), 6.52 (s, 1H), 3.75-3.84 (m, 2H), 3.68 (s, 8H), 3.58 (d, J = 1.3 Hz, 3H), 3.45 (d, J = 1.6 Hz, 4H), 1.97-2.09 (m, 1H), 1.15-1.27 (m, 4H), and 0.69-0.78 (m, 4H) |
| 120 | 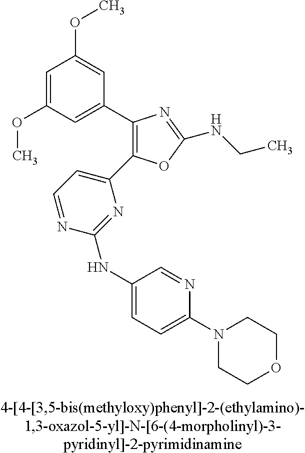 | ¹H NMR (400 MHz, DMSO-d₆) δ ppm 9.26 (s, 1H), 8.32 (d, J = 5.1 Hz, 1H), 8.29 (d, J = 2.2 Hz, 1H), 7.88 (brs, 1H), 7.76 (dd, J = 9.0 and 2.2 Hz, 1H), 6.91 (s, 2H), 6.73 (d, J = 5.3 Hz, 1H), 6.49- 6.58 (m, 2H), 3.69-3.75 (m, 4H), 3.68 (s, 6H), 3.28-3.36 (m, 6H), and 1.19 (t, J = 7.1 Hz, 3H) |
| 121 | 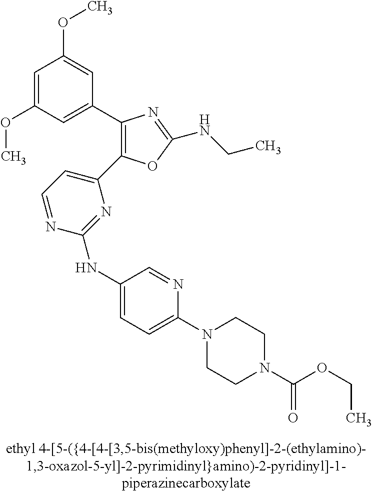 | ¹H NMR (400 MHz, DMSO-d₆) δ ppm 9.26 (s, 1H), 8.56 (s, 1H), 8.32 (d, J = 5.1 Hz, 1H), 8.28 (s, 1H), 7.85-7.93 (m, 1H), 7.73- 7.82 (m, 1H), 7.20 (d, J = 2.7 Hz, 1H), 6.91 (s, 1H), 6.72 (d, J = 5.1 Hz, 1H), 6.55-6.60 (m, 1H), 6.52 (s, 1H), 4.07 (q, J = 7.0 Hz, 2H), 3.68 (s, 6H), 3.44-3.52 (m, 6H), 3.37 (d, J = 5.7 Hz, 3H), and 1.13-1.26 (m, 6H) |
| 122 | 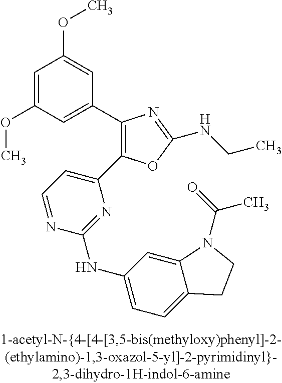 | ¹H NMR (400 MHz, DMSO-d₆) δ ppm 8.56 (s, 1H), 8.37 (d, J = 5.1 Hz, 1H), 7.91 (brs, 1H), 7.77 (d, J = 8.6 Hz, 1H), 7.48 (brs, 1H), 7.30 (d, J = 8.4 Hz, 1H), 6.93 (s, 2H), 6.76 (d, J = 5.1 Hz, 1H), 6.52 (brs, 1H), 4.02 (t, J = 8.2 Hz, 3H), 3.66 (s, 4H), 3.14-3.21 (m, 3H), 2.91 (t, J = 8.1 Hz, 2H), 2.12 (s, 3H), and 1.20 (t, J = 7.1 Hz, 3H) |
| 123 | 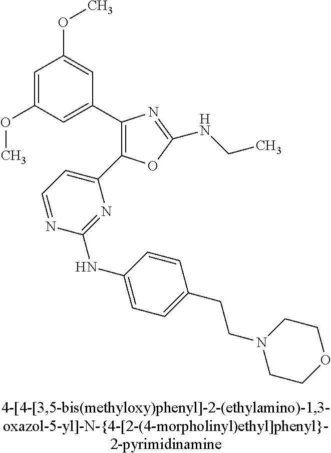 | ¹H NMR (400 MHz, DMSO-d₆) δ ppm 9.37 (s, 1H), 8.36 (d, J = 5.1 Hz, 1H), 7.89 (brs, 1H), 7.47 (d, J = 8.2 Hz, 2H), 6.88-6.95 (m, 4H), 6.76 (d, J = 5.3 Hz, 1H). 6.53 (s, 1H), 3.64-3.72 (m, 6H), 3.57 (d, J = 3.7 Hz, 6H), 2.58-2.69 (m, 3H), 2.35-2.46 (m, 5H), and 1.20 (t, J = 7.1 Hz, 3H) |
| 124 | 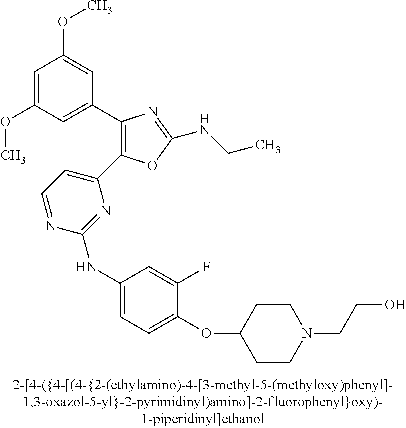 | ¹H NMR (400 MHz, DMSO-d₆) δ ppm 9.51 (s, 1H), 8.37 (d, J = 5.3 Hz, 1H), 7.90 (d, J = 4.6 Hz, 1H), 7.53 (dd, J = 13.2 and 1.5 Hz, 1H), 7.18-7.27 (m, 2H), 7.11 (s, 1H), 6.80-6.89 (m, 1H), 6.73-6.79 (m, 2H), 4.09-4.18 (m, 1H), 3.63- 3.72 (m, 4H), 3.49 (t, J = 6.3 Hz, 2H), 3.17 (s, 3H), 2.68-2.78 (m, 2H), 2.40 (t, J = 6.4 Hz, 2H), 2.17-2.25 (m, 5H), 1.82-1.93 (m, 2H), and 1.20 (t, J = 7.2 Hz, 3H) |
| 125 | 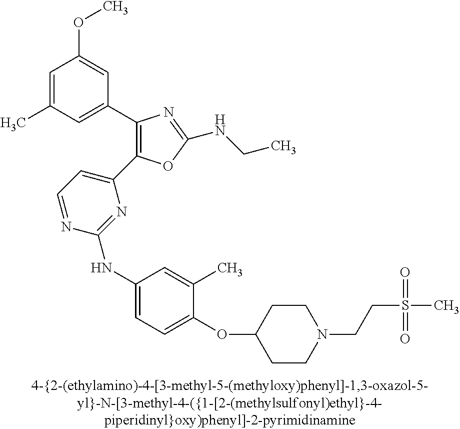 | ¹H NMR (400 MHz, DMSO-d₆) δ ppm 9.19 (s, 1H), 8.34 (d, J = 5.3 Hz, 1H), 7.86 (d, J = 1.8 Hz, 1H), 7.26-7.34 (m, 2H), 7.24 (s, 1H), 7.14 (s, 1H), 6.70-6.76 (m, 2H), 6.60 (d, J = 8.6 Hz, 1H), 3.63 (s, 3H), 3.17 (s, 5H), 3.04 (s, 3H), 2.72 (t, J = 6.7 Hz, 4H), 2.28-2.37 (m, 2H), 2.17 (s, 3H), 1.96 (s, 3H), 1.85-1.93 (m, 2H), 1.61-1.69 (m, 2H), and 1.19 (t, J = 7.1 Hz, 3H) |
| 126 | 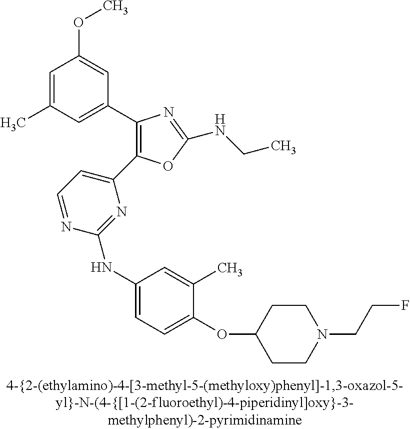 | ¹H NMR (400 MHz, DMSO-d₆) δ ppm 9.19 (s, 1H), 8.34 (d, J = 5.1 Hz, 1H), 7.86 (brs, 1H), 7.25-7.33 (m, 2H), 7.24 (s, 1H), 7.14 (brs, 1H), 6.69-6.75 (m, 2H), 6.60 (d, J = 8.6 Hz, 1H), 4.59 (t, J = 4.8 Hz, 1H), 4.47 (t, J = 4.7 Hz, 1H), 4.19 (brs, 1H), 3.63 (s, 3H), 3.17 (s, 3H), 2.64-2.75 (m, 2H), 2.60 (t, J = 4.6 Hz, 1H), 2.30-2.39 (m, 2H), 2.17 (s, 3H), 1.96 (s, 3H), 1.90 (dd, J = 10.8 and 1.6 Hz, 2H), 1.60-1.70 (m, 2H), and 1.19 (t, J = 7.1 Hz, 3H) |
| 127 | 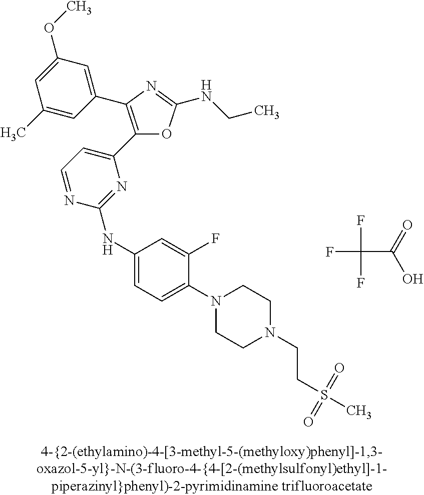 | ¹H NMR (400 MHz, DMSO-d₆) δ ppm 9.59 (s, 1H), 8.38 (d, J = 5.3 Hz, 1H), 7.92 (t, J = 5.4 Hz, 1H), 7.52 (d, J = 15.0 Hz, 1H), 7.27 (d, J = 8.8 Hz, 1H), 7.20 (s, 1H), 7.11 (s, 1H), 6.78 (d, J = 5.9 Hz, 3H), 3.71 (d, J = 7.3 Hz, 3H), 3.64 (s, 6H), 3.31-3.39 (m, 5H), 3.14 (s, 4H), 2.54 (s, 2H), 2.19 (s, 3H), and 1.20 (t, J = 7.1 Hz, 3H) |
| 128 | 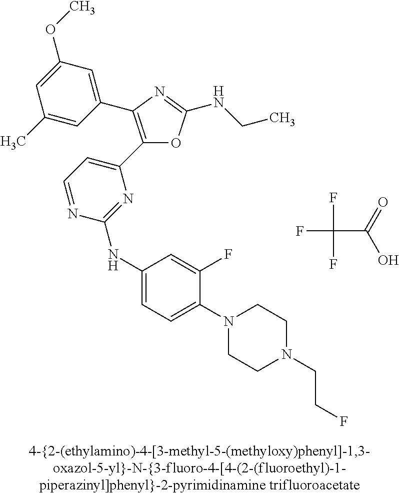 | ¹H NMR (400 MHz, DMSO-d₆) δ ppm 9.58 (s, 1H), 8.39 (d, J = 5.1 Hz, 1H), 7.91 (t, J = 5.5 Hz, 1H), 7.51 (d, J = 14.8 Hz, 1H), 7.28 (d, J = 8.4 Hz, 1H), 7.20 (s, 1H), 7.11 (s, 1H), 6.75-6.82 (m, 3H), 4.90- 4.98 (m, 1H), 4.81-4.86 (m, 1H), 3.52-3.68 (m, 7H), 3.35 (td, J = 13.1 and 7.0 Hz, 6H), 2.96-3.07 (m, 2H), 2.19 (s, 3H), and 1.20 (t, J = 7.1 Hz, 3H) |
| 129 | 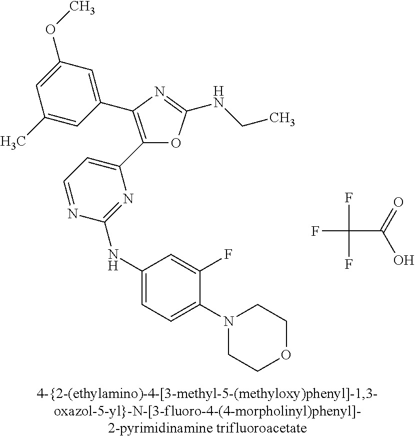 | ¹H NMR (400 MHz, DMSO-d₆) δ ppm 9.54 (s, 1H), 8.36 (d, J = 5.3 Hz, 1H), 7.94 (t, J = 5.1 Hz, 1H), 7.47 (d, J = 15.0 Hz, 1H), 7.26 (d, J = 8.6 Hz, 1H), 7.19 (s, 1H), 7.10 (s, 1H), 6.67-6.81 (m, 3H), 3.69- 3.76 (m, 4H), 3.65 (s, 3H), 3.29- 3.40 (m, J = 6.9, 6.6, 6.5, and 6.5 Hz, 2H), 2.91 (d, J = 3.8 Hz, 4H), 2.19 (s, 3H), and 1.20 (t, J = 7.1 Hz, 3H) |
| 130 | 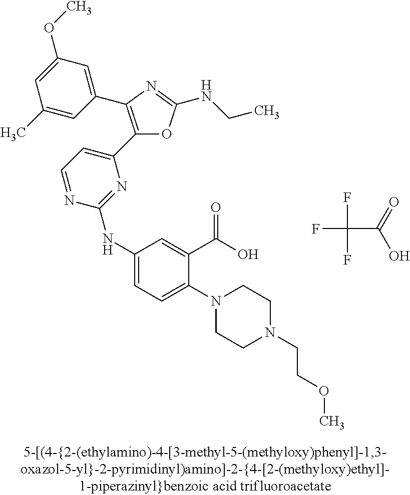 | ¹H NMR (400 MHz, DMSO-d₆) δ ppm 9.70 (s, 1H), 8.40 (d, J = 5.3 Hz, 1H), 8.02 (s, 1H), 7.84-7.94 (m, 2H), 7.20 (s, 1H), 7.11 (s, 1H), 6.94 (d, J = 8.8 Hz, 1H), 6.78-6.83 (m, 2H), 3.71 (d, J = 4.2 Hz, 2H), 3.61-3.66 (m, 5H), 3.45 (brs, 2H), 3.15-3.38 (m, 11H), 2.19 (s, 3H), and 1.20 (t, J = 7.1 Hz, 3H) |
| 131 | 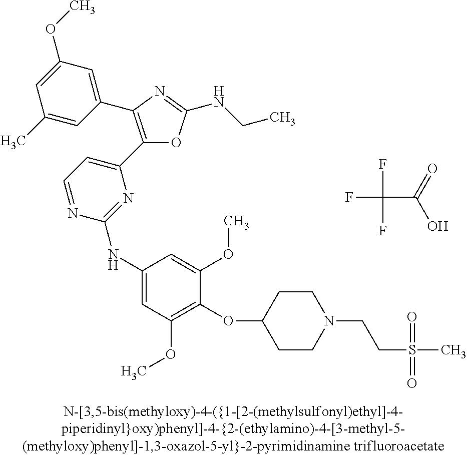 | ¹H NMR (400 MHz, DMSO-d₆) δ ppm 9.43 (brs, 1H), 8.41 (d, J = 5.1 Hz, 1H), 7.83-7.88 (m, 1H), 7.39 (s, 1H), 7.27 (s, 1H), 7.01- 7.09 (m, 2H), 6.80 (d, J = 5.1 Hz, 1H), 6.69 (br. s., 1H), 3.70 (d, J = 7.9 Hz, 3H), 3.51-3.63 (m, 6H), 3.42-3.51 (m, 8H), 3.29-3.40 (m, 3H), 3.11-3.16 (m, 3H), 2.12 (s, 4H), 1.95-2.05 (m, 1H), 1.87-1.94 (m, 1H), and 1.20 (t, J = 7.1 Hz, 3H) |
| 132 | 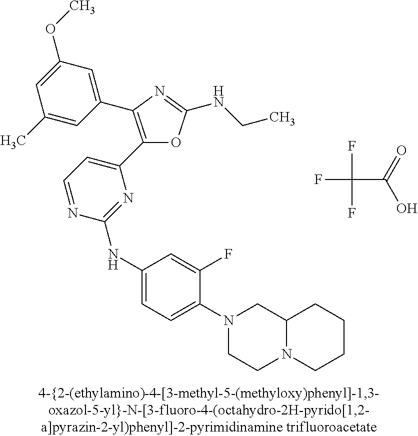 | ¹H NMR (400 MHz, DMSO-d₆) δ ppm 9.58 (s, 1H), 8.38 (d, J = 5.3 Hz, 1H), 7.91 (t, J = 5.6 Hz, 1H), 7.51 (d, J = 15.0 Hz, 1H), 7.28 (d, J = 8.6 Hz, 1H), 7.20 (s, 1H), 7.11 (s, 1H), 6.73-6.84 (m, 3H), 3.64 (s, 3H), 3.30-3.52 (m, 8H), 3.25 (d, J = 11.9 Hz, 1H), 2.92-3.13 (m, 2H), 2.76 (t, J = 11.8 Hz, 1H), 2.19 (s, 3H), 1.82-1.93 (m, 2H), 1.74-1.82 (m, 1H), 1.43-1.58 (m, 2H), and 1.20 (t, J = 7.1 Hz, 3H) |
| 133 | 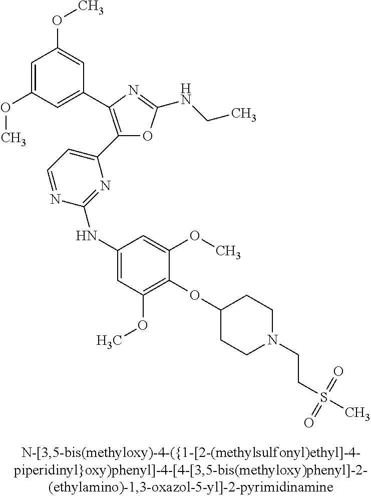 | ¹H NMR (400 MHz, DMSO-d₆) δ ppm 9.35 (s, 1H), 8.40 (d, J = 5.1 Hz, 1H), 7.85 (brs, 1H), 6.98 -7.10 (m, 4H), 6.79 (d, J = 5.3 Hz, 1H), 6.42 (s, 1H), 3.59 (s, 6H), 3.48 (s, 6H), 3.27 (t, J = 6.7 Hz, 3H), 3.16 (d, J = 4.0 Hz, 2H), 3.03 (s, 3H), 2.75-2.82 (m, 2H), 2.69 (t, J = 6.6 Hz, 2H), 2.13 (t, J = 9.1 Hz, 2H), 1.74-1.82 (m, 2H), 1.56-1.66 (m, 2H), and 1.19 (t , J = 7.1 Hz, 3H) |
| 134 | 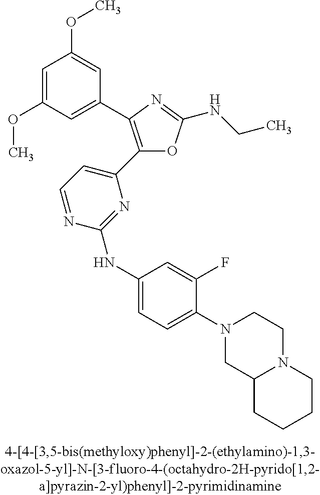 | ¹H NMR (400 MHz, DMSO-d₆) δ ppm 9.48 (s, 1H), 8.37 (d, J = 5.3 Hz, 1H), 7.92 (brs, 1H), 7.46 (d, J = 15.6 Hz, 1H), 7.26 (d, J = 7.9 Hz, 1H), 6.91 (d, J = 1.3 Hz, 2H), 6.77 (d, J = 5.3 Hz, 1H), 6.71 (t, J = 9.3 Hz, 1H), 6.50 (s, 1H), 3.66 (s, 6H), 3.00-3.18 (m, 2H), 2.66-2.80 (m, 3H), 2.2 -2.40 (m, 2H), 1.94- 2.07 (m, 2H), 1.42-1.74 (m, 5H), and 1.11-1.29 (m, 6H) |
| 135 | 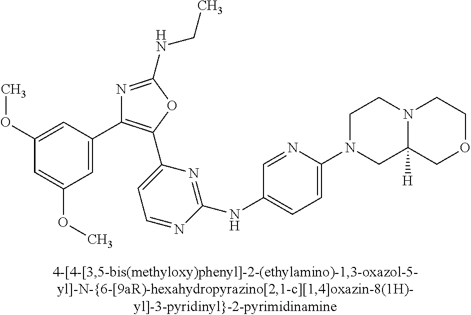 | ¹H NMR (400 MHz, DMSO-d₆) δ ppm 9.23 (s, 1H), 8.32 (d, J = 5.1 Hz, 1H), 8.25 (d, J = 1.5 Hz, 1H), 7.91 (d, J = 3.5 Hz, 1H), 7.69-7.77 (m, 1H), 6.91 (s, 1H), 6.72 (d, J = 5.3 Hz, 1H), 6.49-6.58 (m, 2H), 4.00 (d, J = 12.6 Hz, 1H), 3.88- 3.96 (m, 1H), 3.72-3.82 (m, 3H), 3.67 (s, 5H), 3.50-3.59 (m, 1H), 3.18 (t, J = 10.5 Hz, 1H), 2.75- 2.86 (m, 2H), 2.68 (d, J = 10.6 Hz, 1H), 2.12-2.34 (m, 4H), 1.50 (s, 2H), and 1.19 (t, J = 7.1 Hz, 3H) |

The following compounds may also be made using methods similar to those claimed above:

| Name | Structure |
|---|---|
| N-(3-chloro-4-{[3- (methyloxy)propyl]oxy}phenyl)-4-{2- (ethylamino)-4-[3-(methyloxy)phenyl]-1,3- thiazol-5-yl}-2-pyrimidinamine | 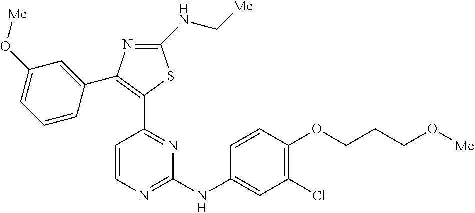 |
| 2-{[4-({4-[4-(3-chloro-5-methylphenyl)-2- (hydroxymethyl)-1,3-thiazol-5-yl]-2- pyrimidinyl}amino)-2- methylphenyl]oxy}ethanol | 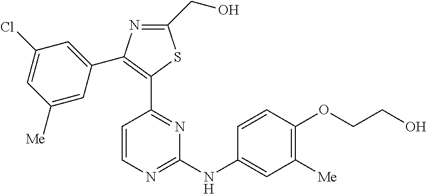 |
| 4-{2-ethyl-4-[3-methyl-5- (methyloxy)phenyl]-1,3-thiazol-5-yl}-N-(4- {[2-(ethyloxy)ethyl]oxy}-3-fluorophenyl)-2- pyrimidinamine | 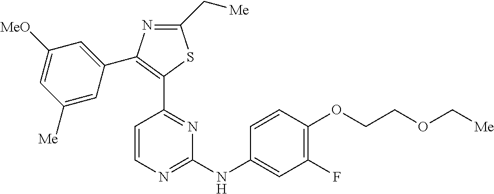 |
| 3-[5-{2-[(3-chloro-4-{[2- (methyloxy)ethyl]oxy}phenyl)amino]-4- pyrimidinyl}-2-(ethylamino)-1,3-thiazol-4- yl]-5-methylphenol | 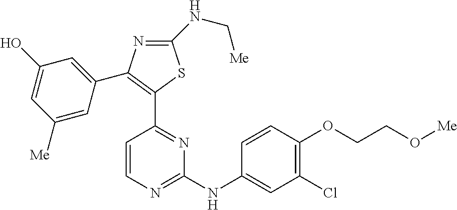 |
| N-[4-({2- [cyclohexyl(methyl)amino]ethyl}oxy)-3- fluorophenyl]-4-{2-(ethylamino)-4-[3- methyl-5-(methyloxy)phenyl]-1,3-thiazol-5- yl}-2-pyrimidinamine | 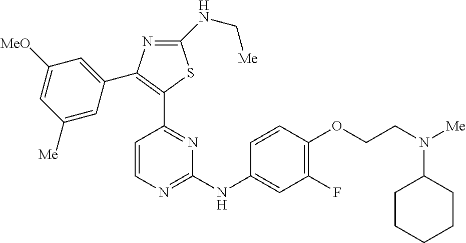 |
| N-(4-{[2-(cyclopentylamino)ethyl]oxy}-3- fluorophenyl)-4-{2-(ethylamino)-4-[3 (methyloxy)phenyl]-1,3-thiazol-5-yl}-2- pyrimidinamine | 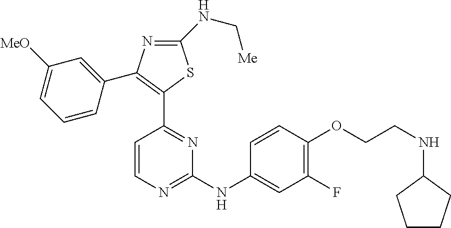 |
| ethyl{5-{2-[(3-methyl-4-{methyl[2-(4- morpholinyl)ethyl]amino}phenyl)amino]-4- pyrimidinyl}-4-[3-(methyloxy)phenyl]-1,3- thiazol-2-yl}amine | 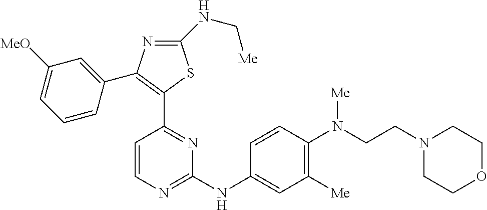 |
| (4-{4-[3-chloro-5-(methyloxy)phenyl]-2- ethyl-1,3-thiazol-5-yl}-2-pyrimidinyl)(3- methyl-4-{methyl[3-(4-methyl-1- piperazinyl)propyl]amino}phenyl)amine | 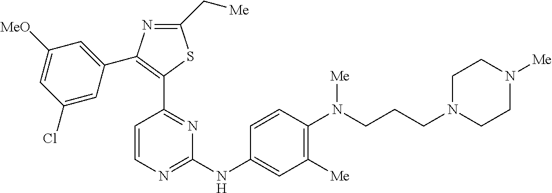 |
| N-(3-fluoro-4-{4-[2-(methylsulfonyl)ethyl]- 1-piperazinyl}phenyl)-4-[4-[3- (methyloxy)phenyl]-2-(4-methyl-1- piperazinyl)-1,3-thiazol-5-yl]-2- pyrimidinamine | 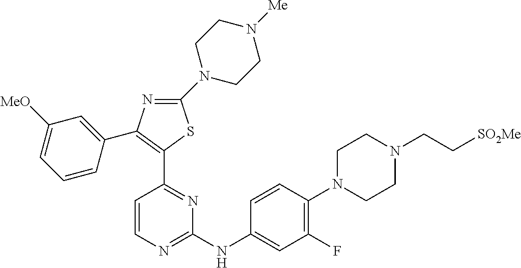 |
| N-[3-chloro-4-({1-[2-(methylsulfonyl)ethyl]- 4-piperidinyl}oxy)phenyl]-4-[4-[3-methyl-5- (methyloxy)phenyl]-2-(4-morpholinyl)-1,3- thiazol-5-yl]-2-pyrimidinamine | 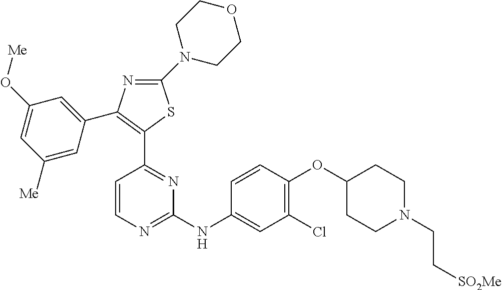 |
| 4-{2-[(cyclopropylmethyl)(methyl)amino]- 4-[3-(methyloxy)phenyl]-1,3-thiazol-5-yl}- N-[3-fluoro-4-({1-[2-(methylsulfonyl)ethyl]- 4-piperidinyl}oxy)phenyl]-2- pyrimidinamine | 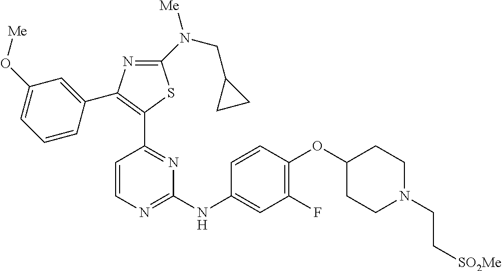 |
| N-({5-{2-[(3-fluoro-4-{4-[2- (methylsulfonyl)ethyl]-1- piperazinyl}phenyl)amino]-4-pyrimidinyl}- 4-[3-methyl-5-(methyloxy)phenyl]-1,3- thiazol-2-yl}methyl)ethanethioamide | 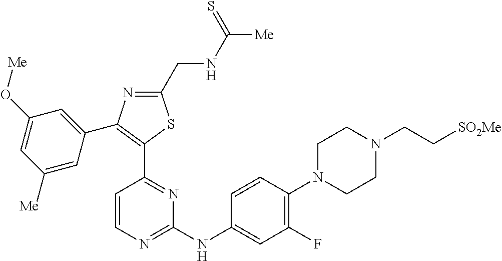 |

### BIOLOGICAL EXAMPLES

Compounds of the present invention were tested for B-RAF protein tyrosine kinase inhibitory activity in substrate phosphorylation assays and cell proliferation assays.

### A. Enzyme Assays:

Compounds of the present invention were tested for B-Raf protein tyrosine kinase inhibitory activity in a B-Raf Accelerated MEK ATPase assay (BRAMA). The BRAMA assay is a high sensitivity assay which measures an intrinsic MEK-mediated ATP hydrolysis uncoupled from downstream ERK phosphorylation by coupling the formation of ADP to NADH oxidation through the enzymes pyruvate kinase and lactate dehydrogenase. When ADP production is initiated by addition of catalytic amounts of an activated RAF enzyme and non-phosphorylated MEK, one observes robust ADP production concomitant with RAF-mediated phosphorylation of MEK. Specific details of the assay are disclosed in: C. Rominger, M. Schaber, E. May. Assay for B-Raf Activity Based on Intrinsic MEK ATPase Activity. Statutory Invention Registration 11/084,993 (March, 2005).

Many of the exemplified compounds of Examples 1-135 were run in the recited assay and the results are reported in the following Table 1. In the following table:
“+” indicates no pIC50 measurement greater than 6 against B-Raf
“++” indicates at least one pIC50 measurement greater than 6 against B-Raf but no measurement greater than pIC50 of 7; and
“+++” indicates at least one pIC50 measurement of greater than 7 against B-Raf.

TABLE 1
B-Raf Activity

| Example | Activity |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | +++ |
| 39 | +++ |
| 40 | +++ |
| 41 | +++ |
| 42 | +++ |
| 43 | +++ |
| 44 | +++ |
| 45 | +++ |
| 46 | +++ |
| 47 | +++ |
| 48 | +++ |
| 49 | +++ |
| 50 | +++ |
| 51 | +++ |
| 52 | +++ |
| 53 | +++ |
| 54 | +++ |
| 55 | +++ |
| 56 | +++ |
| 57 | +++ |
| 58 | +++ |
| 59 | +++ |
| 60 | +++ |
| 61 | +++ |
| 62 | +++ |
| 63 | +++ |
| 64 | +++ |
| 65 | +++ |
| 66 | +++ |
| 67 | +++ |
| 68 | +++ |
| 69 | +++ |
| 70 | +++ |
| 71 | +++ |
| 72 | +++ |
| 73 | +++ |
| 74 | +++ |
| 75 | +++ |
| 76 | +++ |
| 77 | +++ |
| 78 | +++ |
| 79 | +++ |
| 80 | +++ |
| 81 | +++ |
| 82 | +++ |
| 83 | ++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | +++ |
| 96 | +++ |
| 97 | ++ |
| 98 | +++ |
| 99 | +++ |
| 100 | +++ |
| 101 | +++ |
| 102 | +++ |
| 103 | +++ |
| 104 | +++ |
| 105 | +++ |
| 106 | +++ |
| 107 | +++ |
| 108 | +++ |
| 109 | +++ |
| 110 | +++ |
| 111 | +++ |
| 112 | +++ |
| 113 | +++ |
| 114 | +++ |
| 115 | +++ |
| 116 | +++ |
| 117 | +++ |
| 118 | +++ |
| 119 | +++ |
| 120 | +++ |
| 121 | +++ |
| 122 | +++ |
| 123 | +++ |
| 124 | +++ |
| 125 | +++ |
| 126 | +++ |
| 127 | +++ |
| 128 | +++ |
| 129 | +++ |
| 130 | +++ |
| 131 | +++ |
| 132 | +++ |
| 133 | +++ |
| 134 | +++ |
| 135 | +++ |

### B. Cellular Assays

Cell Growth Inhibition Assays Human colon tumor cells (Colo205) were cultured in RPMI (Mediatech 50-020-PB) containing 10% FBS and 1% penicillin-streptomycin. Human melanoma cancer cells (SK-MEL-28) were cultured in EMEM with nonessential amino acids (Mediatech 50-011-PB) containing 10% FBS, 1% sodium pyruvate (JT Baker 3354-04), and 1% penicillin-streptomycin. Human melanoma cancer cells (A375-F11S) were cultured in RPMI (Mediatech 50-020-PB) containing 10% FBS, 1% sodium pyruvate, and 1% penicillin-streptomycin. Human colon cancer cells (HT-29) were cultured in high glucose DMEM (Mediatech 50-013-PB) containing 10% FBS and 1% penicillin-streptomycin. Human melanoma cancer cells (SK-MEL-3) were cultured in McCoy's 5A (50-025-PB) containing 15% FBS, and 1% penicillin-streptomycin. All cell lines were maintained at 37° C. in a humidified 5% CO₂, 95% air incubator. Cells were harvested using trypsin/EDTA (Invitrogen 25200), counted using a hemocytometer, and plated. For 96-well assays (using white full-area NUNC plates cat. #136102), cells were plated in 105 μl at the following densities (cells/well): Colo205, 500; SK-MEL-28, 500; A375P-F11S, 500; HT29, 500; and SK-MEL-3, 500. For 384-well assays (white full-area NUNC plates, cat. #781080), cells were plated in 48 μl at the following densities (cells/well): Colo205, 500; SK-MEL-28, 500; A375P-F11S, 500; HT29, 500; and SK-MEL-3, 500.

The next day, compounds were diluted as follows: For 96-well assays, 13.5 μl of compound in DMSO were diluted using nine (9) serial 1:3 dilutions of 4.5 μl in 9 μl of DMSO. Medium (270 μl/well of RPMI with 10% FBS and 1% penicillin-streptomycin) was added to the plates. Aliquots (7 μl) were added to cells in the final assay giving a final DMSO concentration of 0.2%. For 384-well assays, 15 μl of compound in DMSO were diluted using nine (9) serial 1:3 dilutions of 5 μl in 10 μl of DMSO, followed by a further dilution of 5 μl of compound with 95 μl of medium, of which 2 μl are added to cells in the final assay giving a final DMSO concentration of 0.2%. Cells were incubated at 37° C., 5% CO₂ for 3 days.

Total ATP was measured (as a surrogate estimate of cell number) using CellTiter-Glo® (Promega G7571) reagent. Briefly, plates were removed from the incubator and allowed to equilibrate to room temperature for 30 minutes. CellTiter-Glo® (25 μl or 55 μl for 384-well or 96-well assays, respectively) reagent was added to each well and plates were shaken on an orbital plate shaker for 2 minutes. Plates were incubated without shaking for a further 30 minutes and read on an LJL Analyst GT reader in luminometer mode with an integration time of 0.5 seconds per well. Percent inhibition of cell growth was calculated relative to DMSO vehicle-treated control wells. Concentration of compound required to give 50% inhibition of vehicle-treated control cell growth (IC₅₀) was interpolated using a 4-parameter fit for determining IC₅₀ using the following equation: Y=A+((B−A)/(1+((C/X)̂D))) where X=IC₅₀.

Many of the compounds of Examples 1-135 were run in the recited assay and the results in Colo205 tumor cells are reported in the following Table 2. In the following table:
“+” indicates that the compound showed activity of >5 μM in Colo205 tumor cell lines;
“++” indicates that the compound showed activity of between 500 nM and 5 μM in Colo205 tumor cell lines; and
“+++” indicates that the compound showed activity of less than 500 nM in Colo205 tumor cell lines.

TABLE 2
Activity in Colo205 Tumor Cells

| Example | Activity |
|---|---|
| 1 | +++ |
| 2 | +++ |
| 3 | +++ |
| 4 | +++ |
| 5 | ++ |
| 6 | +++ |
| 7 | +++ |
| 8 | +++ |
| 9 | ++ |
| 10 | +++ |
| 11 | +++ |
| 12 | +++ |
| 13 | ++ |
| 14 | +++ |
| 15 | ++ |
| 16 | ++ |
| 17 | +++ |
| 18 | +++ |
| 19 | +++ |
| 20 | +++ |
| 21 | ++ |
| 22 | +++ |
| 23 | ++ |
| 24 | +++ |
| 25 | ++ |
| 26 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | +++ |
| 40 | +++ |
| 41 | +++ |
| 42 | +++ |
| 43 | ++ |
| 44 | +++ |
| 45 | ++ |
| 46 | +++ |
| 47 | ++ |
| 48 | +++ |
| 49 | ++ |
| 50 | +++ |
| 51 | +++ |
| 52 | +++ |
| 53 | +++ |
| 54 | +++ |
| 55 | +++ |
| 56 | +++ |
| 57 | ++ |
| 58 | +++ |
| 59 | +++ |
| 60 | +++ |
| 61 | +++ |
| 62 | ++ |
| 63 | +++ |
| 64 | +++ |
| 65 | ++ |
| 66 | +++ |
| 67 | ++ |
| 68 | ++ |
| 69 | +++ |
| 70 | +++ |
| 71 | +++ |
| 72 | +++ |
| 73 | ++ |
| 74 | +++ |
| 75 | ++ |
| 76 | ++ |
| 77 | +++ |
| 78 | ++ |
| 79 | + |
| 80 | + |
| 81 | ++ |
| 82 | +++ |
| 83 | ++ |
| 84 | ++ |
| 85 | ++ |
| 86 | ++ |
| 87 | ++ |
| 88 | ++ |
| 89 | ++ |
| 90 | +++ |
| 91 | +++ |
| 92 | ++ |
| 93 | ++ |
| 94 | +++ |
| 95 | ++ |
| 96 | ++ |
| 97 | + |
| 98 | +++ |
| 99 | ++ |
| 100 | +++ |
| 101 | +++ |
| 102 | +++ |
| 103 | ++ |
| 104 | +++ |
| 105 | +++ |
| 106 | +++ |
| 107 | +++ |
| 108 | +++ |
| 109 | ++ |
| 110 | ++ |
| 111 | ++ |
| 112 | +++ |
| 113 | ++ |
| 114 | ++ |
| 115 | +++ |
| 116 | +++ |
| 117 | +++ |
| 118 | ++ |
| 119 | ++ |
| 120 | +++ |
| 121 | + |
| 122 | ++ |
| 123 | ++ |
| 124 | ++ |
| 125 | ++ |
| 126 | ++ |
| 127 | +++ |
| 128 | +++ |
| 129 | +++ |
| 130 | ++ |
| 131 | +++ |
| 132 | ++ |
| 133 | +++ |
| 134 | ++ |
| 135 | ++ |

Many of the compounds of Examples 1-135 were run in the recited assay and the results in SK-MEL-28 tumor cells are reported in the following Table 3. In the following table:
“+” indicates that the compound showed activity of >5 μM in SK-MEL-28 tumor cell lines;
“++” indicates that the compound showed activity of between 500 nM and 5 μM in SK-MEL-28 tumor cell lines; and
“+++” indicates that the compound showed activity of less than 500 nM in SK-MEL-28 tumor cell lines.

TABLE 3
Activity in SK-MEL-28 Tumor Cells

| Example | Activity |
|---|---|
| 1 | ++ |
| 2 | +++ |
| 3 | +++ |
| 4 | +++ |
| 5 | ++ |
| 6 | ++ |
| 7 | +++ |
| 8 | +++ |
| 9 | +++ |
| 10 | +++ |
| 11 | +++ |
| 12 | ++ |
| 13 | ++ |
| 14 | ++ |
| 15 | ++ |
| 16 | ++ |
| 17 | +++ |
| 18 | +++ |
| 19 | +++ |
| 20 | +++ |
| 21 | ++ |
| 22 | +++ |
| 23 | ++ |
| 24 | ++ |
| 25 | ++ |
| 26 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | ++ |
| 40 | ++ |
| 41 | +++ |
| 42 | ++ |
| 43 | + |
| 44 | +++ |
| 45 | ++ |
| 46 | +++ |
| 47 | + |
| 48 | +++ |
| 49 | ++ |
| 50 | ++ |
| 51 | ++ |
| 52 | +++ |
| 53 | ++ |
| 54 | ++ |
| 55 | +++ |
| 56 | ++ |
| 57 | ++ |
| 58 | +++ |
| 59 | ++ |
| 60 | ++ |
| 61 | ++ |
| 62 | ++ |
| 63 | +++ |
| 64 | +++ |
| 65 | ++ |
| 66 | ++ |
| 67 | ++ |
| 68 | ++ |
| 69 | ++ |
| 70 | +++ |
| 71 | +++ |
| 72 | ++ |
| 73 | ++ |
| 74 | +++ |
| 75 | ++ |
| 76 | ++ |
| 77 | ++ |
| 78 | ++ |
| 79 | + |
| 80 | + |
| 81 | ++ |
| 82 | ++ |
| 83 | + |
| 84 | ++ |
| 85 | ++ |
| 86 | ++ |
| 87 | ++ |
| 88 | ++ |
| 89 | ++ |
| 90 | +++ |
| 91 | ++ |
| 92 | ++ |
| 93 | ++ |
| 94 | ++ |
| 95 | ++ |
| 96 | ++ |
| 97 | + |
| 98 | ++ |
| 99 | ++ |
| 100 | +++ |
| 101 | ++ |
| 102 | ++ |
| 103 | ++ |
| 104 | ++ |
| 105 | +++ |
| 106 | ++ |
| 107 | ++ |
| 108 | ++ |
| 109 | ++ |
| 110 | ++ |
| 111 | + |
| 112 | ++ |
| 113 | ++ |
| 114 | ++ |
| 115 | ++ |
| 116 | ++ |
| 117 | ++ |
| 118 | ++ |
| 119 | ++ |
| 120 | ++ |
| 121 | + |
| 122 | ++ |
| 123 | + |
| 124 | ++ |
| 125 | ++ |
| 126 | ++ |
| 127 | +++ |
| 128 | ++ |
| 129 | ++ |
| 130 | ++ |
| 131 | ++ |
| 132 | ++ |
| 133 | ++ |
| 134 | ++ |
| 135 | + |

Many of the compounds of Examples 1-135 were run in the recited assay and the results in A375P tumor cells are reported in the following Table 4. In the following table:
“+” indicates that the compound showed activity of >5 μM in A375P tumor cell lines;
“++” indicates that the compound showed activity of between 500 nM and 5 μM in A375P tumor cell lines; and
“+++” indicates that the compound showed activity of less than 500 nM in A375P tumor cell lines.

TABLE 4
Activity in A375P Tumor Cells

| Example | Activity |
|---|---|
| 1 | +++ |
| 2 | +++ |
| 3 | +++ |
| 4 | +++ |
| 5 | ++ |
| 6 | +++ |
| 7 | ++ |
| 8 | ++ |
| 9 | ++ |
| 10 | +++ |
| 11 | +++ |
| 12 | +++ |
| 13 | ++ |
| 14 | +++ |
| 15 | ++ |
| 16 | ++ |
| 17 | +++ |
| 18 | +++ |
| 19 | +++ |
| 20 | +++ |
| 21 | +++ |
| 22 | +++ |
| 23 | ++ |
| 25 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | +++ |
| 41 | +++ |
| 42 | +++ |
| 43 | +++ |
| 44 | +++ |
| 45 | ++ |
| 46 | +++ |
| 47 | +++ |
| 49 | +++ |
| 51 | ++ |
| 52 | +++ |
| 53 | +++ |
| 54 | ++ |
| 55 | +++ |
| 56 | +++ |
| 57 | +++ |
| 59 | ++ |
| 60 | ++ |
| 61 | +++ |
| 62 | +++ |
| 63 | +++ |
| 64 | +++ |
| 65 | ++ |
| 66 | +++ |
| 67 | +++ |
| 70 | +++ |
| 72 | +++ |
| 74 | +++ |
| 77 | +++ |
| 81 | ++ |
| 82 | + |
| 87 | ++ |
| 89 | ++ |
| 90 | ++ |
| 91 | ++ |
| 92 | ++ |
| 94 | ++ |
| 95 | ++ |
| 96 | ++ |
| 98 | ++ |
| 99 | ++ |
| 100 | +++ |
| 101 | ++ |
| 102 | ++ |
| 103 | ++ |
| 104 | ++ |
| 105 | +++ |
| 106 | +++ |
| 107 | ++ |
| 108 | ++ |
| 112 | ++ |
| 115 | ++ |
| 116 | ++ |
| 117 | ++ |
| 118 | ++ |
| 119 | ++ |
| 120 | ++ |
| 124 | ++ |
| 125 | ++ |
| 127 | ++ |
| 128 | ++ |
| 129 | ++ |
| 131 | ++ |
| 133 | ++ |

Many of the compounds of Examples 1-135 were run in the recited assay and the results in HT-29 tumor cells are reported in the following Table 5. In the following table:
“+” indicates that the compound showed activity of >5 μM in HT-29 tumor cell lines;
“++” indicates that the compound showed activity of between 500 nM and 5 μM in HT-29 tumor cell lines; and
“+++” indicates that the compound showed activity of less than 500 nM in HT-29 tumor cell lines.

TABLE 5
Activity in HT-29 Tumor Cells

| Example | Activity |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | ++ |
| 16 | +++ |
| 17 | +++ |
| 18 | +++ |
| 19 | +++ |
| 20 | +++ |
| 21 | +++ |
| 22 | +++ |
| 23 | +++ |
| 25 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | +++ |
| 41 | +++ |
| 42 | +++ |
| 43 | +++ |
| 44 | +++ |
| 45 | +++ |
| 46 | +++ |
| 47 | +++ |
| 49 | +++ |
| 51 | +++ |
| 52 | +++ |
| 53 | +++ |
| 54 | +++ |
| 55 | +++ |
| 56 | +++ |
| 57 | +++ |
| 59 | +++ |
| 60 | +++ |
| 61 | +++ |
| 62 | +++ |
| 63 | +++ |
| 64 | +++ |
| 65 | ++ |
| 66 | +++ |
| 67 | +++ |
| 70 | +++ |
| 72 | +++ |
| 74 | +++ |
| 77 | +++ |
| 81 | ++ |
| 82 | +++ |
| 87 | ++ |
| 89 | ++ |
| 90 | +++ |
| 91 | ++ |
| 92 | ++ |
| 94 | +++ |
| 95 | ++ |
| 96 | ++ |
| 98 | ++ |
| 99 | ++ |
| 100 | +++ |
| 101 | ++ |
| 102 | +++ |
| 103 | +++ |
| 104 | +++ |
| 105 | +++ |
| 106 | +++ |
| 107 | +++ |
| 108 | ++ |
| 112 | ++ |
| 115 | +++ |
| 116 | ++ |
| 117 | ++ |
| 118 | ++ |
| 119 | ++ |
| 120 | ++ |
| 124 | +++ |
| 125 | ++ |
| 127 | +++ |
| 128 | ++ |
| 129 | ++ |
| 131 | ++ |
| 133 | ++ |

Many of the compounds of Examples 1-135 were run in the recited assay and the results in SK-MEL-3 tumor cells are reported in the following Table 6. In the following table:
“+” indicates that the compound showed activity of >5 μM in SK-MEL-3 tumor cell lines;
“++” indicates that the compound showed activity of between 500 nM and 5 μM in SK-MEL-3 tumor cell lines; and
“+++” indicates that the compound showed activity of less than 500 nM in SK-MEL-3 tumor cell lines.

TABLE 6
Activity in SK-MEL-3 Tumor Cells

| Example | Activity |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 25 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | +++ |
| 41 | +++ |
| 42 | +++ |
| 43 | +++ |
| 44 | +++ |
| 45 | ++ |
| 46 | +++ |
| 47 | +++ |
| 49 | +++ |
| 51 | +++ |
| 52 | +++ |
| 53 | +++ |
| 54 | +++ |
| 55 | +++ |
| 56 | +++ |
| 57 | +++ |
| 59 | +++ |
| 60 | +++ |
| 61 | +++ |
| 62 | +++ |
| 63 | +++ |
| 64 | +++ |
| 65 | ++ |
| 66 | +++ |
| 67 | +++ |
| 70 | +++ |
| 72 | +++ |
| 74 | +++ |
| 77 | +++ |
| 81 | ++ |
| 82 | +++ |
| 87 | ++ |
| 89 | +++ |
| 90 | +++ |
| 91 | +++ |
| 92 | ++ |
| 94 | +++ |
| 95 | ++ |
| 96 | ++ |
| 98 | +++ |
| 99 | ++ |
| 100 | +++ |
| 101 | ++ |
| 102 | +++ |
| 103 | +++ |
| 104 | ++ |
| 105 | +++ |
| 106 | +++ |
| 107 | +++ |
| 108 | ++ |
| 112 | ++ |
| 115 | +++ |
| 116 | +++ |
| 117 | ++ |
| 118 | ++ |
| 119 | ++ |
| 120 | ++ |
| 124 | +++ |
| 125 | ++ |
| 127 | +++ |
| 128 | ++ |
| 129 | ++ |
| 131 | +++ |
| 133 | ++ |

### C. Autophosphorylation Assays

A selection of compounds of the present invention were tested for inhibitory activity against EGFR and ErbB2 kinases in autophosphorylation assays.

The method measures the ability of the isolated enzyme to catalyse the transfer of the gamma-phosphate from ATP onto the tyrosine residue of a biotinylated synthetic peptide referenced “Peptide C” in Brignola, P. S., et al, (2002) J. Biol. Chem. 277(2):1576-1585. The extent of tyrosine phosphorylation was measured using an anti-phosphotyrosine antibody, and quantified by homogenous time-resolved fluorescence (HTRF).

Reactions were performed in black 384-well polystyrene flat-bottom plates in a final volume of 20 ul. Assays were performed by adding 10 μl of each of the following solutions, substrate Mix and enzyme mix: The Substrate mix contained 100 mM 3-[N-morpholino]propanesulfonic acid (MOPS) (pH 7.5), 2 mM MnCl₂, 20 μM ATP, 0.01% Tween-20, 0.1 mg/mL (BSA), 0.8 uM peptide substrate, and 1 mM dithiothreitol. The enzyme mix contained 100 mM MOPS (pH7.5); 0.01% Tween-20; 0.1 mg/mL BSA, and either 0.8 nM EGFR, 10 nM ErbB2, or 1 nM ErbB4

The enzyme mix was added to the compound plates and the plates were incubated at 20° C. for 1 hour. The reactions were allowed to proceed for 90 minutes at 20° C. The reactions were then terminated by the addition of 20 μl 100 mM EDTA to each well. 40 μl/well of HTRF detection mix were added to the assay plates. The final concentrations of the detection reagents were: 100 mM HEPES (pH7.5), 0.1 mg/mL BSA, 15 nM streptavidin-labeled allophycocyanin (PerkinElmer), and 1 nM europium-labeled anti-phosphotyrosine antibody (PerkinElmer). Assay plates were left unsealed and were counted in a Wallac Multilabel Counter 1420 (PerkinElmer).

Compounds under analysis were dissolved in Me₂SO to 1.0 mM and serially diluted 1 to 3 with Me₂SO through twelve dilutions. 1 μl of each concentration was transferred to the corresponding well of an assay plate. This creates a final compound concentration range from 0.00027 to 47.6 μM.

The data for dose responses were plotted as % Inhibition calculated with the data reduction formula 100*(1−(U1−C2)/(C1−C2)) versus concentration of compound where U is the unknown value, C1 is the average control value obtained for 4.76% DMSO, and C2 is the average control value obtained for 0.035 M EDTA. Data were fitted with a curve described by:

y=((Vmax*x)/(K+x))+Y2

where Vmax is the upper asymptote, Y2 is the Y intercept, and K is the IC50. The results for each compound were recorded as pIC50s, calculated as follows:

pIC50 =−Log 10(K)

Selected compounds of the present invention were run in the recited assay and displayed a range of inhibitory potencies. The results are reported in the following Table 7. In the following table:
“+” indicates no pIC50 measurement greater than 6 against EGFR or ErbB2,
“++” indicates at least one pIC50 measurement greater than 6 against EGFR or ErbB2 but no measurement greater than pIC50 of 7; and
“+++” indicates at least one pIC50 measurement of greater than 7 against EGFR or ErbB2.

TABLE 7
EGFR and ErbB2 Activity

| Example | EGFR | ErbB2 |
|---|---|---|
| 4 | + | + |
| 7 | + | + |
| 10 | + | + |
| 27 | + | + |
| 28 | + | + |
| 29 | + | + |
| 30 | + | + |
| 34 | + | + |
| 35 | + | + |
| 36 | + | + |
| 37 | + | + |
| 41 | ++ | + |
| 43 | +++ | +++ |
| 44 | +++ | +++ |
| 46 | +++ | +++ |
| 55 | + | + |
| 61 | + | + |
| 63 | + | + |
| 64 | + | + |
| 70 | + | + |
| 72 | +++ | +++ |
| 74 | + | + |
| 77 | +++ | +++ |
| 78 | ++ | ++ |
| 82 | + | + |
| 87 | + | + |
| 91 | + | + |
| 103 | ++ | +++ |
| 107 | + | + |
| 113 | + | + |
| 118 | + | + |
| 124 | + | + |
| 135 | + | + |

### Pharmaceutical Formulation Example—Preparation of Capsules Containing a Compound of the Invention (Freebase):

Contents in each capsule:
   =60 mg Active Pharmaceutical ingredient (API)+60 mg Avicel+13 mg SSG.
133 mg total powder in a size 0 hard gelatin capsule. The Avicel/SSG weight may be reasonably approximate. Most critical weight is API.

### Procedure:

1. Separate the halves of hard-gelatin capsule and mark/identify each as appropriate/needed.
2. Place the bottom capsule half in capsule filler with the filling funnel on top.
3. Weigh the components (Avicel, Sodium Starch Glycolate (SSG), API) onto a single weigh paper (tared on an analytical balance between each weighing).
4. Record weights of each component.
5. Carefully and thoroughly mix the dry powders on the weigh paper with a small spatula.
6. Carefully transfer the mixed powders into the capsule through the funnel.
7. Place the top half onto the capsule and close until secure (should feel a snap), shake capsule to mix/distribute contents.
8. IF powder begins to near top of capsule, gently tap capsule and powder should settle.
9. Place the capsule into a small appropriately labeled bottle (but large enough to easily remove it).

### Pharmaceutical Formulation Example—Preparation of Tablets Containing a Compound of the Invention (Freebase)

| | Quantity | |
|---|---|---|
| Component | (mg/tablet) | % w/w |
| Core Tablet | | |
| API | 405.0 | 71.6 |
| Lactose monohydrate | 59.0 | 10.4 |
| Polysorbate 80 | 1.0 | 0.2 |
| Povidone | 40.0 | 7.1 |
| Colloidal Silicon Dioxide | 5.5 | 1.0 |
| Crospovidone | 51.0 | 9.0 |
| Magnesium Stearate | 4.5 | 0.8 |
| Purified Water | qs | |
| Film Coating | | |
| Opadry ® Orange, YS-1-13065-A | 17.0 | 3.0 |
| Purified water | qs | |

### Procedure:

1. Sieve Lactose, Silicon dioxide, Crospovidone and half Povidone.

### 2. Add API.

3. Granulate in High Shear Granulator with granulating solution containing dissolved Polysorbate 80 and other half of Povidone in Purified water.
4. Mill using Comil 197, 0.375″ screen.
5. Dry using Fluid Bed Dryer
6. Mill using Comil 197, 0.075″ screen
7. Add Crospofidone, magnesium stearate.
8. Blend 5 minute
9. Compress tablet
10. Aqueous film coat tablet

1. A compound of formula (I): 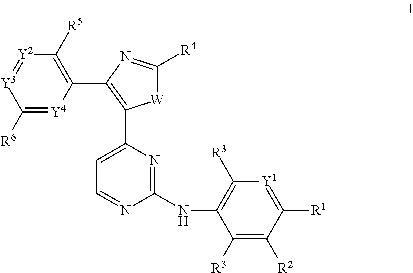
wherein:
R¹ is a moiety i, ii or iii: 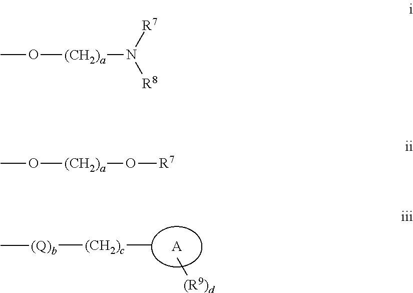
wherein:
a is 2, 3 or 4;
R⁷ and R⁸ are the same or different and are each independently selected from H, alkyl, haloalkyl, alkenyl, alkynyl, C₃₋₆cycloalkyl and C₃₋₆cycloalkenyl;
b is 0 or 1;
Q is selected from —O—, —N(H)— and —N(alkyl)-;
c is 0, 1, 2 or 3;
Ring A is a 4-10 membered N-heterocycle optionally including 1 or 2 additional heteroatoms selected from N, O and S, or 5-10 membered N-heteroaryl optionally including 1 or 2 additional heteroatoms selected from N, O and S;
d is 0, 1 or 2;
each R⁹ is the same or different and is independently selected from halo, alkyl, alkenyl, alkynyl, haloalkyl, oxo, OR¹⁰, R¹²—OR¹⁰, C(O)R¹⁰, CO₂R¹⁰,
C(O)₂-benzyl, CONR¹⁰R¹¹, COR¹²—NR¹⁰R¹¹, COR¹²—OR¹⁰, NR¹⁰R¹¹, R¹²—NR¹⁰R¹¹, N(R¹⁰)C(O)R¹¹, N(R¹⁰)S(O)₂R¹¹, N(R¹⁰)C(O)N(R¹¹), N(R¹⁰)C(S)N(R¹¹), S(O)₃H, R¹²—S(O)₃H, S(O)₂R¹⁰, R¹²—S(O)₂R¹⁰, S(O)₂NR¹⁰R¹¹, CN and R¹²—CN;
R² is selected from H, halo, alkyl, haloalkyl, OR¹⁰, CO₂R¹⁰, S(O)₂R¹⁰, CN and 5-6 membered N-heterocycle optionally including 1 additional heteroatom selected from N, O and S and optionally substituted 1 or 2 times with alkyl or oxo; or
R¹ and R², together with the aromatic ring to which they are bound form a 9 or 10-membered fused, bicyclic heteroaryl having 1, 2 or 3 heteroatoms selected from N, O and S, wherein said fused bicyclic heteroaryl is optionally substituted 1 or 2 times with R⁹, and Y¹ is N or CH;
one R³ is H and the other R³ is H, halo, alkyl, OH or O-alkyl;
Y¹ is N or C—R^{b}, wherein R^{b} is selected from H, halo, alkyl, haloalkyl, OR¹⁰, CO₂R¹⁰,
NR¹⁰R¹¹, S(O)₂R¹⁰ and CN;
W is O or S;
R⁴ is selected from H, alkyl, haloalkyl, alkylene-OH, R¹²—SO₂NR¹³R¹⁴, NR¹³R¹⁴, N(R¹³)R¹²—C₃₋₆cycloalkyl, N(R¹³)(CH₂)ₑ—OR¹⁴, N(R¹³)(CH₂)ₑ—SO₂R¹⁴, R¹²—N(R¹³)SO₂R¹⁴, N(R¹³)phenyl, and 5-6 membered N-linked heterocycle, wherein said N-linked heterocycle optionally includes 1 or 2 additional heteroatoms selected from N, O and S, and wherein said N-linked heterocycle is optionally substituted 1 or 2 times with a substituent selected from alkyl, oxo,
O-alkyl, OH, R¹²—OH, NH₂, N(H)alkyl and N(alkyl)₂;
e is 2, 3 or 4;
each R¹³ is the same or different and is selected from H, alkyl and haloalkyl; and
each R¹⁴ is the same or different and is selected from H, alkyl, haloalkyl, and C₃₋₆cycloalkyl;
Y² is N or R⁶—C;
Y³ is N or R^{a}—C;
Y⁴ is N or R⁵—C;
wherein not more than one of Y², Y³ and Y⁴ is N;
each R⁵ is the same or different and is selected from H, halo and alkyl, wherein when Y⁴ is R⁵—C, at least one R⁵ is H;
R^{a} is selected from H, halo, alkyl, haloalkyl, R¹²—OH and OR¹⁰;
each R⁶ is the same or different and is independently selected from H, halo, alkyl, alkenyl, alkynyl, haloalkyl, R¹²—OH, OR¹⁰ and NR¹⁰R¹¹, wherein at least one R⁶ is not H;
or R⁶ and R^{a} together with the aromatic ring to which they are bonded form an indenyl, naphthyl or a 9 or 10-membered fused bicyclic heteroaryl having 1, 2 or 3 heteroatoms selected from N, O and S, wherein said indenyl, naphthyl or fused bicyclic heteroaryl is optionally substituted 1 or 2 times with an additional substituent selected from alkyl, oxo, O-alkyl, OH, R¹²—OH, NH₂, N(H)alkyl and N(alkyl)₂;
each R¹⁰ and each R¹¹ is the same or different and is independently selected from H, alkyl and haloalkyl; and
each R¹² is the same or different and is independently C₁₋₄alkylene;
or a pharmaceutically acceptable salt thereof.

2. A compound according to claim 1 or a pharmaceutically acceptable salt thereof, wherein R¹ is moiety i:

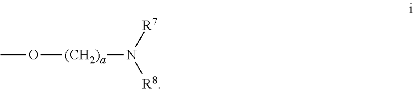

3. (canceled)

4. A compound according to claim 1 or a pharmaceutically acceptable salt thereof, wherein R¹ is moiety iii:

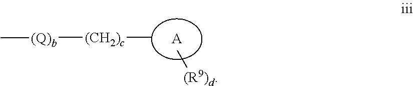

5. (canceled)

6. (canceled)

7. (canceled)

8. (canceled)

9. (canceled)

10. (canceled)

11. (canceled)

12. (canceled)

13. A compound according to claim 1 or a pharmaceutically acceptable salt thereof, wherein W is S.

14. A compound according to claim 1 or a pharmaceutically acceptable salt thereof, wherein R⁴ is selected from H, alkyl, R¹²—OH, R¹²—SO₂NR¹³R¹⁴, NR¹³R¹⁴, N(R¹³)(CH₂)ₑ—OR¹⁴, N(R¹³)(CF₁₂)ₑ—SO₂R¹⁴, R¹²—N(R¹³)SO₂R¹⁴, and 5-6 membered N-linked heterocycle, or any subset thereof, wherein said N-linked heterocycle is optionally substituted 1 or 2 times with a substituent selected from alkyl, oxo, O-alkyl, OH, alkylene-OH, NH₂, N(H)alkyl and N(alkyl)₂.

15. A compound according to claim 1 or a pharmaceutically acceptable salt thereof, wherein Y² is R⁶—C.

16. A compound according to claim 1 or a pharmaceutically acceptable salt thereof, wherein each R⁶ is the same or different and is independently selected from H, halo, alkyl, haloalkyl, R¹²—OH and OR¹⁰.

17. A compound according to claim 1 or a pharmaceutically acceptable salt thereof, wherein Y³ is R^{a}—C, and R^{a} is H.

18. A compound according to claim 1 or a pharmaceutically acceptable salt thereof, wherein Y⁴ is R⁵—C.

19. A compound according to claim 1 or a pharmaceutically acceptable salt thereof, wherein Y² is C—R⁶, Y³ is R^{a}—C, and Y⁴ is R⁵—C.

20. A compound according to claim 1 or a pharmaceutically acceptable salt thereof, wherein Y² is C—R⁶, both R⁶ are O—CH₃, Y³ is H—C, Y⁴ is R⁵—C, and both R⁵ are H.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A pharmaceutical composition comprising a compound according to claim 1 or a pharmaceutically acceptable salt thereof further comprising a pharmaceutically acceptable carrier, diluent or excipient.

35. A method for treating a susceptible neoplasm in a mammal in need thereof, said method comprising administering to the mammal a therapeutically effective amount of a compound according to claim 1 or a pharmaceutically acceptable salt thereof.

36. The method according to claim 35, wherein said susceptible neoplasm is selected from Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

37. (canceled)

38. (canceled)

39. A method for treating melanoma in a mammal in need thereof, said method comprising administering to the mammal a therapeutically effective amount of a compound according to claim 1 or a pharmaceutically acceptable salt thereof.

40. (canceled)

41. A process for preparing a compound according to claim 1 or a salt thereof, comprising reacting a compound of formula (V): 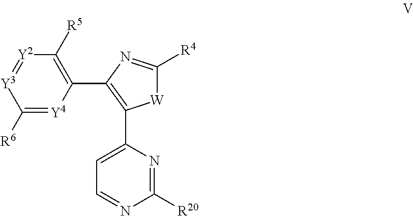
wherein R²⁰ is halo or thiomethyl;
with an aniline of formula (VI): 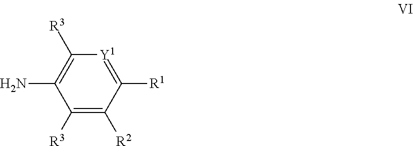
to prepare a compound of formula (I).

42. A process for preparing a compound of formula (I) or a salt thereof comprising reacting a compound of formula (VIII): 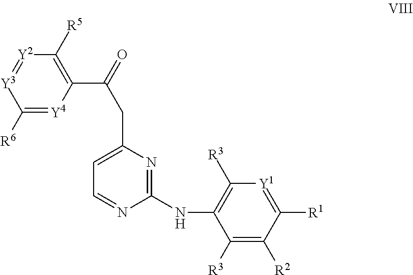
with a suitable brominating agent followed by reaction with one of:
i) a thiourea,
ii) a formamide,
iii) an amide,
iv) a thioamide, or
v) a urea;
to prepare a compound of formula (I).

43. (canceled)

44. (canceled)

45. (canceled)

46. (canceled)
